SEQUENCE LISTING (156 sequences)

SEQ ID NO: 1 - DNA, 516 bp, synthetic construct
  misc_feature 1..516: note = Description of Artificial Sequence: Syntheticpolynucleotide
  source 1..516: mol_type = other DNA
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatc

SEQ ID NO: 2 - protein, 172 aa, synthetic construct
  REGION 1..172: note = Description of Artificial Sequence: Syntheticpolypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 3-4 - skipped (2 entries)

SEQ ID NO: 5 - DNA, 123 bp, synthetic construct
  misc_feature 1..123: note = Description of Artificial Sequence: Syntheticpolynucleotide
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 6 - protein, 41 aa, synthetic construct
  REGION 1..41: note = Description of Artificial Sequence: Syntheticpolypeptide
RSKRSRLLHSDYMNMTPRRPGPTRKHYQPYAPPRDFAAYRS

SEQ ID NO: 7 - DNA, 108 bp, synthetic construct
  misc_feature 1..108: note = Description of Artificial Sequence: Syntheticpolynucleotide
  source 1..108: mol_type = other DNA
agggaccagaggctgccccccgatgcccacaagccccctgggggaggcagtttccggacccccatccaagaggagcaggccgacgcccactccaccctggccaagatc

SEQ ID NO: 8 - protein, 39 aa, synthetic construct
  REGION 1..39: note = Description of Artificial Sequence: Syntheticpolypeptide
RDQRLPPDAHKPPGGGSFRTPIQEEQADAHSTLAKIGSG

SEQ ID NO: 9 - DNA, 15 bp, synthetic construct
  misc_feature 1..15: note = Description of Artificial Sequence: Syntheticoligonucleotide
  source 1..15: mol_type = other DNA
ggatctggccaattg

SEQ ID NO: 10 - protein, 5 aa, synthetic construct
  REGION 1..5: note = Description of Artificial Sequence: Syntheticpeptide
GSGQL

SEQ ID NO: 11 - DNA, 321 bp, synthetic construct
  misc_feature 1..321: note = Description of Artificial Sequence: Syntheticpolynucleotide
  source 1..321: mol_type = other DNA
ggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaa

SEQ ID NO: 12 - protein, 107 aa, synthetic construct
  REGION 1..107: note = Description of Artificial Sequence: Syntheticpolypeptide
GVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 13-14 - skipped (2 entries)

SEQ ID NO: 15 - DNA, 321 bp, synthetic construct
  misc_feature 1..321: note = Description of Artificial Sequence: Syntheticpolynucleotide
  source 1..321: mol_type = other DNA
ggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 16 - protein, 107 aa, synthetic construct; identical to SEQ ID NO: 12
  REGION 1..107: note = Description of Artificial Sequence: Syntheticpolypeptide

SEQ ID NO: 17-18 - skipped (2 entries)

SEQ ID NO: 19 - DNA, 54 bp, synthetic construct
  misc_feature 1..54: note = Description of Artificial Sequence: Syntheticoligonucleotide
  source 1..54: mol_type = other DNA
gagggcagaggcagcctcctgacatgtggggacgtcgaggagaaccctggccca

SEQ ID NO: 20 - protein, 18 aa, synthetic construct
  REGION 1..18: note = Description of Artificial Sequence: Syntheticpeptide
EGRGSLLTCGDVEENPGP

SEQ ID NO: 21-22 - skipped (2 entries)

SEQ ID NO: 23 - DNA, 63 bp, synthetic construct
  misc_feature 1..63: note = Description of Artificial Sequence: Syntheticoligonucleotide
  source 1..63: mol_type = other DNA
atggagttcggattgagctggctgttcctggtggcaatactcaagggcgttcaatgttcacgg

SEQ ID NO: 24 - protein, 21 aa, synthetic construct
  REGION 1..21: note = Description of Artificial Sequence: Syntheticpeptide
MEFGLSWLFLVAILKGVQCSR

SEQ ID NO: 25 - DNA, 318 bp, synthetic construct
  misc_feature 1..318: note = Description of Artificial Sequence: Syntheticpolynucleotide
  source 1..318: mol_type = other DNA
gacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaa

SEQ ID NO: 26 - protein, 106 aa, synthetic construct
  REGION 1..106: note = Description of Artificial Sequence: Syntheticpolypeptide
DIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIK

SEQ ID NO: 27 - DNA, 24 bp, synthetic construct
  misc_feature 1..24: note = Description of Artificial Sequence: Syntheticoligonucleotide
  source 1..24: mol_type = other DNA
ggcggaggaagcggaggtgggggc

SEQ ID NO: 28 - protein, 8 aa, synthetic construct
  REGION 1..8: note = Description of Artificial Sequence: Syntheticpeptide
GGGSGGGG

SEQ ID NO: 29-30 - skipped (2 entries)

SEQ ID NO: 31 - DNA, 336 bp, synthetic construct
  misc_feature 1..336: note = Description of Artificial Sequence: Syntheticpolynucleotide
  source 1..336: mol_type = other DNA
gaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttcc

SEQ ID NO: 32 - protein, 112 aa, synthetic construct
  REGION 1..112: note = Description of Artificial Sequence: Syntheticpolypeptide
EVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSS

SEQ ID NO: 33-34 - skipped (2 entries)

SEQ ID NO: 35 - DNA, 48 bp, synthetic construct
  misc_feature 1..48: note = Description of Artificial Sequence: Syntheticoligonucleotide
  source 1..48: mol_type = other DNA
gaacttcctactcaggggactttctcaaacgttagcacaaacgtaagt

SEQ ID NO: 36 - protein, 16 aa, synthetic construct
  REGION 1..16: note = Description of Artificial Sequence: Syntheticpeptide
ELPTQGTFSNVSTNVS

SEQ ID NO: 37 - DNA, 48 bp, synthetic construct; identical to SEQ ID NO: 35
  misc_feature 1..48: note = Description of Artificial Sequence: Syntheticoligonucleotide
  source 1..48: mol_type = other DNA

SEQ ID NO: 38 - protein, 42 aa, synthetic construct
  REGION 1..42: note = Description of Artificial Sequence: Syntheticpolypeptide
PAPRPPTPAPTIASQPLSLRPEACRPAAGGAVHTRGLDFACD

SEQ ID NO: 39 - DNA, 111 bp, synthetic construct
  misc_feature 1..111: note = Description of Artificial Sequence: Syntheticpolynucleotide
  source 1..111: mol_type = other DNA
atctatatctgggcacctctcgctggcacctgtggagtccttctgctcagcctggttattactctgtactgtaatcaccggaatcgccgccgcgtttgtaagtgtcccagg

SEQ ID NO: 40 - protein, 37 aa, synthetic construct
  REGION 1..37: note = Description of Artificial Sequence: Syntheticpolypeptide
IYIWAPLAGTCGVLLLSLVITLYCNHRNRRRVCKCPR

SEQ ID NO: 41-42 - skipped (2 entries)

SEQ ID NO: 43 - DNA, 336 bp, synthetic construct
  misc_feature 1..336: note = Description of Artificial Sequence: Syntheticpolynucleotide
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgt

SEQ ID NO: 44 - protein, 112 aa, synthetic construct
  REGION 1..112: note = Description of Artificial Sequence: Syntheticpolypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 105 bp, synthetic construct
  misc_feature 1..105: note = Description of Artificial Sequence: Syntheticpolynucleotide
  source 1..105: mol_type = other DNA
acaaaaaagaagtattcatccagtgtgcacgaccctaacggtgaatacatgttcatgagagcagtgaacacagccaaaaaatctagactcacagatgtgacccta

SEQ ID NO: 46 - protein, 35 aa, synthetic construct
  REGION 1..35: note = Description of Artificial Sequence: Syntheticpolypeptide
TKKKYSSSVHDPNGEYMFMRAVNTAKKSRLTDVTL

SEQ ID NO: 47-48 - skipped (2 entries)

SEQ ID NO: 49 - DNA, 126 bp, synthetic construct
  misc_feature 1..126: note = Description of Artificial Sequence: Syntheticpolynucleotide
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 50 - protein, 42 aa, synthetic construct
  REGION 1..42: note = Description of Artificial Sequence: Syntheticpolypeptide
KRGRKKLLYIFKQPFMRPVQTTQEEDGCSCRFPEEEEGGCEL

SEQ ID NO: 51 - DNA, 42 bp, synthetic construct
  misc_feature 1..42: note = Description of Artificial Sequence: Syntheticoligonucleotide
  source 1..42: mol_type = other DNA
atggggagtagcaagagcaagcctaaggaccccagccagcgc

SEQ ID NO: 52 - protein, 14 aa, synthetic construct
  REGION 1..14: note = Description of Artificial Sequence: Syntheticpeptide
MGSSKSKPKDPSQR

SEQ ID NO: 53-54 - skipped (2 entries)

SEQ ID NO: 55 - DNA, 186 bp, synthetic construct
  misc_feature 1..186: note = Description of Artificial Sequence: Syntheticpolynucleotide
  source 1..186: mol_type = other DNA
aaaaaggtggccaagaagccaaccaataaggccccccaccccaagcaggagccccaggagatcaattttcccgacgatcttcctggctccaacactgctgctccagtgcaggagactttacatggatgccagccggtcacccaggaggatggcaaagagagtcgcatctcagtgcaggagagacag

SEQ ID NO: 56 - protein, 62 aa, synthetic construct
  REGION 1..62: note = Description of Artificial Sequence: Syntheticpolypeptide
KKVAKKPTNKAPHPKQEPQEINFPDDLPGSNTAAPVQETLHGCQPVTQEDGKESRISVQERQ

SEQ ID NO: 57 - DNA, 18 bp, synthetic construct
  misc_feature 1..18: note = Description of Artificial Sequence: Syntheticoligonucleotide
  source 1..18: mol_type = other DNA
atgctcgagatgctggag

SEQ ID NO: 58 - protein, 6 aa, synthetic construct
  REGION 1..6: note = Description of Artificial Sequence: Syntheticpeptide
MLEMLE

SEQ ID NO: 59 - DNA, 321 bp, synthetic construct
  misc_feature 1..321: note = Description of Artificial Sequence: Syntheticpolynucleotide
  source 1..321: mol_type = other DNA
ggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaa

SEQ ID NO: 60 - protein, 107 aa, synthetic construct
  REGION 1..107: note = Description of Artificial Sequence: Syntheticpolypeptide
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 61 - DNA, 24 bp, synthetic construct
  misc_feature 1..24: note = Description of Artificial Sequence: Syntheticoligonucleotide
  source 1..24: mol_type = other DNA
agcggaggaggatccggagtggac

SEQ ID NO: 62 - protein, 8 aa, synthetic construct
  REGION 1..8: note = Description of Artificial Sequence: Syntheticpeptide
SGGGSGVD

SEQ ID NO: 63 - DNA, 858 bp, synthetic construct
  misc_feature 1..858: note = Description of Artificial Sequence: Syntheticpolynucleotide
  source 1..858: mol_type = other DNA
gggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggcc

SEQ ID NO: 64 - protein, 286 aa, synthetic construct
  REGION 1..286: note = Description of Artificial Sequence: Syntheticpolypeptide
GFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRA

SEQ ID NO: 65 - DNA, 54 bp, synthetic construct
  misc_feature 1..54: note = Description of Artificial Sequence: Syntheticoligonucleotide
  source 1..54: mol_type = other DNA
gaaggccgagggagcctgctgacatgtggcgatgtggaggaaaacccaggacca

SEQ ID NO: 66 - skipped

SEQ ID NO: 67 - DNA, 63 bp, synthetic construct
  misc_feature 1..63: note = Description of Artificial Sequence: Syntheticoligonucleotide
  source 1..63: mol_type = other DNA
atggagtttggactttcttggttgtttttggtggcaattctgaagggtgtccagtgtagcagg

SEQ ID NO: 68 - DNA, 321 bp, synthetic construct
  misc_feature 1..321: note = Description of Artificial Sequence: Syntheticpolynucleotide
  source 1..321: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 69 - protein, 107 aa, synthetic construct
  REGION 1..107: note = Description of Artificial Sequence: Syntheticpolypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 70 - DNA, 360 bp, synthetic construct
  misc_feature 1..360: note = Description of Artificial Sequence: Syntheticpolynucleotide
  source 1..360: mol_type = other DNA
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 71 - protein, 120 aa, synthetic construct
  REGION 1..120: note = Description of Artificial Sequence: Syntheticpolypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 72 - DNA, 126 bp, synthetic construct
  misc_feature 1..126: note = Description of Artificial Sequence: Syntheticpolynucleotide
  source 1..126: mol_type = other DNA
cccgccccaagaccccccacacctgcgccgaccattgcttctcaacccctgagtttgagacccgaggcctgccggccagctgccggcggggccgtgcatacaagaggactcgatttcgcttgcgac

SEQ ID NO: 73 - DNA, 57 bp, synthetic construct
  misc_feature 1..57: note = Description of Artificial Sequence: Syntheticoligonucleotide
  source 1..57: mol_type = other DNA
gctactaacttcagcctgctgaagcaggctggagacgtggaggagaaccccgggcct

SEQ ID NO: 74 - protein, 19 aa, synthetic construct
  REGION 1..19: note = Description of Artificial Sequence: Syntheticpeptide
ATNFSLLKQAGDVEENPGP

SEQ ID NO: 75 - DNA, 186 bp, synthetic construct
  misc_feature 1..186: note = Description of Artificial Sequence: Syntheticpolynucleotide
  source 1..186: mol_type = other DNA
aaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 76 - protein, 16 aa, synthetic construct
  REGION 1..16: note = Description of Artificial Sequence: Syntheticpeptide
MGSSKSKPKDPSQRLD

SEQ ID NO: 77-78 - skipped (2 entries)

SEQ ID NO: 79 - DNA, 126 bp, synthetic construct; identical to SEQ ID NO: 49
  misc_feature 1..126: note = Description of Artificial Sequence: Syntheticpolynucleotide
  source 1..126: mol_type = other DNA

SEQ ID NO: 80 - protein, 42 aa, synthetic construct; identical to SEQ ID NO: 50
  REGION 1..42: note = Description of Artificial Sequence: Syntheticpolypeptide

SEQ ID NO: 81 - DNA, 348 bp, synthetic construct
  misc_feature 1..348: note = Description of Artificial Sequence: Syntheticpolynucleotide
  source 1..348: mol_type = other DNA
cagatccaactggtgcagtcaggcccggaactgaagaagccaggggagacagtcaaaataagttgtaaagccagcggctacatatttactaattacgggatgaattgggtgaagcaagcgccgggcaaatcctttaaatggatggggtggataaacacatacacaggagagtcaacgtacagcgcggacttcaaaggtcgattcgcgttcagtctcgagaccagcgcgagtacagcttacctccacatcaacgatcttaaaaacgaagacacggcaacctatttttgcgcccggtcaggcggttacgaccctatggactattggggccaagggacctccgttacggta

SEQ ID NO: 82 - protein, 116 aa, synthetic construct
  REGION 1..116: note = Description of Artificial Sequence: Syntheticpolypeptide
QIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTV

SEQ ID NO: 83 - DNA, 50 bp, synthetic construct
  misc_feature 1..50: note = Description of Artificial Sequence: Syntheticoligonucleotide
  source 1..50: mol_type = other DNA
cttcaggcggtggcgggagtggtggaggaggctcaggcggcgggggatca

SEQ ID NO: 84 - protein, 17 aa, synthetic construct
  REGION 1..17: note = Description of Artificial Sequence: Syntheticpeptide
SSGGGGSGGGGSGGGGS

SEQ ID NO: 85 - DNA, 360 bp, synthetic construct
  misc_feature 1..360: note = Description of Artificial Sequence: Syntheticpolynucleotide
  source 1..360: mol_type = other DNA
gacatcgtactgacccaatctcccgctagccttgcagtatccttgggtcaacgcgctacaataagttgccgggctagtgagtccgtagacaactatggcaacaccttcatgcattggtaccaacaaaaaccaggtcagccacccaaacttctcatttacagagcgtctaatctcgaaagcggcatccctgctcgattctctggaagcggaagtagaaccgactttacactgactataaaccccgtcgaagccgatgatgttgccacttattactgtcaacagagcaatgaggacccaccgacattcggtgctggtaccaagctggagttgaaggagtcaaaatacgggcctccctgtccc

SEQ ID NO: 86 - protein, 120 aa, synthetic construct
  REGION 1..120: note = Description of Artificial Sequence: Syntheticpolypeptide
DIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCP

SEQ ID NO: 87 - DNA, 333 bp, synthetic construct
  misc_feature 1..333: note = Description of Artificial Sequence: Syntheticpolynucleotide
  source 1..333: mol_type = other DNA
gatatcgtgctgacccagtccccccctagcctggccatgtccctgggcaaacgggccaccatctcctgcagagcctccgagtccgtgaccatcctcggctcccacctgatctactggtaccagcagaagcccggccagcctcccaccctccttatccagctggccagcaacgtgcagaccggcgtgcccgctagattctccggcagcggctctagaaccgacttcaccctgaccatcgaccccgtggaagaggacgatgtcgccgtgtactattgcctgcagtccagaaccatccctaggacattcggcggaggaaccaagctggagatcaaa

SEQ ID NO: 88 - protein, 111 aa, synthetic construct
  REGION 1..111: note = Description of Artificial Sequence: Syntheticpolypeptide
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 89 - DNA, 45 bp, synthetic construct
  misc_feature 1..45: note = Description of Artificial Sequence: Syntheticoligonucleotide
  source 1..45: mol_type = other DNA
gggggcggtggcagcggtggcggtgggtctgggggcggaggctct

SEQ ID NO: 90 - protein, 15 aa, synthetic construct
  REGION 1..15: note = Description of Artificial Sequence: Syntheticpeptide
GGGGSGGGGSGGGGS

SEQ ID NO: 91 - DNA, 352 bp, synthetic construct
  misc_feature 1..352: note = Description of Artificial Sequence: Syntheticpolynucleotide
  source 1..352: mol_type = other DNA
cagatccagctggtgcagtccggccccgagctgaagaaacccggcgagaccgtgaagatctcctgcaaggccagcggctacaccttcagacactacagcatgaactgggtgaagcaggcccctggcaagggcctgaagtggatgggccggatcaacaccgagtccggcgtgcccatctacgccgacgatttcaagggcagattcgccttcagcgtggagacctccgcctctaccgcctacctggtgatcaacaatctgaaggacgaggacaccgcctcctacttctgcagcaacgactacctgtacagcctggacttctggggccagggcaccgccctgaccgtgagctccg

SEQ ID NO: 92 - protein, 117 aa, synthetic construct
  REGION 1..117: note = Description of Artificial Sequence: Syntheticpolypeptide
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 93 - DNA, 15 bp, synthetic construct
  misc_feature 1..15: note = Description of Artificial Sequence: Syntheticoligonucleotide
  source 1..15: mol_type = other DNA
ggatctggcggccgc

SEQ ID NO: 94 - protein, 5 aa, synthetic construct
  REGION 1..5: note = Description of Artificial Sequence: Syntheticpeptide
GSGGR

SEQ ID NO: 95 - DNA, 54 bp, synthetic construct
  misc_feature 1..54: note = Description of Artificial Sequence: Syntheticoligonucleotide
  source 1..54: mol_type = other DNA
gagggaaggggaagtcttctaacatgcggggacgtggaggaaaatcccgggccc

SEQ ID NO: 96 - DNA, 537 bp, synthetic construct
  misc_feature 1..537: note = Description of Artificial Sequence: Syntheticpolynucleotide
  source 1..537: mol_type = other DNA
gagggaaggggaagtcttctaacatgcggggacgtggaggaaaatcccgggcccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagaccttattcaatctatgcacattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggagaagaacatcaaggaatttttgcagagttttgtacatattgtccaaatgttcatcaacact

SEQ ID NO: 97 - protein, 161 aa, synthetic construct
  REGION 1..161: note = Description of Artificial Sequence: Syntheticpolypeptide
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 98 - DNA, 15 bp, synthetic construct
  misc_feature 1..15: note = Description of Artificial Sequence: Syntheticoligonucleotide
  source 1..15: mol_type = other DNA
atgctcgagcaattg

SEQ ID NO: 99 - protein, 5 aa, synthetic construct
  REGION 1..5: note = Description of Artificial Sequence: Syntheticpeptide
MLEQL

SEQ ID NO: 100 - DNA, 267 bp, synthetic construct
  misc_feature 1..267: note = Description of Artificial Sequence: Syntheticpolynucleotide
  source 1..267: mol_type = other DNA
gaaatgtggcatgaagggttggaagaagcttcaaggctgtacttcggagagaggaacgtgaagggcatgtttgaggttcttgaacctctgcacgccatgatggaacggggaccgcagacactgaaagaaacctcttttaatcaggcctacggcagagacctgatggaggcccaagaatggtgtagaaagtatatgaaatccggtaacgtgaaagacctgactcaggcctgggacctttattaccatgtgttcaggcggatcagtaag

SEQ ID NO: 101 - protein, 89 aa, synthetic construct
  REGION 1..89: note = Description of Artificial Sequence: Syntheticpolypeptide
EMWHEGLEEASRLYFGERNVKGMFEVLEPLHAMMERGPQTLKETSFNQAYGRDLMEAQEWCRKYMKSGNVKDLTQAWDLYYHVFRRISK

SEQ ID NO: 102 - DNA, 12 bp, synthetic construct
  misc_feature 1..12: note = Description of Artificial Sequence: Syntheticoligonucleotide
  source 1..12: mol_type = other DNA
ggcgggcaattg

SEQ ID NO: 103 - protein, 4 aa, synthetic construct
  REGION 1..4: note = Description of Artificial Sequence: Syntheticpeptide
GGQL

SEQ ID NO: 104 - DNA, 318 bp, synthetic construct
  misc_feature 1..318: note = Description of Artificial Sequence: Syntheticpolynucleotide
  source 1..318: mol_type = other DNA
ggcgtccaagtcgaaaccattagtcccggcgatggcagaacatttcctaaaaggggacaaacatgtgtcgtccattatacaggcatgttggaggacggcaaaaagttcgacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctc

SEQ ID NO: 105 - protein, 106 aa, synthetic construct
  REGION 1..106: note = Description of Artificial Sequence: Syntheticpolypeptide
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKL

SEQ ID NO: 106 - DNA, 24 bp, synthetic construct
  misc_feature 1..24: note = Description of Artificial Sequence: Syntheticoligonucleotide
  source 1..24: mol_type = other DNA
tcaggcggtggctcaggtccatgg

SEQ ID NO: 107 - protein, 8 aa, synthetic construct
  REGION 1..8: note = Description of Artificial Sequence: Syntheticpeptide
SGGGSGPW

SEQ ID NO: 108 - DNA, 858 bp, synthetic construct
  misc_feature 1..858: note = Description of Artificial Sequence: Syntheticpolynucleotide
  source 1..858: mol_type = other DNA
ggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagcc

SEQ ID NO: 109 - DNA, 15 bp, synthetic construct
  misc_feature 1..15: note = Description of Artificial Sequence: Syntheticoligonucleotide
  source 1..15: mol_type = other DNA
ggatctggaccgcgg

SEQ ID NO: 110 - protein, 5 aa, synthetic construct
  REGION 1..5: note = Description of Artificial Sequence: Syntheticpeptide
GSGPR

SEQ ID NO: 111 - DNA, 999 bp, synthetic construct
  misc_feature 1..999: note = Description of Artificial Sequence: Syntheticpolynucleotide
  source 1..999: mol_type = other DNA
atgccaccacctcgcctgctgttctttctgctgttcctgacacctatggaggtgcgacctgaggaaccactggtcgtgaaggtcgaggaaggcgacaatgccgtgctgcagtgcctgaaaggcacttctgatgggccaactcagcagctgacctggtccagggagtctcccctgaagccttttctgaaactgagcctgggactgccaggactgggaatccacatgcgccctctggctatctggctgttcatcttcaacgtgagccagcagatgggaggattctacctgtgccagccaggaccaccatccgagaaggcctggcagcctggatggaccgtcaacgtggaggggtctggagaactgtttaggtggaatgtgagtgacctgggaggactgggatgtgggctgaagaaccgctcctctgaaggcccaagttcaccctcagggaagctgatgagcccaaaactgtacgtgtgggccaaagatcggcccgagatctgggagggagaacctccatgcctgccacctagagacagcctgaatcagagtctgtcacaggatctgacaatggcccccgggtccactctgtggctgtcttgtggagtcccacccgacagcgtgtccagaggccctctgtcctggacccacgtgcatcctaaggggccaaaaagtctgctgtcactggaactgaaggacgatcggcctgccagagacatgtgggtcatggagactggactgctgctgccacgagcaaccgcacaggatgctggaaaatactattgccaccggggcaatctgacaatgtccttccatctggagatcactgcaaggcccgtgctgtggcactggctgctgcgaaccggaggatggaaggtcagtgctgtgacactggcatatctgatcttttgcctgtgctccctggtgggcattctgcatctgcagagagccctggtgctgcggagaaagagaaagagaatgactgacccaacaagaaggttt

SEQ ID NO: 112 - protein, 333 aa, synthetic construct
  REGION 1..333: note = Description of Artificial Sequence: Syntheticpolypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 113 - DNA, 321 bp, synthetic construct; identical to SEQ ID NO: 59
  misc_feature 1..321: note = Description of Artificial Sequence: Syntheticpolynucleotide
  source 1..321: mol_type = other DNA

SEQ ID NO: 114 - DNA, 57 bp, synthetic construct
  misc_feature 1..57: note = Description of Artificial Sequence: Syntheticoligonucleotide
  source 1..57: mol_type = other DNA
gcaacgaatttttccctgctgaaacaggcaggggacgtagaggaaaatcctggtcct

SEQ ID NO: 115 - DNA, 185 bp, synthetic construct
  misc_feature 1..185: note = Description of Artificial Sequence: Syntheticpolynucleotide
  source 1..185: mol_type = other DNA
aaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagaca

SEQ ID NO: 116 - DNA, 483 bp, synthetic construct
  misc_feature 1..483: note = Description of Artificial Sequence: Syntheticpolynucleotide
  source 1..483: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagaccttattcaatctatgcacattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggagaagaacatcaaggaatttttgcagagttttgtacatattgtccaaatgttcatcaacact

SEQ ID NO: 117 - DNA, 888 bp, synthetic construct
  misc_feature 1..888: note = Description of Artificial Sequence: Syntheticpolynucleotide
  source 1..888: mol_type = other DNA
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccc

SEQ ID NO: 118 - protein, 296 aa, synthetic construct
  REGION 1..296: note = Description of Artificial Sequence: Syntheticpolypeptide
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 119 - protein, 173 aa, synthetic construct
  REGION 1..173: note = Description of Artificial Sequence: Syntheticpolypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDIQ

SEQ ID NO: 120 - protein, 16 aa, Homo sapiens
MGSNKSKPKDASQRRR

SEQ ID NO: 121 - protein, 91 aa, synthetic construct
  REGION 1..91: note = Description of Artificial Sequence: Syntheticpolypeptide
QLEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 122 - protein, 5 aa, synthetic construct
  REGION 1..5: note = Description of Artificial Sequence: Syntheticpeptide
SGGGS

SEQ ID NO: 123 - protein, 5 aa, synthetic construct
  REGION 1..5: note = Description of Artificial Sequence: Syntheticpeptide
GGGGS

SEQ ID NO: 124 - protein, 5 aa, synthetic construct
  REGION 1..5: note = Description of Artificial Sequence: Syntheticpeptide
  MOD_RES 4: note = Any amino acid
MGCXC

SEQ ID NO: 125 - DNA, 2787 bp, synthetic construct
  misc_feature 1..2787: note = Description of Artificial Sequence: Syntheticpolynucleotide
  source 1..2787: mol_type = other DNA
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcctcgagaggagtaagaggagcaggctcctgcacagtgactacatgaacatgactccccgccgccccgggcccacccgcaagcattaccagccctatgccccaccacgcgacttcgcagcctatcgctccagggaccagaggctgccccccgatgcccacaagccccctgggggaggcagtttccggacccccatccaagaggagcaggccgacgcccactccaccctggccaagatcggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 126 - DNA, 2661 bp, synthetic construct
  misc_feature 1..2661: note = Description of Artificial Sequence: Syntheticpolynucleotide
  source 1..2661: mol_type = other DNA
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcctcgagacaaaaaagaagtattcatccagtgtgcacgaccctaacggtgaatacatgttcatgagagcagtgaacacagccaaaaaatctagactcacagatgtgaccctaggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 127 - DNA, 2169 bp, synthetic construct
  misc_feature 1..2169: note = Description of Artificial Sequence: Syntheticpolynucleotide
  source 1..2169: mol_type = other DNA
atgctcgagaaacggggcagaaagaaactcctgtatatattcaaacaaccatttatgagaccagtacaaactactcaagaggaagatggctgtagctgccgatttccagaagaagaagaaggaggatgtgaactgggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 128 - DNA, 2601 bp, synthetic construct
  misc_feature 1..2601: note = Description of Artificial Sequence: Syntheticpolynucleotide
  source 1..2601: mol_type = other DNA
atggggagtagcaagagcaagcctaaggaccccagccagcgcagagcatgcatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 129 - DNA, 2793 bp, synthetic construct
  misc_feature 1..2793: note = Description of Artificial Sequence: Syntheticpolynucleotide
  source 1..2793: mol_type = other DNA
atggggagtagcaagagcaagcctaaggaccccagccagcgcagagcatgcatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcctcgagaaaaaggtggccaagaagccaaccaataaggccccccaccccaagcaggagccccaggagatcaattttcccgacgatcttcctggctccaacactgctgctccagtgcaggagactttacatggatgccagccggtcacccaggaggatggcaaagagagtcgcatctcagtgcaggagagacagggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 130 - DNA, 2229 bp, synthetic construct
  misc_feature 1..2229: note = Description of Artificial Sequence: Syntheticpolynucleotide
  source 1..2229: mol_type = other DNA
atgctcgagaaaaaggtggccaagaagccaaccaataaggccccccaccccaagcaggagccccaggagatcaattttcccgacgatcttcctggctccaacactgctgctccagtgcaggagactttacatggatgccagccggtcacccaggaggatggcaaagagagtcgcatctcagtgcaggagagacagggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 131 - DNA, 2271 bp, synthetic construct
  misc_feature 1..2271: note = Description of Artificial Sequence: Syntheticpolynucleotide
  source 1..2271: mol_type = other DNA
atggggagtagcaagagcaagcctaaggaccccagccagcgcagagcatgcaaaaaggtggccaagaagccaaccaataaggccccccaccccaagcaggagccccaggagatcaattttcccgacgatcttcctggctccaacactgctgctccagtgcaggagactttacatggatgccagccggtcacccaggaggatggcaaagagagtcgcatctcagtgcaggagagacagggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 132 - DNA, 2742 bp, synthetic construct
  misc_feature 1..2742: note = Description of Artificial Sequence: Syntheticpolynucleotide
  source 1..2742: mol_type = other DNA
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcctcgagaaaaaggtggccaagaagccaaccaataaggccccccaccccaagcaggagccccaggagatcaattttcccgacgatcttcctggctccaacactgctgctccagtgcaggagactttacatggatgccagccggtcacccaggaggatggcaaagagagtcgcatctcagtgcaggagagacagggatctggccaattgggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggacatccaactgacgcaaagcccatctacactcagcgctagcatgggggacagggtcacaatcacgtgctctgcctcaagttccgttaggtttatccattggtatcagcagaaacctggaaaggccccaaaaagactgatctatgataccagcaagctggcttccggagtgccctcaaggttctcaggatctggcagtgggaccgatttcaccctgacaattagcagccttcagccagaggatttcgcaacctattactgtcagcaatgggggtccagcccattcactttcggccaaggaacaaaggtggagataaaaggcggaggaagcggaggtgggggccaggtggaggtgcagctcgtggagtatggcgggggcctggtgcagcctgggggtagtctgaggctctcctgcgctgcctctggctttaacattaaagactactacatacattgggtgcggcaggccccaggcaaagggctcgaatgggtggcctggattgaccctgagaatggtgacactgagtttgtccccaagtttcagggcagagccaccatgagcgctgacacaagcaaaaacactgcttatctccaaatgaatagcctgcgagctgaagatacagcagtctattactgcaagacgggaggattctggggccagggaactctggtgacagttagttccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 133 - DNA, 2688 bp, synthetic construct
  misc_feature 1..2688: note = Description of Artificial Sequence: Syntheticpolynucleotide
  source 1..2688: mol_type = other DNA
atgctcgagatgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaaagcggaggaggatccggagtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggccccgcgggaaggccgagggagcctgctgacatgtggcgatgtggaggaaaacccaggaccaccatggatggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggcggaggaagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgt

SEQ ID NO: 134 - DNA, 3471 bp, synthetic construct
  misc_feature 1..3471: note = Description of Artificial Sequence: Syntheticpolynucleotide
  source 1..3471: mol_type = other DNA
atgctcgagatgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaaagcggaggaggatccggagtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggccccgcgggaaggccgagggagcctgctgacatgtggcgatgtggaggaaaacccaggaccaccatggatggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggcggaggaagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgtatgctcgagatgctggaggctactaacttcagcctgctgaagcaggctggagacgtggaggagaaccccgggcctatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcgtcgagaaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagacag

SEQ ID NO: 135 - DNA, 3456 bp, synthetic construct
  misc_feature 1..3456: note = Description of Artificial Sequence: Syntheticpolynucleotide
  source 1..3456: mol_type = other DNA
atgctcgagatgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaaagcggaggaggatccggagtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggccccgcgggaaggccgagggagcctgctgacatgtggcgatgtggaggaaaacccaggaccaccatggatggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggcggaggaagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgtatgctcgagatgctggaggctactaacttcagcctgctgaagcaggctggagacgtggaggagaaccccgggcctatggggagtagcaagagcaagcctaaggaccccagccagcgcatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcgcggccgctaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 136 - DNA, 3414 bp, synthetic construct
  misc_feature 1..3414: note = Description of Artificial Sequence: Syntheticpolynucleotide
  source 1..3414: mol_type = other DNA
atgctcgagatgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaaagcggaggaggatccggagtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggccccgcgggaaggccgagggagcctgctgacatgtggcgatgtggaggaaaacccaggaccaccatggatggagtttggactttcttggttgtttttggtggcaattctgaagggtgtccagtgtagcagggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggcggaggaagcggaggtgggggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaggatccgaacttcctactcaggggactttctcaaacgttagcacaaacgtaagtcccgccccaagaccccccacacctgcgccgaccattgcttctcaacccctgagtttgagacccgaggcctgccggccagctgccggcggggccgtgcatacaagaggactcgatttcgcttgcgacatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgtatgctcgagatgctggaggctactaacttcagcctgctgaagcaggctggagacgtggaggagaaccccgggcctatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcgcggccgctaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 137 - DNA, 1379 bp, synthetic construct
  misc_feature 1..1379: note = Description of Artificial Sequence: Syntheticpolynucleotide
  source 1..1379: mol_type = other DNA
atggagttcggattgagctggctgttcctggtggcaatactcaagggcgttcaatgttcacggcagatccaactggtgcagtcaggcccggaactgaagaagccaggggagacagtcaaaataagttgtaaagccagcggctacatatttactaattacgggatgaattgggtgaagcaagcgccgggcaaatcctttaaatggatggggtggataaacacatacacaggagagtcaacgtacagcgcggacttcaaaggtcgattcgcgttcagtctcgagaccagcgcgagtacagcttacctccacatcaacgatcttaaaaacgaagacacggcaacctatttttgcgcccggtcaggcggttacgaccctatggactattggggccaagggacctccgttacggtacttcaggcggtggcgggagtggtggaggaggctcaggcggcgggggatcagacatcgtactgacccaatctcccgctagccttgcagtatccttgggtcaacgcgctacaataagttgccgggctagtgagtccgtagacaactatggcaacaccttcatgcattggtaccaacaaaaaccaggtcagccacccaaacttctcatttacagagcgtctaatctcgaaagcggcatccctgctcgattctctggaagcggaagtagaaccgactttacactgactataaaccccgtcgaagccgatgatgttgccacttattactgtcaacagagcaatgaggacccaccgacattcggtgctggtaccaagctggagttgaaggagtcaaaatacgggcctccctgtcccggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgttga

SEQ ID NO: 138 - DNA, 3385 bp, synthetic construct
  misc_feature 1..3385: note = Description of Artificial Sequence: Syntheticpolynucleotide
  source 1..3385: mol_type = other DNA
atggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcgtcgagaaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagacaggtcgagggcgtccaagtcgaaaccattagtcccggcgatggcagaacatttcctaaaaggggacaaacatgtgtcgtccattatacaggcatgttggaggacggcaaaaaggtggacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctcgaagtcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaaccgcgggagggcagaggcagcctcctgacatgtggggacgtcgaggagaaccctggcccaccttggatggagttcggattgagctggctgttcctggtggcaatactcaagggcgttcaatgttcacgggatatcgtgctgacccagtccccccctagcctggccatgtccctgggcaaacgggccaccatctcctgcagagcctccgagtccgtgaccatcctcggctcccacctgatctactggtaccagcagaagcccggccagcctcccaccctccttatccagctggccagcaacgtgcagaccggcgtgcccgctagattctccggcagcggctctagaaccgacttcaccctgaccatcgaccccgtggaagaggacgatgtcgccgtgtactattgcctgcagtccagaaccatccctaggacattcggcggaggaaccaagctggagatcaaagggggcggtggcagcggtggcggtgggtctgggggcggaggctctcagatccagctggtgcagtccggccccgagctgaagaaacccggcgagaccgtgaagatctcctgcaaggccagcggctacaccttcagacactacagcatgaactgggtgaagcaggcccctggcaagggcctgaagtggatgggccggatcaacaccgagtccggcgtgcccatctacgccgacgatttcaagggcagattcgccttcagcgtggagacctccgcctctaccgcctacctggtgatcaacaatctgaaggacgaggacaccgcctcctacttctgcagcaacgactacctgtacagcctggacttctggggccagggcaccgccctgaccgtgagctccgggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgtggatctggcggccgcgagggaaggggaagtcttctaacatgcggggacgtggaggaaaatcccgggcccgagggaaggggaagtcttctaacatgcggggacgtggaggaaaatcccgggcccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagaccttattcaatctatgcacattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggagaagaacatcaaggaatttttgcagagttttgtacatattgtccaaatgttcatcaacacttga

SEQ ID NO: 139 - DNA, 2565 bp, synthetic construct
  misc_feature 1..2565: note = Description of Artificial Sequence: Syntheticpolynucleotide
  source 1..2565: mol_type = other DNA
atgctcgagcaattggaaatgtggcatgaagggttggaagaagcttcaaggctgtacttcggagagaggaacgtgaagggcatgtttgaggttcttgaacctctgcacgccatgatggaacggggaccgcagacactgaaagaaacctcttttaatcaggcctacggcagagacctgatggaggcccaagaatggtgtagaaagtatatgaaatccggtaacgtgaaagacctgactcaggcctgggacctttattaccatgtgttcaggcggatcagtaagggcgggcaattgggcgtccaagtcgaaaccattagtcccggcgatggcagaacatttcctaaaaggggacaaacatgtgtcgtccattatacaggcatgttggaggacggcaaaaagttcgacagtagtagagatcgcaataaacctttcaaattcatgttgggaaaacaagaagtcattaggggatgggaggagggcgtggctcaaatgtccgtcggccaacgcgctaagctcaccatcagccccgactacgcatacggcgctaccggacatcccggaattattccccctcacgctaccttggtgtttgacgtcgaactgttgaagctctcaggcggtggctcaggtccatggggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagctagcagagccggatctggaccgcgggaaggccgagggagcctgctgacatgtggcgatgtggaggaaaacccaggaccaatgccaccacctcgcctgctgttctttctgctgttcctgacacctatggaggtgcgacctgaggaaccactggtcgtgaaggtcgaggaaggcgacaatgccgtgctgcagtgcctgaaaggcacttctgatgggccaactcagcagctgacctggtccagggagtctcccctgaagccttttctgaaactgagcctgggactgccaggactgggaatccacatgcgccctctggctatctggctgttcatcttcaacgtgagccagcagatgggaggattctacctgtgccagccaggaccaccatccgagaaggcctggcagcctggatggaccgtcaacgtggaggggtctggagaactgtttaggtggaatgtgagtgacctgggaggactgggatgtgggctgaagaaccgctcctctgaaggcccaagttcaccctcagggaagctgatgagcccaaaactgtacgtgtgggccaaagatcggcccgagatctgggagggagaacctccatgcctgccacctagagacagcctgaatcagagtctgtcacaggatctgacaatggcccccgggtccactctgtggctgtcttgtggagtcccacccgacagcgtgtccagaggccctctgtcctggacccacgtgcatcctaaggggccaaaaagtctgctgtcactggaactgaaggacgatcggcctgccagagacatgtgggtcatggagactggactgctgctgccacgagcaaccgcacaggatgctggaaaatactattgccaccggggcaatctgacaatgtccttccatctggagatcactgcaaggcccgtgctgtggcactggctgctgcgaaccggaggatggaaggtcagtgctgtgacactggcatatctgatcttttgcctgtgctccctggtgggcattctgcatctgcagagagccctggtgctgcggagaaagagaaagagaatgactgacccaacaagaaggttttga

SEQ ID NO: 140 - DNA, 3957 bp, synthetic construct
  misc_feature 1..3957: note = Description of Artificial Sequence: Syntheticpolynucleotide
  source 1..3957: mol_type = other DNA
atgctcgagatgctggagggagtgcaggtggagactattagccccggagatggcagaacattccccaaaagaggacagacttgcgtcgtgcattatactggaatgctggaagacggcaagaaggtggacagcagccgggaccgaaacaagcccttcaagttcatgctggggaagcaggaagtgatccggggctgggaggaaggagtcgcacagatgtcagtgggacagagggccaaactgactattagcccagactacgcttatggagcaaccggccaccccgggatcattccccctcatgctacactggtcttcgatgtggagctgctgaagctggaaagcggaggaggatccggagtggacgggtttggagatgtgggagccctggaatccctgcggggcaatgccgatctggcttacatcctgtctatggagccttgcggccactgtctgatcattaacaatgtgaacttctgcagagagagcgggctgcggaccagaacaggatccaatattgactgtgaaaagctgcggagaaggttctctagtctgcactttatggtcgaggtgaaaggcgatctgaccgctaagaaaatggtgctggccctgctggaactggctcggcaggaccatggggcactggattgctgcgtggtcgtgatcctgagtcacggctgccaggcttcacatctgcagttccctggggcagtctatggaactgacggctgtccagtcagcgtggagaagatcgtgaacatcttcaacggcacctcttgcccaagtctgggcgggaagcccaaactgttctttattcaggcctgtggaggcgagcagaaagatcacggcttcgaagtggctagcacctcccccgaggacgaatcacctggaagcaaccctgagccagatgcaacccccttccaggaaggcctgaggacatttgaccagctggatgccatctcaagcctgcccacaccttctgacattttcgtctcttacagtactttccctggatttgtgagctggcgcgatccaaagtcaggcagctggtacgtggagacactggacgatatctttgagcagtgggcccattctgaagacctgcagagtctgctgctgcgagtggccaatgctgtctctgtgaaggggatctacaaacagatgccaggatgcttcaactttctgagaaagaaactgttctttaagacctccgcatctagggccccgcgggagggcagaggcagcctcctgacatgtggggacgtcgaggagaaccctggcccaccttggatggagttcggattgagctggctgttcctggtggcaatactcaagggcgttcaatgttcacgggatatcgtgctgacccagtccccccctagcctggccatgtccctgggcaaacgggccaccatctcctgcagagcctccgagtccgtgaccatcctcggctcccacctgatctactggtaccagcagaagcccggccagcctcccaccctccttatccagctggccagcaacgtgcagaccggcgtgcccgctagattctccggcagcggctctagaaccgacttcaccctgaccatcgaccccgtggaagaggacgatgtcgccgtgtactattgcctgcagtccagaaccatccctaggacattcggcggaggaaccaagctggagatcaaagggggcggtggcagcggtggcggtgggtctgggggcggaggctctcagatccagctggtgcagtccggccccgagctgaagaaacccggcgagaccgtgaagatctcctgcaaggccagcggctacaccttcagacactacagcatgaactgggtgaagcaggcccctggcaagggcctgaagtggatgggccggatcaacaccgagtccggcgtgcccatctacgccgacgatttcaagggcagattcgccttcagcgtggagacctccgcctctaccgcctacctggtgatcaacaatctgaaggacgaggacaccgcctcctacttctgcagcaacgactacctgtacagcctggacttctggggccagggcaccgccctgaccgtgagctccgggatccgaacttcctactcaggggactttctcaaacgttagcacaaacgtaagtgaacttcctactcaggggactttctcaaacgttagcacaaacgtaagtatctatatctgggcacctctcgctggcacctgtggagtccttctgctcagcctggttattactctgtactgtaatcaccggaatcgccgccgcgtttgtaagtgtcccagggtcgacagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaagctcttccacctcgtgcaacgaatttttccctgctgaaacaggcaggggacgtagaggaaaatcctggtcctatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgatctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatcgtcgagaaaaaggtggccaagaagccaaccaataaggccccccaccccaagcaggagccccaggagatcaattttcccgacgatcttcctggctccaacactgctgctccagtgcaggagactttacatggatgccaaccggtcacccaggaggatggcaaagagagtcgcatctcagtgcaggagagacaggatctggcggccgcgagggaaggggaagtcttctaacatgcggggacgtggaggaaaatcccgggccctgaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagaccttattcaatctatgcacattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggagaagaacatcaaggaatttttgcagagttttgtacatattgtccaaatgttcatcaacacttga

SEQ ID NO: 141 - protein, 957 aa, synthetic construct
  REGION 1..957: note = Description of Artificial Sequence: Syntheticpolypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDILERSKRSRLLHSDYMNMTPRRPGPTRKHYQPYAPPRDFAAYRSRDQRLPPDAHKPPGGGSFRTPIQEEQADAHSTLAKIGSGGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 912 aa, synthetic construct
  REGION 1..912: note = Description of Artificial Sequence: Syntheticpolypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDILETKKKYSSSVHDPNGEYMFMRAVNTAKKSRLTDVTLGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 748 aa, synthetic construct
  REGION 1..748: note = Description of Artificial Sequence: Syntheticpolypeptide
MLEKRGRKKLLYIFKQPFMRPVQTTQEEDGCSCRFPEEEEGGCELGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 892 aa, synthetic construct
  REGION 1..892: note = Description of Artificial Sequence: Syntheticpolypeptide
MGSSKSKPKDPSQRRACMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDIGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 956 aa, synthetic construct
  REGION 1..956: note = Description of Artificial Sequence: Syntheticpolypeptide
MGSSKSKPKDPSQRRACMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDILEKKVAKKPTNKAPHPKQEPQEINFPDDLPGSNTAAPVQETLHGCQPVTQEDGKESRISVQERQGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 768 aa, synthetic construct
  REGION 1..768: note = Description of Artificial Sequence: Syntheticpolypeptide
MLEKKVAKKPTNKAPHPKQEPQEINFPDDLPGSNTAAPVQETLHGCQPVTQEDGKESRISVQERQGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 782 aa, synthetic construct
  REGION 1..782: note = Description of Artificial Sequence: Syntheticpolypeptide
MGSSKSKPKDPSQRRACKKVAKKPTNKAPHPKQEPQEINFPDDLPGSNTAAPVQETLHGCQPVTQEDGKESRISVQERQGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 939 aa, synthetic construct
  REGION 1..939: note = Description of Artificial Sequence: Syntheticpolypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDILEKKVAKKPTNKAPHPKQEPQEINFPDDLPGSNTAAPVQETLHGCQPVTQEDGKESRISVQERQGSGQLGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIQLTQSPSTLSASMGDRVTITCSASSSVRFIHWYQQKPGKAPKRLIYDTSKLASGVPSRFSGSGSGTDFTLTISSLQPEDFATYYCQQWGSSPFTFGQGTKVEIKGGGSGGGGQVEVQLVEYGGGLVQPGGSLRLSCAASGFNIKDYYIHWVRQAPGKGLEWVAWIDPENGDTEFVPKFQGRATMSADTSKNTAYLQMNSLRAEDTAVYYCKTGGFWGQGTLVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 896 aa, synthetic construct
  REGION 1..896: note = Description of Artificial Sequence: Syntheticpolypeptide
MLEMLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAPREGRGSLLTCGDVEENPGPPWMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 1157 aa, synthetic construct
  REGION 1..1157: note = Description of Artificial Sequence: Syntheticpolypeptide
MLEMLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAPREGRGSLLTCGDVEENPGPPWMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMLEMLEATNFSLLKQAGDVEENPGPMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQ

SEQ ID NO: 151 - protein, 1154 aa, synthetic construct
  REGION 1..1154: note = Description of Artificial Sequence: Syntheticpolypeptide
MLEMLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAPREGRGSLLTCGDVEENPGPPWMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMLEMLEATNFSLLKQAGDVEENPGPMGSSKSKPKDPSQRLDMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDIAAAKRGRKKLLYIFKQPFMRPVQTTQEEDGCSCRFPEEEEGGCEL

SEQ ID NO: 152 - protein, 1138 aa, synthetic construct
  REGION 1..1138: note = Description of Artificial Sequence: Syntheticpolypeptide
MLEMLEGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAPREGRGSLLTCGDVEENPGPPWMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMLEMLEATNFSLLKQAGDVEENPGPMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDIAAAKRGRKKLLYIFKQPFMRPVQTTQEEDGCSCRFPEEEEGGCEL

SEQ ID NO: 153 - protein, 485 aa, synthetic construct
  REGION 1..485: note = Description of Artificial Sequence: Syntheticpolypeptide
MEFGLSWLFLVAILKGVQCSRQIQLVQSGPELKKPGETVKISCKASGYIFTNYGMNWVKQAPGKSFKWMGWINTYTGESTYSADFKGRFAFSLETSASTAYLHINDLKNEDTATYFCARSGGYDPMDYWGQGTSVTVSSGGGGSGGGGSGGGGSDIVLTQSPASLAVSLGQRATISCRASESVDNYGNTFMHWYQQKPGQPPKLLIYRASNLESGIPARFSGSGSRTDFTLTINPVEADDVATYYCQQSNEDPPTFGAGTKLELKESKYGPPCPGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 1135 aa, synthetic construct
  REGION 1..1135: note = Description of Artificial Sequence: Syntheticpolypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEVEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEPREGRGSLLTCGDVEENPGPPWMEFGLSWLFLVAILKGVQCSRDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGGGGSGGGGSGGGGSQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGGREGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 155 - protein, 854 aa, synthetic construct
  REGION 1..854: note = Description of Artificial Sequence: Syntheticpolypeptide
MLEQLEMWHEGLEEASRLYFGERNVKGMFEVLEPLHAMMERGPQTLKETSFNQAYGRDLMEAQEWCRKYMKSGNVKDLTQAWDLYYHVFRRISKGGQLGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLSGGGSGPWGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAGSGPREGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 156 - protein, 1182 aa, synthetic construct
  REGION 1..1182: note = Description of Artificial Sequence: Syntheticpolypeptide
MLEMLEGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSASRAPREGRGSLLTCGDVEENPGPPWMEFGLSWLFLVAILKGVQCSRDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGGGGSGGGGSGGGGSQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGSELPTQGTFSNVSTNVSPAPRPPTPAPTIASQPLSLRPEACRPAAGGAVHTRGLDFACDIYIWAPLAGTCGVLLLSLVITLYCNHRNRRRVCKCPRVDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDIVEKKVAKKPTNKAPHPKQEPQEINFPDDLPGSNTAAPVQETLHGCQPVTQEDGKESRISVQERQGSGGREGRGSLLTCGDVEENPGP

(19) United States
(12) Patent Application Publication
(10) Pub. No.: US 2026/0042813 A1
(43) Pub. Date: Feb. 12, 2026

(54) NATURAL KILLER CELL PRODUCTS AND METHODS

(71) Applicant: BOARD OF REGENTS OF THE UNIVERSITY OF TEXAS SYSTEM, Austin, TX (US)

(72) Inventors: Joseph Henri BAYLE, Houston, TX (US); Xiaomei WANG, Houston, TX (US); David Michael SPENCER, Frisco, TX (US); Wei-Chun CHANG, Glenside, PA (US)

(73) Assignee: BOARD OF REGENTS OF THE UNIVERSITY OF TEXAS SYSTEM, Austin, TX (US)

(21) Appl. No.: 19/296,134

(22) Filed: Aug. 11, 2025

Related U.S. Application Data
(63) Continuation of application No. 17/053,275, now Pat. No. 12,410,231.
(60) Provisional application No. 62/668,223, filed on May 7, 2018.
(60) Provisional application No. 62/756,442, filed on Nov. 6, 2018.
(60) Provisional application No. 62/816,799, filed on Mar. 11, 2019.

(51) Int. Cl. C07K 14/725; A61K 40/15; A61K 40/31; A61K 40/42; C07K 14/54; C12N 5/0783

(52) U.S. Cl. CPC C07K 14/7051; A61K 40/15; A61K 40/31; A61K 40/4205; A61K 40/4215; A61K 40/4217; A61K 40/4224; C07K 14/5443; C12N 5/0646; A61K 2239/31; A61K 2239/38; A61K 2239/48

(57) ABSTRACT

The technology relates generally to the field of immunology and relates in part to compositions and methods for growing and storing modified natural killer cells, including for example, conditional chemical regulation of natural killer cell function. The technology further relates to pharmaceutical compositions and treatment of subjects using modified natural killer cells.

### CROSS-REFERENCE TO RELATED APPLICATIONS

This application is a continuation of U.S. application Ser. No. 17/053,275, filed Nov. 5, 2020, which is a national phase application under 35 U.S.C. § 371 of International Application No. PCT/US2019/030939, filed May 6, 2019, which claims priority to U.S. Application No. 62/668,223, filed May 7, 2018, U.S. Application No. 62/756,442, filed Nov. 6, 2018, and U.S. Application No. 62/816,799, filed Mar. 11, 2019, each of which is incorporated by reference herein in its entirety.

### SEQUENCE LISTING

The instant application contains a Sequence Listing which has been submitted in XML format and is hereby incorporated by reference in its entirety. Said XML copy, created on Aug. 1, 2025, is named MDACP1408UISC1-Sequence-Listing.xml and is 253,441 bytes in size.

### BACKGROUND

Natural Killer (“NK”) cells are lymphoid-derived cells that directly inject toxic proteins into target cells. NK cells are part of the innate immune system and identify target cells that are stressed, such as cancer cells, and infected cells that express stress markers on their cell surface. NKs produce potent, MHC-unrestricted cytotoxicity to eradicate virally infected and transformed cells by a number of mechanisms, including direct release of cytotoxic granules containing perforin and granzymes, induced targets killing via death receptors and NK cell-mediated antibody-dependent cellular cytotoxicity (ADCC). Furthermore, cancerous tumors contain cells that frequently reduce expression of MHC-I proteins that provide an inhibitory signal to NK cells. Additionally, NK cells secrete proinflammatory cytokines/chemokines to recruit and activate effector T cells to the tumor site, modulate activity of antigen-presenting myeloid cells, and modify the tumor microenvironment in favor of an antitumor response. NK cells are thereby attractive as a cell therapy for cancer [Rezvani K, et al., Mol Ther 2017, 25 (8): 1769-1781; Curti A, et al., Blood 2011, 118 (12): 3273-3279; Oberschmidt O, et al., Front Immunol 2017, 8:654].

NK cells offer an attractive alternative to T cell-based therapy. First, allogeneic NK cells are expected to have lower risks for graft-versus-host disease (GVHD) than allogeneic T cells, which even when HLA-matched still posed a risk of GVHD through their native αβ T cell receptor. Moreover, besides the specificity of chimeric antigen receptor (“CAR”), engineered NKs retain their intrinsic ability to recognize and target tumor cells due to their full array of native receptors.

Natural killer cells may be prepared from donor-derived sources for allogeneic transfer to cancer patients [Rubnitz J E, et al., J Clin Oncol 2010, 28 (6): 955-959]. They are educated to recognize a complement of activating and inhibitory cell surface markers primarily to attack ‘self’ tissues that are stressed. The ability to source NK cells from donor peripheral blood or banked umbilical cord blood allows a NK cell therapy to be expanded and standardized for multiple patient infusions [Liu E, et al., Leukemia 2017; Shah N, et al, PLOS One 2013, 8 (10): e76781].

However, there are still major hurdles for clinical application of NK based therapy. Mature human NKs have a relatively limited life-span (estimated half-life 14 days, persistence in vivo around one month for adoptive transferred NKs), compared with years for CAR-T products. Hence, alternative approaches for NK prolonged survival is highly desirable in the field.

### SUMMARY

In another aspect, provided herein is a nucleic acid, comprising a first polynucleotide and a second polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide. In some embodiments, the nucleic acid further comprises a third polynucleotide, and wherein the third polynucleotide encodes either (i) a chimeric antigen receptor (CAR) or T-cell receptor, or (ii) a chimeric pro-apoptotic polypeptide comprising a second ligand binding region and a caspase-9 polypeptide lacking the caspase activation domain (CARD domain), and wherein the ligand binding region of the chimeric polypeptide is different than the second ligand binding domain of the chimeric pro-apoptotic polypeptide. The chimeric pro-apoptotic polypeptide is sometimes referred to herein as a safety switch. In some embodiments, the nucleic acid comprises a polynucleotide sequence encoding a marker (e.g., ΔCD19 polypeptide). In specific embodiments, a polynucleotide encoding a cleavage site, such as a 2A cleavage site or a 2A-like cleavage site, is used to link the polynucleotides. For examples of such cleavage sites, see, e.g., Donnelly et al., Analysis of the aphthovirus 2A/2B polyprotein ‘cleavage’ mechanism indicates not a proteolytic reaction, but a novel translational effect: a putative ribosomal ‘skip.’ J Gen Virol 2001; 82: 1013-1025 and Quintarelli et al., Co-expression of cytokine and suicide genes to enhance the activity and safety of tumor-specific cytotoxic T lymphocytes. Blood 2007; 110: 2793-2802.

In another aspect, provided herein is a nucleic acid, comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric antigen receptor (CAR) or a T cell receptor. In specific embodiments, the CAR targets PSMA, PSCA, Muc1 CD19, ROR1, Mesothelin, GD2, CD123, Muc16, CD33, CD38, CD44v6, Her2/Neu, CD20, CD30, BCMA, PRAME, NY-ESO-1, or EGFRvIII. In particular embodiments, the CAR targets HER-2, PSCA, CD123, or BCMA. In some embodiments, the nucleic acid further comprises a fourth polynucleotide, and wherein the fourth polynucleotide encodes a chimeric pro-apoptotic polypeptide comprising a second ligand binding region and a caspase-9 polypeptide lacking the caspase activation domain (CARD domain), and wherein the ligand binding region of the chimeric polypeptide is different than the second ligand binding domain of the chimeric pro-apoptotic polypeptide. In some embodiments, the nucleic acid comprises a polynucleotide sequence encoding a marker (e.g., ΔCD19 polypeptide). In specific embodiments, a polynucleotide encoding a cleavage site, such as a 2A cleavage site or a 2A-like cleavage site, is used to link the polynucleotides.

In another aspect, provided herein is a nucleic acid, comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric pro-apoptotic polypeptide comprising a second ligand binding region and a caspase-9 polypeptide lacking the caspase activation domain (CARD domain), and wherein the ligand binding region of the chimeric polypeptide is different than the second ligand binding domain of the chimeric pro-apoptotic polypeptide. In some embodiments, the nucleic acid further comprises a fourth polynucleotide, and wherein the fourth polynucleotide encodes a marker polypeptide (e.g., ΔCD19 polypeptide). In specific embodiments, a polynucleotide encoding a cleavage site, such as a 2A cleavage site or a 2A-like cleavage site, is used to link the polynucleotides.

In another aspect, provided herein is a nucleic acid, comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a marker polypeptide (e.g., ΔCD19 polypeptide).

In another aspect, provided herein is a nucleic acid, comprising a first polynucleotide and a second polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide. In some embodiments, the nucleic acid further comprises a third polynucleotide, and wherein the third polynucleotide encodes either (i) a chimeric antigen receptor (CAR) or T-cell receptor, or (ii) a chimeric pro-apoptotic polypeptide comprising a ligand binding region and a caspase-9 polypeptide lacking the caspase activation domain (CARD domain.) In some embodiments, the nucleic acid comprises a polynucleotide sequence encoding a marker (e.g., ΔCD19 polypeptide). In some embodiments, the first polynucleotide further comprises a ligand binding region. In specific embodiments, a polynucleotide encoding a cleavage site, such as a 2A cleavage site or a 2A-like cleavage site, is used to link the polynucleotides.

In another aspect, provided herein is a nucleic acid, comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric antigen receptor (CAR) or a T cell receptor. In specific embodiments, the CAR targets PSMA, PSCA, Muc1 CD19, ROR1, Mesothelin, GD2, CD123, Muc16, CD33, CD38, CD44v6, Her2/Neu, CD20, CD30, BCMA, PRAME, NY-ESO-1, or EGFRvIII. In particular embodiments, the CAR targets HER-2, PSCA, CD123, or BCMA. In some embodiments, the nucleic acid further comprises a fourth polynucleotide, and wherein the fourth polynucleotide encodes a chimeric pro-apoptotic polypeptide comprising a ligand binding region and a caspase-9 polypeptide lacking the caspase activation domain (CARD domain). In some embodiments, the nucleic acid comprises a polynucleotide sequence encoding a marker (e.g., ΔCD19 polypeptide). In some embodiments, the first polynucleotide further comprises a ligand binding region. In specific embodiments, a polynucleotide encoding a cleavage site, such as a 2A cleavage site or a 2A-like cleavage site, is used to link the polynucleotides.

In another aspect, provided herein is a nucleic acid, comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric pro-apoptotic polypeptide comprising a ligand binding region and a caspase-9 polypeptide lacking the caspase activation domain (CARD domain). In some embodiments, the nucleic acid further comprises a fourth polynucleotide, and wherein the fourth polynucleotide encodes a marker polypeptide (e.g., ΔCD19 polypeptide). In some embodiments, the first polynucleotide further comprises a ligand binding region. In specific embodiments, a polynucleotide encoding a cleavage site, such as a 2A cleavage site or a 2A-like cleavage site, is used to link the polynucleotides.

In another aspect, provided herein is a nucleic acid, comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a marker polypeptide (e.g., a ΔCD19 polypeptide). In some embodiments, the first polynucleotide further comprises a ligand binding region.

In some embodiments, a nucleic acid described herein is contained in a vector, such as a viral vector or plasmid. In certain embodiments, a nucleic acid described herein is contained in an adenoviral vector, a retroviral vector, or a lentiviral vector. In some embodiments, a nucleic acid described herein is isolated. In certain embodiments, provided herein is a composition comprising a nucleic acid described herein.

In specific embodiments, a nucleic acid is one described in the Examples described herein below (e.g., Example 3 or 4). For example, in certain embodiments, a nucleic acid described herein is a construct described herein, such as, e.g., the BP2811 or BP2810 construct described herein (e.g., depicted in FIG. 1 and described in the Examples below). In some embodiments, a nucleic acid described herein contains the transgenes of a construct described herein, such as e.g., the BP2811 or BP2810 construct described herein (e.g., depicted in FIG. 1 and described in the Examples below).

In another aspect, provided herein is a modified natural killer (NK) cell comprising the nucleic acid described herein. In a specific embodiment, a modified NK cell is engineered to express a nucleic acid described herein. In another specific embodiment, a modified NK cell(s) is of the type described in the Examples below (e.g. Example 3 or 4, infra).

In another aspect, provided herein is a modified cell(s) comprising a first polynucleotide and a second polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide. In some embodiments, the modified NK cell(s) further comprises a third polynucleotide, and wherein the third polynucleotide encodes either (i) a chimeric antigen receptor (CAR) or T-cell receptor, or (ii) a chimeric pro-apoptotic polypeptide comprising a second ligand binding region and a caspase-9 polypeptide lacking the caspase activation domain (CARD domain), and wherein the ligand binding region of the chimeric polypeptide is different than the second ligand binding domain of the chimeric pro-apoptotic polypeptide. In some embodiments, the nucleic acid comprises a polynucleotide sequence encoding a marker (e.g., ΔCD19 polypeptide).

In another aspect, provided herein is a modified NK cell(s) comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric antigen receptor (CAR) or a T cell receptor. In specific embodiments, the CAR targets PSMA, PSCA, Muc1 CD19, ROR1, Mesothelin, GD2, CD123, Muc16, CD33, CD38, CD44v6, Her2/Neu, CD20, CD30, BCMA, PRAME, NY-ESO-1, or EGFRvIII. In particular embodiments, the CAR targets HER-2, PSCA, CD123, or BCMA. In some embodiments, the modified NK cell(s) further comprises a fourth polynucleotide, and wherein the fourth polynucleotide encodes a chimeric pro-apoptotic polypeptide comprising a ligand binding region and a caspase-9 polypeptide lacking the caspase activation domain (CARD domain), and wherein the ligand binding region of the chimeric polypeptide is different than the second ligand binding domain of the chimeric pro-apoptotic polypeptide. In some embodiments, the nucleic acid comprises a polynucleotide sequence encoding a marker (e.g., ΔCD19 polypeptide).

In another aspect, provided herein is a modified NK cell(s) comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric pro-apoptotic polypeptide comprising a second ligand binding region and a caspase-9 polypeptide lacking the caspase activation domain (CARD domain), and wherein the ligand binding region of the chimeric polypeptide is different than the second ligand binding domain of the chimeric pro-apoptotic polypeptide. In some embodiments, the modified NK cell(s) further comprises a fourth polynucleotide, and wherein the fourth polynucleotide encodes a marker polypeptide (e.g., ΔCD19 polypeptide).

In another aspect, provided herein is a modified NK cell(s) comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising: (1) a ligand binding region; and (2) a signaling region, comprising (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a marker polypeptide (e.g., ΔCD19 polypeptide).

In another aspect, provided herein is a modified NK cell(s) comprising a first polynucleotide and a second polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide. In some embodiments, the modified NK cell(s) further comprises a third polynucleotide, and wherein the third polynucleotide encodes either (i) a chimeric antigen receptor (CAR) or T-cell receptor, or (ii) a chimeric pro-apoptotic polypeptide comprising a ligand binding region and a caspase-9 polypeptide lacking the caspase activation domain (CARD domain). In some embodiments, the first polynucleotide further comprises a ligand binding region. In some embodiments, the nucleic acid comprises a polynucleotide sequence encoding a marker (e.g., ΔCD19 polypeptide).

In another aspect, provided herein is a modified NK cell(s) comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric antigen receptor (CAR) or a T cell receptor. In specific embodiments, the CAR targets PSMA, PSCA, Muc1 CD19, ROR1, Mesothelin, GD2, CD123, Muc16, CD33, CD38, CD44v6, Her2/Neu, CD20, CD30, BCMA, PRAME, NY-ESO-1, or EGFRvIII. In particular embodiments, the CAR targets HER-2, PSCA, CD123, or BCMA. In some embodiments, the modified NK cell(s) further comprises a fourth polynucleotide, and wherein the fourth polynucleotide encodes a chimeric pro-apoptotic polypeptide comprising a second ligand binding region and a caspase-9 polypeptide comprises a polynucleotide sequence encoding a marker (e.g., ΔCD19 polypeptide). In some embodiments, the first polynucleotide further comprises a ligand binding region.

In another aspect, provided herein is a modified NK cell(s) comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a chimeric pro-apoptotic polypeptide comprising a second ligand binding region and a caspase-9 polypeptide lacking the caspase activation domain (CARD domain). In some embodiments, the modified NK cell(s) further comprises a fourth polynucleotide, and wherein the fourth polynucleotide encodes a marker polypeptide (e.g., ΔCD19 polypeptide). In some embodiments, the first polynucleotide further comprises a ligand binding region.

In another aspect, provided herein is a modified NK cell(s) comprising a first polynucleotide, a second polynucleotide and a third polynucleotide, wherein the first polynucleotide encodes a chimeric polypeptide (sometimes referred to herein as a chimeric signaling polypeptide or a chimeric stimulating molecule) comprising a signaling region, wherein the signaling region comprises: (a) a MyD88 polypeptide; (b) a truncated MyD88 polypeptide lacking the TIR domain; (c) a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; (d) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; (e) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or (f) a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40; and wherein the second polynucleotide encodes an IL-15 polypeptide; and wherein the third polynucleotide encodes a marker polypeptide (e.g., ΔCD19 polypeptide). In some embodiments, the first polynucleotide further comprises a ligand binding region.

In specific embodiments, a modified NK cell(s) described herein is (are) cryostored. In some embodiments, a modified NK cell(s) described herein has been cryostored. In some embodiments, a modified NK cell(s) has (have) been stored at a temperature of −150° C. or below.

In specific embodiments, a modified NK cell(s) has (have) not been grown on feeder cells. In specific embodiments, a modified NK cell(s) has (have) not been contacted with exogenous IL-15.

In a specific embodiment, a modified NK cell(s) described herein exhibits one, two, three or more, or all of the activities, functions or both of the modified NK cells described in the Examples described herein (e.g., Examples 3 and 4).

In a specific embodiment, a modified NK cell(s) described herein, which has been transduced with a nucleic acid comprising a polynucleotide encoding a chimeric polypeptide described herein and a polynucleotide encoding a chimeric pro-apoptotic polypeptide described herein, demonstrates enhanced proliferation in vitro relative to a modified NK cell(s), which has been transduced with a nucleic acid comprising a polynucleotide encoding the chimeric pro-apoptotic polypeptide. In another specific embodiment, proliferation of a modified NK cell(s) described herein, which has been transduced with a nucleic acid comprising a polynucleotide encoding a chimeric polypeptide described herein and a polynucleotide encoding a chimeric pro-apoptotic polypeptide described herein, in vitro is significantly enhanced following ligand (e.g., Rim) treatment compared the proliferation of a modified NK cell(s), which has been transduced with a nucleic acid comprising a polynucleotide encoding the chimeric pro-apoptotic polypeptide, treated with ligand (e.g., Rim). In a specific embodiment, the method used to assess the proliferation is the methodology described in Example 3 below.

In a specific embodiment, a modified NK cell(s) described herein, which has been transduced with a nucleic acid comprising a polynucleotide encoding a chimeric polypeptide described herein, a polynucleotide encoding IL-15, and a polynucleotide encoding a chimeric pro-apoptotic polypeptide described herein, demonstrates enhanced persistence in vivo in the presence of ligand (e.g., Rim) relative to the persistence in vivo of a modified NK cell(s), which has been transduced with a nucleic acid comprising a polynucleotide encoding the chimeric polypeptide and a polynucleotide encoding the chimeric pro-apoptotic polypeptide in the presence or absence of the ligand. In a specific embodiment, the method used to assess the NK cell persistence in vivo is the methodology described in Example 3 below.

In a specific embodiment, a modified NK cell(s) described herein, which has been transduced with a first nucleic acid comprising (i) a polynucleotide encoding a chimeric polypeptide described herein, (ii) a polynucleotide encoding IL-15, and (iii) a polynucleotide encoding a chimeric pro-apoptotic polypeptide described herein, and a second nucleic acid comprising a CAR, demonstrates superior killing of target cells at a high effector to taget cell ratio (e.g., an effector to target cell ratio of 1:4, 1:5, 1:6, 1:7, 1:8, 1:9, or 1:10) in the presence of ligand (e.g., Rim) relative to a modified NK cell(s), which has been transduced with a nucleic acid comprising the first nucleic acid or the second nucleic acid. In a specific embodiment, the method used to assess the killing is the methodology described in Example 3 below.

In specific embodiments, a signaling region of a chimeric polypeptide described herein comprises a truncated MyD88 polypeptide lacking the TIR domain and a CD40 cytoplasmic polypeptide region lacking the CD40 extracellular domain. In a specific embodiment, the truncated MyD88 polypeptide lacking the TIR domain comprises the amino acid sequence of SEQ ID NO: 119 or an amino acid sequence that is 90% identical to SEQ ID NO: 119. In another specific embodiment, the truncated MyD88 polypeptide lacking the TIR domain comprises the amino acid sequence of SEQ ID NO:2 or or an amino acid sequence that is 90% identical to SEQ ID NO:2. In another specific embodiment, the CD40 cytoplasmic polypeptide region lacking the CD40 extracellular region comprises the amino acid sequence of SEQ ID NO: 56 or an amino acid sequence that is 90% identical to SEQ ID NO:56.

In some embodiments, a signaling region of a chimeric polypeptide described herein comprises the MyD88 polypeptide. In a specific embodiment, the MyD88 polypeptide comprises the amino acid sequence of SEQ ID NO:118 or an amino acid sequence that is 90% identical to SEQ ID NO: 118.

In certain embodiments, a signal region of a chimeric polypeptide comprises a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions. In a specific embodiment, the MyD88 polypeptide comprises the amino acid sequence of SEQ ID NO: 119 or an amino acid sequence that is 90% identical to SEQ ID NO: 119. In another specific embodiment, the truncated MyD88 polypeptide lacking the TIR domain comprises the amino acid sequence of SEQ ID NO:2 or or an amino acid sequence that is 90% identical to SEQ ID NO: 2.

In some embodiments, a signaling region of a chimeric polypeptide comprises a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions. In one embodiment, the co-stimulatory polypeptide cytoplasmic signaling region is selected from the group consisting of CD27, CD28, ICOS, 4-1BB, RANK/TRANCE-R, and OX40. In another embodiment, the co-stimulatory polypeptide cytoplasmic signaling region is selected from the group consisting of CD28, 4-1BB, OX40, and ICOS. In another embodiment, the co-stimulatory polypeptide cytoplasmic signaling region is selected from the group consisting of CD27, CD28, CD30, ICOS, 4-1BB, CD40, RANK/TRANCE-R, and OX40. In another embodiment, the co-stimulatory polypeptide cytoplasmic signaling region is selected from the group consisting of CD27, CD28, ICOS, 4-1BB, and OX40. In certain embodiments, the co-stimulatory polypeptide lacks an extracellular domain or lacks a functional extracellular domain. In a specific embodiment, the truncated MyD88 polypeptide lacking the TIR domain comprises the amino acid sequence of SEQ ID NO: 119 or an amino acid sequence that is 90% identical to SEQ ID NO: 119. In another embodiment, the truncated MyD88 polypeptide lacking the TIR domain comprises the amino acid sequence of SEQ ID NO: 2 or or an amino acid sequence that is 90% identical to SEQ ID NO: 2. In another embodiment, the truncated MyD88 polypeptide lacking the TIR domain comprises the amino acid sequence of SEQ ID NO: 118 or or an amino acid sequence that is 90% identical to SEQ ID NO: 118.

In certain embodiments, a signal region of a chimeric polypeptide comprises a first co-stimulatory polypeptide cytoplasmic signaling region selected from the group consisting of CD27, CD28, CD30, ICOS, 4-1BB, CD40, RANK/TRANCE-R, and OX40, and a second co-stimulatory polypeptide cytoplasmic signaling region selected from the group consisting of CD27, CD28, CD30, ICOS, 4-1BB, CD40, RANK/TRANCE-R, and OX40. In one embodiment, the signal region comprises a first co-stimulatory polypeptide cytoplasmic signaling region selected from the group consisting of CD27, CD28, ICOS, 4-1BB, CD40, RANK/TRANCE-R, and OX40, and a second co-stimulatory polypeptide cytoplasmic signaling region selected from the group consisting of CD27, CD28, ICOS, 4-1BB, CD40, RANK/TRANCE-R, and OX40. In another embodiment, the signaling region comprises a first co-stimulatory polypeptide cytoplasmic signaling region selected from the group consisting of CD27, CD28, ICOS, 4-1BB, RANK/TRANCE-R, and OX40, and a second co-stimulatory polypeptide cytoplasmic signaling region selected from the group consisting of CD27, CD28, ICOS, 4-1BB, RANK/TRANCE-R, and OX40. In another embodiment, the signaling region comprises a first co-stimulatory polypeptide cytoplasmic signaling region selected from the group consisting of CD28, 4-1BB, OX40, and ICOS, and a second co-stimulatory polypeptide cytoplasmic signaling region selected from the group consisting of CD28, 4-1BB, OX40, and ICOS. In another embodiment, the signaling region comprises a first co-stimulatory polypeptide cytoplasmic signaling region selected from the group consisting of CD27, CD28, CD3, ICOS, 4-1BB, CD40, RANK/TRANCE-R, and OX40, and a second co-stimulatory polypeptide cytoplasmic signaling region selected from the group consisting of CD27, CD28, CD3, ICOS, 4-1BB, CD40, RANK/TRANCE-R, and OX40. In another embodiment, the signaling region comprises a first co-stimulatory polypeptide cytoplasmic signaling region selected from the group consisting of CD27, CD28, ICOS, 4-1BB, RANK/TRANCE-R, and OX40, and a second co-stimulatory polypeptide cytoplasmic signaling region selected from the group consisting of CD27, CD28, ICOS, 4-1BB, RANK/TRANCE-R, and OX40.

In a specific embodiment, an OX40, CD28, ICOS, 4-1BB, MyD88 or CD40 sequence comprises (or consists of) a sequence disclosed herein (e.g., a sequence disclosed in the Examples below).

In some embodiments, a ligand binding domain described herein is a multimeric ligand binding region. In certain embodiments, the ligand binding region described herein comprises an FKBP variant polypeptide, such as a human protein FK506-binding protein (FKBP) 12 (Fv) which contains a single acid substitution of phenylalanine to valine at amino acid position 36. In some embodiments, a ligand binding region described herein comprises multiple copies (e.g., two, three, or more copies) of an FKBP variant polypeptide, such as a human protein FKBP12 (Fv) which contains a single acid substitution of phenylalanine to valine at amino acid position 36. In a specific embodiment, a ligand binding domain described herein comprises two copies of FKBP12v36. In other embodiments, a ligand binding domain described herein comprises an FKBP12 polypeptide and an FKBP-rapamycin-binding region (FRB) polypeptide or an FRB variant polypeptide.

In a specific embodiment, a ligand binding region comprises FKBPV, FKBP″, FKBPV′, FKBV, FRBPwt, or FKB. In certain embodiments, FKBPV, FKBP″, FKBPV′, FKBV, FRBPwt, or FKB comprises a sequence disclosed herein (e.g., a sequence disclosed in the Examples below).

In a specific embodiment, a chimeric pro-apoptotic polypeptide comprises a Δcaspase-9 sequence disclosed herein (e.g. a sequence disclosed in the Examples below).

In a specific embodiment, ΔCD19 comprises (or consists of) a sequence disclosed herein (e.g., a sequence disclosed in the Exmples below).

In specific embodiments, a ligand binding domain described herein binds to a chemical inducer of dimerization or multimerization. In some embodiments, a ligand binding domain of a nucleic acid described herein binds to rimiducid, AP20187, or AP1510. In certain embodiments, a ligand binding domain described herein binds to rapamycin or a rapalog.

In another aspect, provided herein is a method for stimulating an immune response comprising administering modified NK cells described herein to a subject (e.g., a human subject). In one embodiment, the subject has a disease or condition associated with an elevated level of expression of a target antigen expressed by a target cell. In a particular embodiment, a tumor has been detected in the subject administered the modified NK cells.

In another aspect, provided herein pharmaceutical compositions comprising modified NK cells or nucleic acids described herein. In some embodiments, pharmaceutical compositions are provided that are prepared by the methods of the present application. In specific embodiments, modified NK cells described or pharmaceutical compositions comprising modified NK cells described herein may be used in accordance with the methods of stimulating an immune response, methods of treatment, or both.

In another aspect, provided herein is a method for stimulating an immune response comprising administering (i) modified NK cells described herein to a subject (e.g., a human subject) and (ii) a ligand that binds to the ligand binding region of the chimeric polypeptide. In specific embodiments, the ligand is administered after the modified NK cells are administered to the subject. In certain embodiments, the subject has a disease or condition associated with an elevated level of expression of a target antigen expressed by a target cell. In specific embodiments, the ligand is administered to the subject in amount effective to reduce the number or concentration of the target antigen or target cells in the subject. In some embodiments, a tumor has been detected in the subject administered the modified NK cells. In specific embodiments, the ligand is administered to the subject in an amount effective to reduce the size of the tumor in the subject. In specific embodiments, the ligand administered to the subject is rimiducid, AP20187, or AP1510. In other specific embodiments, the ligand administered to the subject is rapamycin or a rapalog.

In another aspect, provided herein is a method for reducing the number of modified NK cells in the event of a negative symptom or condition, comprising administering to a subject (e.g., a human subject) who has been previously been administered a ligand that binds to the ligand binding region of a chimeric pro-apoptotic polypeptide in an amount effective to reduce the number or concentration of the modified NK cells in the subject. In a specific embodiment, the amount administered to the subject is effective to kill at least 30% of the cells that express the chimeric pro-apoptotic polypeptide.

In another specific embodiment, the amount administered to the subject is effective to kill at least 60% of the cells that express the chimeric pro-apoptotic polypeptide. In a specific embodiment, the amount administered to the subject is effective to kill at least 90% of the cells that express the chimeric pro-apoptotic polypeptide. In another specific embodiment, the negative symptom or condition is graft-versus-host disease.

In some embodiments, a subject treated in accordance with the methods described herein has cancer. In certain embodiments, a subject treated in accordance with the methods described herein has been diagnosed as having a hyperproliferative disease. In some embodiments, a subject treated in accordance with the methods described herein has been diagnosed with sickle cell anemia or metachromatic leukodystrophy. In certain embodiments, a subject treated in accordance with the methods described herein has been diagnosed with a condition selected from the group consisting of a primary immune deficiency condition, hemophagocytosis lymphohistiocytosis (HAH) or another hemophagocytic condition, an inherited marrow failure condition, a hemoglobinopathy, a metabolic condition, and an osteoclast condition. In some embodiments, a subject treated in accordance with the methods described herein has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XAP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis. In certain embodiments, a subject treated in accordance with the methods described herein has been diagnosed with leukemia. In some embodiments, a subject treated in accordance with the methods described herein has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.

In specific embodiments, the ligand administered to the subject is rimiducid, AP20187, or AP1510. In other specific embodiments, the ligand administered to the subject is rapamycin or a rapalog.

In certain embodiments, a pharmaceutical composition comprises a ligand that binds to a ligand binding region described herein. In a specific embodiment, a pharmaceutical composition comprises a ligand that binds to a ligand binding region described herein and a pharmaceutically acceptable carrier. In some embodiments, such a pharmaceutical composition may be used to bind to a ligand binding region of a chimeric polypeptide and induce dimerization or multimerization resulting in the activation or enhancement of activation of modified NK cells. In other embodiments, such a pharmaceutical composition may be used to bind to a ligand binding region of a chimeric pro-apoptotic polypeptide and induce dimerization or multimerization resulting in killing of modified NK cells.

In some embodiments, modified Natural Killer cells described herein have one or more of the functions of the Natural Killer cells described in the Examples below, e.g., Example 1, 3 or 4. In some embodiments, modified Natural Killer cells described herein are produced using a method described in the Examples below.

In some embodiments, the subject is a mammal. In some embodiments, the subject is a human.

### BRIEF DESCRIPTION OF THE DRAWINGS

The drawings illustrate certain embodiments of the technology and are not limiting. For clarity and ease of illustration, the drawings are not made to scale, and, in some instances, various aspects may be shown exaggerated or enlarged to facilitate an understanding of particular embodiments.

FIG. 1 provides schematics of expression constructs BP1664, BP2811, BP1526, BP2810, BP1385, and BP606.

FIG. 2 provides a schematic representation of dual switch components. Version 1: MyD88/CD40 is fused at its carboxyl terminus with two copies of Fv (FKBP12v36), and coexpressed with a chimeric caspase 9 polypeptide lacking the CARD domain fused at its amino terminus with Fwt (FKBP12f36, wild type) and FRB (T2098L). Version 2: MyD88/CD40 is fused at its amino terminus with tandem Fwt and FRB, coexpressed with chimeric caspase-9 polypeptide lacking the CARD domain fused at its amino terminus with Fv.

FIG. 3 provides a schematic of a transduction protocol that may be used to transduce natural killer cells. Methods outlined are essentially as discussed in Gene Modification of Human Natural Killer Cells Using a Retroviral Vector. Kellner J N, Cruz C R, Bollard C M, Yvon E S. Methods Mol Biol. 2016; 1441: 203-13.

FIG. 4 provides the results of growth assays in modified NK cells. Right: Graph showing percent CD19-expressing cells. Left: line graph of live cell counts.

FIG. 5 provides the results of proliferation assays in modified NK cells with and without activation of iMyD88/CD40 with rimiducid (AP1903). Left: Graph of percent CD19-expressing cells. Right: graph of CD19 Mean Fluorescence Intensity (MFI).

FIG. 6 provides a graph of IL-15 production in dual switch-modified NK cells.

FIG. 7 provides a graph of Interferon production in dual switch-modified NK cells.

FIGS. 8A-8D provides four graphs of cytokine and chemokine production bu NK cells comprising the dual switch. FIG. 8A: graph of GM-CSF concentration; FIG. 8B: graph of TNF-alpha concentration; FIG. 8C: MIP-1 alpha concentration; FIG. 8D: MIP-1-beta concentration.

FIGS. 9A-9D. FIG. 9A provides a timeline of an in vivo proliferation assay; FIG. 9B provides a table measuring proliferation of modified NK cells in mice. FIG. 9C provides luminescence images of mice used in the in vivo assays. FIG. 9D provides a graph of luminescence of NK cells in treated mice.

FIGS. 10A-10B. FIG. 10A provides a bar graph of percent THP-1 AML tumor cell killing by modified NK cells. FIG. 10B provides a line graph of percent THP-1 AML tumor cell killing by modified NK cells.

FIGS. 11A-11B. FIG. 11A provides a line graph of tumor growth in the presence of modified NK cells. FIG. 11B provides flow cytometry data of THP-GFP AML tumor cells incubated with modified NK cells.

FIGS. 12A-12B. FIG. 12A provides luminescence images of mice treated with modified NK cells. FIG. 12B provides a line graph of luminescence of tumor cells in the treated mice.

FIGS. 13A-13C. FIG. 13A provides a schematic of cryostorage (freezing) and thawing of modified NK cells used in the assays; FIG. 13B provides a graph of THP-luc killing at an E:T of 3:1 NK cells: THP-luciferase targets; FIG. 13C provides a graph of THP-luc killing at an E:T of 1:1 NK cells: THP-luciferase targets.

FIG. 14 provides a graph of caspase-9 activation in modified NK cells, assayed by percent CD19-expressing cells.

FIGS. 15A-15F: Rim-mediated iMC activation increases NK cells proliferation. NK cells were transduced with iMC.iRC9 or iRC9 at day 4 post activation. The cells were further stimulated by irradiated K562 feeder cells 5 days after transduction with/without 1 nM rimiducid. At day 14, cells were counted using Acridine orange (AO) and propidium iodide (“AOPI” staining) (FIG. 15A). FIG. 15B: NK cells were labeled with CellTrace dye the day of transduction. Proliferation was assessed 9 days later. Representative histograms show CellTrace dilution in iRC9.iMC transduced NK cell population (gated on CD56+CD3-CD19+) in the presence of 1 nM rimiducid (dark gray) or not (light gray). FIG. 15C: MFI of CellTrace dye in iRC9 or iRC9.iMC transduced NK cells with or without 1 nM rimiducid. Paired t tests were used to compare indicated groups. * p<0.05; ** p<0.01; NS none significant. In vivo persistence of iRC9.iMC or iRC9.IL-15.iMC transduced NK cells in the absence (FIGS. 15D and 15E) or presence (FIG. 15F) of tumor targets (THP-1 cells) in NSG mice. 2-way ANOVA was used to access differences among groups of E; P<0.01. Multiple T tests were used to assess differences between iRC9 rimiducidand iRC9.IL15.iMC Rim, blue *** P<0.001; and between iRC9.iMC Veh vs iRC9.iMC Rim, red ** P<0.01.

FIGS. 16A-16A-I. Rimiducid-mediated iMC activation enhances NK cells anti-tumor functions. NK cells transduced with iRC9.iMC or iRC9 were tested for their lytic capacity of tumor cell targets. NK cells were co-cultured with HPAC-GFPFfluc (FIG. 16A) or THP-1-GFPFfluc (FIG. 16B) at different effector-to-target (E:T) ratio for 24 hrs. Tumor cells killing percentages were calculated by relative luciferase activity. N=4. (FIGS. 16C and 16D) Flow cytometry analysis of THP-1-GFPFfluc cells co-cultured with iRC9, iRC9.iMC, or iRC9.IL15.iMC transduced or non-transduced (NT) NKs for 2 days with/without rimiducid 1 nM. Representative flow plots were shown at the E:T ratio of 1:1. Live cells were gated by forward, side scatter and fixable viability dye before. THP-1 population was gated as GFP+ population. 3 donors with E:T ratio 3:1, 1:1, 1:3. Data expressed as means with standard errors. 2-way ANOVA was used do comparisons for A, B, D; P<0.0001. (FIGS. 16E-16I) iRC9 or iRC9.iMC transduced NKs were incubated with/without THP-1 targets for 4 hrs (FIG. 16E), or overnight (FIGS. 16F-16I) with or without 1 nM Rim. Percentage of surface CD107a (FIG. 16E), intracellular IFN-γ (FIG. 16F), and TNF-α (FIG. 16G), were measured by flow cytometry. MFI of perforin (FIG. 16H) and granzymeB (FIG. 16I) were measured in NKs co-culture with THP-1 overnight. Transduced NK cells were first gated as CD56+CD19+ population. Paired t test was used to compare groups. * p<0.05; ** p<0.01.

FIG. 17 shows levels of surface CD107a expressed by NK cells transduced with retrovirus encoding iRC9 or iRC9 and iMC when co-cultured with tumor targets K562, THP-1 or Nalm6 or alone in the absence or presence of rimiducid (Rim).

FIGS. 18A-18E Phenotype of iMC transduced NK cells. FIG. 18A: iRC9, iRC9.iMC, iRC9.IL15.iMC NK cells phenotype based on the average expression of DNAM1, NKP30, NKP44, NKP46, NKG2D, CD16, Fas, FasL were characterized by multiparameter flow cytometry analysis. MFI or the percentages of positive cells were normalized according the average value of non-transduced NK cells. FIG. 18B: IFN-γ productions in supernatant by iRC9, iRC9.iMC, or iRC9.IL15.iMC transduced NK cells when co-culture with k562 cells at the E:T ratio 1:1 for 48 hrs. in the presences of variable rimiducid concentrations (0-100 nM). Two-way ANOVAs were used to compare groups. P<0.0001. FIG. 18C: 29-plex cytokine multiplex analysis of supernatant from iMC-iRC9, iMC.IL15, or iRC9 transduced NK cells co-cultured with/without THP-1 in the absence or presence of 1 nM rimiducid. FIGS. 18D and 18E: NT, iRC9, iRC9.iMC, or iRC9.IL15.iMC transduced NK cells, either untreated or treated with 1 nM rimiducid for 6 days. Transgene expression as indicated by CD19 positive percentage. Representative flow plots are shown in FIG. 18D and dot plots for N=4 in FIG. 18E. Paired t tests were used to compare groups. ** p<0.01.

FIGS. 19A-19D Enhancing NK cell anti-tumor efficacy by antibody-dependent cellular cytotoxicity. FIG. 19A: NK cells transduced with iMC.IL15.iRC9 were co-cultured with SKOV3RFP at a 2:1 E:T ratio at variable Herceptin concentrations (0-1200 ng/ml). SKOV3 (RFP, red) proliferation were measured by live-cell imaging using an IncuCyte imager. After 72 h, SKOV3 killing was calculated relative to tumor cells alone. FIG. 19B: IRC9 and iMC.IL15.iRC9 NK cell co-culture with SKOV3RFP at a 1:2 effector to target (E:T) ratio with or without 300 ng/ml Herceptin and 1 nM rimiducid. Two-way ANOVA was used to do comparison. P<0.0001. After 72 h of co-culture SKOV3RFP proliferation was measured by IncuCyte (FIG. 19C). After 71 h, co-culture with different transduced NK cells OE19GFP proliferation was measured by IncuCyte. Two-way ANOVA were used do comparison. P<0.01. (FIG. 19D)

FIGS. 20A-20G iMC activation by rimiducid enhances anti-tumor efficacy of CD123-CAR NK cells against leukemia. FIG. 20A: Activated NK cells were transduced with γ-RV encoding CD123ζ CAR, dual switch DS.IL15 (iRC9.IL15.iMC), or DS.IL15+CD123ζ CAR. CD19 marker was used to check dual switch transduction efficiency, CD34 marker was used to check for CD123ζ CAR transduction efficiency. FIG. 20B: Transduced NK cells were co-cultured with THP-1-GFPFfluc at different E:T ratios in the presence or absence of 1 nM rimiducid. Luciferase activity was determined at 24 hrs. N=4 donors. Multiple t tests were used to compare DS.IL15 Rim and DS.IL15+CD123ζ CAR Rim. ** P<0.01. FIGS. 20C-20D: NSG mice were engrafted i.v. with 10⁷ NKs NT or transduced with CD123ζ CAR, DS.IL15, or DS.IL15+CD123ζ CAR; 3 days following i.v. implantation of 10⁶ THP-1-GFPFfluc tumor cells. 1 mg/kg rimiducid or vehicle was administrated i.p. weekly. BLI was monitored by IVIS. Multiple t tests were used to compare CD123ζ CAR Rim group with NT group. *** P<0.001. FIG. 20E: At day 53 post NK therapy, DS.IL15+CD123ζ CAR Rim group was euthanized. Human NK cells were identified in spleen, bone marrow, and peripheral blood. Compared with DS.IL15+CD123ζ CAR vehicle group euthanized at day 35 post NK therapy (FIG. 20F). FIG. 20G: CD123 surface expression in THP-1-GFPFfluc tumor cells (GFP+ population) in spleen and bone marrow. All other groups were at timepoint day 35 except DS.IL15+CD123ζ CAR Rim group was obtained at day 53. 2-way ANOVA were used do comparison. P=0.47.

FIG. 21 provides a line graph of expansion of THP-1 target cells incubated with modified NK cells transduced with the nucleic acid vectors as discussed in Example 3 below, with lines representing results obtained at 132.5 hours, from top to bottom: tumor only, tumor only+Rim; DS/IL15; DS/IL15+Rim; DS/IL15+CD123ζ CAR; CD123ζ CAR; CD123ζ CAR+Rim; DS/IL15+CD123ζ CAR+Rim.

FIGS. 22A-22G iMC enhanced anti-tumor efficacy of BCMA-CAR NK against THP-1 tumor in NSG mice. FIG. 22A: Transduction efficiency of DS.BCMA.ζ.IL15 NK, which was co-transduced with γ-RV encoding iMC.BCMA.ζ.IL15 and iRC9. FIGS. 22B and 22C: NSG mice were engrafted with 10⁷ NK cells either non-transduced or transduced with DS.BCMA.ζ.IL15 NK 3 days following intravenous (“i.v”). implantation of 10⁶ THP-1-GFPFfluc tumor cells. 1 mg/kg rimiducid or vehicle was administrated intraperitoneal (“i.p”). Five times a week for the first week, and three times a week for the rest time. Bioluminescence (“BLI”) was monitored by IVIS. Multiple t tests were used to compare DS.BCMA.ζ.IL15 NK Rim group with tumor alone group. ** P<0.01, *** P<0.001. FIGS. 22D and 22E: At day 40 to day 48, mice from NT, DS.BCMA.ζ.IL15 NK vehicle or rimiducid groups were euthanized. THP-1-GFPFfluc cells were identified in bone marrow and spleen as GFP+ populations.

FIGS. 22F-22G: Human NK cells were identified in spleen and bone marrow as mCD45-GFP-hCD45+hCD34+ populations. Student t tests weres used to do comparisons. * P<0.05, *** P<0.001.

FIGS. 23A-23C: FIG. 23A shows relative expression of ILT2 and ILT4 in T cells and NK cells. FIG. 23B shows MFI HLA-ABC levels in K562, OE-19, THP-1, HPAC, SKOV3, and ISO cell lines and FIG. 23C shows MFI MICA/B levels in K562, OE-19, THP-1, HPAC, SKOV3, and ISO cell lines.

FIGS. 24A-24C: DS NKs anti-tumor efficacy in vivo. FIG. 24A: NSG mice were tail vein injected 5×10⁶ NK cells double transduced with RV encoding iRC9, iRC9.iMC, or iRC9.IL15.iMC, 5 and 12 days following i.v. implantation of 10⁶ THP-1.GFPluc tumor cells. Rimiducid or vehicle was administered i.p. weekly. Tumor cells growth (D-luciferin substrate) were monitored by IVIS. FIGS. 24B and 24C: Survival curves were plotted. Comparison of survival curves were done by Log-rank (Mantel-Cox) test. P<0.0001.

FIGS. 25A-25D: Safety switch in NKs. NT NK or NK transduced with iRC9, iRC9.iMC, or iRC9.IL15.iMC were administrated with temsirolimus at the concentration 0, 0.1, 1, and 10 nM for 4 hrs. FIG. 25A: Annexin V staining was assessed by flow cytometry analysis. FIG. 25B: Temsirolimus at various concentration was administrated for 24 hrs. 7AAD staining was assessed by flow cytometry. Cells were first gated as CD56+CD19+ for transduced cells, CD56+ for NT non-transduced NK cells. FIGS. 25E-25G: 9 NSG mice engrafted with iRC9.IL15.iMC were randomly divided into two groups. 4 Four mice were applied administered 1 mg/kg temsirolimus i.p., whereas 5 mice were administrated with same volume vehicle. Before injection and 24 hrs. later, firefly luciferase BLI was determined for to measure NK presence in vivo (FIG. 25E). Mice were then euthanized. Spleens were analyzed for the presence of NK cells by flow cytometry. NK cells were identified as hCD45+mCD45− populations (FIG. 25F). Transduced NKcells were identified as hCD45+mCD45−hCD56+hCD19+ populations (FIG. 25G). Student t test was used to do comparisons compare groups. *** P<0.001.

FIGS. 26A-H. Analysis of Dual Switch CAR-T and CAR-NK cells. Lymphocytes were separated from peripheral blood mononuclear cells (PBMCs) derived from three donor and separated into T cell and NK cell preparations by CD56 bead purification. T cells and NK cells were activated by αCD3/αCD28 antibodies and IL-15 followed by K562 cell stimulation respectively, as described previously. Cells were then transduced with BP2818 and BP1385 viruses encoding dual switch constructs iMC-αBCMACAR-IL15 and iC9-αCD19 (a marker for transduction). FIG. 26A: Flow cytometry panels documenting representative transduction efficiencies of CD34 (a CAR marker) and CD19 (a safety switch marker). FIG. 26B: Transduction efficiency for expression of both markers of transduction efficiencies in CAR-T cells and CAR-NK cells in each donor preparation. FIG. 26C: Expression of BCMA in four target tumor cell lines determined by flow cytometry. FIGS. 26D-26G: Co-cultures between non-transduced (NT) T cells and NK cells and Dual Switch transduced BCMA directed CAR-T and CAR-NK cells and target cell lines were prepared at decreasing Effector to Target (E:T) ratios to determine relative efficacy for cell killing. Failure of GFP-labeled tumor cells to grow over the course of 150 hours in the presence and absence of 1 nM rimiducid relative to tumor cells not containing effector cells was determined by Incucyte microscopy. Results were plotted for cocultures of NCIH929 (FIG. 26D), THP-1 (FIG. 26E), U266 (FIG. 26F) and RPMI8226 cells (FIG. 26G). Supernatants from cocultures of tumor cell targets and T and NK cells outlined in FIGS. 26D-26G at E:T of 1:4 were isolated and analysed for cytokine levels by Multiplex analysis (Biorad). Results indicated that dual switch CAR-NK cells activated with rimiducid and exposed to multiple tumor targets express similar levels of inflammatory cytokines IFN-γ, TNF-α and GM-CSF as dual switch CAR-T cells while secreting elevated amounts of IL-13 and IL-5 relative to dual switch CAR-T cells (FIG. 26H). Furthermore, the secretion of chemokines such as MIP1α, MIP1β and MCP1 was elevated in rim-treated dual switch CAR-NK cells relative to similar co-culture of dual switch CAR-T cells supporting the hypothesis that dual switch CAR-NK cells may produce a more pro-inflammatory tumor microenvironment than dual switch CAR-T cells by recruitment of dendritic cells, macrophage and lymphocytes to an anti-tumor response.

FIG. 27: Plasmid DNA constructs were created to generate γ-retroviruses encoding iMC with a CAR and IL-15 each separated by 2A cotranslational cleavage sites (2818) or a constitutively active clone (2819) in which inefficient cleavage at the 2A site between the CAR and MC (lacking Fv) creates a CAR-MC fusion product that is constitutively active.

FIG. 28: 5×10⁵ NK cells derived from three donors were transduced with retrovirus encoding inducible MC (2818) or constitutively active MC (2819) and were cultured with IL-2 in the presence or absence of rimiducid as indicated. At Day 4, NK cell cultures were further stimulated with irradiated K562 cells. Cell counts were determined at the indicated days.

FIGS. 29A-29C: NK cells derived from three donors were transduced with retrovirus encoding inducible MC (2818) or constitutively active MC (2819) and were co-cultured (with IL-2) with BCMA-expressing THP-1 cells (FIG. 29A), RPMI8226 (FIG. 29B) or BCMA-negative Nalm6 cells (FIG. 29C) in the presence or absence of Rimiducid as indicated. In each case the relative amount of NK effectors to tumor targets (E:T) was varied as indicated. Co-cultures containing GFP-labeled tumor targets were incubated in an incucyte microscope and fluorescence monitored over time. Mean fluorescence relative to cultures without NK cells at 100 or 140 hours incubation are indicated.

FIGS. 30A-30C: NK cells derived from three donors were transduced with retrovirus encoding inducible MC (2818) or constitutively active MC (2819) and were co-cultured (with IL-2) with BCMA-expressing THP-1 cells (FIG. 30A), RPMI8226 (FIG. 30B) or BCMA-negative Nalm6 cells (FIG. 30C) in the presence or absence of Rimiducid as indicated. In each case the relative amount of NK effectors to tumor targets (E:T) was varied as indicated. Supernatant was taken after 24 hours of coculture and Interferon-γ levels determined by ELISA.

### DETAILED DESCRIPTION

The present invention relates generally to the field of immunology and relates in part to compositions, and methods for growing and storing modified natural killer cells, including for example, conditional chemical regulation of natural killer cell function. The technology further relates to pharmaceutical compositions and treatment of subjects using modified natural killer cells. Provided herein in some embodiments are allele systems in which the proliferation of human Natural Killer (NK) cells along with their persistence in vivo, production of immune activating cytokines and ability to kill tumor cells can be enhanced by the introduction of a protein-based molecular switch that could be constitutively activated or a protein-based molecular switch that could be responsive to a synthetic ligand. To mitigate cases of NK cell overactivity that may produce toxicity, a second protein-based molecular switch is included to ablate NK cell function by the induction of apoptosis. These dual switch NK cells may be used as a cell therapy for cancer or as an adjunct to other immunotherapies for cancer.

### INTRODUCTION

Natural killer cells (NK cells) are cytotoxic lymphocytes that are part of the innate immune system. NK cells, in general, do not express markers indicative of T or B cells. NK cell markers include CD16 and/or CD56. NK cells act by killing target cells, such as tumor cells, infected cells, and antibody-targeted cells. NK cells may be obtained or isolated from, for example, peripheral blood, bone marrow, and umbilical cord blood, or they can be derived from stem cell populations including CD34 postive hematopoeitic stem cells themselves derived from Induced Pluripotent Stem (IPS) cells that can be genetically modified. The terms “inducible pluripotent stem cell” or “induced pluripotent stem cell” as used herein refers to adult, or differentiated cells, that are “reprogrammed” or induced by genetic (e.g., expression of genes that in turn activates pluripotency), biological (e.g., treatment viruses or retroviruses) and/or chemical (e.g., small molecules, peptides and the like) manipulation to generate cells that are capable of differentiating into many if not all cell types, like embryonic stem cells. Inducible pluripotent stem cells are distinguished from embryonic stem cells in that they achieve an intermediate or terminally differentiated state (e.g., skin cells, bone cells, fibroblasts, and the like) and then are induced to dedifferentiate, thereby regaining some or all of the ability to generate multipotent or pluripotent cells. CD34″ as used herein refers to a cell surface glycoprotein (e.g., sialomucin protein) that often acts as a cell-cell adhesion factor and is involved in T cell entrance into lymph nodes, and is a member of the “cluster of differentiation” gene family.

As mentioned above, the use of allogeneic NK cells has potential in cell therapy. However, several deficiencies limit the widespread use of NK cells as a cell therapy for cancer: 1) NK cells have limited intrinsic potential for proliferation and persistence in vivo [Rezvani K, et al., Mol Ther 2017, 25 (8): 1769-1781; 3; Oberschmidt O, et al., Front Immunol 2017, 8:654; Fujisaki H, et al., Cancer Res 2009, 69 (9): 4010-4017]; 2) NK cells are subject to non-viability and inefficacy when thawed from cryostorage and are therefore generally produced from fresh material and expanded for each use. This property limits there usage only to specialized centers. 3) While the risks of excessive cytokine release and GvHD are far less than those of T cell therapies, there is a potential for off-tumor or systemic toxicity from the grafted cells [Bonifant C L, et al., Mol Ther Oncolytics 2016, 3:16011; Shah N N, et al., Blood 2015, 125 (5): 784-792].

Provided herein are compositions, and methods for growing and storing modified natural killer cells. The compositions and methods provide NK cells that proliferate and persist in vivo. Methods are provided for cryostoring and thawing NK cells, and optionally for growing NK cells in the absence of a feeder layer. NK cells provided in the present embodiments are viable and demonstrate efficacy in vitro and in vivo. In specific embodiments, NK cells provided herein are viable and demonstrate efficacy in vitro and in vivo even after cryopreservation. In some embodiments, the compositions and methods provide the ability to obtain, grow, store, expand, and administer NK cells, including allogeneic NK cells, while reducing certain cytotoxic effects.

In the examples and embodiments described herein, the inventors have established a novel approach to engineering NKs with superior effector functions, such as NK cell proliferation, in vivo persistence, cytokines production, and antitumor activity. In the examples described below NK cells derived from peripheral blood were transduced with retroviral vectors encoding costimulatory molecules, IL-15, a suicide gene, and in some experiments an additional tumor antigen specific CAR. In the experiment where an inducible costimulatory molecule was used, upon rimiducid administration the modified NK cells showed improved expansion in vitro and sustained persistence in NSG mice both in the presence and absence of tumor targets. The activated iMC gene modified NKs function better, in releasing more granules containing perforin, granzyme B, and effective cytokines IFN-γ TNF-α, resulted in effectively killing of either MHC class I high expressing NK resistant tumor cells and MHC class I low expressing NK sensitive tumor cells. Moreover, when combined with a CAR targeting tumor specific antigens, rimiducid-activated iMC modified NKs demonstrated potent antitumor responses against their target, associated with prolonged persistence in vivo and homing to bone marrow and spleen, sites of disease. Thus, surprisingly, the inventors observed that Rimiducid activated iMC modified NKs demonstrated enhanced proliferation, upregulated cytokines productions including IFN-γ and TNF-α, increased cytotoxicity as well as degranulation, perforin and granzyme B expression in response to tumor cell targets.

Furthermore, the inventors observed that CAR-NKs showed superior cytotoxicity against two BCMA antigen high expressing cell lines, when compared to CAR-T cells. Without intending to be limited to any theory, NKs have germline receptors on their surface including a variety of activating receptors and inhibitory receptors that can exert cytotoxicity via non-CAR as well as CAR-mediated modes, this is shown by the killing of tumor cells by non-transduced NKs. Thus, NK cells can provide an advantage in circumstances when heterogeneous tumor targets evade CAR-T therapy with tumor cells having lower antigen expressing on surface. Additionally, it was observed in the examples that CAR-NKs generally produced more cytokines (IFN-γ, TNF-α, GM-CSF, etc.) in response to target cells. This may play pivotal roles in shaping the adaptive immune responses against tumor via modulating the dual switch (“DS”), macrophages, and T cell responses.

Non-limiting examples of chimeric polypeptides useful for inducing cell activation, and related methods for inducing activation including, for example, expression constructs, methods for constructing vectors, and assays for activity or function, may also be found in the following patents and patent applications, each of which is incorporated by reference herein in its entirety for all purposes. U.S. patent application Ser. No. 14/842,710, filed Sep. 1, 2015, published as US2016-0058857-A1 on Mar. 3, 2016, entitled “COSTIMULATION OF CHIMERIC ANTIGEN RECEPTORS BY MYD88 AND CD40 POLYPEPTIDES,” U.S. patent application Ser. No. 14/210,034, filed Mar. 13, 2014, entitled METHODS FOR CONTROLLING T CELL PROLIFERATION, published Sep. 25, 2014 as US2014-0286987-A1; International Patent Application No. PCT/US2014/026734, filed Mar. 13, 2014, published Sep. 25, 2014 as WO2014/151960, by Spencer et al.; U.S. patent application Ser. No. 14/622,018, filed Feb. 13, 2014, entitled METHODS FOR ACTIVATING T CELLS USING AN INDUCIBLE CHIMERIC POLYPEPTIDE, published Feb. 18, 2016 as US2016-0046700-A1; International Patent Application No. PCT/US2015/015829, filed Feb. 13, 2015, published Aug. 20, 2015 as WO2015/123527; U.S. patent application Ser. No. 10/781,384, filed Feb. 18, 2004, entitled INDUCED ACTIVATION OF DENDRITIC CELLS, published Oct. 21, 2004 as US2004-0209836-A1, issued Jun. 29, 2008 as U.S. Pat. No. 7,404,950, by Spencer et al.; International Patent Application No. PCT/US2004/004757, filed Feb. 18, 2004, published Mar. 24, 2005 as WO2004/073641A3; U.S. patent application Ser. No. 12/445,939, filed Oct. 26, 2010, entitled METHODS AND COMPOSITIONS FOR GENERATING AN IMMUNE RESPONSE BY INDUCING CD40 AND PATTERN RECOGNITION RECEPTORS AND ADAPTORS THEREOF, published Feb. 10, 2011 as US2011-0033388-A1, issued Apr. 8, 2014 as U.S. Pat. No. 8,691,210, by Spencer et al.; International Patent Application No. PCT/US2007/081963, filed Oct. 19, 2007, published Apr. 24, 2008 as WO2008/049113; U.S. patent application Ser. No. 13/763,591, filed Feb. 8, 2013, entitled METHODS AND COMPOSITIONS FOR GENERATING AN IMMUNE RESPONSE BY INDUCING CD40 AND PATTERN RECOGNITION RECEPTOR ADAPTERS, published Mar. 27, 2014 as US2014-0087468-A1, issued Apr. 19, 2016 as U.S. Pat. No. 9,315,559, by Spencer et al.; International Patent Application No. PCT/US2009/057738, filed Sep. 21, 2009, published Mar. 25, 2010 as WO201033949; U.S. patent application Ser. No. 13/087,329, filed Apr. 14, 2011, entitled METHODS FOR TREATING SOLID TUMORS, published Nov. 24, 2011 as US2011-0287038-A1, by Slawin et al.; International Patent Application No. PCT/US2011/032572, filed Apr. 14, 2011, published Oct. 20, 2011 as WO2011/130566, by Slawin et al; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; International Patent Application No. PCT/US2016/066371, filed Dec. 13, 2016, published Jun. 22, 2017 as WO2017/106185, by Bayle et al.; and U.S. Provisional Patent Application No. 62/503,565, filed May 9, 2017, entitled METHODS TO AUGMENT OR ALTER SIGNAL TRANSDUCTION, by Bayle et al., each of which is incorporated by reference herein in its entirety for all purposes.

Non-limiting examples of chimeric polypeptides useful for inducing cell death or apoptosis, and related methods for inducing cell death or apoptosis, including expression constructs, methods for constructing vectors, assays for activity or function, and multimerization of the chimeric polypeptides by contacting cells that express inducible chimeric polypeptides with a multimeric compound, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. patent application Ser. No. 13/112,739, filed May 20, 2011, entitled METHODS FOR INDUCING SELECTIVE APOPTOSIS, published Nov. 24, 2011, as US2011-0286980-A1, issued Jul. 28, 2015 as U.S. Pat. No. 9,089,520; U.S. patent application Ser. No. 13/792,135, filed Mar. 10, 2013, entitled MODIFIED CASPASE POLYPEPTIDES AND USES THEREOF, published Sep. 11, 2014 as US2014-0255360-A1, issued Sep. 6, 2016 as U.S. Pat. No. 9,434,935, by Spencer et al.; International Patent Application No. PCT/US2014/022004, filed Mar. 7, 2014, published Oct. 9, 2014 as WO2014/16438; U.S. patent application Ser. No. 14/296,404, filed Jun. 4, 2014, entitled METHODS FOR INDUCING PARTIAL APOPTOSIS USING CASPASE POLYPEPTIDES, published Jun. 2, 2016 as US2016-0151465-A1, by Slawin et al; International Application No. PCT/US2014/040964 filed Jun. 4, 2014, published as WO2014/197638 on Feb. 5, 2015, by Slawin et al.; U.S. patent application Ser. No. 14/640,553, filed Mar. 6, 2015, entitled CASPASE POLYPEPTIDES HAVING MODIFIED ACTIVITY AND USES THEREOF, published Nov. 19, 2015 as US2015-0328292-A1; International Patent Application No. PCT/US2015/019186, filed Mar. 6, 2015, published Sep. 11, 2015 as WO2015/134877, by Spencer et al.; U.S. patent application Ser. No. 14/968,737, filed Dec. 14, 2015, entitled METHODS FOR CONTROLLED ELIMINATION OF THERAPEUTIC CELLS, published Jun. 16, 2016 as US2016-0166613-A1, by Spencer et al.; International Patent Application No. PCT/US2015/065629 filed Dec. 14, 2015, published Jun. 23, 2016 as WO2016/100236, by Spencer et al.; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; and International Patent Application No. PCT/US2016/066371, filed Dec. 13, 2016, published Jun. 22, 2017 as WO2017/106185, by Bayle et al., each of which is incorporated by reference herein in its entirety for all purposes. Multimeric compounds described herein, or pharmaceutically acceptable salts thereof, may be used essentially as discussed in examples provided in these publications, and other examples provided herein.

Thus, provided in some embodiments is a modified NK cell, comprising a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In some embodiments, the NK cell is cryostored. In some embodiments, the modified NK cell has been stored at a temperature of 0° C., −25 degrees C., −50 degrees C., −75 degrees C., −100 degrees C., −125 degrees C., −150 degrees C., −175 degrees C., or −200 degrees C. or below. In some embodiments, the modified cell comprises a polynucleotide that encodes an IL-15 polypeptide. In certain embodiments, the modified NK cell further comprises: one, two, or all of the following: (1) a polynucleotide encoding IL-15, (2) a polynucleotide encoding a chimeric pro-apoptotic polypeptide described herein, or (3) a CAR.

Provided in some embodiments is a method for cryopreserving NK cells, comprising storing modified NK cells at a temperature below −150° C., wherein the NK cells comprise a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

In some embodiments, the modified NK cells may be cryopreserved or frozen at a temperature of 0, −25, −50, −75, −100, −125, −150, −175, or −200 degrees Celsius or below. In some embodiments, the modified NK cells may be thawed following storage at a temperature of 0, −25, −50, −75, −100, −125, −150, −175, or −200 degrees Celsius or below.

Provided in some embodiments is a method for growing NK cells ex vivo, comprising incubating modified NK cells in cell culture medium, wherein the modified NK cells comprise a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In certain embodiments, the modified NK cells further comprise: one, two, or all of the following: (1) a polynucleotide encoding IL-15, (2) a polynucleotide encoding a chimeric pro-apoptotic polypeptide described herein, or (3) a CAR.

Provided in some embodiments is a method for thawing NK cells comprising thawing frozen modified NK cells, wherein the modified NK cells comprise a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40, wherein the modified NK cells have been stored at a temperature of 0° C. or below. In some embodiments, the method comprises the step of transfecting or transducing NK cells with a nucleic acid comprising a polynucleotide encoding the chimeric polypeptide. In certain embodiments, the modified NK cells further comprise: one, two, or all of the following: (1) a polynucleotide encoding IL-15, (2) a polynucleotide encoding a chimeric pro-apoptotic polypeptide described herein, or (3) a CAR. In some embodiments, the method comprises cooling the modified NK cells to a temperature of 0° C. or below. In some embodiments, the method comprises cooling the modified NK cells to a temperature of −150° C. or below. In some embodiments, the method comprises thawing the modified NK cells.

In some embodiments, a method is provided for stimulating an immune response comprising transfecting or transducing a NK cell ex vivo with a nucleic acid comprising a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In some embodiments, the method comprises the step of transfecting or transducing NK cells with a nucleic acid comprising a polynucleotide encoding the chimeric polypeptide. In some embodiments, the immune response is a cytotoxic response. In some embodiments, the immune response is a cytolytic response. In some embodiments, the immune response is an anti-tumor response. In certain embodiments, the modified NK cell further comprises: one, two, or all of the following: (1) a polynucleotide encoding IL-15, (2) a polynucleotide encoding a chimeric pro-apoptotic polypeptide described herein, or (3) a CAR.

In some embodiments, a method is provided for stimulating an immune response comprising administering modified NK cells to a subject, wherein the modified NK cells comprise a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In certain embodiments, the modified NK cells further comprise: one, two, or all of the following: (1) a polynucleotide encoding IL-15, (2) a polynucleotide encoding a chimeric pro-apoptotic polypeptide described herein, or (3) a CAR.

In some embodiments, a method is provided for treating a subject having a disease or condition associated with an elevated expression of a target antigen expressed by a target cell, comprising transplanting an effective amount of modified NK cells into the subject; wherein the modified NK cells comprise a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In certain embodiments, the modified NK cells further comprise: one, two, or all of the following: (1) a polynucleotide encoding IL-15, (2) a polynucleotide encoding a chimeric pro-apoptotic polypeptide described herein, or (3) a CAR. In some embodiments, the target antigen is a tumor antigen.

In some embodiments, a method is provided for reducing the size of a tumor in a subject, comprising transplanting an effective amount of modified NK cells into the subject; wherein the modified NK cells comprise a polynucleotide encoding a chimeric polypeptide, wherein the chimeric polypeptide comprises a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In certain embodiments, the modified NK cell further comprises: one, two, or all of the following: (1) a polynucleotide encoding IL-15, (2) a polynucleotide encoding a chimeric pro-apoptotic polypeptide described herein, or (3) a CAR.

In some embodiments, the method comprises administering an effective amount of a ligand that binds to a multimerizing region of the chimeric costimulating polypeptide to reduce the number or concentration of target antigen or to reduce the number of target cells in the subject. The method comprise transplanting an effective amount of modified NK cells into the subject; wherein the modified NK cells comprise a polynucleotide encoding a chimeric polypeptide, wherein the chimeric polypeptide comprises a ligand binding region; and a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In certain embodiments, the modified NK cell further comprises: one, two, or all of the following: (1) a polynucleotide encoding IL-15, (2) a polynucleotide encoding a chimeric pro-apoptotic polypeptide described herein, or (3) a CAR.

In some embodiments, a method is provided for administering a ligand to a human subject who has undergone cell therapy using modified NK cells, wherein the modified NK cells comprise a polynucleotide encoding a chimeric polypeptide, wherein the chimeric polypeptide comprises a ligand binding region; and a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In certain embodiments, the modified NK cells further comprise: one, two, or all of the following: (1) a polynucleotide encoding IL-15, (2) a polynucleotide encoding a chimeric pro-apoptotic polypeptide described herein, or (3) a CAR.

In some embodiments, a kit is provided, comprising modified natural killer (NK) cells described herein, wherein the modified NK cells have been stored at a temperature of 0° C. or below. In some embodiments, the kit comprises a ligand that binds to a ligand binding region. In some embodiments, the kit comprises an antibody. In some embodiments, the antibody binds to an antigen on a target cell. In some embodiments, the antibody is formulated for priming the NK cells stimulate an immune response against the target cell. In some embodiments, the target cell is a tumor cell.

In some embodiments, the chimeric polypeptide comprises a first ligand binding region and a second ligand region, wherein the first ligand binding region has a different amino acid sequence than the second ligand binding region, and the first and second ligand binding regions bind to a heterodimeric ligand. In some embodiments, the first ligand binding region binds to a first portion of the heterodimeric ligand, and the second ligand binding region binds to a second portion of the heterodimeric ligand. In some embodiments, the chimeric polypeptide of any one of embodiments described above is a first chimeric polypeptide, and the cell comprises a second chimeric polypeptide, the first ligand binding region of the first chimeric polypeptide and a second ligand binding region of the second chimeric polypeptide binds to the second portion of the heterodimeric ligand.

Provided in some embodiments is a modified natural killer (NK) cell, comprising a first and a second polynucleotide, wherein the first polynucleotide encodes a chimeric polypeptide comprising a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In some embodiments, the second polynucleotide encodes an IL-15 polypeptide. In some embodiments, the chimeric polypeptide comprises a ligand binding region.

In some embodiments, a nucleic acid is provided comprising a first and a second polynucleotide, wherein the first polynucleotide encodes a chimeric polypeptide comprising a signaling region, comprising a MyD88 polypeptide; a truncated MyD88 polypeptide lacking the TIR domain; a MyD88 polypeptide and a CD40 cytoplasmic polypeptide region lacking the CD40 extracellular domain; a truncated MyD88 polypeptide lacking the TIR domain and a CD40 cytoplasmic polypeptide region lacking the CD40 extracellular domain; a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40. In some embodiments, the chimeric polypeptide further comprises a ligand binding region. In some embodiments, the second polynucleotide encodes an IL-15 polypeptide. In some embodiments, the nucleic acid comprises a polynucleotide that encodes a chimeric antigen receptor (CAR) or a T-cell receptor. In some embodiments, a nucleic acid is provided comprising a first, a second, and a third polynucleotide, wherein the first polynucleotide encodes a chimeric polypeptide comprising a first ligand binding region, a second ligand binding region, a truncated MyD88 polypeptide lacking the TIR domain and a CD40 cytoplasmic polypeptide region lacking the CD40 extracellular domain, the second polynucleotide encodes an IL-15 polypeptide, and the third polynucleotide encodes a chimeric antigen receptor. In some embodiments the ligand binding region comprises an FKBP12v36 polypeptide. In some embodiments, the nucleic acid is incorporated in a modified NK cell.

In some embodiments, a nucleic acid is provided comprising a first, a second, and a third polynucleotide, wherein the first polynucleotide encodes a chimeric polypeptide comprising a truncated MyD88 polypeptide lacking the TIR domain and a CD40 cytoplasmic polypeptide region lacking the CD40 extracellular domain, the second polynucleotide encodes an IL-15 polypeptide, and the third polynucleotide encodes a chimeric polypeptide comprising a caspase-9 polypeptide lacking the CARD domain. The three polynucleotides may be positioned in various 5 to 3′ order on the nucleic acid. In some embodiments, the chimeric polypeptide further comprises a ligand binding region. In some embodiments, the ligand binding region comprises an FKBP12v36 polypeptide. In some embodiments, the third polynucleotide encodes a chimeric polypeptide comprising a caspase-9 polypeptide lacking the CARD domain comprises a second and a third ligand binding region. In some embodiments, the second ligand binding region comprises an FKBP12 polypeptide and the third ligand binding region comprises an FRB polypeptide or an FRB variant polypeptide. In some embodiments, the nucleic acid comprises a polynucleotide encoding a marker polypeptide, in some embodiments the marker polypeptide comprises a truncated CD19 polypeptide. In some embodiments, the nucleic acid is incorporated in a modified NK cell. In some embodiments, the modified NK cell comprises a second nucleic acid comprising a polynucleotide that encodes a chimeric antigen receptor. In some embodiments, the second nucleic acid comprises a polynucleotide encoding a marker polypeptide, in some embodiments the marker polypeptide comprises a truncated CD19 polypeptide. In some embodiments, a modified NK cell is provided that comprises a first nucleic acid and a second nucleic acid, where the first nucleic acid comprises a first polynucleotide encoding two FKBP12v36 polypeptides, a truncated MyD88 polypeptide, and a CD40 cytoplasmic polypeptide region lacking the CD40 extracellular domain; a second polynucleotide encoding an IL-15 polypeptide, and a third polynucleotide encoding chimeric polypeptide comprising a caspase-9 polypeptide lacking the CARD domain, an FKBP12 polypeptide, and an FRB polypeptide or FRB variant polypeptide, and a fourth nucleic acid comprising a polynucleotide encoding a chimeric antigen receptor, and wherein the first nucleic acid or the second nucleic acid optionally comprise a polynucleotide encoding a marker polypeptide.

In some embodiments, a nucleic acid is provided comprising a first, a second, and a third polynucleotide, wherein the first polynucleotide encodes a chimeric polypeptide comprising a first ligand binding region, a second ligand binding region, a truncated MyD88 polypeptide lacking the TIR domain and a CD40 cytoplasmic polypeptide region lacking the CD40 extracellular domain, the second polynucleotide encodes an IL-15 polypeptide, and the third polynucleotide encodes a chimeric antigen receptor. The three polynucleotides may be positioned in various 5 to 3′ order on the nucleic acid. In some embodiments, the first, second, and third polynucleotides are positioned on the nucleic acid in 5′ to 3′ order of first polynucleotide, third polynucleotide, and second polynucleotide. In some embodiments the first ligand binding region comprises an FKBP12v36 polypeptide and the second ligand binding region comprises an FKBP12v36 polypeptide. In some embodiments, the nucleic acid is incorporated in a modified NK cell. In some embodiments, the modified NK cell comprises a second nucleic acid comprising a polynucleotide that encodes a chimeric polypeptide comprising a caspase-9 polypeptide lacking the CARD domain, and a third and a fourth ligand binding region. In some embodiments, the third ligand binding region comprises an FKBP12 polypeptide and the fourth ligand binding region comprises an FRB polypeptide or an FRB variant polypeptide. In some embodiments, the second nucleic acid comprises a polynucleotide encoding a marker polypeptide, in some embodiments the marker polypeptide comprises a truncated CD19 polypeptide. In some embodiments, a first nucleic acid is provided that comprises a first polynucleotide encoding two FKBP12v36 polypeptides, a truncated MyD88 polypeptide, and a CD40 cytoplasmic polypeptide region lacking the CD40 extracellular domain; a second polynucleotide encoding an IL-15 polypeptide, and a third polynucleotide encoding a chimeric antigen receptor. In some embodiments, a modified cell is provided comprising the first nucleic acid. In some embodiments, the modified cell is a NK cell. In some embodiments, a modified cell is provided comprising the first nucleic acid and a second nucleic acid, where the second nucleic acid comprises a polynucleotide encoding a chimeric polypeptide comprising a caspase-9 polypeptide lacking the CARD domain, an FKBP12 polypeptide, and an FRB polypeptide or FRB variant polypeptide, and wherein the first nucleic acid or the second nucleic acid optionally comprise a polynucleotide encoding a marker polypeptide.

In some embodiments, pharmaceutical compositions are provided that comprise the modified cells or nucleic acids of the present application. In some embodiments, pharmaceutical compositions are provided that are prepared by the methods of the present application.

In some embodiments, the modified NK cell, method, kit, nucleic acid, or pharmaceutical composition of the present embodiments comprise a polynucleotide encoding an IL-15 polypeptide. In some embodiments, the modified NK cell, kit, or pharmaceutical composition is transfected or transduced with a nucleic acid that comprises a polynucleotide encoding an IL-15 polypeptide.

Expression of recombinant vectors encoding gene fusions between a truncation allele of MyD88 (myeloid differentiation primary response 88) and the intracellular signaling domains of certain transmembrane receptors generates signaling nodes that enhance the signaling capacity of MyD88 itself. These chimeric signaling polypeptides may have constitutive activity, or may include multimeric ligand binding regions that, upon binding to a multimeric ligand induce multimerization and activation of the chimeric signaling polypeptide. Immune cells may express the chimeric signaling polypeptide as part of a chimeric antigen receptor polypeptide, or the chimeric signaling polypeptide may be expressed as a separate polypeptide from the antigen recognition polypeptide, for example, the CAR (chimeric antigen receptor) or rTCR (recombinant T cell receptor).

In some embodiments, the natural killer cells are transduced or transfected with a nucleic acid comprising a promoter operably linked to a polynucleotide encoding a chimeric signaling polypeptide, wherein the polypeptide comprises a MyD88 polypeptide or a truncated MyD88 polypeptide lacking a TIR domain; and a CD40 polypeptide cytoplasmic region that is lacking the extracellular domain. In some embodiments, the promoter is operably linked to a polynucleotide encoding a chimeric signaling polypeptide, wherein the polypeptide comprises a multimeric ligand binding region that binds to a multimeric ligand; a MyD88 polypeptide or a truncated MyD88 polypeptide lacking a TIR domain; and a CD40 polypeptide cytoplasmic region that is lacking the extracellular domain. In some embodiments, the natural killer cells are transduced or transfected with a nucleic acid comprising a promoter operably linked to a polynucleotide encoding a chimeric signaling polypeptide, a MyD88 polypeptide or a truncated MyD88 polypeptide lacking a TIR domain; and a co-stimulatory polypeptide cytoplasmic signaling region with the proviso that the co-stimulatory polypeptide cytoplasmic signaling region is not CD40. In some embodiments, the co-stimulatory polypeptide cytoplasmic signaling region is selected from the group consisting of CD27, CD28, ICOS, 4-1BB, RANK/TRANCE-R, OX40, CD30, TweakR, TAC1, BCMA and HVEM cytoplasmic signaling regions and is not a CD40 polypeptide. In some embodiments, the chimeric signaling polypeptide is an inducible chimeric signaling polypeptide comprising a multimeric ligand binding region that binds to a multimeric ligand. In other embodiments, the co-stimulatory polypeptide cytoplasmic signaling region is selected from the group consisting of CD27, CD28, ICOS, 4-1BB, RANK/TRANCE-R, and OX40, and the inducible chimeric signaling polypeptide further comprises a CD40 polypeptide lacking the extracellular region.

Co-stimulatory polypeptide cytoplasmic signaling regions of the inducible chimeric signaling polypeptides and chimeric signaling polypeptides herein may, for example, activate the NF-κB pathway, and are selected from non-CD40 NF-κB inducers such as, for example, CD28 or TNFR family members.

Also provided in some embodiments the NK cells are transduced or transfected with nucleic acids comprising a polynucleotide encoding an inducible chimeric signaling polypeptide, wherein the chimeric signaling polypeptide comprises functional domains, or functional regions. Functional domains or functional regions may be selected from the group consisting of MyD88 polypeptides or truncated MyD88 polypeptides, co-stimulatory polypeptide cytoplasmic signaling regions, multimeric ligand binding regions, and membrane targeting regions. In some embodiments, the functional domains consist of a) one or more multimeric ligand binding regions that bind to a multimeric ligand; b) a MyD88 polypeptide or a truncated MyD88 polypeptide lacking the TIR domain; and c) a costimulatory polypeptide cytoplasmic signaling region. Thus, in some embodiments, the MyD88 polypeptide domain comprises a full length MyD88 polypeptide, in some embodiments, the MyD88 polypeptide domain comprises a truncated MyD88 polypeptide lacking the TIR domain, in some embodiments, the truncated MyD88 polypeptide comprises a polypeptide that comprises the amino acid sequence of SEQ ID NO: 2 or SEQ ID NO: 119, in some embodiments, the truncated MyD88 polypeptide consists of the amino acid sequence of SEQ ID NO: 2 or SEQ ID NO: 119. Also, in some embodiments, the MyD88 polypeptide domain consists of a full length MyD88 polypeptide, in some embodiments, the MyD88 polypeptide domain consists of a truncated MyD88 polypeptide lacking the TIR domain, in some embodiments, the truncated MyD88 polypeptide consists of a polypeptide that comprises the amino acid sequence of SEQ ID NO: 2 or SEQ ID NO: 119. The chimeric signaling polypeptides may also comprise additional polypeptides, which may also be referred to as non-functional polypeptides, such as, for example, 2A polypeptides, marker polypeptides, and linker polypeptides. In some embodiments, the multimeric ligand binding regions comprise FKBP12 variant polypeptides of the present application, such as, for example, FKBP12 variant polypeptides having amino acid substitutions at position 36, and, for example, FKBP12v36. In some embodiments, functional domain (a) comprises two FKBP12 variant polypeptides, such as, for example, FKBP12 variant polypeptides having amino acid substitutions at position 36, and, for example, FKBP12v36. In some examples domain (b) comprises a truncated MyD88 polypeptide lacking the TIR domain, such as, for example, the truncated MyD88 polypeptides of the present application. In some embodiments, the costimulatory polypeptide is a CD40 polypeptide cytoplasmic region lacking the extracellular domain. In some embodiments, the costimulatory polypeptide of domain (c) is selected from the group consisting of CD27, CD28, ICOS, 4-1BB, RANK/TRANCE-R, OX40, CD30, TweakR, TAC1, BCMA and HVEM cytoplasmic signaling regions, or a functional fragment thereof. In other embodiments, the co-stimulatory polypeptide cytoplasmic signaling region is selected from the group consisting of CD27, CD28, ICOS, 4-1BB, RANK/TRANCE-R, and OX40 cytoplasmic signaling regions, and is not a CD40 polypeptide. In other embodiments, the co-stimulatory polypeptide cytoplasmic signaling region is selected from the group consisting of CD27, CD28, ICOS, 4-1BB, RANK/TRANCE-R, and OX40, or a functional fragment thereof. In some embodiments, the co-stimulatory polypeptide cytoplasmic signaling region consists of a cytoplasmic signaling region of a co-stimulatory polypeptide selected from the group consisting of CD27, CD28, ICOS, 4-1BB, RANK/TRANCE-R and OX40, or a functional fragment thereof. In some embodiments, the co-stimulatory polypeptide cytoplasmic signaling region comprises a cytoplasmic signaling region of a co-stimulatory polypeptide selected from the group consisting of CD28, ICOS, 4-1BB, and OX40. In some embodiments, the co-stimulatory polypeptide cytoplasmic signaling region consists of a cytoplasmic signaling region of a co-stimulatory polypeptide selected from the group consisting of CD28, ICOS, 4-1BB, and OX40.

In some embodiments, the NK cell is transduced or transfected with a nucleic acid that comprises a polynucleotide coding for a chimeric signaling polypeptide or an inducible chimeric signaling polypeptide that further comprises a membrane targeting region. In some embodiments, the membrane targeting region is selected from the group consisting of a myristoylation region, a palmitoylation region, a prenylation region, and transmembrane sequences of receptors. In some embodiments, the membrane-targeting region is a myristoylation region.

In some embodiments, the nucleic acid further comprises a polynucleotide encoding a chimeric Caspase-9 polypeptide comprising a multimeric ligand binding region and a Caspase-9 polypeptide.

Provided in some embodiments are modified cells, wherein the cell is transduced or transfected with a nucleic acid of any one of the present embodiments. In some embodiments, the cell is also transduced or transfected with a nucleic acid comprising a polynucleotide coding for a heterologous protein, a marker polypeptide, a chimeric antigen receptor, a recombinant T cell receptor. Also provided in certain embodiments are methods for expressing an inducible chimeric signaling polypeptide, or a chimeric signaling polypeptide in a cell, comprising contacting a nucleic acid of any one of the present embodiments with a cell under conditions in which the nucleic acid is incorporated into the cell, whereby the cell expresses the inducible chimeric signaling polypeptide or the chimeric signaling polypeptide from the incorporated nucleic acid.

Provided in certain embodiments are methods for stimulating a cell-mediated immune response in a subject, comprising administering a modified cell transfected or transduced with a nucleic acid as described herein.

### Costimulation

In some embodiments, the invention provides compositions and methods comprising a NK cell population comprising a costimulatory polypeptide.

The costimulatory polypeptide of the present invention can be inducible or constitutively activated. The costimulatory polypeptide can comprise one or more costimulatory signaling regions such as CD27, ICOS, RANK, TRANCE, CD28, 4-1BB, OX40, DAP10, MyD88, or CD40 or, for example, the cytoplasmic regions thereof. The costimulatory polypeptide can comprise one or more suitable costimulatory signaling regions that activate the signaling pathways activated by CD27, ICOS, RANK, TRANCE, CD28, 4-1BB, OX40, DAP10, MyD88, or CD40. Costimulating polypeptides include any molecule or polypeptide that activates the NF-κB pathway, Akt pathway, and/or p38 pathway of tumor necrosis factor receptor (TNFR) family (i.e., CD40, RANK/TRANCE-R, OX40, 4-1BB) and CD28 family members (CD28, ICOS). More than one costimulating polypeptide or costimulating polypeptide cytoplasmic region may be expressed in the modified T cells discussed herein.

### Costimulation Provided by MyD88 and CD40

In some embodiments, the NK cell population describe herein comprise a costimulatory polypeptide. The costimulatory polypeptide can comprise one or more costimulatory signaling regions that activate the signaling pathways activated by CD27, ICOS, RANK, TRANCE, CD28, 4-1BB, OX40, DAP10, MyD88, or CD40.

Provided herein are NK cells that comprise chimeric signaling polypeptides, including, for example, chimeric signaling polypeptides where the truncated MyD88 polypeptide has also been fused with signaling domains of receptor mediators of costimulation, such as, for example, CD40, CD27, CD28, 4-1BB, OX40, or ICOS.

In some embodiments, the NK cell population described herein comprises a costimulatory polypeptide comprising one or more costimulatory signaling regions that activate the signaling pathways activated by MyD88, CD40 and/or MyD88-CD40 fusion chimeric polypeptide.

MyD88 is an universal adaptor molecule for TLRs and a critical signaling component of the innate immune system, triggering an alert for foreign invaders, priming immune cell recruitment and activation. MyD88 is a cytosolic adapter protein that plays a central role in the innate and adaptive immune response. This protein functions as an essential signal transducer in the interleukin-1 and Toll-like receptor signaling pathways. These pathways regulate that activation of numerous proinflammatory genes. The encoded protein consists of an N-terminal death domain and a C-terminal Toll-interleukin1 receptor domain. MyD88 TIR domain is able to heterodimerize with TLRs and homodimerize with other MyD88 proteins. This in turn results in recruitment and activation of IRAK family kinases through interaction of the death domains (DD) at the amino terminus of MyD88 and IRAK kinases which thereby initiates a signaling pathway that leads to activation of JNK, p38 MAPK (mitogen-activated protein kinase) and NF-κB, a transcription factor that induces expression of cytokine- and chemokine-encoding genes (as well as other genes). MyD88 acts via IRAK1, IRAK2, IRF7 and TRAF6, leading to NF-kappa-B activation, cytokine secretion and the inflammatory response. It also activates IRF1 resulting in its rapid migration into the nucleus to mediate an efficient induction of IFN-beta, NOS2/INOS, and IL12A genes. MyD88-mediated signaling in intestinal epithelial cells is crucial for maintenance of gut homeostasis and controls the expression of the antimicrobial lectin REG3G in the small intestine.

CD40 is an important part of the adaptive immune response, aiding to activate APCs through engagement with its cognate CD40L, in turn polarizing a stronger CTL response. The CD40/CD154 signaling system is an important component in T cell function and B cell-T cell interactions. CD40 signaling proceeds through formation of CD40 homodimers and interactions with TNFR-associated factors (TRAFs), carried out by recruitment of TRAFs to the cytoplasmic domain of CD40, which leads to T cell activation involving several secondary signals such as the NF-κB, JNK and AKT pathways

Inducible MyD88/CD40 (IMC): This activation switch includes a fusion of the signaling domains for MyD88 and the intracellular signaling domain of CD40 together with ligand binding domains derived from FKBP12 that are sensitive to the presence of dimerizing analogs of FK506 and rapamycin, typically rimiducid (also known as AP1903). Ligand binding induces the oligomerization of iMC which nucleates downstream signaling events, leading to gene expression, cytokine production, cell proliferation and cell survival. When coexpressed in T cells with a first-generation Chimeric Antigen Receptor (CAR), iMC provides costimulation that is enhanced by ligand binding.

Fusions of a truncated MyD88 polypeptide, lacking the TIR domain with the intracellular domain of CD40 (“MC”) to produce a chimeric polypeptide amplifies certain signals directed by MyD88. When T cells are transfected or transduced with nucleic acids that encode MC, in combination with a Chimeric Antigen Receptor (CAR), MC delivers potent costimulatory signals that enhance T cell growth, persistence, and cytotoxic activity against cells specifically targeted by the CAR. (see, for example, U.S. patent application Ser. No. 14/842,710, titled Costimulation of Chimeric Antigen Receptors by MyD88 and CD40 polypeptide, by Spencer, D., et al, filed Sep. 1, 2015, published as US-2016-0058857A1 on Mar. 3, 2016; and International Patent Application PCT/US2015/047957, filed Dec. 14, 2015, published as WO/2016/036746 on Mar. 10, 2016, all incorporated herein by reference in their entireties).

In some embodiments, inducible chimeric signaling polypeptides may comprise a truncated MyD88 polypeptide lacking the TIR domain, a cytoplasmic signaling domain selected from the group consisting of CD40, CD28, 4-1BB, OX-40, and ICOS, and a multimeric ligand binding region such as an FKBP12 multimeric ligand binding region, for example, a wild type FKBP12 multimeric ligand binding region (Fwt) or a FKBP12 variant polypeptide that is inducible with the dimerizing small molecule AP1903 (rimiducid) or AP20187, such as, for example, a FKBP12 variant polypeptide that has an amino acid substitution at amino acid 36, substituting a different amino acid for the phenylalanine residue at position 36, for example, valine (FKBP12v36, Fv). The inducible forms of these fusions generate differential activity to transduce activating signals to the NF-κB family of transcription factors when activated with rimiducid. NF-κB is a key mediator of costimulation, cell survival and cytokine production.

In some embodiments, the cell populations provided herein comprise NK cells designed to provide constitutively active therapy. In some embodiments, the NK cells comprise a nucleic acid comprising a first polynucleotide encoding the CAR, and a second polynucleotide encoding a chimeric signaling polypeptide. In some embodiments, the second polynucleotide is positioned 5′ of the first polynucleotide. In some embodiments, the second polynucleotide is positioned 3′ of the first polynucleotide. In some embodiments, a third polynucleotide encoding a linker polypeptide is positioned between the first and second polynucleotides. Where the third polynucleotide is positioned 3′ of the first polynucleotide and 5′ of the second polynucleotide, the linker polypeptide, may remain intact following translation, or may separate the polypeptides encoded by the first and second polynucleotides during, or after translation. In some embodiments, the linker polypeptide is a 2A polypeptide, which may separate the polypeptides encoded by the first and second polynucleotides during, or after translation. High level costimulation is provided constitutively through an alternate mechanism in which a leaky 2A cotranslational sequence, for example one derived from porcine teschovirus-1 (P2A), is used to separate the CAR from the chimeric signaling polypeptide. Where the 2A separation is incomplete, for example from a leaky 2A sequence, most of the expressed chimeric signaling polypeptide molecules are separated from the chimeric antigen receptor polypeptide and may remain cytosolic, and some portion or the chimeric signaling polypeptide molecules remain attached, or linked, to the CAR.

By “constitutively active” is meant that the chimeric stimulating molecule's (e.g., chimeric signaling polypeptide's) NK cell activation activity, as demonstrated herein, is active in the absence of an inducer. Constitutively active chimeric stimulating molecules in the present application (e.g., chimeric signaling polypeptides) do not comprise a multimeric ligand binding region, or a functional multimeric ligand binding region, and are not inducible by AP1903, AP20187, or other CID.

In some embodiments, the chimeric signaling polypeptide comprises a truncated MyD88 polypeptide and a CD40 polypeptide lacking the extracellular domain, or two costimulatory polypeptides cytoplasmic signaling regions. In some embodiments, the chimeric signaling polypeptide comprises two costimulatory polypeptides cytoplasmic signaling regions, such as, for example, 4-1BB and CD28, or one, or two or more costimulatory polypeptide cytoplasmic signaling regions selected from the group consisting of CD27, ICOS, RANK, TRANCE, CD28, 4-1BB, OX40, DAP10. In some embodiments, the chimeric signaling polypeptide comprises a MyD88 polypeptide or a truncated MyD88 polypeptide and a costimulatory polypeptide cytoplasmic signaling region selected from the group consisting of CD27, ICOS, RANK, TRANCE, CD28, 4-1BB, OX40, DAP10.

Also provided in some embodiments, are cell populations provided herein that comprise an inducible safety switch, to stop, or reduce the level of, the therapy when needed. In some embodiments, immune cells, such as NK cells, express a chimeric antigen receptor, and a chimeric signaling polypeptide comprising, for example, a truncated MyD88 polypeptide and a CD40 polypeptide lacking the extracellular domain, or two costimulatory polypeptides cytoplasmic signaling regions

Costimulation in T cells that express chimeric antigen receptors by MyD88 and CD40 polypeptides, and by chimeric signaling polypeptides comprising costimulatory polypeptide cytoplasmic signaling regions is discussed in U.S. patent application Ser. No. 14/842,710, filed Sep. 1, 2015, published as US2016-0058857-A1 on Mar. 3, 2016, entitled “Costimulation of Chimeric Antigen Receptors by MyD88 and CD40 Polypeptides,” and to in U.S. Provisional Patent Application Ser. No. 62/503,565, filed May 9, 2017, entitled “Methods to Augment or Alter Signal Transduction.”

Non-limiting examples of chimeric polypeptides useful for inducing cell activation, and related methods for inducing CAR-T cell activation including, for example, expression constructs, methods for constructing vectors, and assays for activity or function, may also be found in the following patents and patent applications, each of which is incorporated by reference herein in its entirety for all purposes. U.S. patent application Ser. No. 14/210,034, filed Mar. 13, 2014, entitled METHODS FOR CONTROLLING T CELL PROLIFERATION, published Sep. 25, 2014 as US2014-0286987-A1; International Patent Application No. PCT/US2014/026734, filed Mar. 13, 2014, published Sep. 25, 2014 as WO2014/151960, by Spencer et al.; U.S. patent application Ser. No. 14/622,018, filed Feb. 13, 2014, entitled METHODS FOR ACTIVATING T CELLS USING AN INDUCIBLE CHIMERIC POLYPEPTIDE, published Feb. 18, 2016 as US2016-0046700-A1; International Patent Application No. PCT/US2015/015829, filed Feb. 13, 2015, published Aug. 20, 2015 as WO2015/123527; U.S. patent application Ser. No. 10/781,384, filed Feb. 18, 2004, entitled INDUCED ACTIVATION OF DENDRITIC CELLS, published Oct. 21, 2004 as US2004-0209836-A1, issued Jun. 29, 2008 as U.S. Pat. No. 7,404,950, by Spencer et al.; International Patent Application No. PCT/US2004/004757, filed Feb. 18, 2004, published Mar. 24, 2005 as WO2004/073641A3; U.S. patent application Ser. No. 12/445,939, filed Oct. 26, 2010, entitled METHODS AND COMPOSITIONS FOR GENERATING AN IMMUNE RESPONSE BY INDUCING CD40 AND PATTERN RECOGNITION RECEPTORS AND ADAPTORS THEREOF, published Feb. 10, 2011 as US2011-0033388-A1, issued Apr. 8, 2014 as U.S. Pat. No. 8,691,210, by Spencer et al.; International Patent Application No. PCT/US2007/081963, filed Oct. 19, 2007, published Apr. 24, 2008 as WO2008/049113; U.S. patent application Ser. No. 13/763,591, filed Feb. 8, 2013, entitled METHODS AND COMPOSITIONS FOR GENERATING AN IMMUNE RESPONSE BY INDUCING CD40 AND PATTERN RECOGNITION RECEPTOR ADAPTERS, published Mar. 27, 2014 as US2014-0087468-A1, issued Apr. 19, 2016 as U.S. Pat. No. 9,315,559, by Spencer et al.; International Patent Application No. PCT/US2009/057738, filed Sep. 21, 2009, published Mar. 25, 2010 as WO201033949; U.S. patent application Ser. No. 13/087,329, filed Apr. 14, 2011, entitled METHODS FOR TREATING SOLID TUMORS, published Nov. 24, 2011 as US2011-0287038-A1, by Slawin et al.; International Patent Application No. PCT/US2011/032572, filed Apr. 14, 2011, published Oct. 20, 2011 as WO2011/130566, by Slawin et al; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/047957, published as WO2016/036746 on Mar. 10, 2016, entitled COSTIMULATION OF CHIMERIC ANTIGEN RECEPTORS BY MYD88 AND CD40 POLYPEPTIDES; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; International Patent Application No. PCT/US2016/066371, filed Dec. 13, 2016, published Jun. 22, 2017 as WO2017/106185, by Bayle et al.; International Patent Application No. PCT/US2018/031689, filed May 8, 2018, entitled METHODS TO AUGMENT OR ALTER SIGNAL TRANSDUCTION, published Nov. 15, 2018 as WO2018/208849, by Bayle et al., each of which is incorporated by reference herein in its entirety, including all text, tables and drawings, for all purposes.

### IL-15

In a specific embodiment, IL-15 encoded by a polynucleotide described herein may by any naturally occurring interleukin-15 (e.g., mammalian IL-15), including the immature or precursor and mature forms. . . . Non-limiting examples of GeneBank Accession Nos. for the amino acid sequence of various species of native mammalian interleukin-15 include NP_000576 (human, immature form), CAA62616 (human, immature form), NP_001009207 (Felis catus, immature form), AAB94536 (rattus, immature form), AAB41697 (rattus, immature form), NP_032383 (Mus musculus, immature form), AAR19080 (canine), AAB60398 (macaca mulatta, immature form), AAI00964 (human, immature form), AAH23698 (Mus musculus, immature form), and AAH18149 (human). In a specific embodiment, IL-15 encoded by a polynucleotide described herein is an immature/precursor form of a naturally occurring human IL-15, which comprises an IL-15 signal peptide. In another embodiment, IL-15 encoded by a polynucleotide described herein is a mature form of a naturally occurring human IL-15.

In some embodiments, IL-15 encoded by a polynucleotide described herein is a derivative of a mammalian IL-15. The IL-15 derivative may include: (a) a polypeptide that is at least 40%, 45%, 50%, 55%, 60%, 65%, 70%, 75%, 80%, 85%, 90%, 95%, 98% or 99% identical to a naturally occurring mammalian IL-15 polypeptide; (b) a polypeptide encoded by a nucleic acid sequence that is at least 40%, 45%, 50%, 55%, 60%, 65%, 70%, 75%, 80%, 85%, 90%, 95%, 98% or 99% identical a nucleic acid sequence encoding a naturally occurring mammalian IL-15 polypeptide; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to a naturally occurring mammalian IL-15 polypeptide; (d) a polypeptide encoded by nucleic acids can hybridize under high, moderate or typical stringency hybridization conditions to nucleic acids encoding a naturally occurring mammalian IL-15 polypeptide; (e) a polypeptide encoded by a nucleic acid sequence that can hybridize under high, moderate or typical stringency hybridization conditions to a nucleic acid sequence encoding a fragment of a naturally occurring mammalian IL-15 polypeptide of at least 20 contiguous amino acids, at least 30 contiguous amino acids, at least 40 contiguous amino acids, at least 50 contiguous amino acids, at least 100 contiguous amino acids, or at least 150 contiguous amino acids; or (f) a fragment of a naturally occurring mammalian IL-15 polypeptide. IL-15 derivatives also include a polypeptide that comprises the amino acid sequence of a naturally occurring mature form of a mammalian IL-15 polypeptide and a heterologous signal peptide amino acid sequence. In a specific embodiment, an IL-15 derivative is a derivative of a naturally occurring human IL-15 polypeptide. In another embodiment, an IL-15 derivative is a derivative of an immature or precursor form of naturally occurring human IL-15 polypeptide. In another embodiment, an IL-15 derivative is a derivative of a mature form of naturally occurring human IL-15 polypeptide.

In a specific embodiment, a polynucleotide encoding IL-15 comprises the nucleotide sequence of any naturally occurring nucleic acid sequences encoding IL-15 (e.g., mammalian interleukin-15), including the immature or precursor and mature forms. Non-limiting examples of GeneBank Accession Nos. for the nucleotide sequence of various species of native mammalian IL-15 include NM_000585 (human), NM_008357 (Mus musculus), and RNU69272 (Rattus norvegicus). In a specific embodiment, a polynucleotide encoding IL-15 comprises a nucleotide sequence encoding a naturally occurring human IL-15 (e.g., a mature or immature/precursor form of human IL-15).

In another embodiment, a polynucleotide encoding IL-15 comprises the nucleotide sequence of a derivative of a naturally occurring nucleic acid sequences encoding mammalian interleukin-15, including the immature or precursor and mature forms.

In a specific embodiment, a polynucleotide sequence encoding IL-15 comprises (or consists of) the nucleotide sequence of SEQ ID NO: 96. In another specific embodiment, IL-15 comprises (or consists of) the amino acid sequence of SEQ ID NO: 97.

### Safety Switches

Genetically-modified NK cells of the invention may express a safety switch, also known as an inducible suicide gene or suicide switch, which can be used to eradicate the NK cells in vivo if desired e.g. if GVHD develops. In some examples, NK cells that express a chimeric antigen receptor are provided to the patient that trigger an adverse event, such as off-target toxicity. In some therapeutic instances, a patient might experience a negative symptom during therapy using chimeric antigen receptor-modified cells. In some cases, these therapies have led to side effects due, in part, to non-specific attacks on healthy tissue. In some examples, the therapeutic NK cells may no longer be needed, or the therapy is intended for a specified amount of time, for example, the therapeutic NK cells may work to decrease the tumor cell, or tumor size, and may no longer be needed. Therefore, in some embodiments are provided nucleic acids, cells, and methods wherein the modified NK cell also expresses an inducible Caspase-9 polypeptide. If there is a need, for example, to reduce the number of chimeric antigen receptor modified NK cells, an inducible ligand may be administered to the patient, thereby inducing apoptosis of the modified T cells.

These switches respond to a trigger, such as a pharmacological agent, which is supplied when it is desired to eradicate the NK cells, and which leads to cell death (e.g. by triggering necrosis or apoptosis). These agents can lead to expression of a toxic gene product, but a more rapid response can be obtained if the genetically-modified NK cells already express a protein which is switched into a toxic form in response to the agent.

In some embodiments, a safety switch is based on a pro-apoptotic protein that can be triggered by administering a chemical inducer of dimerization to a subject. If the pro-apoptotic protein is fused to a polypeptide sequence which binds to the chemical inducer of dimerization, delivery of this chemical inducer can bring two pro-apoptotic proteins into proximity such that they trigger apoptosis. For instance, Caspase-9 can be fused to a modified human FK-binding protein which can be induced to dimerize in response to the pharmacological agent rimiducid (AP1903). The use of a safety switch based on a human pro-apoptotic protein, such as, for example, Caspase-9 minimizes the risk that cells expressing the switch will be recognized as foreign by a human subject's immune system.

Delivery of rimiducid to a subject can therefore trigger apoptosis of T cells which express the caspase-9 switch.

Inducible Caspase 9 (iC9): This proapoptotic switch includes a fusion of caspase-9 with FKBP12 or derivatives. It is latent in the absence of ligand but drives dimerization of the Initiator caspase, caspase-9, from the intrinsic pathway for cell apoptosis. Dimerization leads to caspase-9 activation, cleavage and activation of the effector caspase, caspase-3, and rapid cell death by apoptosis. Inducible Caspase-9 has utility as a safety switch in cell therapies to block toxic responses.

Rapamycin and rapalog sensitive switches—Rapamycin is a macrolide that binds with subnanomolar affinity with FKBP12 and simultaneously with the target of rapamycin mTOR. An 89-amino acid domain derived from mTOR, FRB, is sufficient to dimerize with an FKBP12-rapamycin complex. Fusion of FKBP in tandem with FRB together with a signaling domain facilitates homodimerization and activates signaling in the presence of the heterodimerizer rapamycin or analogs of rapamycin, generically termed rapalogs. Rapamycin and certain rapalogs are cell-permeable, stable in vivo and bind their targets with high affinity and specificity.

Rapamycin/rapalog-sensitive switches iRC9 and iRMC—Fusion of FKBP12-FRB to the amino terminus of Caspase-9 generates a rapamycin-sensitive safety switch that operates with high efficiency and dose sensitivity. Fusion of FKBP12-FRB with MyD88/CD40 generates a rapamycin or rapalog sensitive costimulatory switch. The FKBP and FRB components can be put in tandem in either an FRB-FKBP or FKBP-FRB orientation and can be fused with the MC signaling components at the amino or carboxy terminus.

FKBP12-allele specific binding by rimiducid. Rimiducid binds with high affinity (˜0.1 nM) to the valine 36 allele of FKBP12 but with low affinity (˜500 nM) to the wild-type phenylalanine 36 FKBP12 allele. Rapamycin and rapalogs can bind to either FKBP allele.

Non-immunosuppressive C7-rapamycin analogs. The natural target of rapamycin, mTOR is essential for cell growth and rapamycin is immunosuppressive at low dose (˜1 nM). Rapalogs replacing the methoxy group at C7 with groups that have more bulk, typified by BPC015, bind with mTOR with low affinity. Mutation of the FRB in iRC9 or iRMC (or similar) to substitute threonine 2098 with leucine accommodates the derivatized rapalog and permits high affinity dimerization and signaling.

Orthogonal use of rimiducid and rapamycin sensitive switches to generate dual switch NK cells. IRC9 contains the rimiducid-insensitive F36 allele of FKBP12 and can be coexpressed with iMC in T cells. Doses of rimiducid capable of activating iMC and driving costimulation are incapable of activating the proapoptotic iRC9 switch. Rapamycin or rapalogs can activate the safety switch. Similarly, coexpression of iRMC with iC9 can generate dual switch CAR-T cells with the opposite specificity. Rapalogs are obligate heterodimerizers and can bind to but not activate the iC9 switch containing only FKBP12. Rimiducid can dimerize and activate the safety switch.

Caspase-9 switches are described in Di Stasi et al. (2011) supra; see also Yagyu et al. (2015) Mol Ther 23 (9): 1475-85; Rossigloni et al. (2018) Cancer Gene Ther doi.org/10.1038/s41417-018-0034-1; Jones et al. (2014) Front Pharmacol doi.org/10.3389/fphar.2014.00254; U.S. Pat. No. 9,434,935, issued Sep. 16, 2016, entitled Modified Caspase Polypeptides and Uses Thereof; U.S. Pat. No. 9,913,882, issued Mar. 13, 2018, entitled Methods for Inducing Partial Apoptosis Using Caspase Polypeptides; U.S. Pat. No. 9,393,292, issued Jul. 19, 2016, entitled Methods for Inducing Selective Apoptosis; and patent application US2015/0328292, published Nov. 19, 2015, entitled Caspase Polypeptides Having Modified Activity and Uses Thereof. Suicide switches may also be based on Fas or on HSV thymidine kinase.

Examples of ligand inducers for the switches include, for example, those discussed in Kopytek, S. J., et al., Chemistry & Biology 7:313-321 (2000) and in Gestwicki, J. E., et al., Combinatorial Chem. & High Throughput Screening 10:667-675 (2007); Clackson T (2006) Chem Biol Drug Des 67:440-2; Clackson, T., in Chemical Biology: From Small Molecules to Systems Biology and Drug Design (Schreiber, s., et al., eds., Wiley, 2007)

The ligand binding regions incorporated in the safety switches may comprise the FKBP12v36 modified FKBP12 polypeptide, or other suitable FKBP12 variant polypeptides, including variant polypeptides that bind to AP1903, or other synthetic homodimerizers such as, for example, AP20187 or AP2015. Variants may include, for example, an FKBP region that has an amino acid substitution at position 36 selected from the group consisting of valine, leucine, isoleuceine and alanine (Clackson T, et al., Proc Natl Acad Sci USA. 1998, 95:10437-10442). AP1903, also known as rimiducid, (CAS Index Name: 2-Piperidinecarboxylic acid, 1-[(2S)-1-oxo-2-(3, 4,5-trimethoxyphenyl)butyl]-, 1,2-ethanediylbis [imino (2-oxo-2,1-ethanediyl)oxy-3,1-phenylene [(1R)-3-(3,4-dimethoxyphenyl) propylidene]] ester, [2S-[1 (R*),2R*[S*[S*[1 (R*),2R*]]]]]-(9CI) CAS Registry Number: 195514-63-7; Molecular Formula: C78H98N4O20 Molecular Weight: 1411.65), is a synthetic molecule that has proven safe in healthy volunteers (Iuliucci J D, et al., J Clin Pharmacol. 2001, 41: 870-879).

Provided in some embodiments are safety switches such as, for example, the safety switches discussed in Di Stasi et al. (2011) supra, which consists of the sequence of the human FK506-binding protein (FKBP12) (GenBank AH002 818) with an F36V mutation, connected through a SGGGS linker to a modified human caspase 9 (CASP9) which lacks its endogenous caspase activation and recruitment domain. The F36V mutation increases the binding affinity of FKBP12 to synthetic homodimerizers AP20187 and AP1903 (rimiducid).

The safety switch may comprise a modified Caspase-9 polypeptide having modified activity, such as, for example, reduced basal activity in the absence of the homodimerizer ligand. Modified Caspase-9 polypeptides are discussed in, for example, U.S. Pat. No. 9,913,882 and US-2015-0328292, supra, and may include, for example, amino acid substitutions at position 330 (e.g., D330E or D330A) or, for example, amino acid substitutions at position 450 (e.g., N405Q), or combinations thereof, including, for example, D330E-N405Q and D330A-N405Q. Caspase-9 polypeptide with lower basal activity have been described previously, e.g. in U.S. Pat. Nos. 9,434,935, 9,932,572 and 9,913,882, and U.S. Patent Application Nos. 62/668,223, 62/756,442, 62/816,799, 15/901,556, 15/888,948.

In a specific embodiment, provided herein is a pharmaceutical composition comprising a dimerizing or multimerizing ligand. In another specific embodiment, provided herein is a pharmaceutical composition comprising an effective amount of a dimerizing or multimerizing ligand.

In a specific embodiment, provided herein is a pharmaceutical composition comprising a dimerizing or multimerizing ligand and a pharmaceutically acceptable carrier. In another specific embodiment, provided herein is a pharmaceutical composition comprising a pharmaceutically acceptable carrier and an effective amount of a dimerizing or multimerizing ligand.

An effective amount of a pharmaceutical composition, such as the dimerizing or multimerizing ligand presented herein, would be the amount that achieves this selected result of inducing apoptosis in the Caspase-9-expressing cells NK cells, such that over 60%, 70%, 80%, 85%, 90%, 95%, or 97%, or that under 80%, 70%, 60%, 50%, 40%, 30%, 20%, or 10% of the therapeutic cells are killed. The term is also synonymous with “sufficient amount.” Any appropriate assay may be used to determine the percent of therapeutic cells that are killed. An assay may include the steps of obtaining a first sample from a subject before administration of the dimerizing or multimerizing ligand and obtaining a second sample from the subject after administration of the dimerizing or multimerizing ligand and comparing the number or concentration of therapeutic cells in the first and second samples to determine the percent of therapeutic cells that are killed. One can empirically determine the effective amount of a particular composition presented herein without necessitating undue experimentation.

Non-limiting examples of chimeric polypeptides useful for inducing cell death or apoptosis, and related methods for inducing cell death or apoptosis, including expression constructs, methods for constructing vectors, assays for activity or function, and multimerization of the chimeric polypeptides by contacting cells that express inducible chimeric polypeptides with a multimeric compound, or a pharmaceutically acceptable salt thereof, that binds to the multimerizing region of the chimeric polypeptides both ex vivo and in vivo, administration of expression vectors, cells, or multimeric compounds described herein, or pharmaceutically acceptable salts thereof, to subjects, and administration of multimeric compounds described herein, or pharmaceutically acceptable salts thereof, to subjects who have been administered cells that express the inducible chimeric polypeptides, may also be found in the following patents and patent applications, each of which is incorporated by reference herein in its entirety for all purposes. U.S. patent application Ser. No. 13/112,739, filed May 20, 2011, entitled METHODS FOR INDUCING SELECTIVE APOPTOSIS, published Nov. 24, 2011, as US2011-0286980-A1, issued Jul. 28, 2015 as U.S. Pat. No. 9,089,520; U.S. patent application Ser. No. 13/792,135, filed Mar. 10, 2013, entitled MODIFIED CASPASE POLYPEPTIDES AND USES THEREOF, published Sep. 11, 2014 as US2014-0255360-A1, issued Sep. 6, 2016 as U.S. Pat. No. 9,434,935, by Spencer et al.; International Patent Application No. PCT/US2014/022004, filed Mar. 7, 2014, published Oct. 9, 2014 as WO2014/16438; U.S. patent application Ser. No. 14/296,404, filed Jun. 4, 2014, entitled METHODS FOR INDUCING PARTIAL APOPTOSIS USING CASPASE POLYPEPTIDES, published Jun. 2, 2016 as US2016-0151465-A1, by Slawin et al; International Application No. PCT/US2014/040964 filed Jun. 4, 2014, published as WO2014/197638 on Feb. 5, 2015, by Slawin et al.; U.S. patent application Ser. No. 14/640,553, filed Mar. 6, 2015, entitled CASPASE POLYPEPTIDES HAVING MODIFIED ACTIVITY AND USES THEREOF, published Nov. 19, 2015 as US2015-0328292-A1; International Patent Application No. PCT/US2015/019186, filed Mar. 6, 2015, published Sep. 11, 2015 as WO2015/134877, by Spencer et al.; U.S. patent application Ser. No. 14/968,737, filed Dec. 14, 2015, entitled METHODS FOR CONTROLLED ELIMINATION OF THERAPEUTIC CELLS, published Jun. 16, 2016 as US2016-0166613-A1, by Spencer et al.; International Patent Application No. PCT/US2015/065629 filed Dec. 14, 2015, published Jun. 23, 2016 as WO2016/100236, by Spencer et al.; U.S. patent application Ser. No. 14/968,853, filed Dec. 14, 2015, entitled METHODS FOR CONTROLLED ACTIVATION OR ELIMINATION OF THERAPEUTIC CELLS, published Jun. 23, 2016 as US2016-0175359-A1, by Spencer et al.; International Patent Application No. PCT/US2015/065646, filed Dec. 14, 2015, published Sep. 15, 2016 as WO2016/100241, by Spencer et al.; U.S. patent application Ser. No. 15/377,776, filed Dec. 13, 2016, entitled DUAL CONTROLS FOR THERAPEUTIC CELL ACTIVATION OR ELIMINATION, published Jun. 15, 2017 as US2017-0166877-A1., by Bayle et al.; and International Patent Application No. PCT/US2016/066371, filed Dec. 13, 2016, published Jun. 22, 2017 as WO2017/106185, by Bayle et al., each of which is incorporated by reference herein in its entirety, including all text, tables and drawings, for all purposes. Multimeric compounds described herein, or pharmaceutically acceptable salts thereof, may be used essentially as discussed in examples provided in these publications, and other examples provided herein.

As used herein, the term “pharmaceutically or pharmacologically acceptable” refers to molecular entities and compositions that do not produce adverse, allergic, or other untoward reactions when administered to an animal or a human.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells presented herein, its use in therapeutic compositions is contemplated. Supplementary active ingredients also can be incorporated into the compositions. In some embodiments, the subject is a mammal.

By “kill” or “killing” as in a percent of cells killed, is meant the death of a cell through apoptosis, as measured using any method known for measuring apoptosis. The term may also refer to cell ablation.

### Chemical Induction of Dimerization—Dual Switch

Chemical Induction of Dimerization (CID) with small molecules is an effective technology used to generate switches of protein function to alter cell physiology. A high specificity, efficient dimerizer is rimiducid (AP1903), which has two identical, protein-binding surfaces arranged tail-to-tail, each with high affinity and specificity for a mutant of FKBP12: FKBP12 (F36V) (FKBP12v36, F_{V36} or Fᵥ), Attachment of one or more Fv domains onto one or more cell signaling molecules that normally rely on homodimerization can convert that protein to rimiducid control. Homodimerization with rimiducid is used in the context of an inducible caspase safety switch, and an inducible activation switch for cellular therapy, where MyD88 and a costimulatory polypeptide cytoplasmic region are used to stimulate immune activity.

Another CID that may be used to activate the inducible chimeric signaling polypeptides is based on a heterodimerizer, such as Rapamycin, or a rapamycin analog (“rapalog”). In these embodiments, the multimeric ligand binding region provided in the inducible chimeric signaling polypeptides, or the multimeric ligand binding region provided in the inducible chimeric caspase polypeptides binds to Rapamycin or a rapalog and does not bind to rimiducid. Rapamycin binds to FKBP12, and its variants, and can induce heterodimerization of signaling domains that are fused to FKBP12 by binding to both FKBP12 and to polypeptides that contain the FKBP-rapamycin-binding (FRB) domain of mTOR.

In some embodiments, a dual switch is provided where the nucleic acid that encodes the inducible chimeric signaling polypeptide also encodes an inducible chimeric caspase polypeptide, for example, an inducible chimeric caspase 9 polypeptide. In some embodiments, modified cells are provided that express an inducible chimeric signaling polypeptide and an inducible chimeric caspase polypeptide, for example, an inducible chimeric caspase 9 polypeptide. The multimeric ligand binding regions provided in these two distinct polypeptides are different. In one example, the inducible chimeric signaling polypeptide comprises an FRB multimeric ligand binding domain, and the inducible chimeric caspase 9 polypeptide comprises an FKBP12 variant that binds to rimiducid. In this example, a dual control system is provided. Contacting the cells with rapamycin or a rapalog induces the immune cell activity by multimerizing the inducible chimeric signaling polypeptide. Contacting the cells with rimiducid induces apoptosis by multimerizing the inducible chimeric caspase polypeptide. In other embodiments, a rapamycin or rapalog-inducible pro-apoptotic polypeptide, such as, for example, Caspase-9 or a rapamycin or rapalog-inducible chimeric signaling polypeptide, such as, for example, MyD88/4-1BB, OX40, ICOS, or CD28, (iM-X) is used in combination with a rimiducid-inducible pro-apoptotic polypeptide, such as, for example, Caspase-9, or a rimiducid-inducible iM-X, to produce dual switches. These dual switches can be used to control both cell proliferation and activity, and apoptosis selectively by administration of either of two distinct ligand inducers.

The multimerizing regions, such as FKBP12/FRB, FRB/FKBP12, and FKBP12v36, may be located amino terminal to the pro-apoptotic polypeptide or signaling polypeptide, or, in other examples, may be located carboxyl terminal to the pro-apoptotic polypeptide or signaling polypeptide. Additional polypeptides, such as, for example, linker polypeptides, stem polypeptides, spacer polypeptides, or in some examples, marker polypeptides, may be located between the multimerizing region and the pro-apoptotic polypeptide or costimulatory polypeptide, in the chimeric polypeptides.

As used here, the term “rapalog” is meant as an analog of the natural antibiotic rapamycin. Certain rapalogs in the present embodiments have properties such as stability in serum, a poor affinity to wildtype FRB (and hence the parent protein, mTOR, leading to reduction or elimination of immunosuppressive properties), and a relatively high affinity to a mutant FRB domain. For commercial purposes, in certain embodiments, the rapalogs have useful scaling and production properties. Examples of rapalogs include, but are not limited to, S-o,p-dimethoxyphenyl (DMOP)-rapamycin; R-Isopropoxyrapamycin; C7-Isobutyloxyrapamycin; S-Butanesulfonamidorap (AP23050); 40-(S)-Fluoro-Rapamycin; 40-(S)-Chloro-Rapamycin; 40-(S)-Bromo-Rapamycin; 40-(S)-Iodo-Rapamycin; 40-(S)-Amino-Rapamycin; 40-(S)-Fluoro-7-(S)-DMOP-Rapamycin; 40-(S)-Chloro-7-(S)-DMOP-Rapamycin; 40-(S)-Iodo-7-(S)-DMOP-Rapamycin; 40-(S)-Azide-7-(S)-DMOP-Rapamycin; 40-(R)-p-Bromomethylbenzoyl-Rapamycin; 40-(R)-p-Chloromethylbenzoyl-Rapamycin; 40-(R)-((4-methylpiperazin-1-yl) p-methylbenzoyl)-Rapamycin; di-p-Bromomethylbenzoyl-Rapamycin; and 40-(S)—N-(3-(4-methylpiperazin-1-yl) propyl)-Rapamycinamine (see, e.g., U.S. Patent Application Publication No. US2017/0166877, which is incorporated by reference herein).

The term “FRB” refers to the FKBP12-Rapamycin-Binding (FRB) domain (residues 2015-2114 encoded within mTOR), and analogs thereof. In certain embodiments, FRB variants are provided. The properties of an FRB variant are stability (some variants are more labile than others) and ability to bind to various rapalogs. Based on the crystal structure conjugated to rapamycin, there are 3 key rapamycin-interacting residues that have been most analyzed, K2095, T2098, and W2101. Mutation of all three leads to an unstable protein that can be stabilized in the presence of rapamycin or some rapalogs. This feature can be used to further increase the signal: noise ratio in some applications. Examples of mutants are discussed in Bayle et al (06) Chem & Bio 13:99-107; Stankunas et al (07) Chembiochem 8:1162-1169; and Liberles S (97) PNAS 94:7825-30). Examples of FRB regions of the present embodiments include, but are not limited to, KLW (with L2098); KTF (with F2101); and KLF (L2098, F2101). Heterodimerization is discussed in, for example, Belshaw, P., et al., PNAS 93:4604-4607 (1996). Additional compositions and methods are discussed, for example, in U.S. patent application Ser. No. 14/968,737, titled Methods for Controlled Elimination of Therapeutic Cells by Spencer, D., et al., filed Dec. 14, 2015, published as US-2016-0166613A1 on Jun. 16, 2016; International Patent Application PCT/US2015/065629, published as WO2016/100236 on Jun. 23, 2016; U.S. patent application Ser. No. 14/968,853 titled Methods for Controlled Activation or Elimination of Therapeutic Cells, by Spencer, D., et al., filed Dec. 14, 2015, published as US-2016-0175359A1 on Jun. 23, 2016; International Patent Application PCT/US2015/065646 filed Dec. 14, 2015, published as WO2016/100241 on Sep. 15, 2016; International Patent Application PCT/US2015/065629, filed Dec. 14, 2015, published as WO2016/100236 on Jun. 23, 2016; International Patent Application PCT/US2016/066371, filed Dec. 13, 2016, titled Dual Controls for Therapeutic Cell Activation or Elimination, by Bayle, J. H., et al.; and U.S. patent application Ser. No. 15/377,776, filed Dec. 13, 2016, titled Dual Controls for Therapeutic Cell Activation or Elimination, by Bayle, J. H., et al., each of which is hereby incorporated by reference herein in its entirety.

The ligands used are capable of binding to two or more of the ligand binding domains. The chimeric proteins may be able to bind to more than one ligand when they contain more than one ligand binding domain. The ligand is typically a non-protein or a chemical. Exemplary ligands include, but are not limited to FK506 (e.g., FK1012).

Other ligand binding regions may be, for example, dimeric regions, or modified ligand binding regions with a wobble substitution, such as, for example, FKBP12 (V36): The human 12 kDa FK506-binding protein with an F36 to V substitution, the complete mature coding sequence (amino acids 1-107), provides a binding site for synthetic dimerizer drug rimiducid (Jemal, A. et al., CA Cancer J. Clinic. 58, 71-96 (2008); Scher, H. I. and Kelly, W. K., Journal of Clinical Oncology 11, 1566-72 (1993)). Two tandem copies of the protein may also be used in the construct so that higher-order oligomers are induced upon cross-linking by rimiducid.

FKBP12 variants may also be used in the FKBP12/FRB multimerizing regions. Variants used in these fusions, in some embodiments, will bind to rapamycin, or rapalogs, but will bind to less affinity to rimiducid than, for example, FKBP12v36. Examples of FKBP12 variants include those from many species, including, for example, yeast. In one embodiment, the FKBP12 variant is FKBP12.6 (calstablin).

Other heterodimers are contemplated in the present application. In one embodiment, a calcineurin-A polypeptide, or region may be used in place of the FRB multimerizing region. In some embodiments, the first unit of the first multimerizing region is a calcineurin-A polypeptide. In some embodiments, the first unit of the first multimerizing region is a calcineurin-A polypeptide region and the second unit of the first multimerizing region is a FKBP12 or FKBP12 variant multimerizing region. In some embodiments, the first unit of the first multimerizing region is a FKBP12 or FKBP12 variant multimerizing region and the second unit of the first multimerizing region is a calcineurin-A polypeptide region. In these embodiments, the first ligand comprises, for example, cyclosporine.

### Chimeric Antigen Receptors and Cell Therapy

In some examples of cell therapy, NK cells, such as, for example, NK cells that comprise the chimeric polypeptides of the present application may comprise a chimeric antigen receptor. These modified NK cells may be used for cell therapy.

Chimeric antigen receptors (CARs) are artificial receptors designed to convey antigen specificity to T cells. They generally include an antigen-specific component, a transmembrane component, and an intracellular component selected to activate the cell and provide specific immunity. Chimeric antigen receptor-expressing NK cells may be used in various therapies, including cancer therapies. While effective against tumors, in some cases these therapies have led to side effects due, in part to non-specific attacks on healthy tissue. Thus, a method for controllable NK cell therapy is needed that provides a strong immunotherapeutic response and avoids toxic side effects.

By “chimeric antigen receptor” or “CAR” is meant, for example, a chimeric polypeptide which comprises a polypeptide sequence that recognizes a target antigen (an antigen-recognition domain) linked to a transmembrane polypeptide and intracellular domain polypeptide selected to activate the cell and provide specific immunity. The antigen-recognition domain may be a single-chain variable fragment (scFv), or may, for example, be derived from other molecules such as, for example, a T cell receptor or Pattern Recognition Receptor. The intracellular domain comprises at least one polypeptide which causes activation of the cell, such as, for example, but not limited to, CD3 zeta, and, for example, co-stimulatory molecules, for example, but not limited to, CD28, OX40 and 4-1BB. In a specific embodiment, CD28, OX40, ICOS, 4-1BB, or CD3 zeta comprises (or consists) of a sequence disclosed herein (e.g., a sequence disclosed in the Examples below). The term “chimeric antigen receptor” may also refer to chimeric receptors that are not derived from antibodies but are chimeric T cell receptors. These chimeric T cell receptors may comprise a polypeptide sequence that recognizes a target antigen, where the recognition sequence may be, for example, but not limited to, the recognition sequence derived from a T cell receptor or a scFv. The intracellular domain polypeptides are those that act to activate the T cell. Chimeric T cell receptors are discussed in, for example, Gross, G., and Eshar, Z., FASEB Journal 6:3370-3378 (1992), and Zhang, Y., et al., PLOS Pathogens 6:1-13 (2010).

### Transmembrane Regions

A chimeric protein herein may include a single-pass or multiple pass transmembrane sequence (e.g., at the N-terminus or C-terminus of the chimeric protein). Single pass transmembrane regions are found in certain CD molecules, tyrosine kinase receptors, serine/threonine kinase receptors, TGFβ, BMP, activin and phosphatases. Single pass transmembrane regions often include a signal peptide region and a transmembrane region of about 20 to about 25 amino acids, many of which are hydrophobic amino acids and can form an alpha helix. A short track of positively charged amino acids often follows the transmembrane span to anchor the protein in the membrane. Multiple pass proteins include ion pumps, ion channels, and transporters, and include two or more helices that span the membrane multiple times. All or substantially all of a multiple pass protein sometimes is incorporated in a chimeric protein. Sequences for single pass and multiple pass transmembrane regions are known and can be selected for incorporation into a chimeric protein molecule.

In some embodiments, the transmembrane domain is fused to the extracellular domain of the CAR. In one embodiment, the transmembrane domain that naturally is associated with one of the domains in the CAR is used. In other embodiments, a transmembrane domain that is not naturally associated with one of the domains in the CAR is used. In some instances, the transmembrane domain can be selected or modified by amino acid substitution (e.g., typically charged to a hydrophobic residue) to avoid binding of such domains to the transmembrane domains of the same or different surface membrane proteins to minimize interactions with other members of the receptor complex.

Transmembrane domains may, for example, be derived from the alpha, beta, or zeta chain of the T cell receptor, CD3-ε, CD3 ζ, CD4, CD5, CD8, CD8α, CD9, CD16, CD22, CD28, CD33, CD38, CD64, CD80, CD86, CD134, CD137, or CD154. Or, in some examples, the transmembrane domain may be synthesized de novo, comprising mostly hydrophobic residues, such as, for example, leucine and valine. In certain embodiments a short polypeptide linker may form the linkage between the transmembrane domain and the intracellular domain of the chimeric antigen receptor. The chimeric antigen receptors may further comprise a stalk, that is, an extracellular region of amino acids between the extracellular domain and the transmembrane domain. For example, the stalk may be a sequence of amino acids naturally associated with the selected transmembrane domain. In some embodiments, the chimeric antigen receptor comprises a CD8 transmembrane domain, in certain embodiments, the chimeric antigen receptor comprises a CD8 transmembrane domain, and additional amino acids on the extracellular portion of the transmembrane domain, in certain embodiments, the chimeric antigen receptor comprises a CD8 transmembrane domain and a CD8 stalk. In a specific embodiment, a CD8 transmembrane comprises (or consists of) a sequence disclosed herein (e.g., a sequence disclosed in the Examples below). In another specific embodiment, a CD8 stalk comprises (or consists of) a sequence disclosed herein (e.g., a sequence disclosed in the Examples below). The chimeric antigen receptor may further comprise a region of amino acids between the transmembrane domain and the cytoplasmic domain, which are naturally associated with the polypeptide from which the transmembrane domain is derived.

### Target Antigens

Chimeric antigen receptors bind to target antigens. When assaying NK cell activation in vitro or ex vivo, target antigens may be obtained or isolated from various sources. The target antigen, as used herein, is an antigen or immunological epitope on the antigen, which is crucial in immune recognition and ultimate elimination or control of the disease-causing agent or disease state in a mammal. The immune recognition may be cellular and/or humoral. In the case of intracellular pathogens and cancer, immune recognition may, for example, be a T lymphocyte response.

The target antigen may be derived or isolated from, for example, a pathogenic microorganism such as viruses including HIV, (Korber et al, eds HIV Molecular Immunology Database, Los Alamos National Laboratory, Los Alamos, N. Mex. 1977) influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV) and the like. Target antigen may be derived or isolated from pathogenic bacteria such as, for example, from Chlamydia (U.S. Pat. No. 5,869,608), Mycobacteria, Legionella, Meningiococcus, Group A Streptococcus, Salmonella, Listeria, Hemophilus influenzae (U.S. Pat. No. 5,955,596) and the like). Target antigen may be derived or isolated from, for example, pathogenic yeast including Aspergillus, invasive Candida (U.S. Pat. No. 5,645,992), Nocardia, Histoplasmosis, Cryptosporidia and the like. Target antigen may be derived or isolated from, for example, a pathogenic protozoan and pathogenic parasites including but not limited to Pneumocystis carinii, Trypanosoma, Leishmania (U.S. Pat. No. 5,965,242), Plasmodium (U.S. Pat. No. 5,589,343) and Toxoplasma gondii.

The term “antigen” as used herein is defined as a molecule that provokes an immune response. This immune response may involve either antibody production, or the activation of specific immunologically competent cells, or both. An antigen can be derived from organisms, subunits of proteins/antigens, killed or inactivated whole cells or lysates. Therefore, any macromolecules, including virtually all proteins or peptides, can serve as antigens. Furthermore, antigens can be derived from recombinant or genomic DNA, including, for example, any DNA that contains nucleotide sequences or partial nucleotide sequences of a pathogenic genome or a gene or a fragment of a gene for a protein that elicits an immune response results in synthesis of an antigen.

Target antigen includes an antigen associated with a preneoplastic or hyperplastic state. Target antigen may also be associated with, or causative of cancer. Such target antigen may be, for example, tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D and the like (GenBank Accession No. M29540), MART-1 (Kawakarni et al, J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987) TRP-1 (gp75) (Cohen et al Nucleic Acid Res. 18:2807-2808, 1990; U.S. Pat. No. 5,840,839), NY-ESO-1 (Chen et al PNAS 94:1914-1918, 1997), TRP-2 (Jackson et al EMBOJ, 11:527-535, 1992), TAG72, KSA, CA-125, CD-123, PSA, HER-2/neu/c-erb/B2, (U.S. Pat. No. 5,550,214), BRC-I, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each. A tumor antigen is any antigen such as, for example, a peptide or polypeptide, that triggers an immune response in a host against a tumor. The tumor antigen may be a tumor-associated antigen, which is associated with a neoplastic tumor cell.

In one type of chimeric antigen receptor (CAR), the variable heavy (VH) and light (VL) chains for a tumor-specific monoclonal antibody are fused in-frame with the CD3 zeta chain (ζ) from the T cell receptor complex. The VH and VL are generally connected together using a flexible glycine-serine linker, and then attached to the transmembrane domain by a spacer (CH2CH3) to extend the scFv away from the cell surface so that it can interact with tumor antigens. Following transduction, NK cells now express the CAR on their surface, and upon contact and ligation with a tumor antigen, signal through the CD3 zeta chain inducing cytotoxicity and cellular activation.

### Immune Cell Therapy and Inducible Chimeric Signaling Polypeptides

In some embodiments, NK cells are modified to express a chimeric antigen receptor that comprises a single chain antibody variable fragment (scFv) fused with a transmembrane domain containing linker region and an intracellular domain derived from the CD3 zeta component. In natural T cells signals from CD3zeta drive the initial activation of the T cell through signaling to the NF-ATc transcription factor. These signals are necessary to drive target cell killing in cytotoxic T lymphocytes and synergize with costimulatory signaling pathways to drive the robust cell proliferation of T cell immune response. The T cells may be modified by transduction or transfection with a nucleic acid that expresses the CAR in the absence of any coding region for a chimeric signaling polypeptide. Or, in other examples, the polynucleotide that encodes the CAR may be provided as part of a nucleic acid that also comprises a polynucleotide that encodes a chimeric signaling polypeptide.

For inducible co-stimulation, the CAR-NK cells are also modified, for example, by transfection or transduction of the cells with a nucleic acid that expresses an inducible chimeric signaling polypeptide. For example, in some embodiments, the polypeptide is inducible based on a drug inducible mediator of costimulatory signaling in which FKBP12 in two copies is fused with MyD88. FKBP12 is a small (107 amino acid) prolylyl isomerase that is also the ligand for the natural antibiotic and immunosuppressant macrolides rapamycin, FK-506 and ascomycin. In certain embodiments, a single mutant of FKBP12 substituting valine at amino acid 36 for phenylalanine (Fv) confers inducibility to Fv fusions with the synthetic ligand rimiducid (AP1903) by homodimerization of FKBP12 moieties. MyD88 is critical mediator of signals in the innate immune response downstream of Toll-Like Receptors (TLR) typically in myeloid cells but also in lymphocytes. These signals activate transcription regulators including the family of NF-κB factors.

Rimiducid is a tail-to-tail linkage of a high affinity synthetic ligand specific for Fv and not wild-type FKBP12. It is a dimerizing ligand because it can simultaneously bind with two Fv moieties. The drug-directed dimerizing event thereby juxtaposes the fused MyD88 moieties which initiates robust signal transduction. This is demonstrated in retroviral construct 1810 and is denoted iM.

The Fv-MyD88 fusion is linked with a second costimulatory signaling domain derived from the intracellular domain of CD40 to generate iMC. iMC has been utilized to activate proliferation and cytokine production in myeloid cells and in CAR-T cells and retroviral vector BP2212 is an example of an iMC-CAR expression construct.

Alternative signaling domains, when fused with iMyD88, generate distinct signaling outcomes. In some embodiments, a construct expresses an inducible fusion of MyD88 with the intracellular domain of CD28, the canonical costimulatory receptor for the CD80/CD86 ligands of antigen presenting cells. In some embodiments, construct BP1815 fuses iMyD88 with the intracellular signaling domain of 4-1BB (also called CD137, a costimulatory receptor present in activated T cells). In some embodiments, construct BP1801 expresses iMyD88 fused with the signaling domain of OX40. OX40 is a member of the Tumor Necrosis Factor Receptor (TNFR) superfamily that signal to NF-κB through TRAF proteins. In some embodiments, construct BP1802 expresses iMyD88 fused with the signaling domain of ICOS (Inducible COSstimulator, also called CD278) a member of the CD28 family which signals to NF-κB through a mechanism distinct from TNF-R family members. In some embodiments, Construct BP1800 expresses iMyD88 fused with the signaling domains of OX40 and CD28.

### Immune Cell Therapy and Constitutive Chimeric Signaling Polypeptides

Immune cell therapies may also be designed to provide constitutively active therapy, such as constitutively active CAR-T cells or constitutively active NK-cells, but provide an inducible safety switch, to stop, or reduce the level of, the therapy when needed. In some embodiments, immune cells, such as CAR-T cells or CAR-NK cells, express a chimeric antigen receptor, and a chimeric signaling polypeptide comprising a truncated MyD88 polypeptide and a stimulating polypeptide. In this format, the multimeric ligand binding region is not fused with the truncated MyD88 polypeptide. High level costimulation is provided constitutively through an alternate mechanism in which a leaky 2A cotranslational sequence, for example one derived from porcine teschovirus-1 (P2A), is used to separate the CAR from the chimeric MyD88 polypeptide. When the chimeric MyD88 polypeptide is a MyD88-CD40 polypeptide, most MC remains cytosolic but the leakiness in the P2A sequence retains a portion (estimated to be about 10%) of MC fused with the CAR and thereby expressed at the plasma membrane. This membrane proximal expression produces a high level of signaling activity.

In some embodiments, the modified cells comprise a non-inducible chimeric polypeptide that is not induced by contact with a ligand inducer, or dimerizer, or CID, such as, for example, rimiducid AP20187, or AP1510. In some embodiments, the modified cells comprise a chimeric polypeptide that does not bind rapamycin, a rapalog, rimiducid, AP20187, or AP1510. In some embodiments, the modified cells comprise a chimeric polypeptide that does not comprise a multimeric ligand binding region, and does not comprise, for example, an FKBP12 polypeptide region or an FRB region, or variants thereof. In some embodiments, the chimeric polypeptide does not have a multimeric ligand binding region, and does not have an FKBP12 polypeptide region, or FRB region, or variants thereof. In some embodiments, the chimeric polypeptide does not have a functional multimeric ligand binding region.

The inducible component in these modified cells is an inducible Caspase-9 polypeptide, for example, an Fv fusion with caspase 9 (iC9) that rapidly induces apoptosis, or programmed cell death, in a rimiducid dependent fashion. This iC9 safety switch can thereby be deployed to block adverse events that may result from CAR-NK therapy such as graft versus host disease or cytokine release syndrome. In animal studies inducible caspase-9 polypeptide-expressing NK cells containing MyD88-CD40 (MC) produce robust anti-tumor effects that may have toxic effects on the animals that necessitate rimiducid treatment to remove the most active CAR-NK cells. The toxic effects are consistent with a cytokine release syndrome that is likely due to excessive production of inflammatory cytokines such as TNF-α and IL-6.

Also provided herein are chimeric signaling polypeptides that do not include a multimeric ligand binding region. These polypeptides provide constitutive NK cell activation activity; the polypeptides may be provided in immune cells, such as NK cells, in which an inducible apoptotic polypeptide, such as Caspase 9 may be expressed.

### Expansion and Storage of Natural Killer Cells and Natural Killer Cells in Cell Therapy

In some embodiments of the present application, the expression of MC in NK cells increases the rate of NK cell proliferation in culture. In some embodiments, the activation of iMC or iRMC with their respective dimerizing ligand, or the constitutively activated MC, increases the tumor cell killing activity of NK cells in culture. In some embodiments, the dimerization of iMC with rimiducid, or the constitutively activated MC, activates the production of cytokines that stimulate NK cell function, but are not normally produced by NK cells, including IL-12 p70, IL-2, and IL-15. In some embodiments, activation of MC in NK cells stimulates NK cell proliferation and antitumor efficacy in a whole animal model. In some embodiments, expression and activation of MC prior to cryostorage generates resistance to the inactivating effect of cryostorage on NK cell function. In some embodiments, activation of the iRC9 safety switch with rapamycin causes rapid NK cell apoptosis.

In some embodiments, natural killer cells of the present application are used in cell therapy targeting cancer. NK cells are currently in clinical trials as anti-tumor cell therapies. Because NK cells grow poorly in vivo, multiple cell infusions are typically necessary to generate therapeutic effect [Ciurea S O, et al., Phase 1 clinical trial using mbIL21 ex vivo-expanded donor-derived NK cells after haploidentical transplantation. Blood 2017, 130 (16): 1857-1868]. Generation of dual-switch NK (DS NK) cells augmented with the iMC switch or MC may facilitate NK cell proliferation and anti-tumor efficacy with dimerizing-drug dosage. Incidences of off-tumor targeting, cytokine release syndrome or GvHD can be quickly and efficiently reversed with activation of the safety switch. The resistance of DS NK cells to cryostorage will make possible the large-scale production of standardized lots for cell therapy.

In some embodiments, NK cells of the present application may express chimeric antigen receptors, and in certain embodiments, the chimeric antigen receptors may be under the control of a dual-switch system. Addition of a Chimeric Antigen Receptor containing a fusion of a single chain variable fragment (for target specificity) a transmembrane domain and an intracellular signaling domain to promote NK cell activation may produce target-specific NK cells in which proliferation and cytokine production is ligand inducible while safety is promoted by the caspase-9 safety switch.

In some embodiments, NK cells of the present application may be modified for use in therapeutic antibody therapy. NK cells express the CD16 Fcγ-receptor that binds to the constant region of lgG-immunoglobulins. CD16 links with NK cell signaling proteins that activate cells for target killing [Shevtsov M, et al. Front Immunol 2016, 7: 492.15]. Preincubation or co-infusion of DS NK cells with tumor-target specific therapeutic antibodies may generate tumor specific responses augmented by inducible MC and made safer by the proapoptotic switch.

In some embodiments, NK cells of the present application may be used as an adjunct to checkpoint inhibition, and in certain embodiments, under the control of a dual-switch system. Tumor infiltrating lymphocytes (TILs) are frequently inhibited by activation of checkpoint inhibitory receptors, including CTLA4, PD1 and LAG3. Blockade of these receptors with their tumor-resident ligands can reactivate these TILs and produce robust clinical responses. A subset of tumor cells can reduce the CTL response by downregulating MHC-I surface expression. These cells are subject to attack by NK cells. Inclusion of DS NK cells with checkpoint inhibition may enhance the rate of tumor elimination.

In some embodiments, NK cells of the present application may be used as an adjunct to CAR-T or TCR-T therapy, and in certain embodiments, under the control of a dual-switch system. CD19-specific CAR-T cells and T cell products expressing tumor specific TCRs produce robust clinical responses, yet tumor relapses are common [Jackson H J, et al., Driving CAR T-cells forward. Nat Rev Clin Oncol 2016, 13 (6): 370-383; Brudno J N, et al., Chimeric antigen receptor T-cell therapies for lymphoma. Nat Rev Clin Oncol 2018, 15 (1): 31-46; Wang J, et al., Acute lymphoblastic leukemia relapse after CD19-targeted chimeric antigen receptor T cell therapy. J Leukoc Biol 2017, 102 (6): 1347-1356]. Frequently, these relapses are the result of loss of the epitope for the CAR in a small population of tumor that later proliferates [Grupp S A, et al., Chimeric antigen receptor-modified T cells for acute lymphoid leukemia. N Engl J Med 2013, 368 (16): 1509-1518; Wang J, et al., Acute lymphoblastic leukemia relapse after CD19-targeted chimeric antigen receptor T cell therapy. J Leukoc Biol 2017, 102 (6): 1347-1356]. Inclusion of long-lasting NK cells coincident or following CAR-T cell infusion may target this minor population with unspecific but highly tumor-responsive DS NK cells.

Provided in certain embodiments are methods for stimulating a cell-mediated immune response in a subject, comprising administering a modified cell transfected or transduced with a nucleic acid that expresses an inducible chimeric signaling polypeptide of the present embodiments to the subject; and administering an effective amount of a multimeric ligand that binds to the multimeric ligand binding region to stimulate a cell-mediated immune response in the subject. In some embodiments, the modified cell expresses a chimeric antigen receptor, an inducible chimeric antigen receptor polypeptide, or a recombinant T cell receptor, that binds to a target cell. In some embodiments, the target cell is a tumor cell. In some embodiments, the number or concentration of target cells in the subject is reduced following administration of the modified cell and the multimeric ligand. In some embodiments, the methods further comprise measuring the number or concentration of target cells in a first sample obtained from the subject before administering the modified cell or ligand, measuring the number or concentration of target cells in a second sample obtained from the subject after administration of the modified cell and ligand, and determining an increase or decrease of the number or concentration of target cells in the second sample compared to the number or concentration of target cells in the first sample. In some embodiments, the number of target cells is reduced 10, 20, 30, 40, 50, 60, 70, 80, 90, or 95% or more in the second sample, compared to the number or concentration of target cells in the first sample.

In some embodiments, the reduction in the number or concentration of target cells is determined in vitro, or ex vivo, using a cytotoxicity assay, and cytotoxicity of the modified NK cells is compared to either a control group of target cells that are not contacted with NK cells, a control group of non-modified NK cells, or a control group of modified NK cells that do not comprise the inducible co-stimulatory polypeptide discussed herein. In some embodiments, the amount of cytotoxicity induced by contacting the target cells with the modified NK cells is 10, 20, 30, 40, 50, 60, 70, 80, 90, or 95% or more greater than the amount of cytotoxicity induced by contacting the target cells with a control, or in target cells alone. In some embodiments, the amount of cytotoxicity induced by contacting the target cells with the modified NK cells is 2, 3, 4, 5, 6, 7, 8, 9, 10, 20, 30, 40, 50, 60, 70, 80, 90, 100, 200, 300, 400, 500, 600, 700, 800, 900, or 100 fold or more than the amount of cytotoxicity induced by contacting the target cells with a control, or in target cells alone. In some embodiments, the amount of cytotoxicity induced by contacting the target cells with the modified NK cells in the presence of the ligand inducer, such as, for example, rimiducid, is 10, 20, 30, 40, 50, 60, 70, 80, 90, or 95% or more greater than the amount of cytotoxicity induced by contacting the target cells with a control, or in target cells alone. In some embodiments, the amount of cytotoxicity induced by contacting the target cells with the modified NK cells in the presence of the ligand inducer, such as, for example, rimiducid, is 2, 3, 4, 5, 6, 7, 8, 9, 10, 20, 30, 40, 50, 60, 70, 80, 90, 100, 200, 300, 400, 500, 600, 700, 800, 900, or 100 fold or more than the amount of cytotoxicity induced by contacting the target cells with a control, or in target cells alone.

In some embodiments, an additional dose of ligand is administered to the subject. In some embodiments, an effective amount of multimeric ligand is an amount effective to reduce the number or concentration of target cells or to reduce the symptoms of cytotoxicity. In some embodiments, the cell-mediated response is a T cell-mediated response, a NK-cell mediated response, or a NK-T cell mediated response.

Also provided in some embodiments are methods for treating a subject having a disease or condition associated with expression of a target antigen, comprising administering a multimeric ligand that binds to a multimeric ligand binding region, wherein modified NK cells circulating in the subject express (i) an inducible chimeric signaling polypeptide of the present embodiments and chimeric antigen receptor that binds to the target antigen; or (ii) an inducible chimeric antigen receptor polypeptide of the present embodiments that binds to the target antigen, wherein the target antigen is present on target cells circulating in the subject; and wherein the number or concentration of target cells in the subject is reduced following administration of the multimeric ligand. In some embodiments, the target antigen is expressed by a tumor cell, and the chimeric antigen receptor or the inducible chimeric antigen receptor polypeptide binds to the tumor cell. In some embodiments, following administration of the multimeric ligand, the number or concentration of target cells in the subject is determined, and (i) the administration of the multimeric ligand is discontinued or (ii) an additional dose of multimeric ligand is administered that is lower than the previous dose of multimeric ligand administered. In some embodiments, following administration of the multimeric ligand, the number or concentration of target cells in the subject is determined, and an additional dose of multimeric ligand is administered that is higher than the previous dose of multimeric ligand administered. In some embodiments, the additional dose of multimeric ligand is greater than the previous dose, in some embodiments, the additional dose of multimeric ligand is 10%, 20%, 30%, 40%, 50%, 60%, 70%, 80%, 90%, 100%, 125%, 150%, 175%, 200%, 225%, 250%, 275%, 300%, 400%, 500%, 600%, 700%, 800%, or 1000% greater than the previous dose.

In some embodiments, the multimeric ligand that binds to the multimeric ligand binding region is rimiducid or AP21087

Also provided in some embodiments are methods for stimulating a cell-mediated immune response in a subject, comprising administering an effective amount of modified cells that have been transduced or transfected with a nucleic acid of the present embodiments that express a chimeric signaling polypeptide of the present embodiments to the subject. In some embodiments, the cell-mediated immune response is directed against a target cell. In some embodiments, the modified cell comprises a chimeric antigen receptor, a chimeric antigen receptor polypeptide of the present embodiments, or a recombinant T cell receptor, that binds to an antigen on a target cell. In some embodiments, the target cell is a tumor cell. In some embodiments, the number or concentration of target cells in the subject is reduced following administration of the modified cells. In some embodiments, an additional dose of modified cells is administered to the subject. In some embodiments, the -mediated response is a T cell-mediated response, a NK cell-mediated response, or a NK-T cell-mediated response. Also provided in certain embodiments are methods for providing anti-tumor immunity to a subject, comprising administering to the subject an effective amount of a modified cell that expresses a chimeric signaling polypeptide of any one of the present embodiments. Also provided in certain embodiments are methods for treating a subject having a disease or condition associated with an elevated expression of a target antigen, comprising administering to the subject an effective amount of a modified cell of any one of the present embodiments. In some embodiments, the target antigen is a tumor antigen. Also provided in certain embodiments are methods for reducing the size of a tumor in a subject, comprising administering a modified cell of any one of the present embodiments to the subject, wherein the modified cell comprises a chimeric antigen receptor, a chimeric antigen receptor polypeptide of the present embodiments, or a recombinant T cell receptor, comprising an antigen recognition moiety that binds to an antigen on the tumor.

An “antigen recognition moiety” may be any polypeptide or fragment thereof, such as, for example, an antibody fragment variable domain, either naturally-derived, or synthetic, which binds to an antigen. Examples of antigen recognition moieties include, but are not limited to, polypeptides derived from antibodies, such as, for example, single chain variable fragments (scFv), Fab, Fab′, F(ab′)2, and Fv fragments; polypeptides derived from T Cell receptors, such as, for example, TCR variable domains; and any ligand or receptor fragment that binds to the extracellular cognate protein.

In some embodiments, the modified cell comprises a chimeric Caspase-9 polypeptide comprising a multimeric ligand binding region and a Caspase-9 polypeptide. In some embodiments, the method further comprises administering a multimeric ligand that binds to the multimeric ligand binding region to the subject following administration of the modified cells to the subject. In some embodiments, after administration of the multimeric ligand, the number of modified cells comprising the chimeric Caspase-9 polypeptide is reduced.

In some embodiments of the present methods, the subject has been diagnosed as having a tumor. In some embodiments, the subject has cancer. In some embodiments, the subject has a solid tumor. In some embodiments, the cancer is present in the blood or bone marrow of the subject. In some embodiments, the subject has a blood or bone marrow disease. In some embodiments, the subject has been diagnosed with any condition or condition that can be alleviated by stem cell transplantation. In some embodiments, the subject has been diagnosed with sickle cell anemia or metachromatic leukodystrophy. In some embodiments, the subject has been diagnosed with a condition selected from the group consisting of a primary immune deficiency condition, hemophagocytosis lymphohistiocytosis (HAH) or other hemophagocytic condition, an inherited marrow failure condition, a hemoglobinopathy, a metabolic condition, and an osteoclast condition. In some embodiments, the subject has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XAP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis. In some embodiments, the subject has been diagnosed with leukemia. In some embodiments, the subject has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.

The term “cancer” as used herein is defined as a hyperproliferation of cells whose unique trait-loss of normal controls—results in unregulated growth, lack of differentiation, local tissue invasion, and metastasis. Examples include but are not limited to, melanoma, non-small cell lung, small-cell lung, lung, hepatocarcinoma, leukemia, retinoblastoma, astrocytoma, glioblastoma, gum, tongue, neuroblastoma, head, neck, breast, pancreatic, prostate, renal, bone, testicular, ovarian, mesothelioma, cervical, gastrointestinal, lymphoma, brain, colon, sarcoma or bladder.

The term “hyperproliferative disease” is defined as a disease that results from a hyperproliferation of cells. Exemplary hyperproliferative diseases include, but are not limited to cancer or autoimmune diseases. Other hyperproliferative diseases may include vascular occlusion, restenosis, atherosclerosis, or inflammatory bowel disease.

The terms “blood disease”, “blood disease” and/or “diseases of the blood” as used herein, refers to conditions that affect the production of blood and its components, including but not limited to, blood cells, hemoglobin, blood proteins, the mechanism of coagulation, production of blood, production of blood proteins, the like and combinations thereof. Non-limiting examples of blood diseases include anemias, leukemias, lymphomas, hematological neoplasms, albuminemias, haemophilias and the like.

The term “bone marrow disease” as used herein, refers to conditions leading to a decrease in the production of blood cells and blood platelets. In some bone marrow diseases, normal bone marrow architecture can be displaced by infections (e.g., tuberculosis) or malignancies, which in turn can lead to the decrease in production of blood cells and blood platelets. Non-limiting examples of bone marrow diseases include leukemias, bacterial infections (e.g., tuberculosis), radiation sickness or poisoning, apnocytopenia, anemia, multiple myeloma and the like.

The term “patient” or “subject” are interchangeable, and, as used herein include, but are not limited to, an organism or animal; a mammal, including, e.g., a human, non-human primate (e.g., monkey), mouse, pig, cow, goat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate.

### Engineering Expression Constructs

Expression constructs that express the present chimeric antigen receptors, chimeric signaling polypeptides, and inducible safety switches are provided herein. In some examples, one or more polypeptide is said to be “operatively linked.” In general, the term “operably linked” is meant to indicate that the promoter sequence is functionally linked to a second sequence, wherein the promoter sequence initiates and mediates transcription of the DNA corresponding to the second sequence.

As used herein, the term “cDNA” is intended to refer to DNA prepared using messenger RNA (mRNA) as template. The advantage of using a cDNA, as opposed to genomic DNA or DNA polymerized from a genomic, non- or partially processed RNA template, is that the cDNA primarily contains coding sequences of the corresponding protein. There are times when the full or partial genomic sequence is used, such as where the non-coding regions are required for optimal expression or where non-coding regions such as introns are to be targeted in an antisense strategy.

As used herein, the term “polypeptide” is defined as a chain of amino acid residues, usually having a defined sequence. As used herein the term polypeptide may be interchangeable with the term “proteins”.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. The term “therapeutic construct” may also be used to refer to the expression construct or transgene. The expression construct or transgene may be used, for example, as a therapy to treat hyperproliferative diseases or disorders, such as cancer, thus the expression construct or transgene is a therapeutic construct or a prophylactic construct. As used herein with reference to a disease, disorder or condition, the terms “treatment”, “treat”, “treated”, or “treating” refer to prophylaxis and/or therapy.

Expression constructs may comprise one or more isolated nucleic acids. The term “isolation” as applied to a nucleic acid refers to the separation of one region of a nucleotide sequence from other regions of the nucleotide sequence. Thus, isolated nucleic acids are isolated from chromosomes. Isolation may, for example, be performed using an amplification reaction, such as, for example, PCR; in other examples, nucleic acids may be isolated from the cells from which they naturally are found. A pool of isolated nucleic acids may be enriched in nucleic acid segments containing only sequences for a particular region of interest. In some embodiments, isolated nucleic acids are shorter than full length sequences encoding an entire protein.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra.

In certain examples, a polynucleotide coding for the chimeric antigen receptor, is included in the same vector, such as, for example, a viral or plasmid vector, as a polynucleotide coding for a second polypeptide. This second polypeptide may be, for example, a chimeric signaling polypeptide, an inducible caspase polypeptide, as discussed herein, or a marker polypeptide. In these examples, the construct may be designed with one promoter operably linked to a nucleic acid comprising a polynucleotide coding for the two polypeptides, linked by a 2A polypeptide. In this example, the first and second polypeptides are separated during translation, resulting in two polypeptides, or, in examples including a leaky 2A, either one, or two polypeptides. In other examples, the two polypeptides may be expressed separately from the same vector, where each nucleic acid comprising a polynucleotide coding for one of the polypeptides is operably linked to a separate promoter. In yet other examples, one promoter may be operably linked to the two polynucleotides, directing the production of two separate RNA transcripts, and thus two polypeptides; in one example, the promoter may be bi-directional, and the coding regions may be in opposite directions 5′-3′. Therefore, the expression constructs discussed herein may comprise at least one, or at least two promoters.

In some embodiments, a nucleic acid construct is contained within a viral vector. In certain embodiments, the viral vector is a retroviral vector. In certain embodiments, the viral vector is an adenoviral vector or a lentiviral vector. It is understood that in some embodiments, a cell is contacted with the viral vector ex vivo, and in some embodiments, the cell is contacted with the viral vector in vivo. Thus, an expression construct may be inserted into a vector, for example a viral vector or plasmid. The steps of the methods provided may be performed using any suitable method; these methods include, without limitation, methods of transducing, transforming, or otherwise providing nucleic acid to the cell, described herein.

As used herein, the term “gene” is defined as a functional protein-, polypeptide-, or peptide-encoding unit. As will be understood, this functional term includes genomic sequences, cDNA sequences, and smaller engineered gene segments that express, or are adapted to express, proteins, polypeptides, domains, peptides, fusion proteins and/or mutants.

A “nucleic acid” as used herein generally refers to a molecule (one, two or more strands) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompasses the terms “oligonucleotide” and “polynucleotide,” each as a subgenus of the term “nucleic acid.”

As used herein, the term “polynucleotide” is defined as a chain of nucleotides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, polynucleotides include mutations of the polynucleotides, include but are not limited to, mutation of the nucleotides, or nucleosides by methods well known in the art. A nucleic acid may comprise one or more polynucleotides.

Nucleic acids may be, be at least, be at most, or be about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides, or any range derivable therein, in length.

Nucleic acids herein provided may have regions of identity or complementarity to another nucleic acid. It is contemplated that the region of complementarity or identity can be at least 5 contiguous residues, though it is specifically contemplated that the region is, is at least, is at most, or is about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 contiguous nucleotides.

“Function-conservative variants” are proteins or enzymes in which a given amino acid residue has been changed without altering overall conformation and function of the protein or enzyme, including, but not limited to, replacement of an amino acid with one having similar properties, including polar or non-polar character, size, shape and charge. Conservative amino acid substitutions for many of the commonly known non-genetically encoded amino acids are well known in the art. Conservative substitutions for other non-encoded amino acids can be determined based on their physical properties as compared to the properties of the genetically encoded amino acids.

Amino acids other than those indicated as conserved may differ in a protein or enzyme so that the percent protein or amino acid sequence similarity between any two proteins of similar function may vary and can be, for example, at least 70%, at least 80%, at least 90%, and at least 95%, as determined according to an alignment scheme. As referred to herein, “sequence similarity” means the extent to which nucleotide or protein sequences are related. The extent of similarity between two sequences can be based on percent sequence identity and/or conservation. “Sequence identity” herein means the extent to which two nucleotide or amino acid sequences are invariant. “Sequence alignment” means the process of lining up two or more sequences to achieve maximal levels of identity (and, in the case of amino acid sequences, conservation) for the purpose of assessing the degree of similarity. Numerous methods for aligning sequences and assessing similarity/identity are known in the art such as, for example, the Cluster Method, wherein similarity is based on the MEGALIGN algorithm, as well as BLASTN, BLASTP, and FASTA. When using any of these programs, the settings may be selected that result in the highest sequence similarity.

As used herein, the term “promoter” is defined as a DNA sequence recognized by the synthetic machinery of the cell, or introduced synthetic machinery, required to initiate the specific transcription of a gene. In some embodiments, the promoter is a developmentally regulated promoter. The term “developmentally regulated promoter” as used herein refers to a promoter that acts as the initial binding site for RNA polymerase to transcribe a gene which is expressed under certain conditions that are controlled, initiated by or influenced by a developmental program or pathway. As used herein, the term “under transcriptional control,” “operably linked,” or “operatively linked” is defined as the promoter is in the correct location and orientation in relation to the nucleic acid to control RNA polymerase initiation and expression of the gene. In general, the term “operably linked” is meant to indicate that the promoter sequence is functionally linked to a second sequence, wherein, for example, the promoter sequence initiates and mediates transcription of the DNA corresponding to the second sequence.

The particular promoter employed to control the expression of a polynucleotide sequence of interest is not believed to be important, so long as it is capable of directing the expression of the polynucleotide in the targeted cell. Thus, where a human cell is targeted the polynucleotide sequence-coding region may, for example, be placed adjacent to and under the control of a promoter that is capable of being expressed in a human cell. Generally speaking, such a promoter might include either a human or viral promoter. Promoters may be selected that are appropriate for the vector used to express the CARs and other polypeptides provided herein.

In various embodiments, where, for example, the expression vector is a retrovirus, an example of an appropriate promoter is the Murine Moloney leukemia virus promoter. In other embodiments, the promoter may be, for example, the (CMV) immediate early gene promoter, the SV40 early promoter, the Rous sarcoma virus long terminal repeat, β-actin, rat insulin promoter and glyceraldehyde-3-phosphate dehydrogenase can be used to obtain high-level expression of the coding sequence of interest. The use of other viral or mammalian cellular or bacterial phage promoters which are well known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized.

Promoters, and other regulatory elements, are selected such that they are functional in the desired cells or tissue. In addition, this list of promoters should not be construed to be exhaustive or limiting; other promoters that are used in conjunction with the promoters and methods disclosed herein.

The nucleic acids discussed herein may comprise one or more polynucleotides. In some embodiments, one or more polynucleotides may be described as being positioned, or “is” “5” or “3” of another polynucleotide, or positioned in “5′ to 3′ order”. The reference 5′ to 3′ in these contexts is understood to refer to the direction of the coding regions of the polynucleotides in the nucleic acid, for example, where a first polynucleotide is positioned 5′ of a second polynucleotide and connected with a third polynucleotide encoding a non-cleave able linker polypeptide, the translation product would result in the polypeptide encoded by the first polynucleotide positioned at the amino terminal end of a larger polypeptide comprising the translation products of the first, third, and second polynucleotides.

In yet other examples, two polypeptides, such as, for example, the chimeric stimulating molecule or a MyD88/CD40 chimeric antigen receptor polypeptide, and a second polypeptide, may be expressed in a cell using two separate vectors. The cells may be co-transfected or co-transformed with the vectors, or the vectors may be introduced to the cells at different times.

The polypeptides may vary in their order, from the amino terminus to the carboxy terminus. For example, in the chimeric stimulating molecule, the order of the MyD88 polypeptide, CD40 polypeptide, and any additional polypeptide, may vary. In the chimeric antigen receptor, the order of the MyD88 polypeptide, CD40 polypeptide, and any additional polypeptide, such as, for example, the CD3 ζ polypeptide may vary. The order of the various domains may be assayed using methods such as, for example, those discussed herein, to obtain the optimal expression and activity.

In some embodiments, where an expression construct encodes a MyD88 polypeptide, the polypeptide may be a portion of the full-length MyD88 polypeptide. By MyD88, or MyD88 polypeptide, is meant the polypeptide product of the myeloid differentiation primary response gene 88, for example, but not limited to the human version, cited as NCBI Gene ID 4615. In some embodiments, an expression construct encodes a portion of the MyD88 polypeptide lacking the TIR domain. In some embodiments, the expression construct encodes a portion of the MyD88 polypeptide containing the DD (death domain) or the DD and intermediary domains. By “truncated,” is meant that the protein is not full length and may lack, for example, a domain. For example, a truncated MyD88 is not full length and may, for example, be missing the TIR domain. In some embodiments, the truncated MyD88 polypeptide has an amino acid sequence of SEQ ID NO: 2 or SEQ ID NO: 119, or a functionally equivalent fragment thereof. In some embodiments, the truncated MyD88 polypeptide is encoded by the nucleotide sequences of SEQ ID NO: 1, or a functionally equivalent fragment thereof. A functionally equivalent portion of the MyD88 polypeptide has substantially the same ability to stimulate intracellular signaling as the polypeptide of SEQ ID NO 118, with at least 50%, 60%, 70%, 80%, 90%, or 95% of the activity of the polypeptide of SEQ ID NO: 118. By a nucleic acid sequence coding for “truncated MyD88” is meant the nucleic acid sequence coding for a truncated MyD88 polypeptide, the term may also refer to the nucleic acid sequence including the portion coding for any amino acids added as an artifact of cloning, including any amino acids coded for by the linkers.

It is understood that where a method or construct refers to a truncated MyD88 polypeptide, the method may also be used, or the construct designed to refer to another MyD88 polypeptide, such as a full length MyD88 polypeptide. Where a method or construct refers to a full length MyD88 polypeptide, the method may also be used, or the construct designed to refer to a truncated MyD88 polypeptide. In the methods herein, in which a chimeric polypeptide comprises a MyD88 polypeptide (or portion thereof) and a CD40 polypeptide (or portion thereof), the MyD88 polypeptide of the chimeric polypeptide may be located either upstream or downstream from the CD40 polypeptide. In certain embodiments, the MyD88 polypeptide (or portion thereof) is located upstream of the CD40 polypeptide (or portion thereof). As used herein, the term “functionally equivalent,” as it relates to MyD88, or a portion thereof, for example, refers to a MyD88 polypeptide that stimulates a cell-signaling response or a nucleic acid encoding such a MyD88 polypeptide. “Functionally equivalent” refers, for example, to a MyD88 polypeptide that is lacking a TIR domain but is capable of stimulating a cell-signaling response.

In certain embodiments, a modified cell populations comprise a nucleic acid molecule that comprises a promoter operably linked to a first polynucleotide encoding a chimeric stimulating molecule, wherein the chimeric stimulating molecule comprises (i) a MyD88 polypeptide or a truncated MyD88 polypeptide lacking the TIR domain; and (ii) a CD40 cytoplasmic polypeptide region lacking the CD40 extracellular domain, and wherein the chimeric stimulating molecule does not include a membrane targeting region; and
b) a second polynucleotide encoding a T cell receptor, a T cell receptor-based chimeric antigen receptor, or a chimeric antigen receptor; and
c) a third polynucleotide encoding a chimeric Caspase-9 polypeptide comprising a multimeric ligand binding region and a Caspase-9 polypeptide. It is understood that the order of the polynucleotides may vary and may be tested to determine the suitability of the construct for any particular method, thus, the nucleic acid may include the polynucleotides in the varying orders, which also take into account a variation in the order of the MyD88 polypeptide or truncated MyD88 polypeptide-encoding sequence and the CD40 cytoplasmic polypeptide region-encoding sequence in the first polynucleotide. Thus, the first polynucleotide may encode a polypeptide having and order of MyD88/CD40, truncatedMyD88/CD40, CD40/MyD88, or CD40/truncated MyD88. And, the nucleic acid may include the first through third polynucleotides in any of the following orders, where 1, 2, 3, indicate a first, second, or third order of the polynucleotides in the nucleic acid from the 5′ to 3′ direction. It is understood that other polynucleotides, such as those that code for a 2A polypeptide, for example, may be present between the three listed polynucleotides; this Table is meant to designate the order of the first through third polynucleotides:

TABLE 1

| First polynucleotide | | |
|---|---|---|
| encoding a Chimeric | Second polynucleotide | |
| stimulating molecule | encoding a T cell | Third |
| comprising MyD88 or | receptor, a T cell | polynucleotide |
| truncated MyD88 and | receptor-based chimeric | encoding a |
| CD40 cytoplasmic | antigen receptor, or a | chimeric caspse-9 |
| polypeptide region. | chimeric antigen receptor. | polypeptide. |
| 1 | 2 | 3 |
| 1 | 3 | 2 |
| 2 | 1 | 3 |
| 3 | 1 | 2 |
| 2 | 3 | 1 |
| 3 | 2 | 1 |

Similarly, the nucleic acids may include only two of the polynucleotides, coding for two of the polypeptides provided in the table above. In some examples, a cell is transfected or transduced with a nucleic acid comprising the three polynucleotides included in Table A above. In other examples, a cell is transfected or transduced with a nucleic acid that encodes two of the polynucleotides, coding for two of the polypeptides, as provided, for example, in Table B.

TABLE 2

| First polynucleotide | | |
|---|---|---|
| encoding a Chimeric | Second polynucleotide | |
| stimulating molecule | encoding a T cell | Third |
| comprising MyD88 or | receptor, a T cell | polynucleotide |
| truncated MyD88 and | receptor-based chimeric | encoding a |
| CD40 cytoplasmic | antigen receptor, or a | chimeric caspse-9 |
| polypeptide region. | chimeric antigen receptor. | polypeptide. |
| 1 | 2 | |
| 1 | | 2 |
| 2 | 1 | |
| | 1 | 2 |
| 2 | | 1 |
| | 2 | 1 |

In some embodiments, the cell is transfected or transduced with the nucleic acid that encodes two of the polynucleotides, and the cell also comprises a nucleic acid comprising a polynucleotide coding for the third polypeptide. For example, a cell may comprise a nucleic acid comprising the first and second polynucleotides, and the cell may also comprise a nucleic acid comprising a polynucleotide coding for a chimeric Caspase-9 polypeptide. Also, a cell may comprise a nucleic acid comprising the first and third polynucleotides, and the cell may also comprise a nucleic acid comprising a polynucleotide coding for a T cell receptor, a T cell receptor-based chimeric antigen receptor, or a chimeric antigen receptor.

The steps of the methods provided may be performed using any suitable method; these methods include, without limitation, methods of transducing, transforming, or otherwise providing nucleic acid to the cell, presented herein. In some embodiments, the truncated MyD88 peptide is encoded by the nucleotide sequence of SEQ ID NO: 1 (with or without DNA linkers or has the amino acid sequence of SEQ ID NO:2 or SEQ ID NO: 119). In some embodiments, the CD40 cytoplasmic polypeptide region is encoded by a polynucleotide sequence in SEQ ID NO: 56.

### Vectors

It is understood that the vectors provided herein may be modified using methods known in the art to vary the position or order of the regions, to substitute one region for another. For example, a vector comprising a polynucleotide encoding a chimeric signaling polypeptide comprising truncated MC may be substituted with a polynucleotide encoding chimeric signaling polypeptide comprising one, or two or more co-stimulatory polypeptide cytoplasmic signaling regions such as, for example, those selected from the group consisting of CD27, CD28, 4-1BB, OX40, ICOS, RANK, TRANCE, and DAP10. The polynucleotide encoding the CAR may also be modified so that the scFv region may be substituted with one having the same, or different target specificity; the transmembrane region may be substituted with a different transmembrane region; a stalk polypeptide may be added. Polynucleotides encoding marker polypeptides may be included within or separate from one of the polypeptides; polynucleotides encoding additional polypeptides coding for safety switches may be added, polynucleotides coding for linker polypeptides, or non-coding polynucleotides or spacers may be added, or the order of the polynucleotides 5′ to 3′ may be changed.

The vectors provided in the present application may be modified as discussed herein, for example, to substitute polynucleotides coding for regions of the chimeric antigen receptor, for example, the CD19-specific scFV, or other scFvs provided, with a scFv directed against other target antigens, such as, for example, BCMA, CD33, NKG2D, PSMA, PSCA, MUC1, CD19, ROR1, Mesothelin, GD2, CD123, MUC16, Her2/Neu, CD20, CD30, PRAME, NY-ESO-1, and EGFRvIII. scFvs that target these antigens are known in the art, see, e.g., Berahovich et al., 2018 Cancers 10, 323; Wang et al., 2015, Molecular Therapy 23 (1): 184-191; Han et al. 2018, Am J Cancer Res 8 (1): 106-119; Qin et al., 2017; Hematol & Oncol. 10:68; Jachimowicz et al. 2011, Molecular Cancer Therapeutics 10 (6): 1036-1045; Schau et al. 2019, Scientific reports 9:3299; Han et al. 2017, Clin Cancer Res 23 (13): 3385-3395; Nejatollahi et al., 2013, Journal of Oncology Vol. 2013, Article ID 839831; Kugler et al, 2010 BJH Vol 150, 574-586. In a specific embodiment, a PSCA scFv comprises (or consists of) a sequence(s) disclosed herein (e.g., VH and VL sequences disclosed in the Examples below). In another specific embodiment, a CD123 scFv comprises (or consists of) a sequence(s) disclosed herein (e.g., VH and VL sequences disclosed in the Examples below). In another specific embodiment, a BCMA scFv comprises (or consists of) a sequence(s) disclosed herein (e.g., VH and VL sequences disclosed in the Examples below). The vector may also be modified with appropriate substitutions of each polypeptide region, as discussed herein. The vector may be modified to remove the inducible caspase-9 safety switch (1), to position the inducible caspase-9 safety switch to a position 3′ of the MyD88-CD40 polypeptide (**), to substitute the inducible caspase-9 safety switch with a different inducible caspase polypeptide-based switch, or to substitute the inducible caspase-9 safety switch with a different polypeptide safety switch.

The vectors provided herein may be modified to substitute the MyD88-CD40 (MC) portions with one, or two or more co-stimulatory polypeptide cytoplasmic signaling regions such as, for example, those selected from the group consisting of CD27, CD28, 4-1BB, OX40, ICOS, RANK, TRANCE, and DAP10. Co-stimulating polypeptides may comprise, but are not limited to, the amino acid sequences provided herein, and may include functional conservative mutations, including deletions or truncations, and may comprise amino acid sequences that are 70%, 75%, 80%, 85%, 90%, 95% or 100% identical to the amino acid sequences provided herein.

The vectors provided herein may be modified to substitute a polynucleotide coding for a linker sequence, where the linker polypeptide is not a 2A polypeptide, between the CAR polypeptide and the MC polypeptide or other co-stimulatory polypeptide. For example, nucleic acids provided herein may comprise, a polynucleotide coding for a MC polypeptide, or a co-stimulatory polypeptide signaling region 3′ of a polynucleotide coding for the CD34 portion of the CAR, where the two polynucleotides are separated by a polynucleotide coding for a 2A linker, or, where the two polynucleotides are not separated by a polynucleotide coding for a 2A linker. In some embodiments, the two polynucleotides may be separated by a polynucleotide coding for a linker polypeptide having, for example, about 5 to 20 amino acids, or, for example, about 6 to 10 amino acids, where the linker polypeptide does not comprise a 2A polypeptide sequence.

### Selectable Markers

In certain embodiments, the expression constructs contain nucleic acid constructs whose expression is identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin and histidinol are useful selectable markers. Alternatively, enzymes such as Herpes Simplex Virus thymidine kinase (tk) are employed. Immunologic surface markers containing the extracellular, non-signaling domains or various proteins (e.g. CD34, CD19, LNGFR) also can be employed, permitting a straightforward method for magnetic or fluorescence antibody-mediated sorting. The selectable marker employed is not believed to be important, so long as it is capable of being expressed simultaneously with the nucleic acid encoding a gene product. Further examples of selectable markers include, for example, reporters such as GFP, EGFP, β-gal or chloramphenicol acetyltransferase (CAT). In certain embodiments, the marker protein, such as, for example, CD19 is used for selection of the cells for transfusion, such as, for example, in immunomagnetic selection. As discussed herein, a CD19 marker is distinguished from an anti-CD19 antibody, or, for example, an scFv, TCR, or other antigen recognition moiety that binds to CD19.

In certain embodiments, the marker polypeptide is linked to the inducible chimeric stimulating molecule. For example, the marker polypeptide may be linked to the inducible chimeric stimulating molecule via a polypeptide sequence, such as, for example, a cleavable 2A-like sequence.

The NK cells provided herein may express a cell surface transgene marker, present on an expression vector that expresses the CAR, or, in some embodiments, present on an expression vector that encodes a protein other than the CAR, such as, for example a pro-apoptotic polypeptide safety switch, such as i-Casp9, or the costimulatory polypeptide that is co-expressed with the CAR.

In one embodiment, the cell surface transgene marker is a truncated CD19 (ΔCD19) polypeptide (Di Stasi et al. (2011) supra, that comprises a human CD19 truncated at amino acid 333 to remove most of the intracytoplasmic domain. The extracellular CD19 domain can still be recognised (e.g. in flow cytometry, FACS or MACS) but the potential to trigger intracellular signalling is minimised. CD19 is normally expressed by B cells, rather than by T cells or NK, so selection of CD19+ T cells permits the genetically-modified NK cells to be separated from unmodified donor NK cells.

In some embodiments, a polypeptide may be included in the polypeptide, for example, the CAR encoded by the expression vector to aid in sorting cells. In some embodiments, the expression vectors used to express the chimeric antigen receptors or chimeric stimulating molecules provided herein further comprise a polynucleotide that encodes the 16 amino acid CD34 minimal epitope. In some embodiments, such as certain embodiments provided in the examples herein, the CD34 minimal epitope is incorporated at the amino terminal position of the CD8 stalk.

### Linker Polypeptides

Linker polypeptides include, for example, cleavable and non-cleavable linker polypeptides. Non-cleavable polypeptides may include, for example, any polypeptide that may be operably linked between the MyD88-CD40 chimeric polypeptide, the MyD88 polypeptide, the CD40 polypeptide, or the costimulatory polypeptide cytoplasmic signaling region and the CD34 portion of the chimeric antigen receptor. Linker polypeptides include those for example, consisting of about 2 to about 30 amino acids, (e.g., furin cleavage site, (GGGGS)ₙ). In some embodiments, the linker polypeptide consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids. In some embodiments, the linker polypeptide consists of about 18 to 22 amino acids. In some embodiments, the linker polypeptide consists of 20 amino acids. In a specific embodiment, the linker comprises (consists of) a sequence disclosed herein (e.g., a sequence disclosed in the Examples below). In some embodiments, cleavable linkers include linkers that are cleaved by an enzyme exogenous to the modified cells in the population, for example, an enzyme encoded by a polynucleotide that is introduced into the cells by transfection or transduction, either at the same time or a different time as the polynucleotide that encodes the linker. In some embodiments, cleavable linkers include linkers that are cleaved by an enzyme endogenous to the modified cells in the population, including, for example, enzymes that are naturally expressed in the cell, and enzymes encoded by polynucleotides native to the cell, such as, for example, lysozyme.

### 2A Peptide Bond-Skipping Sequences

2A-like sequences, or “peptide bond-skipping” 2A sequences, are derived from, for example, many different viruses, including, for example, from Thosea asigna. These sequences are sometimes also known as “peptide skipping sequences.” When this type of sequence is placed within a cistron, between two polypeptides that are intended to be separated, the ribosome appears to skip a peptide bond, in the case of Thosea asigna sequence; the bond between the Gly and Pro amino acids at the carboxy terminal “P-G-P” is omitted. This may, leave two to three polypeptides, for example, an inducible chimeric pro-apoptotic polypeptide and a chimeric antigen receptor, or, for example, a marker polypeptide and an inducible chimeric pro-apoptotic polypeptide. When this sequence is used, the polypeptide that is encoded 5′ of the 2A sequence may end up with additional amino acids at the carboxy terminus, including the Gly residue and any upstream residues in the 2A sequence. The peptide that is encoded 3′ of the 2A sequence may end up with additional amino acids at the amino terminus, including the Pro residue and any downstream residues following the 2A sequence. In some embodiments, the cleavable linker is a 2A polypeptide derived from porcine teschovirus-1 (P2A). In some embodiments, the 2A cotranslational sequence is a 2A-like sequence. In some embodiments, the 2A cotranslational sequence is T2A (thosea asigna virus 2A), F2A (foot and mouth disease virus 2A), P2A (porcine teschovirus-1 2A), BmCPV 2A (cytoplasmic polyhedrosis virus 2A) BmIFV 2A (flacherie virus of B. mori 2A), or E2A (equine rhinitis A virus 2A). In some embodiments, the 2A cotranslational sequence is T2A-GSG, F2A-GSG, P2A-GSG, or E2A-GSG. In some embodiments, the 2A cotranslational sequence is selected from the group consisting of T2A, P2A and F2A. By “cleavable linker” is meant that the linker is cleaved by any means, including, for example, non-enzymatic means, such as peptide skipping, or enzymatic means. (Donnelly, M L 2001, J. Gen. Virol. 82: 1013-25). In a specific embodiment, a 2TA comprises (or consists of) a sequence disclosed herein. comprises (consists of) a sequence disclosed herein (e.g., a sequence disclosed in the Examples below).

The 2A-like sequences are sometimes “leaky” in that some of the polypeptides are not separated during translation, and instead, remain as one long polypeptide following translation. One theory as to the cause of the leaky linker, is that the short 2A sequence occasionally may not fold into the required structure that promotes ribosome skipping (a “2A fold”). In these instances, ribosomes may not miss the proline peptide bond, which then results in a fusion protein. To reduce the level of leakiness, and thus reduce the number of fusion proteins that form, a GSG (or similar) linker may be added to the amino terminal side of the 2A polypeptide; the GSG linker blocks secondary structures of newly-translated polypeptides from spontaneously folding and disrupting the ‘2A fold’.

In certain embodiments, a 2A linker includes the amino acid sequence of SEQ ID NO: 20. In certain embodiments, the 2A linker further includes a GSG amino acid sequence at the amino terminus of the polypeptide, in other embodiments, the 2A linker includes a GSGPR amino acid sequence at the amino terminus of the polypeptide. Thus, by a “2A” sequence, the term may refer to a 2A sequence in an example described herein or may also refer to a 2A sequence as listed herein further comprising a GSG or GSGPR sequence at the amino terminus of the linker.

In some embodiments, the linker, for example, the 2A linker, is cleaved in about 10, 20, 30, 40, 50, 60, 70, 75, 80, 85, 90, 95, 98, or 99% of the chimeric antigen receptors, that is, the chimeric antigen receptor portion is separated from the chimeric MyD88 and CD40, the MyD88 polypeptide, the CD40 polypeptide, or the costimulatory polypeptide cytoplasmic signaling region, such as, CD28, OX40, 4-1 BB or the like. In other embodiments the 2A linker is cleaved in about 75, 80, 85, 90, 95, 98, or 99% of the chimeric antigen receptors. In some embodiments, the 2A linker is cleaved in about 80-99% of the chimeric antigen receptors. In some embodiments, the 2A linker is cleaved in about 90% of the chimeric antigen receptors. In some embodiments, a constitutive active chimeric antigen receptor polypeptide is present in the modified cells, where the 2A linker is not cleaved, that is, the chimeric antigen receptor portion is linked to the chimeric MyD88 and CD40, the MyD88 polypeptide, the CD40 polypeptide, or the costimulatory polypeptide cytoplasmic signaling region, such as, CD28, OX40, 4-1BB or the like, representing about 1, 2, 5, 10, 15, 20, 25, 30, 40, 50, 60, 70, 80, or 90% of the chimeric antigen receptor polypeptide. In other embodiments the 2A linker is not cleaved in about 5, 10, 15, 20, or 25% of the chimeric antigen receptors. In some embodiments, the 2A linker is not cleaved in about 5-20% of the chimeric antigen receptors. In some embodiments, the 2A linker is not cleaved in about 10% of the chimeric antigen receptors.

### Membrane-Targeting

A membrane-targeting sequence provides for transport of the chimeric protein to the cell surface membrane, where the same or other sequences can encode binding of the chimeric protein to the cell surface membrane. Molecules in association with cell membranes contain certain regions that facilitate the membrane association, and such regions can be incorporated into a chimeric protein molecule to generate membrane-targeted molecules. For example, some proteins contain sequences at the N-terminus or C-terminus that are acylated, and these acyl moieties facilitate membrane association. Such sequences are recognized by acyltransferases and often conform to a particular sequence motif. Certain acylation motifs are capable of being modified with a single acyl moiety (often followed by several positively charged residues (e.g. human c-Src: M-G-S—N—K—S—K—P—K-D-A-S-Q-R—R—R, SEQ ID NO:120) to improve association with anionic lipid head groups) and others are capable of being modified with multiple acyl moieties. For example, the N-terminal sequence of the protein tyrosine kinase Src can comprise a single myristoyl moiety. Dual acylation regions are located within the N-terminal regions of certain protein kinases, such as a subset of Src family members (e.g., Yes, Fyn, Lck) and G-protein alpha subunits. Such dual acylation regions often are located within the first eighteen amino acids of such proteins, and conform to the sequence motif Met-Gly-Cys-Xaa-Cys, where the Met is cleaved, the Gly is N-acylated and one of the Cys residues is S-acylated. The Gly often is myristoylated and a Cys can be palmitoylated. Acylation regions conforming to the sequence motif Cys-Ala-Ala-Xaa (so called “CAAX boxes”), which can be modified with C15 or C10 isoprenyl moieties, from the C-terminus of G-protein gamma subunits and other proteins (e.g., World Wide Web address ebi.ac.uk/interpro/DisplaylproEntry?ac=IPR001230) also can be utilized. These and other acylation motifs include, for example, those discussed in Gauthier-Campbell et al., Molecular Biology of the Cell 15:2205-2217 (2004); Glabati et al., Biochem. J. 303: 697-700 (1994) and Zlakine et al., J. Cell Science 110:673-679 (1997), and can be incorporated in chimeric molecules to induce membrane localization. In some embodiments, a chimeric polypeptide comprising a costimulatory polypeptide cytoplasmic signaling region provided herein comprises a membrane-targeting region, and optionally, a multimeric ligand binding region, in some embodiments, chimeric MyD88, chimeric truncated MyD88, chimeric MyD88-CD40, or chimeric truncated MyD88-CD40, polypeptides provided herein, comprise a membrane-targeting region, and optionally, a multimeric ligand binding region. In some embodiments, the membrane-targeting region comprises a myristoylation region. In some embodiments, the membrane-targeting region is selected from the group consisting of myristoylation-targeting sequence, palmitoylation-targeting sequence, prenylation sequences (i.e., farnesylation, geranyl-geranylation, CAAX Box), protein-protein interaction motifs or transmembrane sequences (utilizing signal peptides) from receptors. Examples include those discussed in, for example, ten Klooster J P et al, Biology of the Cell (2007) 99, 1-12, Vincent, S., et al., Nature Biotechnology 21:936-40, 1098 (2003). In a specific embodiment, a myristolyation peptide comprises (or consists of) a sequence disclosed herein (e.g., a sequence disclosed in the Examples disclosed herein).

Where a polypeptide does not include a membrane-targeting region, or lacks a membrane-targeting region, such as certain chimeric polypeptides provided herein, the polypeptide does not include a region that provides for transport of the chimeric protein to a cell membrane. The polypeptide may, for example, not include a sequence that transports the polypeptide to the cell surface membrane, or the polypeptide may, for example, include a dysfunctional membrane-targeting region, that does not transport the polypeptide to the cell surface membrane, for example, a myristoylation region that includes a proline that disrupts the function of the myristoylation-targeting region. (see, for example, Resh, M. D., Biochim. Biophys. Acta. 1451: 1-16 (1999)). Polypeptides that are not transported to the membrane are considered to be cytoplasmic polypeptides.

### Methods of Gene Transfer/Genetic Modification of NK Cells

In order to mediate the effect of the transgene expression in a cell, it will be necessary to transfer the expression constructs into a cell. Such transfer may employ viral or non-viral methods of gene transfer. This section provides a discussion of methods and compositions of gene transfer.

A transformed cell comprising an expression vector is generated by introducing into the cell the expression vector. Suitable methods for polynucleotide delivery for transformation of an organelle, a cell, a tissue or an organism for use with the current methods include virtually any method by which a polynucleotide (e.g., DNA) can be introduced into an organelle, a cell, a tissue or an organism.

The terms “cell,” “cell line,” and “cell culture” as used herein may be used interchangeably. All of these terms also include their progeny, which are any and all subsequent generations. It is understood that all progeny may not be identical due to deliberate or inadvertent mutations. As used herein, the term “ex vivo” refers to “outside” the body. The terms “ex vivo” and “in vitro” can be used interchangeably herein.

The term “transfection” and “transduction” are interchangeable and refer to the process by which an exogenous nucleic acid sequence is introduced into a eukaryotic host cell. Transfection (or transduction) can be achieved by any one of a number of means including electroporation, microinjection, gene gun delivery, retroviral infection, lipofection, superfection and the like.

Any appropriate method may be used to transfect or transform the cells, for example, the NK cells, or to administer the nucleotide sequences or compositions of the present methods. Certain non-limiting examples are presented herein. In some embodiments, the virsl vector is an SFG-based viral vector, as discussed in Tey et al. (2007) Biol Blood Marrow Transpl 13: 913-24 and by Di Stasi et al., (2011) N Engl J Med 365: 1673-83 (2011).

NK cells that are genetically modified as disclosed herein are useful for administering to subjects who can benefit from donor lymphocyte administration. These subjects will typically be humans, so the invention will typically be performed using human NK cells.

The modified cells may be obtained from a donor, or may be cells obtained from the patient, for example, the cells may be autologous, syngeneic, or allogeneic. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. By “therapeutic cell” is meant a cell used for cell therapy, that is, a cell administered to a subject to treat or prevent a condition or disease.

By “obtained or prepared” as, for example, in the case of cells, is meant that the cells or cell culture are isolated, purified, or partially purified from the source, where the source may be, for example, umbilical cord blood, bone marrow, or peripheral blood. The terms may also apply to the case where the original source, or a cell culture, has been cultured and the cells have replicated, and where the progeny cells are now derived from the original source.

Peripheral blood: The term “peripheral blood” as used herein, refers to cellular components of blood (e.g., red blood cells, white blood cells and platelets), which are obtained or prepared from the circulating pool of blood and not sequestered within the lymphatic system, spleen, liver or bone marrow.

Umbilical cord blood: Umbilical cord blood is distinct from peripheral blood and blood sequestered within the lymphatic system, spleen, liver or bone marrow. The terms “umbilical cord blood”, “umbilical blood” or “cord blood”, which can be used interchangeably, refers to blood that remains in the placenta and in the attached umbilical cord after child birth. Cord blood often contains stem cells including hematopoietic cells.

The term “allogeneic” as used herein, refers to HLA or MHC loci that are antigenically distinct between the host and donor cells. Thus, cells or tissue transferred from the same species can be antigenically distinct. Syngeneic mice can differ at one or more loci (congenics) and allogeneic mice can have the same background. The term “autologous” means a cell, nucleic acid, protein, polypeptide, or the like derived from the same individual to which it is later administered. The modified cells of the present methods may, for example, be autologous cells, such as, for example, autologous NK cells.

As used herein, the term “syngeneic” refers to cells, tissues or animals that have genotypes that are identical or closely related enough to allow tissue transplant, or are immunologically compatible. For example, identical twins or animals of the same inbred strain. Syngeneic and isogeneic can be used interchangeably.

Donor NK cells can be cultured prior to being genetically modified by any suitable method known in the art including the one described herein.

The NK cells can be transduced using a viral vector encoding polynucleotides of the present application. Suitable transduction techniques may involve fibronectin fragment CH-296. As an alternative to transduction using a viral vector, cells can be transfected with any suitable method known in the art such as with DNA encoding the suicide switch of interest and a cell surface transgene marker of interest e.g. using calcium phosphate, cationic polymers (such as PEI), magnetic beads, electroporation and commercial lipid-based reagents such as Lipofectamine™ and Fugene™. One result of the transduction/transfection step is that various donor NK cells will now be genetically-modified NK cells which can express the suicide switch of interest.

In some embodiments, the viral vector used for transduction is the retroviral vector disclosed by Tey et al. (2007) Biol Blood Marrow Transpl 13:913-24 and by Di Stasi et al. (2011) supra. This vector is based on Gibbon ape leukemia virus (Gal-V) pseudotyped retrovirus encoding an iCasp9 suicide switch and a ΔCD19 cell surface transgene marker (see further below). It can be produced in the PG13 packaging cell line, as discussed by Tey et al. (2007) supra. Other viral vectors encoding the desired proteins can also be used. In some embodiments, retroviral vectors that can provide a high copy number of proviral integrants per cell are used for transduction.

After transduction/transfection, cells can be separated from transduction/transfection materials and cultured again, to permit the genetically-modified NK cells to expand. TNK cells can be expanded so that a desired minimum number of genetically-modified NK cells is achieved.

Genetically-modified NK cells can then be selected from the population of cells which has been obtained. The suicide switch will usually not be suitable for positive selection of desired NK cells, so in some embodiments, the genetically-modified NK cells should express a cell surface transgene marker of interest. Cells which express this surface marker can be selected e.g. using immunomagnetic techniques. For instance, paramagnetic beads conjugated to monoclonal antibodies which recognise the cell surface transgene marker of interest can be used, for example, using a CliniMACS system (available from Miltenyi Biotec).

In an alternative procedure, genetically-modified NK cells are selected after a step of transduction, are cultured, and are then fed. Thus, the order of transduction, feeding, and selection can be varied.

The result of these procedures is a composition containing donor NK cells which have been genetically modified, and which can thus express, e.g. the costimulatory polypeptide and/or the suicide switch of interest (and, typically, the cell surface transgene marker of interest). These genetically-modified NK cells can be administered to a recipient, but they will usually be cryopreserved (optionally after further expansion) before being administered.

### Methods for Treating a Disease

The present methods also encompass methods of treatment or prevention of a disease where administration of cells by, for example, infusion, may be beneficial.

The cells described herein may be used for cell therapy. The cells may be from a donor or may be cells obtained from the patient. The cells may, for example, be used in regeneration, for example, to replace the function of diseased cells. The cells may also be modified to express a heterologous gene so that biological agents may be delivered to specific microenvironments such as, for example, diseased bone marrow or metastatic deposits. The cells provided in the present application contain a safety switch that may be valuable in a situation where following cell therapy, the activity of the therapeutic cells needs to be removed. increased, or decreased. For example, where progenitor NK cells that express a chimeric antigen receptor are provided to the patient, in some situations there may be an adverse event, such as inappropriate differentiation of the cell into a more mature cell type, or an undesired invitation into another tissue off-target toxicity. Ceasing the administration of the ligand would return the therapeutic NK cells to a non-activated state, remaining at a low, non-toxic, level of expression. Or, for example, where it is necessary to remove the therapeutic cells. The therapeutic cell may work to decrease the tumor cell, or tumor size, and may no longer be needed. In this situation, administration of the ligand may cease, and the therapeutic cells would no longer be activated. If the tumor cells return, or the tumor size increases following the initial therapy, the ligand may be administered again, in order to activate the chimeric antigen receptor-expressing NK cells, and re-treat the patient. In some embodiments, cells are transfected or transduced with nucleic acids that encode the chimeric signaling polypeptides and inducible chimeric signaling polypeptides of the present application.

By “therapeutic cell” is meant a cell used for cell therapy, that is, a cell administered to a subject to treat or prevent a condition or disease. Therapeutic cells may, for example, be NK cells. The therapeutic cells may be, for example, any cell administered to a patient for a desired therapeutic result. The therapeutic cells may be, for example, immune cells such as, for example, T cells, natural killer cells, B cells, tumor infiltrating lymphocytes, or macrophages, or a combination thereof; the therapeutic cells may be, for example, peripheral blood cells, hematopoietic progenitor cells, bone marrow cells, or tumor cells. To further improve the tumor microenvironment to be more immunogenic, the treatment may be combined with one or more adjuvants (e.g., IL-12, TLRs, IDO inhibitors, etc.). In some embodiments, the cells may be delivered to treat a solid tumor, such as, for example, delivery of the cells to a tumor bed.

Also provided in some embodiments are nucleic acid vaccines, such as DNA vaccines, wherein the vaccine comprises a nucleic acid comprising a polynucleotide that encodes an inducible, or constitutive chimeric signaling polypeptide of the present application. The vaccine may be administered to a subject, thereby transforming or transducing target cells in vivo.

As used herein, the term “vaccine” refers to a formulation that contains a composition presented herein which is in a form that is capable of being administered to an animal. Typically, the vaccine comprises a conventional saline or buffered aqueous solution medium in which the composition is suspended or dissolved. In this form, the composition can be used conveniently to prevent, ameliorate, or otherwise treat a condition. Upon introduction into a subject, the vaccine is able to provoke an immune response including, but not limited to, the production of antibodies, cytokines and/or other cellular responses.

An effective amount of the pharmaceutical composition, such as the multimeric ligand presented herein, would be the amount that achieves this selected result of activating the inducible chimeric signaling polypeptide-expressing NK cells, such that over 60%, 70%, 80%, 85%, 90%, 95%, or 97%, or that under 80%, 70%, 60%, 50%, 40%, 30%, 20%, or 10% of the therapeutic cells are activated. The term is also synonymous with “sufficient amount.” The effective amount may also be the amount that achieves the desired therapeutic response, such as, the reduction of tumor size, the decrease in the level of tumor cells, or the decrease in the level of CD19-expressing leukemic cells, compared to the time before the ligand inducer is administered.

By administering a modified cell, it is understood that an effective amount of modified cells is administered.

To determine if an effective amount of ligand or modified cells is administered, any means of assaying or measuring the number of target cells, or amount of target antigen, or size of a tumor may be used to determine whether the number of target cells, amount of target antigen or size of a tumor has increased, decreased, or remained the same. Samples, images, or other means of measurement taken before administration of the modified cells or ligand may be used to compare with samples, images, or other means of measurement taken after administration of the modified cells or ligand. Thus, for example, to determine whether the amount or concentration of cells expressing a target antigen has increased, decreased, or remained the same, a first sample may be obtained from a subject before administration of the ligand or modified cells, and a second sample may be obtained from a subject after administration of the ligand or modified cells. The amount or concentration of cells expressing the target antigen in the first sample may be compared with the amount or concentration of cells expressing the target antigen in the second sample, in order to determine whether the amount or concentration of cells expressing the target antigen has increased, decreased, or remained the same following administration of the ligand or modified cell.

The effective amount for any particular application can vary depending on such factors as the disease or condition being treated, the particular composition being administered, the size of the subject, and/or the severity of the disease or condition. One can empirically determine the effective amount of a particular composition presented herein.

The terms “contacted” and “exposed,” when applied to a cell, tissue or organism, are used herein to describe the process by which the pharmaceutical composition and/or another agent, such as for example a chemotherapeutic or radiotherapeutic agent, are delivered to a target cell, tissue or organism or are placed in direct juxtaposition with the target cell, tissue or organism. To achieve cell killing or stasis, the pharmaceutical composition and/or additional agent(s) are delivered to one or more cells in a combined amount effective to kill the cell(s) or prevent them from dividing.

The administration of the pharmaceutical composition may precede, be concurrent with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein, and one or more agents may be employed.

Diseases that may be treated or prevented include diseases caused by viruses, bacteria, yeast, parasites, protozoa, cancer cells and the like. The pharmaceutical composition (transduced NK cells, expression vector, expression construct, etc.) may be used as a generalized immune enhancer (NK cell activating composition or system) and as such has utility in treating diseases. Exemplary diseases that can be treated and/or prevented include, but are not limited, to infections of viral etiology such as HIV, influenza, Herpes, viral hepatitis, Epstein Bar, polio, viral encephalitis, measles, chicken pox, Papilloma virus etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

Preneoplastic or hyperplastic states which may be treated or prevented using the pharmaceutical composition (transduced NK cells, expression vector, expression construct, etc.) include but are not limited to preneoplastic or hyperplastic states such as colon polyps, Crohn's disease, ulcerative colitis, breast lesions and the like.

Cancers, including solid tumors, which may be treated using the pharmaceutical composition include, but are not limited to primary or metastatic melanoma, adenocarcinoma, squamous cell carcinoma, adenosquamous cell carcinoma, thymoma, lymphoma, sarcoma, lung cancer, liver cancer, non-Hodgkin's lymphoma, Hodgkin's lymphoma, leukemias, uterine cancer, breast cancer, prostate cancer, ovarian cancer, pancreatic cancer, colon cancer, multiple myeloma, neuroblastoma, NPC, bladder cancer, cervical cancer and the like.

Other hyperproliferative diseases, including solid tumors, that may be treated using the NK cell and other therapeutic cell activation system presented herein include, but are not limited to rheumatoid arthritis, inflammatory bowel disease, osteoarthritis, leiomyomas, adenomas, lipomas, hemangiomas, fibromas, vascular occlusion, restenosis, atherosclerosis, pre-neoplastic lesions (such as adenomatous hyperplasia and prostatic intraepithelial neoplasia), carcinoma in situ, oral hairy leukoplakia, or psoriasis.

Methods of treatment may include methods for prophylactic or therapeutic purposes. When provided prophylactically, the pharmaceutical composition, for example, the expression construct, expression vector, fused protein, transduced cells, activated immune cells, transduced or loaded immune cells, is provided in advance of any detected or reported symptom. The prophylactic administration of pharmaceutical composition serves to prevent or ameliorate any subsequent infection or disease. When provided therapeutically, the pharmaceutical composition is provided at or after the onset of a symptom of infection or disease. Thus, the compositions presented herein may be provided either prior to the anticipated exposure to a disease-causing agent or disease state or after the initiation of the infection or disease. Thus, provided herein are methods for prophylactic treatment of solid tumors such as those found in cancer, or for example, but not limited to, prostate cancer, using the nucleic acids and ligands discussed herein.

For example, methods are provided of prophylactically preventing or reducing the size of a tumor, or reducing the concentration or number of target cells, in a subject comprising administering a nucleic acid comprising a promoter operably linked to a polynucleotide that encodes a chimeric signaling polypeptide, and a nucleic acid that encodes a CAR or a recombinant TCR. The chimeric signaling polypeptide may, or may not comprise a membrane targeting region, and may optionally be inducible or constitutive. The chimeric signaling polypeptide may also be provided as part of the CAR polypeptide. The nucleic acid, and optionally the multimeric ligand are administered in an amount effect to prevent or reduce the size of a tumor, or the concentration or number of target cells in a subject. The term multimerization region or multimeric ligand binding region may be used in place of the term ligand binding region for purposes of this application.

Solid tumors from any tissue or organ may be treated using the present methods, including, for example, any tumor expressing PSA, for example, PSMA, in the vasculature, for example, solid tumors present in, for example, lungs, bone, liver, prostate, or brain, and also, for example, in breast, ovary, bowel, testes, colon, pancreas, kidney, bladder, neuroendocrine system, soft tissue, boney mass, and lymphatic system. Other solid tumors that may be treated include, for example, glioblastoma, and malignant myeloma.

### Combination Therapies

In order to increase the effectiveness of the modified cells presented herein, it may be desirable to combine these compositions and methods with an agent effective in the treatment of the disease.

In certain embodiments, anti-cancer agents may be used in combination with the present methods. An “anti-cancer” agent is capable of negatively affecting cancer in a subject, for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the lifespan of a subject with a cancer. Anti-cancer agents include, for example, chemotherapy agents (chemotherapy), radiotherapy agents (radiotherapy), a surgical procedure (surgery), immune therapy agents (immunotherapy), genetic therapy agents (gene therapy), hormonal therapy, other biological agents (biotherapy) and/or alternative therapies.

In some embodiments, the modified NK cells described herein are used in combination with other cell therapies, such as, for example, T cells, tumor infiltrating lymphocytes, natural killer cells, TCR-expressing cells, natural killer T cells, or progenitor cells, such as, for example, hematopoietic stem cells, mesenchymal stromal cells, stem cells, pluripotent stem cells, and embryonic stem cells

The terms “mesenchymal stromal cell” or “bone marrow derived mesenchymal stromal cell” as used herein, refer to multipotent stem cells that can differentiate ex vivo, in vitro and in vivo into adipocytes, osteoblasts and chondroblasts, and may be further defined as a fraction of mononuclear bone marrow cells that adhere to plastic culture dishes in standard culture conditions, are negative for hematopoietic lineage markers and are positive for CD73, CD90 and CD105.

The term “embryonic stem cell” as used herein, refers to pluripotent stem cells derived from the inner cell mass of the blastocyst, an early-stage embryo of between 50 to 150 cells. Embryonic stem cells are characterized by their ability to renew themselves indefinitely and by their ability to differentiate into derivatives of all three primary germ layers, ectoderm, endoderm and mesoderm. Pluripotent is distinguished from multipotent in that pluripotent cells can generate all cell types, while multipotent cells (e.g., adult stem cells) can only produce a limited number of cell types.

Tumor infiltrating lymphocytes (TILs) refer to T cells having various receptors which infiltrate tumors and kill tumor cells in a targeted manor.

In further embodiments antibiotics can be used in combination with the pharmaceutical composition to treat and/or prevent an infectious disease. Such antibiotics include, but are not limited to, amikacin, aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

More generally, such an agent would be provided in a combined amount with modified cells effective to kill or inhibit proliferation of a cancer cell and/or microorganism. This process may involve contacting the cell(s) with an agent(s) and the pharmaceutical composition at the same time or within a period of time wherein separate administration of the pharmaceutical composition and an agent to a cell, tissue or organism produces a desired therapeutic benefit. This may be achieved by contacting the cell, tissue or organism with a single composition or pharmacological formulation that includes both the pharmaceutical composition and one or more agents, or by contacting the cell with two or more distinct compositions or formulations, wherein one composition includes the pharmaceutical composition and the other includes one or more agents.

The administration of the pharmaceutical composition may precede, be concurrent with and/or follow the other agent(s) by intervals ranging from minutes to weeks. In embodiments where the pharmaceutical composition and other agent(s) are applied separately to a cell, tissue or organism, one would generally ensure that a significant period of time did not expire between the times of each delivery, such that the pharmaceutical composition and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the pharmaceutical composition. In other aspects, one or more agents may be administered from substantially simultaneously, about 1 minute, to about 24 hours to about 7 days to about 1 to about 8 weeks or more, and any range derivable therein, prior to and/or after administering the expression vector. Yet further, various combination regimens of the pharmaceutical composition presented herein, and one or more agents may be employed.

In some embodiments, the methods further comprise administering a chemotherapeutic agent. In some embodiments, the composition, ligand, and the chemotherapeutic agent are administered in an amount effective to treat cancer, such as, for example, prostate cancer, in the subject. In some embodiments, the composition or the nucleotide sequences, the ligand, and the chemotherapeutic agent are administered in an amount effective to treat cancer in the subject. In some embodiments, the chemotherapeutic agent is selected from the group consisting of carboplatin, estramustine phosphate (Emcyt), and thalidomide. In some embodiments, the chemotherapeutic agent is a taxane. The taxane may be, for example, selected from the group consisting of docetaxel (Taxotere), paclitaxel, and cabazitaxel. In some embodiments, the taxane is docetaxel. In some embodiments, the chemotherapeutic agent is administered at the same time or within one week after the administration of the cell, nucleic acid or the ligand. In other embodiments, the chemotherapeutic agent is administered after the administration of the ligand. In other embodiments, the chemotherapeutic agent is administered from 1 to 4 weeks or from 1 week to 1 month, 1 week to 2 months, or 1 week to 3 months after the administration of the ligand. In other embodiments, the methods further comprise administering the chemotherapeutic agent from 1 to 4 weeks, or from 1 week to 1 month, 1 week to 2 months, or 1 week to 3 months before the administration of the cell or nucleic acid. In some embodiments, the chemotherapeutic agent is administered at least 2 weeks before administering the cell or nucleic acid. In some embodiments, the chemotherapeutic agent is administered at least 1 month before administering the cell or nucleic acid. In some embodiments, the chemotherapeutic agent is administered after administering the multimeric ligand. In some embodiments, the chemotherapeutic agent is administered at least 2 weeks after administering the multimeric ligand. In some embodiments, wherein the chemotherapeutic agent is administered at least 1 month after administering the multimeric ligand.

In some embodiments, the chemotherapeutic agent may be Taxotere (docetaxel), or another taxane, such as, for example, cabazitaxel. The chemotherapeutic may be administered either before, during, or after treatment with the cells and inducer. For example, the chemotherapeutic may be administered about 1 year, 11, 10, 9, 8, 7, 6, 5, or 4 months, or 18, 17, 16, 15, 14, 13, 12, 11, 10, 9, 8, 7, 6, 5, 4, 3, 2, weeks or 1 week prior to administering the first dose of activated nucleic acid. Or, for example, the chemotherapeutic may be administered about 1 week or 2, 3, 4, 5, 6, 7, 8, 9, 10, 11, 12, 13, 14, 15, 16, 17, or 18 weeks or 4, 5, 6, 7, 8, 9, 10, or 11 months or 1 year after administering the first dose of cells or inducer.

Administration of a chemotherapeutic agent may comprise the administration of more than one chemotherapeutic agent. For example, cisplatin may be administered in addition to Taxotere or other taxane, such as, for example, cabazitaxel.

In some embodiments, the methods further comprise administering two or more chemotherapeutic agents. In some embodiments, the chemotherapeutic agents are selected from the group consisting of carboplatin, Estramustine phosphate, and thalidomide. In some embodiments, at least one chemotherapeutic agent is a taxane. The taxane may be, for example, selected from the group consisting of docetaxel, paclitaxel, and cabazitaxel. In some embodiments, the taxane is docetaxel. In some embodiments, the chemotherapeutic agents are administered at the same time or within one week after the administration of the cell, nucleic acid or the ligand. In other embodiments, the chemotherapeutic agents are administered after the administration of the ligand. In other embodiments, the chemotherapeutic agents are administered from 1 to 4 weeks or from 1 week to 1 month, 1 week to 2 months, or 1 week to 3 months after the administration of the ligand. In other embodiments, the methods further comprise administering the chemotherapeutic agents from 1 to 4 weeks or from 1 week to 1 month, 1 week to 2 months, or 1 week to 3 months before the administration of the cell or nucleic acid.

### Optimized and Personalized Therapeutic Treatment

The dosage and administration schedule of the ligand inducer may be optimized by determining the level of the disease or condition to be treated. For example, the size of any remaining solid tumor, or the level of targeted cells such as, for example, tumor cells that may remain in the patient, may be determined.

For example, determining that a patient has clinically relevant levels of tumor cells, or a solid tumor, after initial therapy, provides an indication to a clinician that it may be necessary to activate the chimeric-antigen receptor-expressing NK cells by activating the cells by administering the multimeric ligand. In another example, determining that a patient has a reduced level of tumor cells or reduced tumor size after treatment with the multimeric ligand may indicate to the clinician that no additional dose of the multimeric ligand is needed. Similarly, after treatment with the multimeric ligand, determining that the patient continues to exhibit disease or condition symptoms, or suffers a relapse of symptoms may indicate to the clinician that it may be necessary to administer at least one additional dose of multimeric ligand. The term “dosage” is meant to include both the amount of the dose and the frequency of administration, such as, for example, the timing of the next dose. The term “dosage level” refers to the amount of the multimeric ligand administered in relation to the body weight of the subject. Thus, increasing the dosage level would mean increasing the amount of the ligand administered relative to the subject's weight. In addition, increasing the concentration of the dose administered, such as, for example, when the multimeric ligand is administered using a continuous infusion pump would mean that the concentration administered (and thus the amount administered) per minute, or second, is increased.

Thus, for example, in certain embodiments where cells that express an inducible chimeric signaling polypeptide or an inducible chimeric antigen receptor polypeptide are administered to a patient, the methods comprise determining the presence or absence of a tumor size increase and/or increase in the number of tumor cells in a subject relative to the tumor size and/or the number of tumor cells following administration of the multimeric ligand, and administering an additional dose of the multimeric ligand to the subject in the event the presence of a tumor size increase and/or increase in the number of tumor cells is determined. In these embodiments, for example, the patient is initially treated with the therapeutic cells and ligand according to the methods provided herein. Following the initial treatment, the size of the tumor, or the number of tumor cells, may decrease relative to the time prior to the initial treatment. At a certain time after this initial treatment, the patient is again tested, or the patient may be continually monitored for disease symptoms. If it is determined that the size of the tumor, or the number of tumor cells, for example, is increased relative to the time just after the initial treatment, then the ligand may be administered for an additional dose. This monitoring and treatment schedule may continue, because the therapeutic cells that express inducible chimeric signaling polypeptides remain in the patient, although in a relatively inactive state in the absence of additional ligand.

In other examples where cells that express an inducible chimeric signaling polypeptide or an inducible chimeric antigen receptor polypeptide are administered to a patient, and where the target cell is not a tumor cell, the methods comprise determining the presence or absence of the concentration or number of target cells in a subject relative to the concentration or number of target cells following administration of the multimeric ligand, and administering an additional dose of the multimeric ligand to the subject in the event the presence of an increase in the concentration or number of target cells is determined. For this analysis, the concentration or number of target cells refers to the concentration or number of target cells in a sample obtained from the patient. these embodiments, for example, the patient is initially treated with the therapeutic cells and ligand according to the methods provided herein. Following the initial treatment, the concentration or the number of target cells may decrease relative to the time prior to the initial treatment. At a certain time after this initial treatment, the patient is again tested, or the patient may be continually monitored for disease symptoms. If it is determined that the concentration or number of target cells, for example, is increased relative to the time just after the initial treatment, then the ligand may be administered for an additional dose. This monitoring and treatment schedule may continue, because the therapeutic cells that express inducible chimeric signaling polypeptides remain in the patient, although in a relatively inactive state in the absence of additional ligand.

An indication of adjusting or maintaining a subsequent drug dose, such as, for example, a subsequent dose of the multimeric ligand, and/or the subsequent drug dosage, can be provided in any convenient manner. An indication may be provided in tabular form (e.g., in a physical or electronic medium) in some embodiments. For example, the size of the tumor cell, or the number or level of tumor cells in a sample may be provided in a table, and a clinician may compare the symptoms with a list or table of stages of the disease. The clinician then can identify from the table an indication for subsequent drug dose. In certain embodiments, an indication can be presented (e.g., displayed) by a computer, after the symptoms are provided to the computer (e.g., entered into memory on the computer). For example, this information can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

Methods as presented herein include without limitation the delivery of an effective amount of a modified cell, a nucleic acid, an expression construct encoding the same, or the multimeric ligand. An “effective amount” of the pharmaceutical composition, cell, nucleic acid, expression construct, or multimeric ligand, generally, is defined as that amount sufficient to detectably and repeatedly to achieve the stated desired result, for example, to ameliorate, reduce, minimize or limit the extent of the disease or its symptoms. Other more rigorous definitions may apply, including elimination, eradication or cure of disease. In some embodiments there may be a step of monitoring the biomarkers to evaluate the effectiveness of treatment and to control toxicity.

### Enhancement of an Immune Response

In certain embodiments, an immune cell activation strategy is contemplated, that incorporates the manipulation of signaling co-stimulatory polypeptides that activate biological pathways, for example, immunological pathways, such as, for example, NF-κB pathways, Akt pathways, and/or p38 pathways. This immune cell activation system can be used in conjunction with or without standard vaccines to enhance the immune response. For example, release of IFNγ by IL-12-stimulated NK cells can lead to upregulation of MHC on target cells along with improvements in antigen presentation. Furthermore, the release of chemokines such as MCP1, XCL1, XCL2, CCL5 and CXCL10 by activated NK cells in an anti-tumor response can recruit and induce the differentiation of dendritic cells and induce the expansion of an adaptive immune response by native T lymphocytes as well as recruiting macrophages and other immune cells as part of an overall inflammatory response (Bottcher et al. (2018) Cell 172: p1022-1037.

In certain embodiments the cells are in an animal, such as human, non-human primate, cow, horse, pig, sheep, goat, dog, cat, or rodent. The subject may be, for example, an animal, such as a mammal, for example, a human, non-human primate, cow, horse, pig, sheep, goat, dog, cat, or rodent. The subject may be, for example, human, for example, a patient suffering from an infectious disease, and/or a subject that is immunocompromised, or is suffering from a hyperproliferative disease.

In further embodiments, the expression construct and/or expression vector can be utilized as a composition or substance that activates cells. Such a composition that “activates cells” or “enhances the activity cells” refers to the ability to stimulate one or more activities associated with cells. For example, a composition, such as the expression construct or vector of the present methods, can stimulate upregulation of co-stimulatory molecules on cells, induce nuclear translocation of NF-kappaB in cells, activate toll-like receptors in cells, or other activities involving cytokines or chemokines.

The expression construct, expression vector and/or transduced cells can enhance or contribute to the effectiveness of a vaccine by, for example, enhancing the immunogenicity of weaker antigens such as highly purified or recombinant antigens, reducing the amount of antigen required for an immune response, reducing the frequency of immunization required to provide protective immunity, improving the efficacy of vaccines in subjects with reduced or weakened immune responses, such as newborns, the aged, and immunocompromised individuals, and enhancing the immunity at a target tissue, such as mucosal immunity, or promote cell-mediated or humoral immunity by eliciting a particular cytokine profile.

In certain embodiments, the cell is also contacted with an antigen. Often, the cell is contacted with the antigen ex vivo. Sometimes, the cell is contacted with the antigen in vivo. In some embodiments, the cell is in a subject and an immune response is generated against the antigen. Sometimes, the immune response is a cytotoxic T-lymphocyte (CTL) immune response. Sometimes, the immune response is generated against a tumor antigen. In certain embodiments, the cell is activated without the addition of an adjuvant.

In some embodiments, the cell is transduced with the nucleic acid ex vivo and administered to the subject by intradermal administration. In some embodiments, the cell is transduced with the nucleic acid ex vivo and administered to the subject by subcutaneous administration. Sometimes, the cell is transduced with the nucleic acid ex vivo. Sometimes, the cell is transduced with the nucleic acid in vivo.

In certain embodiments, the cell can be transduced ex vivo or in vivo with a nucleic acid that encodes the chimeric protein. The cell may be sensitized to the antigen at the same time the cell is contacted with the multimeric ligand, or the cell can be pre-sensitized to the antigen before the cell is contacted with the multimerization ligand. In some embodiments, the cell is contacted with the antigen ex vivo. In certain embodiments the cell is transduced with the nucleic acid ex vivo and administered to the subject by intradermal administration, and sometimes the cell is transduced with the nucleic acid ex vivo and administered to the subject by subcutaneous administration. The antigen may be a tumor antigen, and the CTL immune response can be induced by migration of the cell to a draining lymph node. A tumor antigen is any antigen such as, for example, a peptide or polypeptide, that triggers an immune response in a host. The tumor antigen may be a tumor-associated antigen that is associated with a neoplastic tumor cell.

The term “immunocompromised” as used herein is defined as a subject that has reduced or weakened immune system. In some embodiments, an immunocompromised individual or subject is a subject that has a reduced or weakened immune response. Such individuals may also include a subject that has undergone chemotherapy or any other therapy resulting in a weakened immune system, a transplant recipient, a subject currently taking immunosuppressants, an aging individual, or any individual that has a reduced and/or impaired CD4 T helper cells. It is contemplated that the present methods can be utilized to enhance the amount and/or activity of CD4 T helper cells in an immunocompromised subject.

Challenge with Target Antigens

In specific embodiments, prior to administering the transduced cell, the cells may be challenged with antigens (also referred herein as “target antigens”). After challenge, the transduced, loaded cells are administered to the subject parenterally, intradermally, intranodally, or intralymphatically. Additional parenteral routes include, but are not limited to subcutaneous, intramuscular, intraperitoneal, intravenous, intraarterial, intramyocardial, transendocardial, transepicardial, intrathecal, intraprotatic, intratumor, and infusion techniques.

The target antigen, as used herein, is an antigen or immunological epitope on the antigen, which is crucial in immune recognition and ultimate elimination or control of the disease-causing agent or disease state in a mammal. The immune recognition may be cellular and/or humoral. In the case of intracellular pathogens and cancer, immune recognition may, for example, be a T lymphocyte response.

The target antigen may be derived or isolated from, for example, a pathogenic microorganism such as viruses including HIV, (Korber et al, eds HIV Molecular Immunology Database, Los Alamos National Laboratory, Los Alamos, N. Mex. 1977) influenza, Herpes simplex, human papilloma virus (U.S. Pat. No. 5,719,054), Hepatitis B (U.S. Pat. No. 5,780,036), Hepatitis C (U.S. Pat. No. 5,709,995), EBV, Cytomegalovirus (CMV) and the like. Target antigen may be derived or isolated from pathogenic bacteria such as, for example, from Chlamydia (U.S. Pat. No. 5,869,608), Mycobacteria, Legionella, Meningiococcus, Group A Streptococcus, Salmonella, Listeria, Hemophilus influenzae (U.S. Pat. No. 5,955,596) and the like.

Target antigen may be derived or isolated from, for example, pathogenic yeast including Aspergillus, invasive Candida (U.S. Pat. No. 5,645,992), Nocardia, Histoplasmosis, Cryptosporidia and the like.

Target antigen may be derived or isolated from, for example, a pathogenic protozoan and pathogenic parasites including but not limited to Pneumocystis carinii, Trypanosoma, Leishmania (U.S. Pat. No. 5,965,242), Plasmodium (U.S. Pat. No. 5,589,343) and Toxoplasma gondii.

Target antigen includes an antigen associated with a preneoplastic or hyperplastic state. Target antigen may also be associated with, or causative of cancer. Such target antigen may be, for example, tumor specific antigen, tumor associated antigen (TAA) or tissue specific antigen, epitope thereof, and epitope agonist thereof. Such target antigens include but are not limited to carcinoembryonic antigen (CEA) and epitopes thereof such as CAP-1, CAP-1-6D and the like (GenBank Accession No. M29540), MART-1 (Kawakarni et al, J. Exp. Med. 180:347-352, 1994), MAGE-1 (U.S. Pat. No. 5,750,395), MAGE-3, GAGE (U.S. Pat. No. 5,648,226), GP-100 (Kawakami et al Proc. Nat'l Acad. Sci. USA 91:6458-6462, 1992), MUC-1, MUC-2, point mutated ras oncogene, normal and point mutated p53 oncogenes (Hollstein et al Nucleic Acids Res. 22:3551-3555, 1994), PSMA (Israeli et al Cancer Res. 53:227-230, 1993), tyrosinase (Kwon et al PNAS 84:7473-7477, 1987) TRP-1 (gp75) (Cohen et al Nucleic Acid Res. 18:2807-2808, 1990; U.S. Pat. No. 5,840,839), NY-ESO-1 (Chen et al PNAS 94:1914-1918, 1997), TRP-2 (Jackson et al EMBOJ, 11:527-535, 1992), TAG72, KSA, CA-125, PSA, HER-2/neu/c-erb/B2, (U.S. Pat. No. 5,550,214), BRC-I, BRC-II, bcr-abl, pax3-fkhr, ews-fli-1, modifications of TAAs and tissue specific antigen, splice variants of TAAs, epitope agonists, and the like. Other TAAs may be identified, isolated and cloned by methods known in the art such as those disclosed in U.S. Pat. No. 4,514,506. Target antigen may also include one or more growth factors and splice variants of each.

An antigen may be expressed more frequently in cancer cells than in non-cancer cells. The antigen may result from contacting the modified dendritic cell with a prostate specific membrane antigen, for example, a prostate specific membrane antigen (PSMA) or fragment thereof.

### Cytokine Measurement for Optimized and Personalized Treatment

Cytokines are a large and diverse family of polypeptide regulators produced widely throughout the body by cells of diverse origin. Cytokines are small secreted proteins, including peptides and glycoproteins, which mediate and regulate immunity, inflammation, and hematopoiesis. They are produced de novo in response to an immune stimulus. Cytokines generally (although not always) act over short distances and short time spans and at low concentration. They generally act by binding to specific membrane receptors, which then signal the cell via signaling proteins, often tyrosine kinases of the Janus family or coupled G proteins to alter cell behavior (e.g., gene expression). Responses to cytokines include, for example, increasing or decreasing expression of membrane proteins (including cytokine receptors), proliferation, blockage or promotion of apoptosis, differentiation and secretion of effector molecules.

The term “cytokine” is a general description of a large family of proteins and glycoproteins. Other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on cells that secrete them (autocrine action), on nearby cells (paracrine action), or in some instances on distant cells (endocrine action).

Examples of cytokines include, without limitation, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18 and the like), interferons (e.g., IFN-beta, IFN-gamma and the like), tumor necrosis factors (e.g., TNF-alpha, TNF-beta and the like), lymphokines, monokines and chemokines; growth factors (e.g., transforming growth factors (e.g., TGF-alpha, TGF-beta and the like)); colony-stimulating factors (e.g. GM-CSF, granulocyte colony-stimulating factor (G-CSF) etc.); IP-10, MCP-1, and the like.

A cytokine often acts via a cell-surface receptor counterpart. Subsequent cascades of intracellular signaling then alter cell functions. This signaling may include upregulation and/or downregulation of several genes and their transcription factors, resulting in the production of other cytokines, an increase in the number of surface receptors for other molecules, or the suppression of their own effect by feedback inhibition.

Treatment using immune system activating cells discussed herein may be optimized by determining the concentration of certain cytokine biomarkers, such as, for example, cytokines discussed herein, including, for example, IL-2, IP-10, IL-5, and MCP-1, and, for example, IL-6, IL6-sR, or VCAM-1 during the course of treatment. In a specific embodiment, a cytokine, such as one, two, three or more of those described in the Examples below (e.g., Example 3, infra) is measured during one, two or all of these timepoints: prior to, during, or subsequent to treatment described herein (e.g., administration of modified NK cells). IL-6 refers to interleukin 6. IL-6sR refers to the IL-6 soluble receptor, the levels of which often correlate closely with levels of IL-6. VCAM-1 refers to vascular cell adhesion molecule. Different patients having different stages or types of cancer, may react differently to various therapies. The response to treatment may be monitored by following the cytokine concentrations or levels in various body fluids or tissues. The determination of the concentration, level, or amount of a cytokine polypeptide may include detection of the full-length polypeptide, or a fragment or variant thereof. The fragment or variant may be sufficient to be detected by, for example, immunological methods, mass spectrometry, nucleic acid hybridization, and the like. Optimizing treatment for individual patients may help to avoid side effects as a result of overdosing, may help to determine when the treatment is ineffective and to change the course of treatment, or may help to determine when doses may be increased. Technology discussed herein optimizes therapeutic methods for treating solid tumor cancers by allowing a clinician to track a biomarker, such as, for example, IL-6, IL-6sR, or VCAM-1, and determine whether a subsequent dose of a drug or vaccine for administration to a subject may be maintained, reduced or increased, and to determine the timing for the subsequent dose. Technology discussed herein optimizes therapeutic methods for treating solid tumor cancers by allowing a clinician to assay the level of or track a biomarker, such as, for example, IL-2, IP-10, IL-5, or MCP-1, and determine whether a subsequent dose of a drug or vaccine for administration to a subject may be maintained, reduced or increased, to determine the timing for the subsequent dose, or to determine which inducible or constitutive chimeric signaling polypeptide should be selected for the therapeutic cells.

Treatment for solid tumor cancers, including, for example, prostate cancer, may also be optimized by determining the concentration of urokinase-type plasminogen activator receptor (uPAR), hepatocyte growth factor (HGF), epidermal growth factor (EGF), or vascular endothelial growth factor (VEGF) during the course of treatment. Different patients having different stages or types of cancer, may react differently to various therapies. The levels of uPAR, HGF, EGF, and VEGF may show systemic perturbation of hypoxic factors in serum, which may indicate a positive response to treatment. Thus, the response to treatment may be monitored, for example, by following the one or more cytokine concentrations or levels in various body fluids or tissues.

Optimizing treatment for individual patients may help to avoid side effects as a result of over activity of the modified cells, may help to determine when the treatment is ineffective and to change the course of treatment, or may help to determine when doses may be increased. Technology discussed herein optimizes therapeutic methods for treating diseases, e.g. cancers, by allowing a clinician to track a biomarker, such as, for example, a cytokine, and determine whether a subsequent dose of a modified cell and/or ligand for administration to a subject may be maintained, reduced or increased, and to determine the timing for the subsequent dose.

Cytokines may be detected as full-length (e.g., whole) proteins, polypeptides, metabolites, messenger RNA (mRNA), complementary DNA (cDNA), and various intermediate products and fragments of the foregoing (e.g., cleavage products (e.g., peptides, mRNA fragments)). For example, IL-6 protein may be detected as the complete, full-length molecule or as any fragment large enough to provide varying levels of positive identification. Such a fragment may comprise amino acids numbering less than 10, from 10 to 20, from 20 to 50, from 50 to 100, from 100 to 150, from 150 to 200 and above. Likewise, VCAM-1 protein can be detected as the complete, full-length amino acid molecule or as any fragment large enough to provide varying levels of positive identification. Such a fragment may comprise amino acids numbering less than 10, from 10 to 20, from 20 to 50, from 50 to 100, from 100 to 150 and above.

In certain embodiments, cytokine mRNA may be detected by targeting a complete sequence or any sufficient fragment for specific detection. An mRNA fragment may include fewer than 10 nucleotides or any larger number. A fragment may comprise the 3′ end of the mRNA strand with any portion of the strand, the 5′ end with any portion of the strand, and any center portion of the strand.

Detection may be performed using any suitable method, including, without limitation, mass spectrometry (e.g., matrix-assisted laser desorption ionization mass spectrometry (MALDI-MS), electrospray mass spectrometry (ES-MS)), electrophoresis (e.g., capillary electrophoresis), high performance liquid chromatography (HPLC), nucleic acid affinity (e.g., hybridization), amplification and detection (e.g., real-time or reverse-transcriptase polymerase chain reaction (RT-PCR)), and antibody assays (e.g., antibody array, enzyme-linked immunosorbant assay (ELISA)). Examples of IL-6 and other cytokine assays include, for example, those provided by Millipore, Inc., (Milliplex Human Cytokine/Chemokine Panel). Examples of IL6-sR assays include, for example, those provided by Invitrogen, Inc. (Soluble IL-6R: (Invitrogen Luminex® Bead-based assay)). Examples of VCAM-1 assays include, for example, those provided by R & D Systems ((CD106) ELISA development Kit, DuoSet from R&D Systems (#DY809)).

As used herein, “hybridization”, “hybridizes” or “capable of hybridizing” is understood to mean forming a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but preclude hybridization of random sequences. Stringent conditions tolerate little, if any, mismatch between a nucleic acid and a target strand. Such conditions are known, and are often used for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.5 M NaCl at temperatures of about 42 degrees C. to about 70 degrees C. It is understood that the temperature and ionic strength of a desired stringency are determined in part by the length of the particular nucleic acid(s), the length and nucleobase content of the target sequence(s), the charge composition of the nucleic acid(s), and the presence or concentration of formamide, tetramethylammonium chloride or other solvent(s) in a hybridization mixture.

It is understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization may be employed to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20 degrees C. to about 50 degrees C. The low or high stringency conditions may be further modified to suit a particular application.

### Sources of Biomarkers

In some embodiments, treatment using immune system activating cells discussed herein may be optimized by determining the concentration of certain biomarkers. Biomarkers levels can be used to optimize therapeutic methods for treating diseases, e.g. cancers, by for example, determining whether a subsequent dose of a modified cell and/or ligand administration to a subject may be maintained, reduced or increased, and to determine the timing for the subsequent dose.

The presence, absence or amount of a biomarker can be determined within a subject (e.g., in situ) or outside a subject (e.g., ex vivo). In some embodiments, presence, absence or amount of a biomarker can be determined in cells (e.g., differentiated cells, stem cells), and in certain embodiments, presence, absence or amount of a biomarker can be determined in a substantially cell-free medium (e.g., in vitro). The term “identifying the presence, absence or amount of a biomarker in a subject” as used herein refers to any method known in the art for assessing the biomarker and inferring the presence, absence or amount in the subject (e.g., in situ, ex vivo or in vitro methods).

A fluid or tissue sample often is obtained from a subject for determining presence, absence or amount of biomarker ex vivo. Non-limiting parts of the body from which a tissue sample may be obtained include leg, arm, abdomen, upper back, lower back, chest, hand, finger, fingernail, foot, toe, toenail, neck, rectum, nose, throat, mouth, scalp, face, spine, throat, heart, lung, breast, kidney, liver, intestine, colon, pancreas, bladder, cervix, testes, muscle, skin, hair, tumor or area surrounding a tumor, and the like, in some embodiments. A tissue sample can be obtained by any suitable method known in the art, including, without limitation, biopsy (e.g., shave, punch, incisional, excisional, curettage, fine needle aspirate, scoop, scallop, core needle, vacuum assisted, open surgical biopsies) and the like, in certain embodiments. Examples of a fluid that can be obtained from a subject includes, without limitation, blood, cerebrospinal fluid, spinal fluid, lavage fluid (e.g., bronchoalveolar, gastric, peritoneal, ductal, ear, arthroscopic), urine, interstitial fluid, feces, sputum, saliva, nasal mucous, prostate fluid, lavage, semen, lymphatic fluid, bile, tears, sweat, breast milk, breast fluid, fluid from region of inflammation, fluid from region of muscle wasting and the like, in some embodiments.

A sample from a subject may be processed prior to determining presence, absence or amount of a biomarker. For example, a blood sample from a subject may be processed to yield a certain fraction, including without limitation, plasma, serum, buffy coat, red blood cell layer and the like, and biomarker presence, absence or amount can be determined in the fraction. In certain embodiments, a tissue sample (e.g., tumor biopsy sample) can be processed by slicing the tissue sample and observing the sample under a microscope before and/or after the sliced sample is contacted with an agent that visualizes a biomarker (e.g., antibody). In some embodiments, a tissue sample can be exposed to one or more of the following non-limiting conditions: washing, exposure to high salt or low salt solution (e.g., hypertonic, hypotonic, isotonic solution), exposure to shearing conditions (e.g., sonication, press (e.g., French press)), mincing, centrifugation, separation of cells, separation of tissue and the like. In certain embodiments, a biomarker can be separated from tissue and the presence, absence or amount determined in vitro. A sample also may be stored for a period of time prior to determining the presence, absence or amount of a biomarker (e.g., a sample may be frozen, cryopreserved, maintained in a preservation medium (e.g., formaldehyde)).

A sample can be obtained from a subject at any suitable time of collection before or after the modified cell is delivered to the subject. For example, a sample may be collected within about one hour after a modified cell is delivered to a subject (e.g., within about 5, 10, 15, 20, 25, 30, 35, 40, 45, 55 or 60 minutes of delivering a drug), within about one day after a modified cell is delivered to a subject (e.g., within about 2, 3, 4, 5, 6, 7, 8, 9, 10, 11, 12, 13, 14, 15, 16, 17, 18, 19, 20, 21, 22, 23 or 24 hours of delivering a drug) or within about two weeks after a modified cell is delivered to a subject (e.g., within about 2, 3, 4, 5, 6, 7, 8, 9, 10, 11, 12, 13 or 14 days of delivering the drug). A collection may be made on a specified schedule including hourly, daily, semi-weekly, weekly, bi-weekly, monthly, bi-monthly, quarterly, and yearly, and the like, for example. If a modified cell is administered continuously over a time period (e.g., infusion), the delay may be determined from the first moment of drug is introduced to the subject, from the time the drug administration ceases, or a point in-between (e.g., administration time frame midpoint or other point).

### Biomarker Detection

The presence, absence or amount of one or more biomarkers may be determined by any suitable method known in the art, and non-limiting determination methods are discussed herein. Determining the presence, absence or amount of a biomarker sometimes comprises use of a biological assay. In a biological assay, one or more signals detected in the assay can be converted to the presence, absence or amount of a biomarker. Converting a signal detected in the assay can comprise, for example, use of a standard curve, one or more standards (e.g., internal, external), a chart, a computer program that converts a signal to a presence, absence or amount of biomarker, and the like, and combinations of the foregoing.

Biomarker detected in an assay can be full-length biomarker, a biomarker fragment, an altered or modified biomarker (e.g., biomarker derivative, biomarker metabolite), or sum of two or more of the foregoing, for example. Modified biomarkers often have substantial sequence identity to a biomarker discussed herein. For example, percent identity between a modified biomarker and a biomarker discussed herein may be in the range of 15-20%, 20-30%, 31-40%, 41-50%, 51-60%, 61-70%, 71-80%, 81-90% and 91-100%, (e.g. 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 percent identity). A modified biomarker often has a sequence (e.g., amino acid sequence or nucleotide sequence) that is 90% or more identical to a sequence of a biomarker discussed herein. Percent sequence identity can be determined using alignment methods known in the art.

Detection of biomarkers may be performed using any suitable method known in the art, including, without limitation, mass spectrometry, antibody assay (e.g., ELISA), nucleic acid affinity, microarray hybridization, Northern blot, reverse PCR and RT-PCR. For example, RNA purity and concentration may be determined spectrophotometrically (260/280>1.9) on a Nanodrop 1000. RNA quality may be assessed using methods known in the art (e.g., Agilent 2100 Bioanalyzer; RNA 6000 Nano LabChip® and the like).

### EXAMPLES

Examples herein that discuss the methods for transforming or transfecting cells in vitro, or ex vivo, provide examples of, but do not limit, the use of nucleic acids that express chimeric polypeptides. Examples of the delivery of the transduced or transfected cells, and ligand inducer, to laboratory animals or human subjects provide examples of, but do not limit, the direct administration of nucleic acids expressing chimeric polypeptides, tumor antigens, and ligand inducer to subjects in need thereof.

The present examples provide non-limiting examples of the expression of chimeric polypeptides comprising signaling regions in Natural Killer cells. The modified NK cells may be used to improve growth, storage, and efficacy of NK cells, and may be used, for example, for conditional chemical regulation of NK cell function.

The examples set forth below illustrate certain embodiments and do not limit the technology.

### Example 1: Expression of Chimeric Polypeptides in Natural Killer Cells

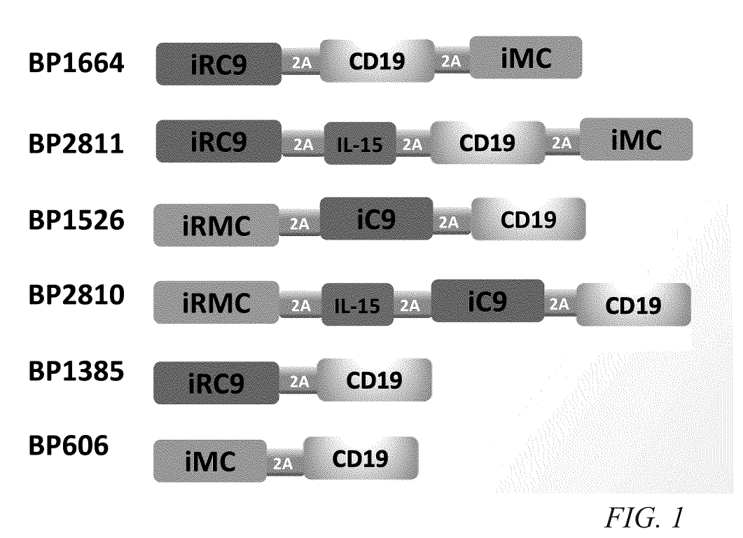

FIG. 1—Schematic representations of expression constructs used to transduce NK cells. Genes encoding the MC go signal are indicated in green, the proapoptotic C9 signal in red. These genes are encoded in the pSFG γ-retrovirus vector. The letter i indicates that the gene is inducible by fusion of the signaling domain with FKBP12v36 (i alone) or FKBP12f36-FRB (IR). IL-15 indicates the gene encoding interleukin-15, a cytokine that normally not produced by NK cells, but which acts in a normal inflammatory immune response to promote NK cell growth and survival. Ectopic expression of this transgene acts in an autocrine manner to further augment DS NK cell activity. CD19 encodes the extracellular and transmembrane domains, but deleting intracellular signaling domains of the CD19 protein. This is used as a molecular marker to identify transduced NK cell populations.

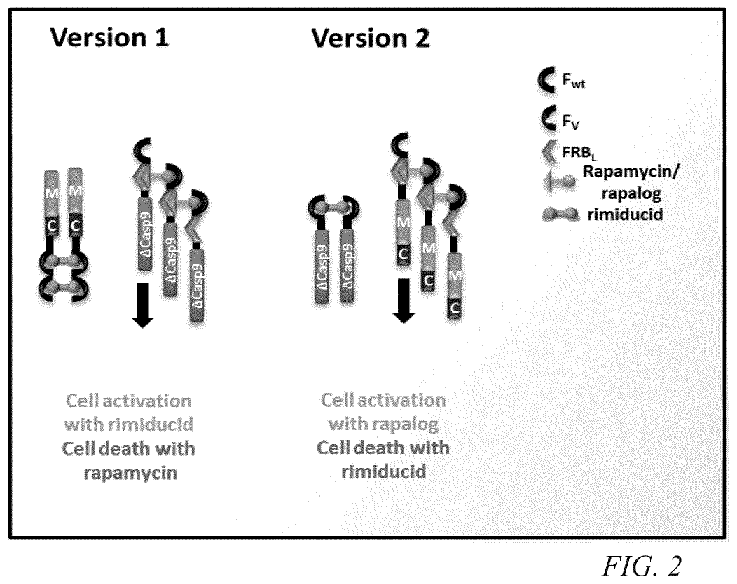

FIG. 2—Schematic representation of dual switch components. Version 1. MyD88/CD40 is fused at its carboxy terminus with two copies of FKBP12v36 (Fv). This is co-expressed with caspase-9 lacking the CARD domain fused at its amino terminus with tandem FKBP12f36 (Fwt) and FRB. Version 2. MyD88/CD40 is fused at its amino terminus with tandem FKBP12f36 (Fwt) and FRB (T2098L). This is co-expressed with caspase-9 lacking the CARD domain fused at its amino terminus with FKBP12v36.

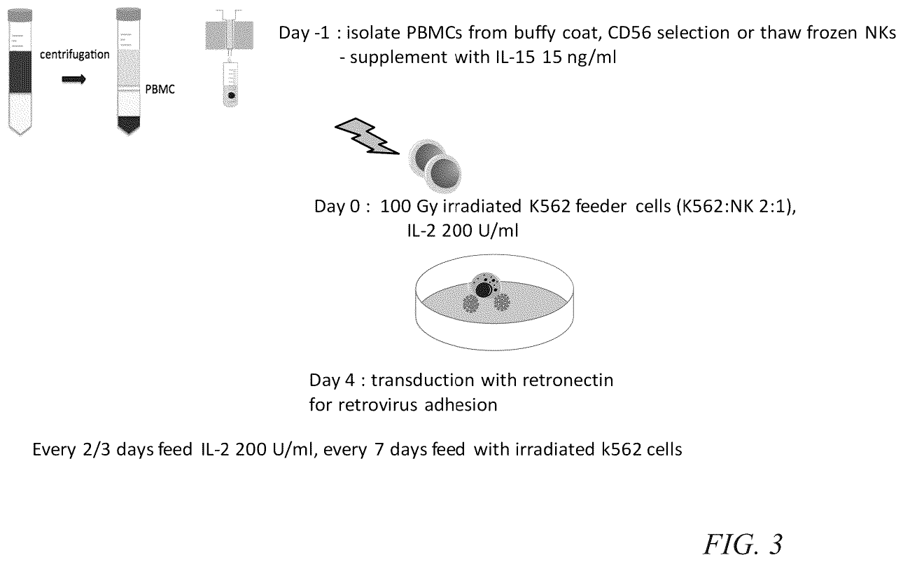

FIG. 3—Methods to isolate, culture and transduce Natural Killer cells. Cell components from human blood sourced from healthy adult donors was separated by centrifugation in Ficoll with the ‘buffy coat’ further fractionated to provide peripheral blood mononuclear cells (T and B lymphocytes, NK cells and monocytes). NK cells were isolated by selection with a column of magnetic beads fused with antibody to CD56 expressed on NK cells. IL-15 (15 ng/ml) was added to activate the NK cells and they were plated on a feeder layer of K562 myelogenous leukemia cells previously irradiated to block their growth and further supplemented with interleukin-2 (200 units/mL). Cells were transduced with retrovirus (FIG. 1) seated by centrifugation on retronectin coated plates. On the next day NK cells were fed every 48-72 hours with fresh feeder cells and IL-2 and without supplementary IL-15. Modification of NK cells was performed essentially as discussed in Kellner, J. N., et al., Methods Mol. Biol. 2016, 1441:203-13.

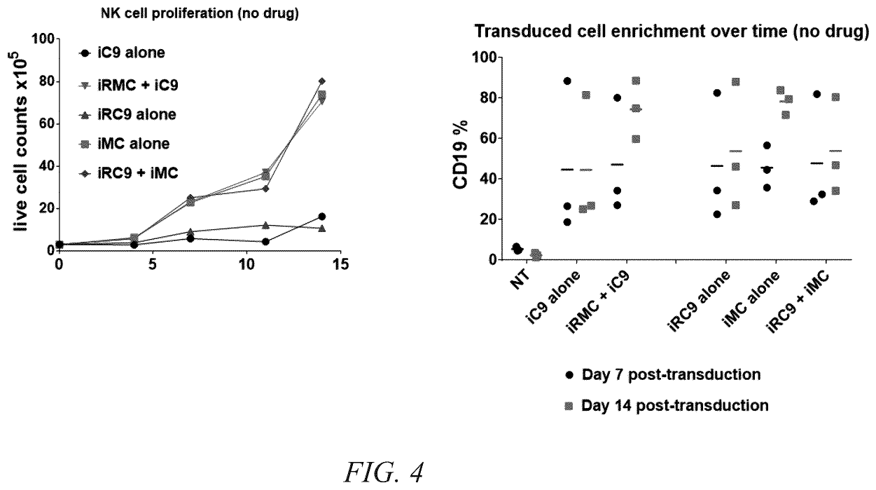

FIG. 4—MC expression leads to enhanced NK cell growth. (left) NK cells derived from three separate blood donors were transduced to express the indicated genes. NT indicates not transduced NK cells. The proportion of cells marked with CD19 was determined after 7 and 14 days of culture. An increase in the proportion of transduced cells at 14 days indicated selective outgrowth of the MC containing populations. (right) In a separate experiment, total live cell counts were taken at 2-4 day intervals for two weeks. Cells expressing MC even without added rimiducid selectively outgrew NK cells expressing only the safety switch. The presence of the iRMC chimeric polypeptide provided a growth advantage to the modified NK cells. In the presence of Interleuken-2 in culture, tonic iMC signaling promoted proliferation and survival.

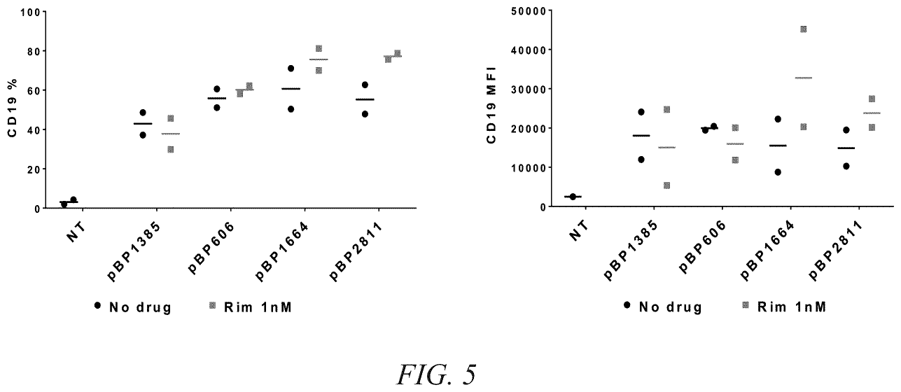

FIG. 5—NK cell proliferation with iMC activation. NK cells from two separate donors were transduced with 1385 (IRC9 alone), 606 (IMC alone), 1664 (iMC+iRC9) or 2811 (iMC+iRC9+IL-15). Cultures were split and left untreated (black) or 1 nM rimiducid added. Transduced NK cells (marked with CD19) were incubated with rimiducid for six days and the number of transduced NK cells relative to untransduced NK cells was assessed. An increase in the proportion of transduced cells after 6 days with drug treatment indicated selective outgrowth of activated iMC containing populations. MFI=Mean fluorescence intensity of CD19 in flow cytometry analysis, indicating a proportional growth advantage of highly expressing cells. Modified NK cells comprising the iMC or iRMC chimeric polypeptides had a growth advantage over NK cells that do not comprise iMC or iRMC chimeric polypeptides.

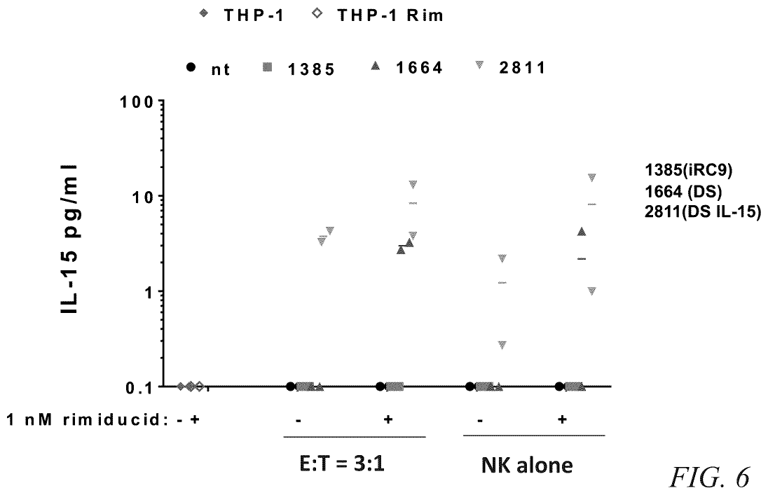

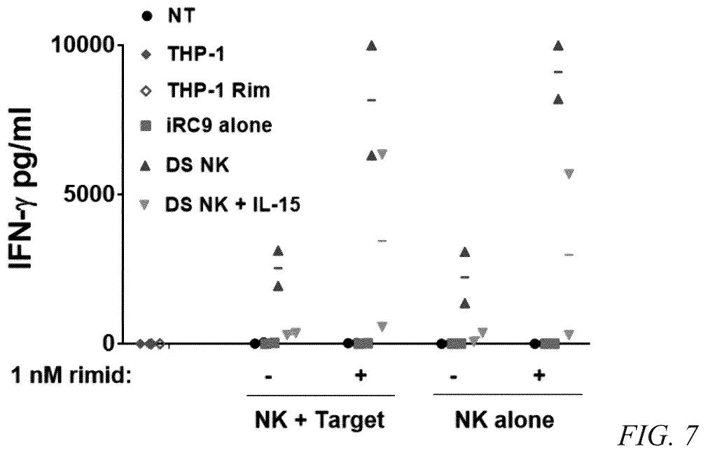

FIG. 6—IL-15 production in dual switch NK cells. NK cells derived from two donors were cultured in the presence of tumor target THP1 at an effector to target of 3:1 which activates NK cells naturally or NK cells were incubated alone in the presence or absence of 1 nM rimiducid. IL-15 levels in culture supernatant after 48 hours was assessed by multiplex bead analysis. Cytokine levels beneath the range of detection in the ELISA assay were assigned as 0.1 pg/ml. Without rimiducid, only transduced cells with IL-15 as a transgene produced IL-15. Stimulation of iMC activated endogenous IL-15 production. In the presence of rimiducid, modified NK cells that expressed iMC, nonmodified by an IL-15 transgene, also produced IL-15.

FIG. 7—InterferonY′ production in dual switch NK cells. NK cells derived from two donors were cultured in the presence of tumor target THP1 at an effector to target of 3:1 which activates NK cells naturally in the presence or absence of 1 nM rimiducid. IFNγ levels in culture supernatant after 48 hours was assessed by multiplex bead analysis. Stimulation of iMC activated IFNγ production. Rimiducid-dependent iMC activation led to high-level cytokine secretion. The modified cells demonstrated a relatively muted production of pro-inflammatory cytokines, such as TNF-α and IL-6.

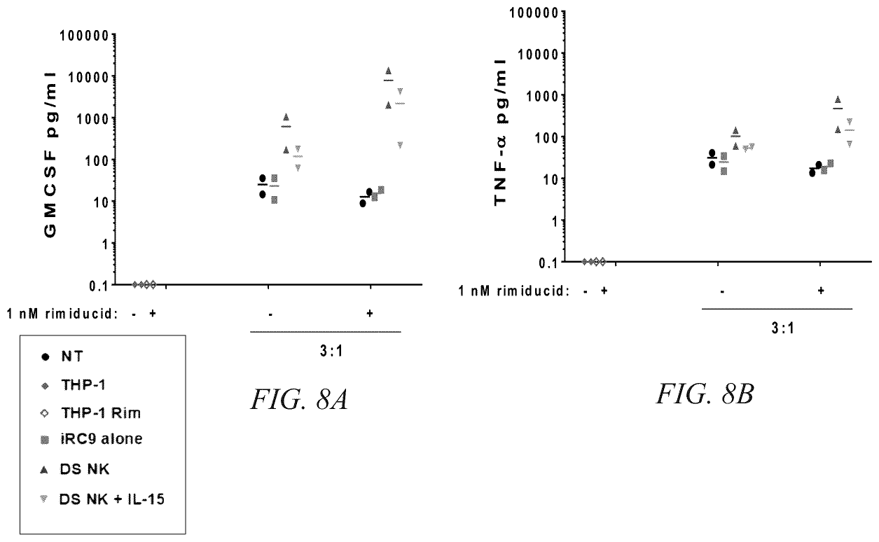

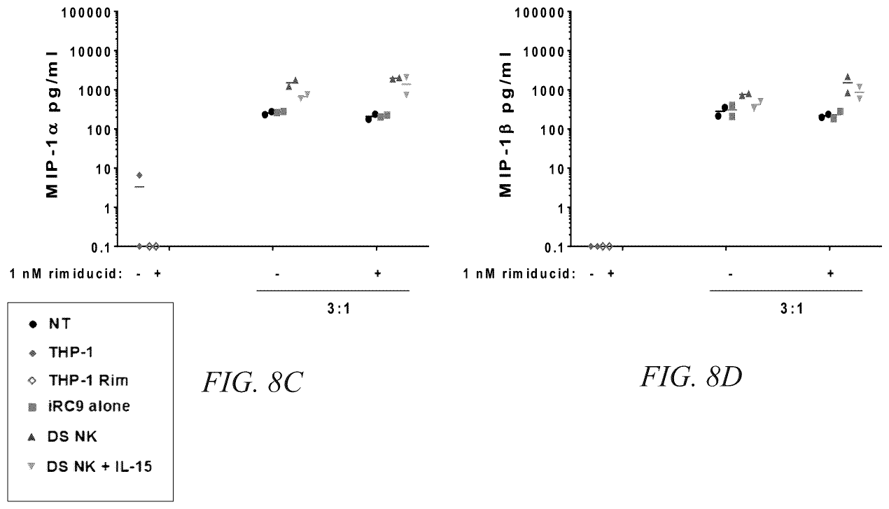

FIGS. 8A-8D—Cytokine and chemokine production in dual switch NK cells. NK cells derived from two donors were cultured in the presence of tumor target THP1 at an effector to target of 3:1 which activates NK cells naturally in the presence or absence of 1 nM rimiducid. GM-CSF (FIG. 8A), TNF-α (FIG. 8B) and the chemokines MIP1a (FIG. 8C) and MIP1β (FIG. 8D) in culture supernatant after 48 hours was assessed by multiplex bead analysis. Activation of iMC stimulated production of these factors already produced by activated NK cells.

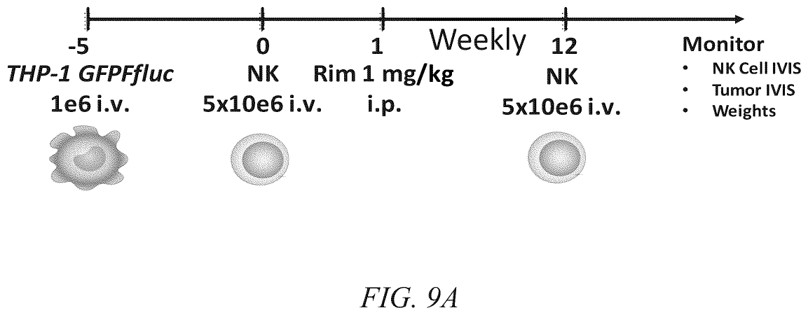

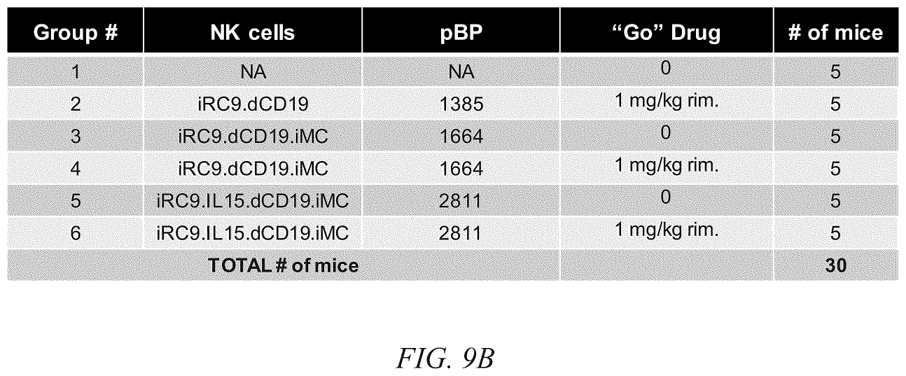

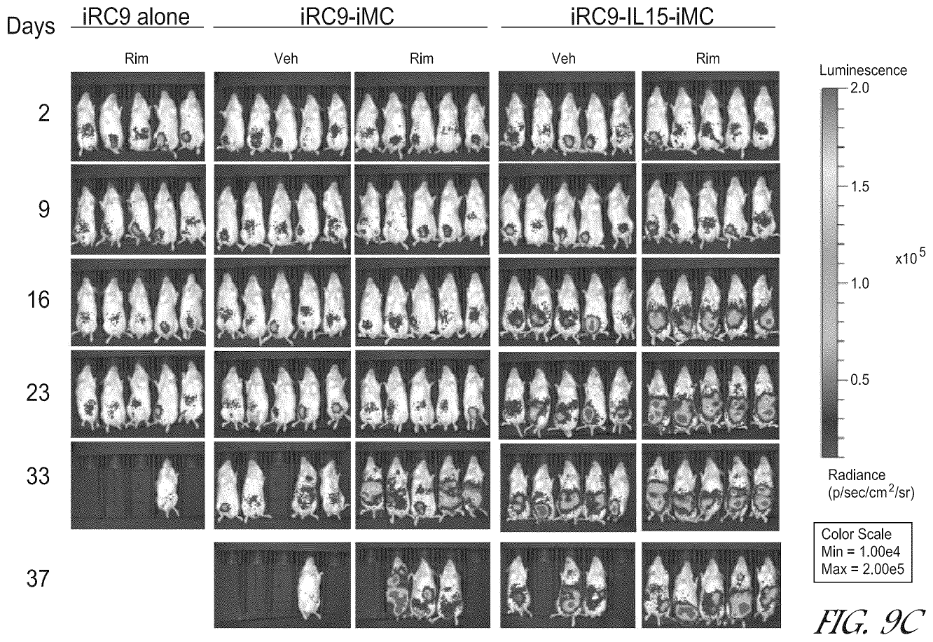

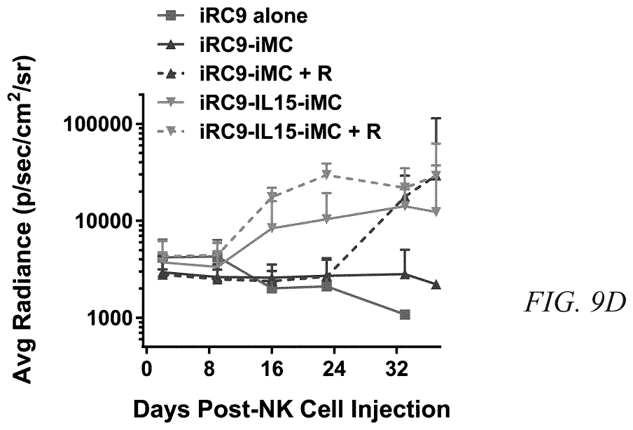

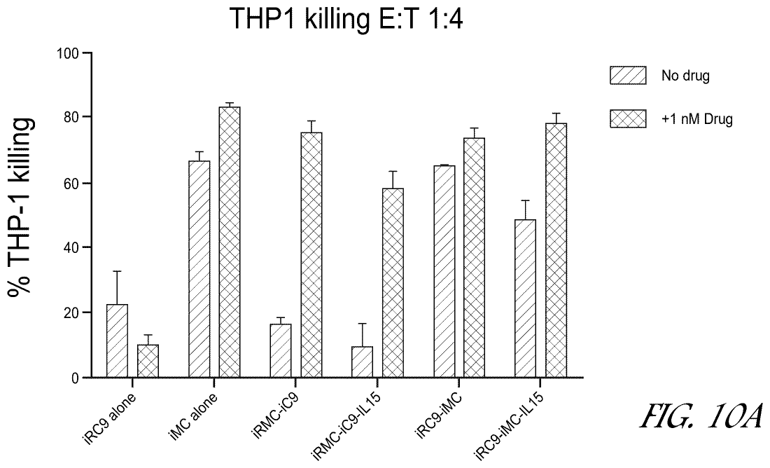

FIGS. 9A-9D—Proliferation of DS NK cells in vivo. FIG. 9A: Schematic describing engraftment of NSG immunodeficient mice with human THP1 tumor cells labeled with a GFP-Firefly luciferase reporter followed 5 days later by infusion with human DS NK cells labeled with Renilla luciferase. Rimiducid was added one day afterward and weekly thereafter at 1 mg/kg bodyweight. FIG. 9B: Table depicting groups of mice engrafted with NK cells transduced with expression viruses for iRC9 alone, with iMC or with iMC and autocrine IL-15. FIG. 9C: IVIS images of NK cells levels by biologic light imaging (BLI) of ren-luciferase activity with coelentrazine. FIG. 9D: Quantitation of BLI radiance for each mouse group at each timepoint in FIG. 9C. NK proliferation was evident with iMC and IL-15 presence and was further stimulated by activation of iMC with rimiducid. IL-15 augmented iMC-driven expansion in vivo.

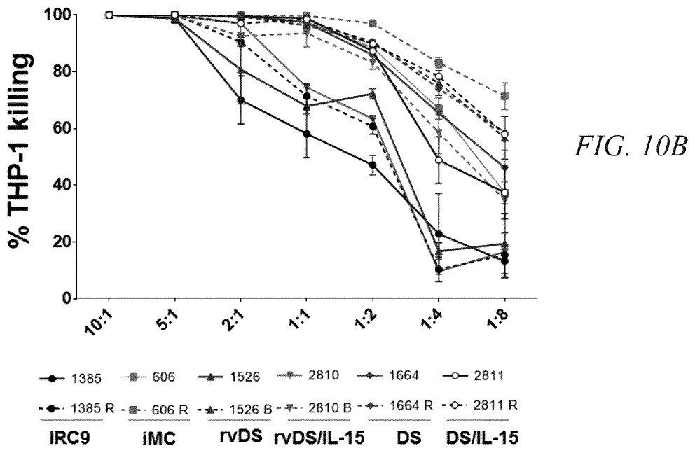

FIGS. 10A-10B—NK cell killing is enhanced by MC induction in vitro. THP1 AML tumor cells labeled with GFP luciferase were incubated for 24 hours alone (for reference) or in the presence of NK cells transduced with the indicated retroviral expression constructs. FIG. 10A: Specific lysis calculated by THP1 luciferase when targeted by NK cells at an effector to target ratio (E:T) of 1:4. Cells containing iMC alone have robust killing activity that is enhanced by rimiducid activity. iRMC containing NK cells have low basal killing activity without BPC-015 but are greatly enhanced for killing by addition of this drug. FIG. 10B: Plot of killing for each construct with and without activating ligand at decreasing E:T ratio. In this set of assays, the iRMC-expressing NK cells appeared to show a greater dependence on ligand than the iMC-expressing NK cells.

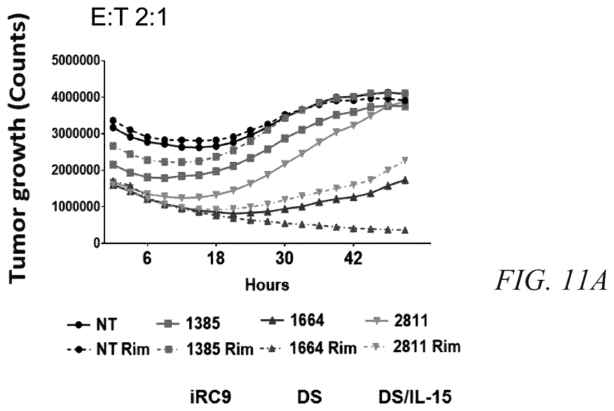

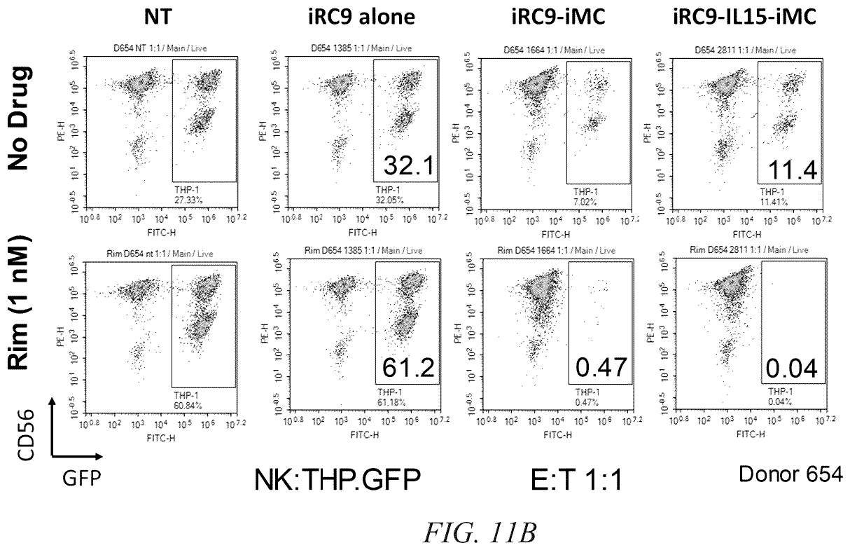

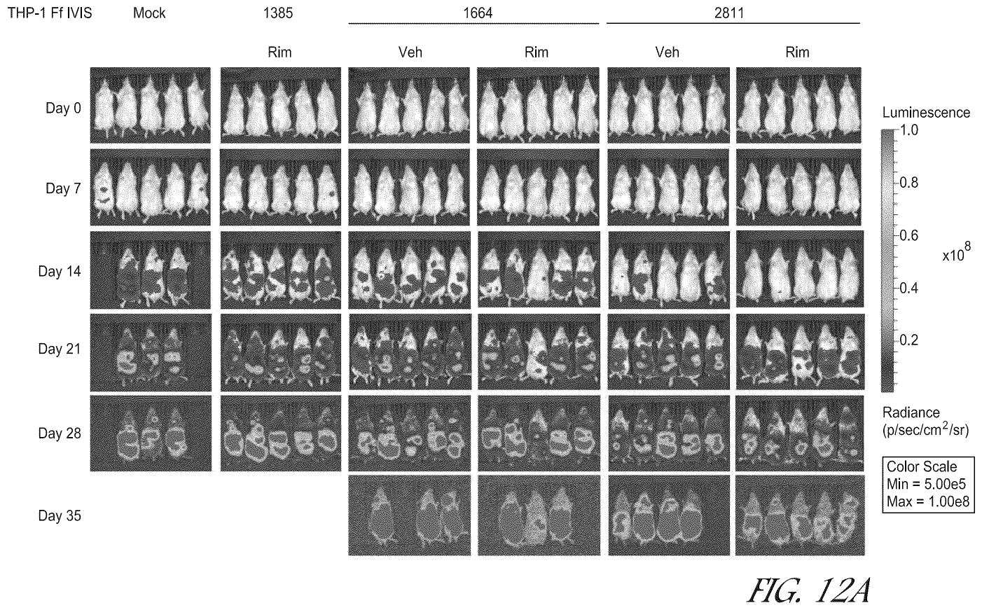

FIGS. 11A-11B—NK cell killing is enhanced by iMC in vitro. FIG. 11A: HPAC-GFP pancreatic cancer cells were incubated with NK cells transduced with the indicated retroviral expression constructs at an E:T of 2:1. Cells were placed in an incucyte real time live imaging chamber and GFP fluorescence measured every 3 hours for 2 days to indicate HPAC cell growth or control by the NK cells. iMC enabled DS NK cells controlled HPAC growth and this was enhanced by stimulation with 1 nM rimiducid. FIG. 11B: THP1-GFP AML cells were incubated with transduced NK cells at an E:T of 1:1 for 48 hours and the presence of GFP positive populations detected by flow cytometry. iMC enabled DS NK cells rapidly killed the target and this was enhanced by stimulation with 1 nM rimiducid. iMC-dependent activation improved NK-mediated HPAC (E:T 2:1) and THP-1 (1:1) tumor control.

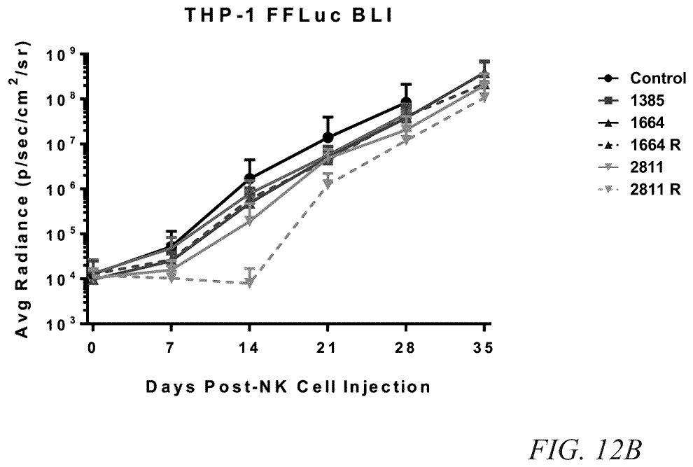

FIGS. 12A-12B-Anti-tumor efficacy of DS NK cells. The protocol for combining DS NK cells with THP1-ffLUC labeled tumor cells is as described in FIGS. 9A and 9B. FIG. 12A: IVIS images of THP1 levels by biologic light imaging (BLI) of firefly-luciferase activity with luciferin. FIG. 12B: Quantitation of BLI radiance for each mouse group at each timepoint in FIG. 12A. Tumor expansion is delayed with iMC and IL-15 presence and is further stimulated by activation of iMC with rimiducid. IL-15 and activation of iMC combined to increase the control of tumor expansion.

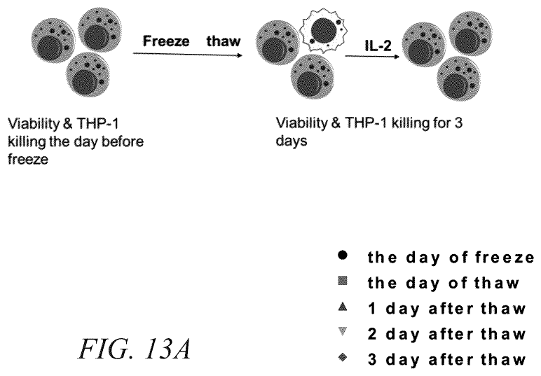

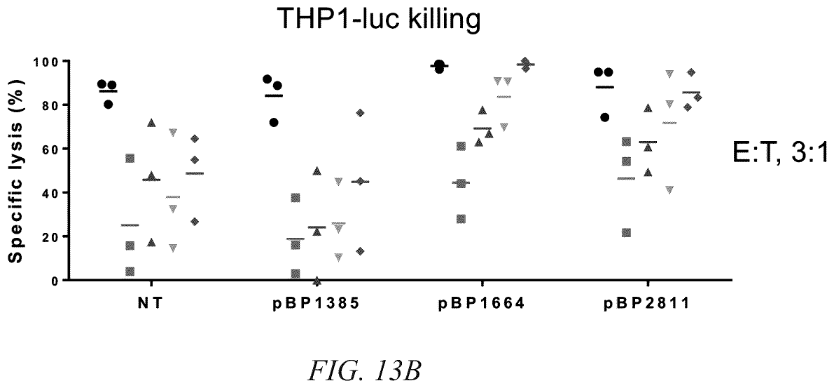

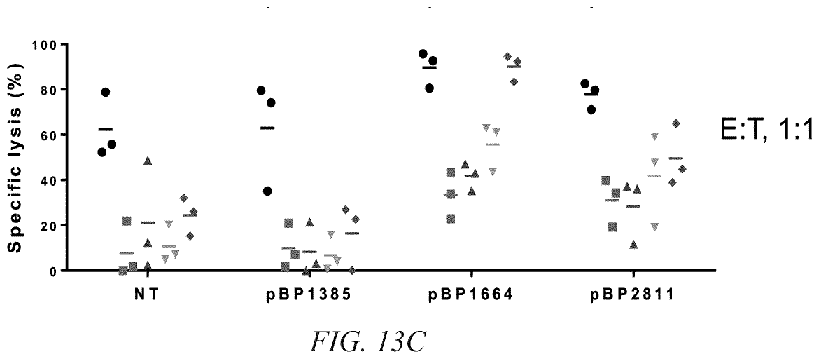

FIGS. 13A-13C-iMC blocks NK cell inefficacy following cryostorage. FIG. 13A: Transduced NK cells were maintained in standard culture conditions. Cells were slowly frozen in 90% fetal calf serum/10% dimethylsulfoxide and stored below −150° C. for up to four weeks. Overall NK viability after freeze-thaw was similar for each group. The graphs provided in this figure (FIGS. 13B and 13C) relate to efficacy. Cell viability was poor in for each transductant immediately following thaw and replating but recovered over three days of standard culture. NK cells prior to and after the indicated period of recovery from freeze/thaw were cultured with THP1-luciferase targets for 24 hours at an E:T of 3:1 (FIG. 13B) or 1:1 (FIG. 13C) to assess killing efficacy. DS NK cells with iMC were capable of regaining their potency over 48 to 72 hours after cryostorage while cells lacking iMC lacked efficacy.

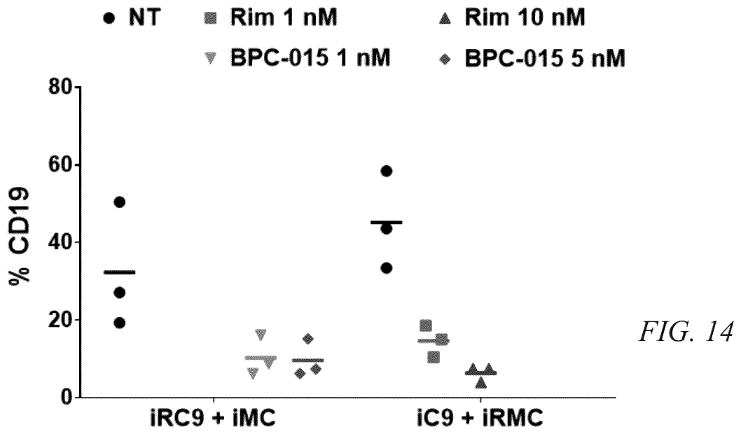

FIG. 14—Caspase-9 activation ablates DS NK cells. NK cells from three separate donors were transduced with recombinant retroviruses 1664 (iMC+iRC9) or 1531 (iC9+iRMC) and expanded. Cultures were split and left untreated (black) or dimerizing drugs BPC015 or rimiducid added. After 24 hours NK cells were assayed for presence of the CD19 marker cotransduced with the switches. Drug-specific loss of the transduced cell population indicated targeted apoptosis by activation of the safety switch. Activation with cognate drug reduced viability of transduced NK cell in either dual switch configuration (iRMC+iC9, or iRC9+iMC). MFI=Mean fluorescence intensity of CD19 in flow cytometry, indicating a proportional growth advantage of highly expressing cells.

### Example 2. Examples of Particular Nucleic Acid and Amino Acid Sequences

The following tables include examples of polypeptide and nucleotide sequences coding for the polypeptides of the chimeric signaling polypeptides. It is understood that sequences of individual polypeptides provided in these examples, such as, for example, the truncated MyD88 polypeptides, co-stimulatory polypeptide cytoplasmic signaling regions, FKBP12 variant regions, and caspase polypeptides, may be used to construct other expression vectors that encode chimeric signaling polypeptides of the present embodiments.

TABLE
Plasmid C: pBP1800-SFG-MyD88.CD28.OX40.Fv.Fv.T2A.
aPSCAscFv.CD34e.CD8stm.zeta

| | SEQ | | SEQ | |
|---|---|---|---|---|
| Fragment | ID # | Nucleotide | ID # | Peptide |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGGA | | RPGASVGRLLDLLTKLGRDD |
| | | CTTTGAGTACTTGGAGATCCGGCAACTGGAGAC | | VLLELGPSIEEDCQKYILKQ |
| | | ACAAGCGGACCCCACTGGCAGGCTGCTGGACGC | | QQEEAEKPLQVAAVDSSVPR |
| | | CTGGCAGGGACGCCCTGGCGCCTCTGTAGGCCG | | TAELAGITTLDDPLGHMPER |
| | | ACTGCTCGATCTGCTTACCAAGCTGGGCCGCGA | | FDAFICYCPSDI |
| | | CGACGTGCTGCTGGAGCTGGGACCCAGCATTGA | | |
| | | GGAGGATTGCCAAAAGTATATCTTGAAGCAGCA | | |
| | | GCAGGAGGAGGCTGAGAAGCCTTTACAGGTGGC | | |
| | | CGCTGTAGACAGCAGTGTCCCACGGACAGCAGA | | |
| | | GCTGGCGGGCATCACCACACTTGATGACCCCCT | | |
| | | GGGGCATATGCCTGAGCGTTTCGATGCCTTCATC | | |
| | | TGCTATTGCCCCAGCGACATC | | |
| Linker | 3 | CTCGAG | 4 | LE |
| CD28 | 5 | AGGAGTAAGAGGAGCAGGCTCCTGCACAGTGAC | 6 | RSKRSRLLHSDYMNMTPRR |
| signaling | | TACATGAACATGACTCCCCGCCGCCCCGGGCCC | | PGPTRKHYQPYAPPRDFAA |
| domain | | ACCCGCAAGCATTACCAGCCCTATGCCCCACCA | | YRS |
| | | CGCGACTTCGCAGCCTATCGCTCC | | |
| OX40 | 7 | AGGGACCAGAGGCTGCCCCCCGATGCCCACAAG | 8 | RDQRLPPDAHKPPGGGSFR |
| signaling | | CCCCCTGGGGGAGGCAGTTTCCGGACCCCCATC | | TPIQEEQADAHSTLAKIGS |
| domain | | CAAGAGGAGCAGGCCGACGCCCACTCCACCCTG | | G |
| | | GCCAAGATC | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCVVHYTGMLEDGKKVDSSR |
| | | TCGTCCATTATACAGGCATGTTGGAGGACGGCAA | | DRNKPFKFMLGKQEVIRGWE |
| | | AAAGGTGGACAGTAGTAGAGATCGCAATAAACCT | | EGVAQMSVGQRAKLTISPDY |
| | | TTCAAATTCATGTTGGGAAAACAAGAAGTCATTA | | AYGATGHPGIIPPHATLVFD |
| | | GGGGATGGGAGGAGGGCGTGGCTCAAATGTCCGT | | VELLKLE |
| | | CGGCCAACGCGCTAAGCTCACCATCAGCCCCGA | | |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCVVHYTGMLEDGKKVDSSR |
| | | GTGGTGCACTACACCGGGATGCTTGAAGATGGA | | DRNKPFKFMLGKQEVIRGWE |
| | | AAGAAAGTTGATTCCTCCCGGGACAGAAACAAGC | | EGVAQMSVGQRAKLTISPDY |
| | | CCTTTAAGTTTATGCTAGGCAAGCAGGAGGTGAT | | AYGATGHPGIIPPHATLVFD |
| | | CCGAGGCTGGGAAGAAGGGGTTGCCCAGATGAG | | VELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQC |
| Peptide | | GCAATACTCAAGGGCGTTCAATGTTCACGG | | SR |
| PSCA(A11) | 25 | GACATCCAACTGACGCAAAGCCCATCTACACTCA | 26 | DIQLTQSPSTLSASMGDRVTI |
| V_{L} | | GCGCTAGCATGGGGGACAGGGTCACAATCACGT | | TCSASSSVRFIHWYQQKPGKA |
| | | GCTCTGCCTCAAGTTCCGTTAGGTTTATCCATTG | | PKRLIYDTSKLASGVPSRFSG |
| | | GTATCAGCAGAAACCTGGAAAGGCCCCAAAAAG | | SGSGTDFTLTISSLQPEDFAT |
| | | ACTGATCTATGATACCAGCAAGCTGGCTTCCGGA | | YYCQQWGSSPFTFGQGTKVEI |
| | | GTGCCCTCAAGGTTCTCAGGATCTGGCAGTGGG | | K |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | |
| | | GGTGACACTGAGTTTGTCCCCAAGTTTCAGGGCA | | LQMNSLRAEDTAVYYCKTGG |
| | | GAGCCACCATGAGCGCTGACACAAGCAAAAACA | | FWGQGTLVTVSS |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLRP |
| | | GCACAAACGTAAGT | | EACRPAAGGAVHTRGLDFACD |
| CD8 | 39 | ATCTATATCTGGGCACCTCTCGCTGGCACCTGTG | 40 | IYIWAPLAGTCGVLLLSLVI |
| transmembrane | | GAGTCCTTCTGCTCAGCCTGGTTATTACTCTGTA | | TLYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKR |
| | | GAGCTCAATCTAGGACGAAGAGAGGAGTACGAT | | RGRDPEMGGKPRRKNPQEG |
| | | GTTTTGGACAAGAGACGTGGCCGGGACCCTGAG | | LYNELQKDKMAEAYSEIGM |
| | | ATGGGGGGAAAGCCGAGAAGGAAGAACCCTCAG | | KGERRRGKGHDGLYQGLST |
| | | GAAGGCCTGTACAATGAACTGCAGAAAGATAAGA | | ATKDTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid E: pBP1802-SFG-MyD88.ICOS.Fv.Fv.T2A.aPSCAscFv.CD34e.CD8stm.zeta

| | SEQ | | SEQ | |
|---|---|---|---|---|
| Fragment | ID # | Nucleotide | ID # | Peptide |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGGA | | RPGASVGRLLDLLTKLGRDD |
| | | CTTTGAGTACTTGGAGATCCGGCAACTGGAGAC | | VLLELGPSIEEDCQKYILKQ |
| | | ACAAGCGGACCCCACTGGCAGGCTGCTGGACGC | | QQEEAEKPLQVAAVDSSVPR |
| | | CTGGCAGGGACGCCCTGGCGCCTCTGTAGGCC | | TAELAGITTLDDPLGHMPER |
| | | GACTGCTCGATCTGCTTACCAAGCTGGGCCGCG | | FDAFICYCPSDI |
| | | ACGACGTGCTGCTGGAGCTGGGACCCAGCATTG | | |
| | | AGGAGGATTGCCAAAAGTATATCTTGAAGCAGCA | | |
| | | GCAGGAGGAGGCTGAGAAGCCTTTACAGGTGGC | | |
| | | CGCTGTAGACAGCAGTGTCCCACGGACAGCAGA | | |
| | | GCTGGCGGGCATCACCACACTTGATGACCCCCT | | |
| | | GGGGCATATGCCTGAGCGTTTCGATGCCTTCATC | | |
| | | TGCTATTGCCCCAGCGACATC | | |
| Linker | 3 | CTCGAG | 4 | LE |
| ICOS | 45 | ACAAAAAAGAAGTATTCATCCAGTGTGCACGACC | 46 | TKKKYSSSVHDPNGEYMFM |
| signaling | | CTAACGGTGAATACATGTTCATGAGAGCAGTGAA | | RAVNTAKKSRLTDVTL |
| domain | | CACAGCCAAAAAATCTAGACTCACAGATGTGACC | | |
| | | CTA | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCVVHYTGMLEDGKKVDSSR |
| | | TCGTCCATTATACAGGCATGTTGGAGGACGGCAA | | DRNKPFKFMLGKQEVIRGWE |
| | | AAAGGTGGACAGTAGTAGAGATCGCAATAAACCT | | EGVAQMSVGQRAKLTISPDY |
| | | TTCAAATTCATGTTGGGAAAACAAGAAGTCATTAG | | AYGATGHPGIIPPHATLVFDV |
| | | GGGATGGGAGGAGGGCGTGGCTCAAATGTCCGT | | ELLKLE |
| | | CGGCCAACGCGCTAAGCTCACCATCAGCCCCGA | | |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCVVHYTGMLEDGKKVDSSR |
| | | GTGGTGCACTACACCGGGATGCTTGAAGATGGA | | DRNKPFKFMLGKQEVIRGWE |
| | | AAGAAAGTTGATTCCTCCCGGGACAGAAACAAGC | | EGVAQMSVGQRAKLTISPDY |
| | | CCTTTAAGTTTATGCTAGGCAAGCAGGAGGTGAT | | AYGATGHPGIIPPHATLVFD |
| | | CCGAGGCTGGGAAGAAGGGGTTGCCCAGATGAG | | VELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQC |
| Peptide | | GCAATACTCAAGGGCGTTCAATGTTCACGG | | SR |
| PSCA(A11) | 25 | GACATCCAACTGACGCAAAGCCCATCTACACTCA | 26 | DIQLTQSPSTLSASMGDRVTI |
| V_{L} | | GCGCTAGCATGGGGGACAGGGTCACAATCACGT | | TCSASSSVRFIHWYQQKPGK |
| | | GCTCTGCCTCAAGTTCCGTTAGGTTTATCCATTG | | APKRLIYDTSKLASGVPSRFS |
| | | GTATCAGCAGAAACCTGGAAAGGCCCCAAAAAG | | GSGSGTDFTLTISSLQPEDFA |
| | | ACTGATCTATGATACCAGCAAGCTGGCTTCCGGA | | TYYCQQWGSSPFTFGQGTK |
| | | GTGCCCTCAAGGTTCTCAGGATCTGGCAGTGGG | | VEIK |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | LQMNSLRAEDTAVYYCKTGG |
| | | GGTGACACTGAGTTTGTCCCCAAGTTTCAGGGCA | | FWGQGTLVTVSS |
| | | GAGCCACCATGAGCGCTGACACAAGCAAAAACA | | |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLR |
| | | GCACAAACGTAAGT | | PEACRPAAGGAVHTRGLDFA |
| | | | | CD |
| CD8 | 39 | ATCTATATCTGGGCACCTCTCGCTGGCACCTGTG | 40 | IYIWAPLAGTCGVLLLSLVIT |
| transmembrane | | GAGTCCTTCTGCTCAGCCTGGTTATTACTCTGTA | | LYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTAT |
| | | GAAGGCCTGTACAATGAACTGCAGAAAGATAAGA | | KDTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid H: pBP1815-SFG-4-1BB.Fv.Fv.T2A.aPSCAscFv.CD34e.CD8stm.zeta

| | SEQ | | SEQ | |
|---|---|---|---|---|
| Fragment | ID # | Nucleotide | ID # | Peptide |
| Leader | 47 | ATGCTCGAG | 48 | MLE |
| 4-1BB/CD137 | 49 | AAACGGGGCAGAAAGAAACTCCTGTATATATTCA | 50 | KRGRKKLLYIFKQPFMRPV |
| signaling | | AACAACCATTTATGAGACCAGTACAAACTACTCAA | | QTTQEEDGCSCRFPEEEEG |
| domain | | GAGGAAGATGGCTGTAGCTGCCGATTTCCAGAA | | GCEL |
| | | GAAGAAGAAGGAGGATGTGAACTG | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCVVHYTGMLEDGKKVDSSR |
| | | TCGTCCATTATACAGGCATGTTGGAGGACGGCAA | | DRNKPFKFMLGKQEVIRGWE |
| | | AAAGGTGGACAGTAGTAGAGATCGCAATAAACCT | | EGVAQMSVGQRAKLTISPDY |
| | | TTCAAATTCATGTTGGGAAAACAAGAAGTCATTAG | | AYGATGHPGIIPPHATLVFD |
| | | GGGATGGGAGGAGGGCGTGGCTCAAATGTCCGT | | VELLKLE |
| | | CGGCCAACGCGCTAAGCTCACCATCAGCCCCGA | | |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCVVHYTGMLEDGKKVDSSR |
| | | GTGGTGCACTACACCGGGATGCTTGAAGATGGA | | DRNKPFKFMLGKQEVIRGWE |
| | | AAGAAAGTTGATTCCTCCCGGGACAGAAACAAGC | | EGVAQMSVGQRAKLTISPDY |
| | | CCTTTAAGTTTATGCTAGGCAAGCAGGAGGTGAT | | AYGATGHPGIIPPHATLVFD |
| | | CCGAGGCTGGGAAGAAGGGGTTGCCCAGATGAG | | VELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQC |
| Peptide | | GCAATACTCAAGGGCGTTCAATGTTCACGG | | SR |
| PSCA(A11) | 25 | GACATCCAACTGACGCAAAGCCCATCTACACTCA | 26 | DIQLTQSPSTLSASMGDRVT |
| V_{L} | | GCGCTAGCATGGGGGACAGGGTCACAATCACGT | | ITCSASSSVRFIHWYQQKPG |
| | | GCTCTGCCTCAAGTTCCGTTAGGTTTATCCATTG | | KAPKRLIYDTSKLASGVPSR |
| | | GTATCAGCAGAAACCTGGAAAGGCCCCAAAAAG | | FSGSGSGTDFTLTISSLQPE |
| | | ACTGATCTATGATACCAGCAAGCTGGCTTCCGGA | | DFATYYCQQWGSSPFTFGQG |
| | | GTGCCCTCAAGGTTCTCAGGATCTGGCAGTGGG | | TKVEIK |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | LQMNSLRAEDTAVYYCKTGG |
| | | GGTGACACTGAGTTTGTCCCCAAGTTTCAGGGCA | | FWGQGTLVTVSS |
| | | GAGCCACCATGAGCGCTGACACAAGCAAAAACA | | |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLRP |
| | | GCACAAACGTAAGT | | EACRPAAGGAVHTRGLDFAC |
| | | | | D |
| CD8 | 39 | ATCTATATCTGGGCACCTCTCGCTGGCACCTGTG | 40 | IYIWAPLAGTCGVLLLSLVI |
| transmembrane | | GAGTCCTTCTGCTCAGCCTGGTTATTACTCTGTA | | TLYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKR |
| | | GAGCTCAATCTAGGACGAAGAGAGGAGTACGAT | | RGRDPEMGGKPRRKNPQEG |
| | | GTTTTGGACAAGAGACGTGGCCGGGACCCTGAG | | LYNELQKDKMAEAYSEIGM |
| | | ATGGGGGGAAAGCCGAGAAGGAAGAACCCTCAG | | KGERRRGKGHDGLYQGLST |
| | | GAAGGCCTGTACAATGAACTGCAGAAAGATAAGA | | DATKTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid L: pBP2205-SFG-Myr-MyD88.Fv.Fv.T2A.aPSCAscFv.CD34e.CD8stm.zeta

| | SEQ | | SEQ | |
|---|---|---|---|---|
| Fragment | ID # | Nucleotide | ID # | Peptide |
| Myristoylation | 51 | ATGGGGAGTAGCAAGAGCAAGCCTAAGGACCCC | 52 | MGSSKSKPKDPSQR |
| peptide | | AGCCAGCGC | | |
| Linker | 53 | AGAGCATGC | 54 | RAC |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGG | | RPGASVGRLLDLLTKLGRDD |
| | | ACTTTGAGTACTTGGAGATCCGGCAACTGGAGAC | | VLLELGPSIEEDCQKYILKQ |
| | | ACAAGCGGACCCCACTGGCAGGCTGCTGGACGC | | QQEEAEKPLQVAAVDSSVPR |
| | | CTGGCAGGGACGCCCTGGCGCCTCTGTAGGCC | | TAELAGITTLDDPLGHMPER |
| | | GACTGCTCGATCTGCTTACCAAGCTGGGCCGCG | | FDAFICYCPSDI |
| | | ACGACGTGCTGCTGGAGCTGGGACCCAGCATTG | | |
| | | AGGAGGATTGCCAAAAGTATATCTTGAAGCAGCA | | |
| | | GCAGGAGGAGGCTGAGAAGCCTTTACAGGTGGC | | |
| | | CGCTGTAGACAGCAGTGTCCCACGGACAGCAGA | | |
| | | GCTGGCGGGCATCACCACACTTGATGACCCCCT | | |
| | | GGGGCATATGCCTGAGCGTTTCGATGCCTTCATC | | |
| | | TGCTATTGCCCCAGCGACATC | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCVVHYTGMLEDGKKVDSSR |
| | | TCGTCCATTATACAGGCATGTTGGAGGACGGCAA | | DRNKPFKFMLGKQEVIRGWE |
| | | AAAGGTGGACAGTAGTAGAGATCGCAATAAACCT | | EGVAQMSVGQRAKLTISPDY |
| | | TTCAAATTCATGTTGGGAAAACAAGAAGTCATTAG | | AYGATGHPGIIPPHATLVFD |
| | | GGGATGGGAGGAGGGCGTGGCTCAAATGTCCGT | | VELLKLE |
| | | CGGCCAACGCGCTAAGCTCACCATCAGCCCCGA | | |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCWHYTGMLEDGKKVDSSRD |
| | | GTGGTGCACTACACCGGGATGCTTGAAGATGGA | | RNKPFKFMLGKQEVIRGWEE |
| | | AAGAAAGTTGATTCCTCCCGGGACAGAAACAAGC | | GVAQMSVGQRAKLTISPDYA |
| | | CCTTTAAGTTTATGCTAGGCAAGCAGGAGGTGAT | | YGATGHPGIIPPHATLVFDV |
| | | CCGAGGCTGGGAAGAAGGGGTTGCCCAGATGAG | | ELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQCS |
| Peptide | | GCAATACTCAAGGGCGTTCAATGTTCACGG | | R |
| PSCA(A11) | 25 | GACATCCAACTGACGCAAAGCCCATCTACACTCA | 26 | DIQLTQSPSTLSASMGDRVT |
| V_{L} | | GCGCTAGCATGGGGGACAGGGTCACAATCACGT | | ITCSASSSVRFIHWYQQKPG |
| | | GCTCTGCCTCAAGTTCCGTTAGGTTTATCCATTG | | KAPKRLIYDTSKLASGVPSR |
| | | GTATCAGCAGAAACCTGGAAAGGCCCCAAAAAG | | FSGSGSGTDFTLTISSLQPE |
| | | ACTGATCTATGATACCAGCAAGCTGGCTTCCGGA | | DFATYYCQQWGSSPFTFGQG |
| | | GTGCCCTCAAGGTTCTCAGGATCTGGCAGTGGG | | TKVEIK |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | LQMNSLRAEDTAVYYCKTGG |
| | | GGTGACACTGAGTTTGTCCCCAAGTTTCAGGGCA | | FWGQGTLVTVSS |
| | | GAGCCACCATGAGCGCTGACACAAGCAAAAACA | | |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLRP |
| | | GCACAAACGTAAGT | | EACRPAAGGAVHTRGLDFAC |
| | | | | D |
| CD8 | 39 | ATCTATATCTGGGCACCTCTCGCTGGCACCTGTG | 40 | IYIWAPLAGTCGVLLLSLVIT |
| transmembrane | | GAGTCCTTCTGCTCAGCCTGGTTATTACTCTGTA | | LYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTATK |
| | | GAAGGCCTGTACAATGAACTGCAGAAAGATAAGA | | DTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid M: pBP2206-SFG-Myr-MyD88.CD40.Fv.Fv.T2A.aPSCAscFv.CD34e.CD8stm.zeta

| | SEQ | | SEQ | |
|---|---|---|---|---|
| Fragment | ID # | Nucleotide | ID # | Peptide |
| Myristoylation | 51 | ATGGGGAGTAGCAAGAGCAAGCCTAAGGACCCC | 52 | MGSSKSKPKDPSQR |
| peptide | | AGCCAGCGC | | |
| Linker | 53 | AGAGCATGC | 54 | RAC |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGG | | RPGASVGRLLDLLTKLGRDD |
| | | ACTTTGAGTACTTGGAGATCCGGCAACTGGAGAC | | VLLELGPSIEEDCQKYILKQ |
| | | ACAAGCGGACCCCACTGGCAGGCTGCTGGACGC | | QQEEAEKPLQVAAVDSSVPR |
| | | CTGGCAGGGACGCCCTGGCGCCTCTGTAGGCC | | TAELAGITTLDDPLGHMPER |
| | | GACTGCTCGATCTGCTTACCAAGCTGGGCCGCG | | FDAFICYCPSDI |
| | | ACGACGTGCTGCTGGAGCTGGGACCCAGCATTG | | |
| | | AGGAGGATTGCCAAAAGTATATCTTGAAGCAGCA | | |
| | | GCAGGAGGAGGCTGAGAAGCCTTTACAGGTGGC | | |
| | | CGCTGTAGACAGCAGTGTCCCACGGACAGCAGA | | |
| | | GCTGGCGGGCATCACCACACTTGATGACCCCCT | | |
| | | GGGGCATATGCCTGAGCGTTTCGATGCCTTCATC | | |
| | | TGCTATTGCCCCAGCGACATC | | |
| Linker | 3 | CTCGAG | 4 | LE |
| CD40 | 55 | AAAAAGGTGGCCAAGAAGCCAACCAATAAGGCC | 56 | KKVAKKPTNKAPHPKQEPQE |
| signaling | | CCCCACCCCAAGCAGGAGCCCCAGGAGATCAAT | | INFPDDLPGSNTAAPVQETL |
| domain | | TTTCCCGACGATCTTCCTGGCTCCAACACTGCTG | | HGCQPVTQEDGKESRISVQER |
| | | CTCCAGTGCAGGAGACTTTACATGGATGCCAGC | | Q |
| | | CGGTCACCCAGGAGGATGGCAAAGAGAGTCGCA | | |
| | | TCTCAGTGCAGGAGAGACAG | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCWHYTGMLEDGKKVDSSRD |
| | | TCGTCCATTATACAGGCATGTTGGAGGACGGCAA | | RNKPFKFMLGKQEVIRGWEE |
| | | AAAGGTGGACAGTAGTAGAGATCGCAATAAACCT | | GVAQMSVGQRAKLTISPDY |
| | | TTCAAATTCATGTTGGGAAAACAAGAAGTCATTAG | | |
| | | GGGATGGGAGGAGGGCGTGGCTCAAATGTCCGT | | AYGATGHPGIIPPHATLVFDV |
| | | CGGCCAACGCGCTAAGCTCACCATCAGCCCCGA | | ELLKLE |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCVVHYTGMLEDGKKVDSSR |
| | | GTGGTGCACTACACCGGGATGCTTGAAGATGGA | | DRNKPFKFMLGKQEVIRGWE |
| | | AAGAAAGTTGATTCCTCCCGGGACAGAAACAAGC | | EGVAQMSVGQRAKLTISPDY |
| | | CCTTTAAGTTTATGCTAGGCAAGCAGGAGGTGAT | | AYGATGHPGIIPPHATLVFD |
| | | CCGAGGCTGGGAAGAAGGGGTTGCCCAGATGAG | | VELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQCS |
| Peptide | | GCAATACTCAAGGGCGTTCAATGTTCACGG | | R |
| PSCA(A11) | 25 | GACATCCAACTGACGCAAAGCCCATCTACACTCA | 26 | DIQLTQSPSTLSASMGDRVTI |
| V_{L} | | GCGCTAGCATGGGGGACAGGGTCACAATCACGT | | TCSASSSVRFIHWYQQKPGKA |
| | | GCTCTGCCTCAAGTTCCGTTAGGTTTATCCATTG | | PKRLIYDTSKLASGVPSRFSG |
| | | GTATCAGCAGAAACCTGGAAAGGCCCCAAAAAG | | SGSGTDFTLTISSLQPEDFA |
| | | ACTGATCTATGATACCAGCAAGCTGGCTTCCGGA | | TYYCQQWGSSPFTFGQGTK |
| | | GTGCCCTCAAGGTTCTCAGGATCTGGCAGTGGG | | VEIK |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | LQMNSLRAEDTAVYYCKTGG |
| | | GGTGACACTGAGTTTGTCCCCAAGTTTCAGGGCA | | FWGQGTLVTVSS |
| | | GAGCCACCATGAGCGCTGACACAAGCAAAAACA | | |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLRP |
| | | GCACAAACGTAAGT | | EACRPAAGGAVHTRGLDFACD |
| CD8 | 39 | ATCTATATCTGGGCACCTCTCGCTGGCACCTGTG | 40 | IYIWAPLAGTCGVLLLSLVITL |
| transmembrane | | GAGTCCTTCTGCTCAGCCTGGTTATTACTCTGTA | | YCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTAT |
| | | GAAGGCCTGTACAATGAACTGCAGAAAGATAAGA | | KDTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid N: pBP2208-SFG-CD40.Fv.Fv.T2A.aPSCAscFv.CD34e.CD8stm.zeta

| | SEQ | | SEQ | |
|---|---|---|---|---|
| Fragment | ID # | Nucleotide | ID # | Peptide |
| Leader | 47 | ATGCTCGAG | 48 | MLE |
| CD40 | 55 | AAAAAGGTGGCCAAGAAGCCAACCAATAAGGCC | 56 | KKVAKKPTNKAPHPKQEPQE |
| signaling | | CCCCACCCCAAGCAGGAGCCCCAGGAGATCAAT | | INFPDDLPGSNTAAPVQETLH |
| domain | | TTTCCCGACGATCTTCCTGGCTCCAACACTGCTG | | GCQPVTQEDGKESRISVQER |
| | | CTCCAGTGCAGGAGACTTTACATGGATGCCAGC | | Q |
| | | CGGTCACCCAGGAGGATGGCAAAGAGAGTCGCA | | |
| | | TCTCAGTGCAGGAGAGACAG | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCVVHYTGMLEDGKKVDSSR |
| | | TCGTCCATTATACAGGCATGTTGGAGGACGGCAA | | DRNKPFKFMLGKQEVIRGWE |
| | | AAAGGTGGACAGTAGTAGAGATCGCAATAAACCT | | EGVAQMSVGQRAKLTISPDY |
| | | TTCAAATTCATGTTGGGAAAACAAGAAGTCATTAG | | AYGATGHPGIIPPHATLVFD |
| | | GGGATGGGAGGAGGGCGTGGCTCAAATGTCCGT | | VELLKLE |
| | | CGGCCAACGCGCTAAGCTCACCATCAGCCCCGA | | |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCVVHYTGMLEDGKKVDSSR |
| | | GTGGTGCACTACACCGGGATGCTTGAAGATGGA | | DRNKPFKFMLGKQEVIRGWE |
| | | AAGAAAGTTGATTCCTCCCGGGACAGAAACAAGC | | EGVAQMSVGQRAKLTISPDY |
| | | CCTTTAAGTTTATGCTAGGCAAGCAGGAGGTGAT | | AYGATGHPGIIPPHATLVFD |
| | | CCGAGGCTGGGAAGAAGGGGTTGCCCAGATGAG | | VELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQCS |
| Peptide | | GCAATACTCAAGGGCGTTCAATGTTCACGG | | R |
| PSCA(A11) | 25 | GACATCCAACTGACGCAAAGCCCATCTACACTCA | 26 | DIQLTQSPSTLSASMGDRVTI |
| V_{L} | | GCGCTAGCATGGGGGACAGGGTCACAATCACGT | | TCSASSSVRFIHWYQQKPGKA |
| | | GCTCTGCCTCAAGTTCCGTTAGGTTTATCCATTG | | PKRLIYDTSKLASGVPSRFSG |
| | | GTATCAGCAGAAACCTGGAAAGGCCCCAAAAAG | | SGSGTDFTLTISSLQPEDFA |
| | | ACTGATCTATGATACCAGCAAGCTGGCTTCCGGA | | TYYCQQWGSSPFTFGQGTK |
| | | GTGCCCTCAAGGTTCTCAGGATCTGGCAGTGGG | | VEIK |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | LQMNSLRAEDTAVYYCKTGG |
| | | GGTGACACTGAGTTTGTCCCCAAGTTTCAGGGCA | | FWGQGTLVTVSS |
| | | GAGCCACCATGAGCGCTGACACAAGCAAAAACA | | |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLRP |
| | | GCACAAACGTAAGT | | EACRPAAGGAVHTRGLDFAC |
| | | | | D |
| CD8 | 39 | ATCTATATCTGGGCACCTCTCGCTGGCACCTGTG | 40 | IYIWAPLAGTCGVLLLSLVIT |
| transmembrane | | GAGTCCTTCTGCTCAGCCTGGTTATTACTCTGTA | | LYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQL |
| | | GCGTACCAGCAGGGCCAGAACCAGCTCTATAAC | | YNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMK |
| | | ATGGGGGGAAAGCCGAGAAGGAAGAACCCTCAG | | GERRRGKGHDGLYQGLSTA |
| | | GAAGGCCTGTACAATGAACTGCAGAAAGATAAGA | | TKDTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid O: pBP2209-SFG-Myr-CD40.Fv.Fv.T2A.aPSCAscFv.CD34e.CD8stm.zeta

| | SEQ | | SEQ | |
|---|---|---|---|---|
| Fragment | ID # | Nucleotide | ID # | Peptide |
| Myristoylation | 51 | ATGGGGAGTAGCAAGAGCAAGCCTAAGGACCCC | 52 | MGSSKSKPKDPSQR |
| peptide | | AGCCAGCGC | | |
| Linker | 53 | AGAGCATGC | 54 | RAC |
| CD40 | 55 | AAAAAGGTGGCCAAGAAGCCAACCAATAAGGCC | 56 | KKVAKKPTNKAPHPKQEPQE |
| signaling | | CCCCACCCCAAGCAGGAGCCCCAGGAGATCAAT | | INFPDDLPGSNTAAPVQETLH |
| domain | | TTTCCCGACGATCTTCCTGGCTCCAACACTGCTG | | GCQPVTQEDGKESRISVQER |
| | | CTCCAGTGCAGGAGACTTTACATGGATGCCAGC | | Q |
| | | CGGTCACCCAGGAGGATGGCAAAGAGAGTCGCA | | |
| | | TCTCAGTGCAGGAGAGACAG | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCVVHYTGMLEDGKKVDSSR |
| | | TCGTCCATTATACAGGCATGTTGGAGGACGGCAA | | DRNKPFKFMLGKQEVIRGWE |
| | | AAAGGTGGACAGTAGTAGAGATCGCAATAAACCT | | EGVAQMSVGQRAKLTISPDY |
| | | TTCAAATTCATGTTGGGAAAACAAGAAGTCATTAG | | |
| | | GGGATGGGAGGAGGGCGTGGCTCAAATGTCCGT | | AYGATGHPGIIPPHATLVFDV |
| | | CGGCCAACGCGCTAAGCTCACCATCAGCCCCGA | | ELLKLE |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCVVHYTGMLEDGKKVDSSR |
| | | GTGGTGCACTACACCGGGATGCTTGAAGATGGA | | DRNKPFKFMLGKQEVIRGWE |
| | | AAGAAAGTTGATTCCTCCCGGGACAGAAACAAGC | | EGVAQMSVGQRAKLTISPDY |
| | | CCTTTAAGTTTATGCTAGGCAAGCAGGAGGTGAT | | AYGATGHPGIIPPHATLVFD |
| | | CCGAGGCTGGGAAGAAGGGGTTGCCCAGATGAG | | VELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQCS |
| Peptide | | GCAATACTCAAGGGCGTTCAATGTTCACGG | | R |
| PSCA(A11) | 25 | GACATCCAACTGACGCAAAGCCCATCTACACTCA | 26 | DIQLTQSPSTLSASMGDRVTI |
| V_{L} | | GCGCTAGCATGGGGGACAGGGTCACAATCACGT | | TCSASSSVRFIHWYQQKPGKA |
| | | GCTCTGCCTCAAGTTCCGTTAGGTTTATCCATTG | | PKRLIYDTSKLASGVPSRFSG |
| | | GTATCAGCAGAAACCTGGAAAGGCCCCAAAAAG | | SGSGTDFTLTISSLQPEDFA |
| | | ACTGATCTATGATACCAGCAAGCTGGCTTCCGGA | | TYYCQQWGSSPFTFGQGTK |
| | | GTGCCCTCAAGGTTCTCAGGATCTGGCAGTGGG | | VEIK |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | LQMNSLRAEDTAVYYCKTGG |
| | | GGTGACACTGAGTTTGTCCCCAAGTTTCAGGGCA | | FWGQGTLVTVSS |
| | | GAGCCACCATGAGCGCTGACACAAGCAAAAACA | | |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLRP |
| | | GCACAAACGTAAGT | | EACRPAAGGAVHTRGLDFACD |
| CD8 | 39 | ATCTATATCTGGGCACCTCTCGCTGGCACCTGTG | 40 | IYIWAPLAGTCGVLLLSLVIT |
| transmembrane | | GAGTCCTTCTGCTCAGCCTGGTTATTACTCTGTA | | LYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMK |
| | | ATGGGGGGAAAGCCGAGAAGGAAGAACCCTCAG | | GERRRGKGHDGLYQGLSTAT |
| | | GAAGGCCTGTACAATGAACTGCAGAAAGATAAGA | | KDTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid P: pBP2212-SFG-MyD88.CD40.Fv.Fv.T2A.aPSCAscFv.CD34e.CD8stm.zeta

| | SEQ | | SEQ | |
|---|---|---|---|---|
| Fragment | ID # | Nucleotide | ID # | Peptide |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGG | | RPGASVGRLLDLLTKLGRDD |
| | | ACTTTGAGTACTTGGAGATCCGGCAACTGGAGAC | | VLLELGPSIEEDCQKYILKQQ |
| | | ACAAGCGGACCCCACTGGCAGGCTGCTGGACGC | | QEEAEKPLQVAAVDSSVPRT |
| | | CTGGCAGGGACGCCCTGGCGCCTCTGTAGGCC | | AELAGITTLDDPLGHMPERFD |
| | | GACTGCTCGATCTGCTTACCAAGCTGGGCCGCG | | AFICYCPSDI |
| | | ACGACGTGCTGCTGGAGCTGGGACCCAGCATTG | | |
| | | AGGAGGATTGCCAAAAGTATATCTTGAAGCAGCA | | |
| | | GCAGGAGGAGGCTGAGAAGCCTTTACAGGTGGC | | |
| | | CGCTGTAGACAGCAGTGTCCCACGGACAGCAGA | | |
| | | CCTCGCGCCCATCACCACACTTCATGACCCCCT | | |
| | | GGGGCATATGCCTGAGCGTTTCGATGCCTTCATC | | |
| | | TGCTATTGCCCCAGCGACATC | | |
| Linker | 3 | CTCGAG | 4 | LE |
| CD40 | 55 | AAAAAGGTGGCCAAGAAGCCAACCAATAAGGCC | 56 | KKVAKKPTNKAPHPKQEPQE |
| signaling | | CCCCACCCCAAGCAGGAGCCCCAGGAGATCAAT | | INFPDDLPGSNTAAPVQETLH |
| domain | | TTTCCCGACGATCTTCCTGGCTCCAACACTGCTG | | GCQPVTQEDGKESRISVQER |
| | | CTCCAGTGCAGGAGACTTTACATGGATGCCAGC | | Q |
| | | CGGTCACCCAGGAGGATGGCAAAGAGAGTCGCA | | |
| | | TCTCAGTGCAGGAGAGACAG | | |
| Linker | 9 | GGATCTGGCCAATTG | 10 | GSGQL |
| FKBP_{v′} | 11 | GGCGTCCAAGTCGAAACCATTAGTCCCGGCGAT | 12 | GVQVETISPGDGRTFPKRGQ |
| | | GGCAGAACATTTCCTAAAAGGGGACAAACATGTG | | TCVVHYTGMLEDGKKVDSSR |
| | | TCGTCCATTATACAGGCATGTTGGAGGACGGCAA | | DRNKPFKFMLGKQEVIRGWE |
| | | AAAGGTGGACAGTAGTAGAGATCGCAATAAACCT | | EGVAQMSVGQRAKLTISPDY |
| | | TTCAAATTCATGTTGGGAAAACAAGAAGTCATTAG | | AYGATGHPGIIPPHATLVFD |
| | | GGGATGGGAGGAGGGCGTGGCTCAAATGTCCGT | | VELLKLE |
| | | CGGCCAACGCGCTAAGCTCACCATCAGCCCCGA | | |
| | | CTACGCATACGGCGCTACCGGACATCCCGGAAT | | |
| | | TATTCCCCCTCACGCTACCTTGGTGTTTGACGTC | | |
| | | GAACTGTTGAAGCTCGAA | | |
| Linker | 13 | GTCGAG | 14 | VE |
| FKBPᵥ | 15 | GGAGTGCAGGTGGAGACTATCTCCCCAGGAGAC | 16 | GVQVETISPGDGRTFPKRGQ |
| | | GGGCGCACCTTCCCCAAGCGCGGCCAGACCTGC | | TCWHYTGMLEDGKKVDSSR |
| | | GTGGTGCACTACACCGGGATGCTTGAAGATGGA | | DRNKPFKFMLGKQEVIRGWE |
| | | AAGAAAGTTGATTCCTCCCGGGACAGAAACAAGC | | EGVAQMSVGQRAKLTISPDY |
| | | CCTTTAAGTTTATGCTAGGCAAGCAGGAGGTGAT | | AYGATGHPGIIPPHATLVFDV |
| | | CCGAGGCTGGGAAGAAGGGGTTGCCCAGATGAG | | ELLKLE |
| | | TGTGGGTCAGAGAGCCAAACTGACTATATCTCCA | | |
| | | GATTATGCCTATGGTGCCACTGGGCACCCAGGC | | |
| | | ATCATCCCACCACATGCCACTCTCGTCTTCGATG | | |
| | | TGGAGCTTCTAAAACTGGAA | | |
| Linker | 17 | CCGCGG | 18 | PR |
| T2A | 19 | GAGGGCAGAGGCAGCCTCCTGACATGTGGGGAC | 20 | EGRGSLLTCGDVEENPGP |
| | | GTCGAGGAGAACCCTGGCCCA | | |
| Linker | 21 | CCTTGG | 22 | PW |
| Signal | 23 | ATGGAGTTCGGATTGAGCTGGCTGTTCCTGGTG | 24 | MEFGLSWLFLVAILKGVQCS |
| Peptide | | GCAATACTCAAGGGCGTTCAATGTTCACGG | | R |
| PSCA(A11) | 25 | GACATCCAACTGACGCAAAGCCCATCTACACTCA | 26 | DIQLTQSPSTLSASMGDRVTI |
| V_{L} | | GCGCTAGCATGGGGGACAGGGTCACAATCACGT | | TCSASSSVRFIHWYQQKPGK |
| | | GCTCTGCCTCAAGTTCCGTTAGGTTTATCCATTG | | APKRLIYDTSKLASGVPSRFS |
| | | GTATCAGCAGAAACCTGGAAAGGCCCCAAAAAG | | GSGSGTDFTLTISSLQPEDFA |
| | | ACTGATCTATGATACCAGCAAGCTGGCTTCCGGA | | TYYCQQWGSSPFTFGQGTK |
| | | GTGCCCTCAAGGTTCTCAGGATCTGGCAGTGGG | | VEIK |
| | | ACCGATTTCACCCTGACAATTAGCAGCCTTCAGC | | |
| | | CAGAGGATTTCGCAACCTATTACTGTCAGCAATG | | |
| | | GGGGTCCAGCCCATTCACTTTCGGCCAAGGAAC | | |
| | | AAAGGTGGAGATAAAA | | |
| Flex | 27 | GGCGGAGGAAGCGGAGGTGGGGGC | 28 | GGGSGGGG |
| Linker | 29 | CAGGTG | 30 | QV |
| PSCA(A11) | 31 | GAGGTGCAGCTCGTGGAGTATGGCGGGGGCCT | 32 | EVQLVEYGGGLVQPGGSLRL |
| V_{H} | | GGTGCAGCCTGGGGGTAGTCTGAGGCTCTCCTG | | SCAASGFNIKDYYIHWVRQA |
| | | CGCTGCCTCTGGCTTTAACATTAAAGACTACTAC | | PGKGLEWVAWIDPENGDTEF |
| | | ATACATTGGGTGCGGCAGGCCCCAGGCAAAGGG | | VPKFQGRATMSADTSKNTAY |
| | | CTCGAATGGGTGGCCTGGATTGACCCTGAGAAT | | LQMNSLRAEDTAVYYCKTGG |
| | | GGTGACACTGAGTTTGTCCCCAAGTTTCAGGGCA | | FWGQGTLVTVSS |
| | | GAGCCACCATGAGCGCTGACACAAGCAAAAACA | | |
| | | CTGCTTATCTCCAAATGAATAGCCTGCGAGCTGA | | |
| | | AGATACAGCAGTCTATTACTGCAAGACGGGAGGA | | |
| | | TTCTGGGGCCAGGGAACTCTGGTGACAGTTAGTT | | |
| | | CC | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 37 | GAACTTCCTACTCAGGGGACTTTCTCAAACGTTA | 38 | PAPRPPTPAPTIASQPLSLRP |
| | | GCACAAACGTAAGT | | EACRPAAGGAVHTRGLDFAC |
| | | | | D |
| CD8 | 39 | ATCTATATCTGGGCACCTCTCGCTGGCACCTGTG | 40 | IYIWAPLAGTCGVLLLSLVIT |
| transmembrane | | GAGTCCTTCTGCTCAGCCTGGTTATTACTCTGTA | | LYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTATK |
| | | GAAGGCCTGTACAATGAACTGCAGAAAGATAAGA | | DTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| STOP | | TGA | | STOP |

TABLE
Plasmid Q: pBP414--pSFG-FKBP.ΔC9.T2A-αCD19.Q.CD8stm.ζ

| | SEQ | | SEQ | |
|---|---|---|---|---|
| Fragment | ID # | Nucleotide | ID # | Peptide |
| Leader | 57 | ATGCTCGAGATGCTGGAG | 58 | MLEMLE |
| FKBP″ | 59 | GGAGTGCAGGTGGAGACTATTAGCCCCGGAGAT | 60 | GVQVETISPGDGRTFPKRGQ |
| | | GGCAGAACATTCCCCAAAAGAGGACAGACTTGC | | TCVVHYTGMLEDGKKFDSSR |
| | | GTCGTGCATTATACTGGAATGCTGGAAGACGGCA | | DRNKPFKFMLGKQEVIRGWE |
| | | AGAAGGTGGACAGCAGCCGGGACCGAAACAAGC | | EGVAQMSVGQRAKLTISPDY |
| | | CCTTCAAGTTCATGCTGGGGAAGCAGGAAGTGAT | | AYGATGHPGIIPPHATLVFD |
| | | CCGGGGCTGGGAGGAAGGAGTCGCACAGATGT | | VELLKLE |
| | | CAGTGGGACAGAGGGCCAAACTGACTATTAGCC | | |
| | | CAGACTACGCTTATGGAGCAACCGGCCACCCCG | | |
| | | GGATCATTCCCCCTCATGCTACACTGGTCTTCGA | | |
| | | TGTGGAGCTGCTGAAGCTGGAA | | |
| Linker | 61 | AGCGGAGGAGGATCCGGAGTGGAC | 62 | SGGGSGVD |
| Δcaspase9 | 63 | GGGTTTGGAGATGTGGGAGCCCTGGAATCCCTG | 64 | GFGDVGALESLRGNADLAYIL |
| | | CGGGGCAATGCCGATCTGGCTTACATCCTGTCTA | | SMEPCGHCLIINNVNFCRES |
| | | TGGAGCCTTGCGGCCACTGTCTGATCATTAACAA | | GLRTRTGSNIDCEKLRRRFS |
| | | TGTGAACTTCTGCAGAGAGAGCGGGCTGCGGAC | | SLHFMVEVKGDLTAKKMVLA |
| | | CAGAACAGGATCCAATATTGACTGTGAAAAGCTG | | LLELARQDHGALDCCVVVIL |
| | | CGGAGAAGGTTCTCTAGTCTGCACTTTATGGTCG | | SHGCQASHLQFPGAVYGTDG |
| | | AGGTGAAAGGCGATCTGACCGCTAAGAAAATGG | | CPVSVEKIVNIFNGTSCPSL |
| | | TGCTGGCCCTGCTGGAACTGGCTCGGCAGGACC | | GGKPKLFFIQACGGEQKDHG |
| | | ATGGGGCACTGGATTGCTGCGTGGTCGTGATCC | | FEVASTSPEDESPGSNPEPD |
| | | TGAGTCACGGCTGCCAGGCTTCACATCTGCAGTT | | ATPFQEGLRTFDQLDAISSL |
| | | CCCTGGGGCAGTCTATGGAACTGACGGCTGTCC | | PTPSDIFVSYSTFPGFVSWR |
| | | AGTCAGCGTGGAGAAGATCGTGAACATCTTCAAC | | DPKSGSWYVETLDDIFEQWA |
| | | GGCACCTCTTGCCCAAGTCTGGGCGGGAAGCCC | | HSEDLQSLLLRVANAVSVKG |
| | | AAACTGTTCTTTATTCAGGCCTGTGGAGGCGAGC | | IYKQMPGCFNFLRKKLFFKT |
| | | AGAAAGATCACGGCTTCGAAGTGGCTAGCACCT | | SASRA |
| | | CCCCCGAGGACGAATCACCTGGAAGCAACCCTG | | |
| | | AGCCAGATGCAACCCCCTTCCAGGAAGGCCTGA | | |
| | | GGACATTTGACCAGCTGGATGCCATCTCAAGCCT | | |
| | | GCCCACACCTTCTGACATTTTCGTCTCTTACAGTA | | |
| | | CTTTCCCTGGATTTGTGAGCTGGCGCGATCCAAA | | |
| | | GTCAGGCAGCTGGTACGTGGAGACACTGGACGA | | |
| | | TATCTTTGAGCAGTGGGCCCATTCTGAAGACCTG | | |
| | | CAGAGTCTGCTGCTGCGAGTGGCCAATGCTGTC | | |
| | | TCTGTGAAGGGGATCTACAAACAGATGCCAGGAT | | |
| | | GCTTCAACTTTCTGAGAAAGAAACTGTTCTTTAAG | | |
| | | ACCTCCGCATCTAGGGCC | | |
| Linker | 17 | CCGCGG | 18 | PR |
| T2A | 65 | GAAGGCCGAGGGAGCCTGCTGACATGTGGCGAT | 20 | EGRGSLLTCGDVEENPGP |
| | | GTGGAGGAAAACCCAGGACCA | | |
| Linker | 66 | CCATGG | 22 | PW |
| Signal | 67 | ATGGAGTTTGGACTTTCTTGGTTGTTTTTGGTGG | 24 | MEFGLSWLFLVAILKGVQCS |
| Peptide | | CAATTCTGAAGGGTGTCCAGTGTAGCAGG | | R |
| FMC63 VL | 68 | GACATCCAGATGACACAGACTACATCCTCCCTGT | 69 | DIQMTQTTSSLSASLGDRVTI |
| | | CTGCCTCTCTGGGAGACAGAGTCACCATCAGTTG | | SCRASQDISKYLNWYQQKPD |
| | | CAGGGCAAGTCAGGACATTAGTAAATATTTAAATT | | GTVKLLIYHTSRLHSGVPSR |
| | | GGTATCAGCAGAAACCAGATGGAACTGTTAAACT | | FSGSGSGTDYSLTISNLEQE |
| | | CCTGATCTACCATACATCAAGATTACACTCAGGA | | DIATYFCQQGNTLPYTFGGG |
| | | GTCCCATCAAGGTTCAGTGGCAGTGGGTCTGGA | | TKLEIT |
| | | ACAGATTATTCTCTCACCATTAGCAACCTGGAGC | | |
| | | AAGAAGATATTGCCACTTACTTTTGCCAACAGGG | | |
| | | TAATACGCTTCCGTACACGTTCGGAGGGGGGAC | | |
| | | TAAGTTGGAAATAACA | | |
| Flex | 27 | GGCGGAGGAAGCGGAGGTGGGGGC | 28 | GGGSGGGG |
| FMC63 VH | 70 | GAGGTGAAACTGCAGGAGTCAGGACCTGGCCTG | 71 | EVKLQESGPGLVAPSQSLSV |
| | | GTGGCGCCCTCACAGAGCCTGTCCGTCACATGC | | TCTVSGVSLPDYGVSWIRQP |
| | | ACTGTCTCAGGGGTCTCATTACCCGACTATGGTG | | PRKGLEWLGVIWGSETTYYN |
| | | TAAGCTGGATTCGCCAGCCTCCACGAAAGGGTC | | SALKSRLTIIKDNSKSQVFLK |
| | | TGGAGTGGCTGGGAGTAATATGGGGTAGTGAAA | | MNSLQTDDTAIYYCAKHYYY |
| | | CCACATACTATAATTCAGCTCTCAAATCCAGACTG | | GGSYAMDYWGQGTSVTVSS |
| | | ACCATCATCAAGGACAACTCCAAGAGCCAAGTTT | | |
| | | TCTTAAAAATGAACAGTCTGCAAACTGATGACAC | | |
| | | AGCCATTTACTACTGTGCCAAACATTATTACTACG | | |
| | | GTGGTAGCTATGCTATGGACTACTGGGGTCAAG | | |
| | | GAACCTCAGTCACCGTCTCCTCA | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 72 | CCCGCCCCAAGACCCCCCACACCTGCGCCGACC | 38 | PAPRPPTPAPTIASQPLSLRP |
| | | ATTGCTTCTCAACCCCTGAGTTTGAGACCCGAGG | | EACRPAAGGAVHTRGLDFAC |
| | | CCTGCCGGCCAGCTGCCGGCGGGGCCGTGCAT | | D |
| | | ACAAGAGGACTCGATTTCGCTTGCGAC | | |
| CD8 | 39 | ATCTATATCTGGGCACCTCTCGCTGGCACCTGTG | 40 | IYIWAPLAGTCGVLLLSLVITL |
| transmembrane | | GAGTCCTTCTGCTCAGCCTGGTTATTACTCTGTA | | YCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTATK |
| | | GAAGGCCTGTACAATGAACTGCAGAAAGATAAGA | | DTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |

TABLE
Plasmid R: pBP0844--pSFG-FKBP.ΔC9.T2A-αCD19.Q.CD8stm.ζ.2A-MC

| | SEQ | | SEQ | |
|---|---|---|---|---|
| Fragment | ID # | Nucleotide | ID # | Peptide |
| Leader | 57 | ATGCTCGAGATGCTGGAG | 58 | MLEMLE |
| FKBP″ | 59 | GGAGTGCAGGTGGAGACTATTAGCCCCGGAGAT | 60 | GVQVETISPGDGRTFPKRGQ |
| | | GGCAGAACATTCCCCAAAAGAGGACAGACTTGC | | TCVVHYTGMLEDGKKFDSSR |
| | | GTCGTGCATTATACTGGAATGCTGGAAGACGGCA | | DRNKPFKFMLGKQEVIRGWE |
| | | AGAAGGTGGACAGCAGCCGGGACCGAAACAAGC | | EGVAQMSVGQRAKLTISPDY |
| | | CCTTCAAGTTCATGCTGGGGAAGCAGGAAGTGAT | | AYGATGHPGIIPPHATLVFD |
| | | CCGGGGCTGGGAGGAAGGAGTCGCACAGATGT | | VELLKLE |
| | | CAGTGGGACAGAGGGCCAAACTGACTATTAGCC | | |
| | | CAGACTACGCTTATGGAGCAACCGGCCACCCCG | | |
| | | GGATCATTCCCCCTCATGCTACACTGGTCTTCGA | | |
| | | TGTGGAGCTGCTGAAGCTGGAA | | |
| Linker | 61 | AGCGGAGGAGGATCCGGAGTGGAC | 62 | SGGGSGVD |
| Δcaspase9 | 63 | GGGTTTGGAGATGTGGGAGCCCTGGAATCCCTG | 64 | GFGDVGALESLRGNADLAYIL |
| | | CGGGGCAATGCCGATCTGGCTTACATCCTGTCTA | | SMEPCGHCLIINNVNFCRES |
| | | TGGAGCCTTGCGGCCACTGTCTGATCATTAACAA | | GLRTRTGSNIDCEKLRRRFS |
| | | TGTGAACTTCTGCAGAGAGAGCGGGCTGCGGAC | | SLHFMVEVKGDLTAKKMVLA |
| | | CAGAACAGGATCCAATATTGACTGTGAAAAGCTG | | LLELARQDHGALDCCVVVIL |
| | | CGGAGAAGGTTCTCTAGTCTGCACTTTATGGTCG | | SHGCQASHLQFPGAVYGTDG |
| | | AGGTGAAAGGCGATCTGACCGCTAAGAAAATGG | | CPVSVEKIVNIFNGTSCPSLG |
| | | TGCTGGCCCTGCTGGAACTGGCTCGGCAGGACC | | GKPKLFFIQACGGEQKDHGF |
| | | ATGGGGCACTGGATTGCTGCGTGGTCGTGATCC | | EVASTSPEDESPGSNPEPDA |
| | | TGAGTCACGGCTGCCAGGCTTCACATCTGCAGTT | | TPFQEGLRTFDQLDAISSLPT |
| | | CCCTGGGGCAGTCTATGGAACTGACGGCTGTCC | | PSDIFVSYSTFPGFVSWRDP |
| | | AGTCAGCGTGGAGAAGATCGTGAACATCTTCAAC | | KSGSWYVETLDDIFEQWAHS |
| | | GGCACCTCTTGCCCAAGTCTGGGCGGGAAGCCC | | EDLQSLLLRVANAVSVKGIYK |
| | | AAACTGTTCTTTATTCAGGCCTGTGGAGGCGAGC | | QMPGCFNFLRKKLFFKTSAS |
| | | AGAAAGATCACGGCTTCGAAGTGGCTAGCACCT | | RA |
| | | CCCCCGAGGACGAATCACCTGGAAGCAACCCTG | | |
| | | AGCCAGATGCAACCCCCTTCCAGGAAGGCCTGA | | |
| | | GGACATTTGACCAGCTGGATGCCATCTCAAGCCT | | |
| | | GCCCACACCTTCTGACATTTTCGTCTCTTACAGTA | | |
| | | CTTTCCCTGGATTTGTGAGCTGGCGCGATCCAAA | | |
| | | GTCAGGCAGCTGGTACGTGGAGACACTGGACGA | | |
| | | TATCTTTGAGCAGTGGGCCCATTCTGAAGACCTG | | |
| | | CAGAGTCTGCTGCTGCGAGTGGCCAATGCTGTC | | |
| | | TCTGTGAAGGGGATCTACAAACAGATGCCAGGAT | | |
| | | GCTTCAACTTTCTGAGAAAGAAACTGTTCTTTAAG | | |
| | | ACCTCCGCATCTAGGGCC | | |
| Linker | 17 | CCGCGG | 18 | PR |
| T2A | 65 | GAAGGCCGAGGGAGCCTGCTGACATGTGGCGAT | 20 | EGRGSLLTCGDVEENPGP |
| | | GTGGAGGAAAACCCAGGACCA | | |
| Linker | 66 | CCATGG | 22 | PW |
| Signal | 67 | ATGGAGTTTGGACTTTCTTGGTTGTTTTTGGTGG | 24 | MEFGLSWLFLVAILKGVQCS |
| Peptide | | CAATTCTGAAGGGTGTCCAGTGTAGCAGG | | R |
| FMC63 VL | 68 | GACATCCAGATGACACAGACTACATCCTCCCTGT | 69 | DIQMTQTTSSLSASLGDRVTI |
| | | CTGCCTCTCTGGGAGACAGAGTCACCATCAGTTG | | SCRASQDISKYLNWYQQKPD |
| | | CAGGGCAAGTCAGGACATTAGTAAATATTTAAATT | | GTVKLLIYHTSRLHSGVPSRF |
| | | GGTATCAGCAGAAACCAGATGGAACTGTTAAACT | | SGSGSGTDYSLTISNLEQEDI |
| | | CCTGATCTACCATACATCAAGATTACACTCAGGA | | ATYFCQQGNTLPYTFGGGTK |
| | | GTCCCATCAAGGTTCAGTGGCAGTGGGTCTGGA | | LEIT |
| | | ACAGATTATTCTCTCACCATTAGCAACCTGGAGC | | |
| | | AAGAAGATATTGCCACTTACTTTTGCCAACAGGG | | |
| | | TAATACGCTTCCGTACACGTTCGGAGGGGGGAC | | |
| | | TAAGTTGGAAATAACA | | |
| Flex | 27 | GGCGGAGGAAGCGGAGGTGGGGGC | 28 | GGGSGGGG |
| FMC63 VH | 70 | GAGGTGAAACTGCAGGAGTCAGGACCTGGCCTG | 71 | EVKLQESGPGLVAPSQSLSV |
| | | GTGGCGCCCTCACAGAGCCTGTCCGTCACATGC | | TCTVSGVSLPDYGVSWIRQP |
| | | ACTGTCTCAGGGGTCTCATTACCCGACTATGGTG | | PRKGLEWLGVIWGSETTYYN |
| | | TAAGCTGGATTCGCCAGCCTCCACGAAAGGGTC | | SALKSRLTIIKDNSKSQVFLK |
| | | TGGAGTGGCTGGGAGTAATATGGGGTAGTGAAA | | MNSLQTDDTAIYYCAKHYYY |
| | | CCACATACTATAATTCAGCTCTCAAATCCAGACTG | | GGSYAMDYWGQGTSVTVSS |
| | | ACCATCATCAAGGACAACTCCAAGAGCCAAGTTT | | |
| | | TCTTAAAAATGAACAGTCTGCAAACTGATGACAC | | |
| | | AGCCATTTACTACTGTGCCAAACATTATTACTACG | | |
| | | GTGGTAGCTATGCTATGGACTACTGGGGTCAAG | | |
| | | GAACCTCAGTCACCGTCTCCTCA | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 72 | CCCGCCCCAAGACCCCCCACACCTGCGCCGACC | 38 | PAPRPPTPAPTIASQPLSLRP |
| | | ATTGCTTCTCAACCCCTGAGTTTGAGACCCGAGG | | EACRPAAGGAVHTRGLDFAC |
| | | CCTGCCGGCCAGCTGCCGGCGGGGCCGTGCAT | | D |
| | | ACAAGAGGACTCGATTTCGCTTGCGAC | | |
| CD8 | 39 | ATCTATATCTGGGCACCTCTCGCTGGCACCTGTG | 40 | IYIWAPLAGTCGVLLLSLVIT |
| transmembrane | | GAGTCCTTCTGCTCAGCCTGGTTATTACTCTGTA | | LYCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTATK |
| | | GAAGGCCTGTACAATGAACTGCAGAAAGATAAGA | | DTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| Leader | 57 | ATGCTCGAGATGCTGGAG | 58 | MLEMLE |
| P2A | 73 | GCTACTAACTTCAGCCTGCTGAAGCAGGCTGGA | 74 | ATNFSLLKQAGDVEENPGP |
| | | GACGTGGAGGAGAACCCCGGGCCT | | |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGG | | RPGASVGRLLDLLTKLGRDD |
| | | ACTTTGAGTACTTGGAGATCCGGCAACTGGAGAC | | VLLELGPSIEEDCQKYILKQQ |
| | | ACAAGCGGACCCCACTGGCAGGCTGCTGGACGC | | QEEAEKPLQVAAVDSSVPRT |
| | | CTGGCAGGGACGCCCTGGCGCCTCTGTAGGCC | | AELAGITTLDDPLGHMPERFD |
| | | GACTGCTCGATCTGCTTACCAAGCTGGGCCGCG | | AFICYCPSDI |
| | | ACGACGTGCTGCTGGAGCTGGGACCCAGCATTG | | |
| | | AGGAGGATTGCCAAAAGTATATCTTGAAGCAGCA | | |
| | | GCAGGAGGAGGCTGAGAAGCCTTTACAGGTGGC | | |
| | | CGCTGTAGACAGCAGTGTCCCACGGACAGCAGA | | |
| | | GCTGGCGGGCATCACCACACTTGATGACCCCCT | | |
| | | GGGGCATATGCCTGAGCGTTTCGATGCCTTCATC | | |
| | | TGCTATTGCCCCAGCGACATC | | |
| Linker | 13 | GTCGAG | 14 | VE |
| CD40 | 75 | AAAAAGGTGGCCAAGAAGCCAACCAATAAGGCC | 56 | KKVAKKPTNKAPHPKQEPQE |
| | | CCCCACCCCAAGCAGGAGCCCCAGGAGATCAAT | | INFPDDLPGSNTAAPVQETLH |
| | | TTTCCCGACGATCTTCCTGGCTCCAACACTGCTG | | GCQPVTQEDGKESRISVQER |
| | | CTCCAGTGCAGGAGACTTTACATGGATGCCAACC | | Q |
| | | GGTCACCCAGGAGGATGGCAAAGAGAGTCGCAT | | |
| | | CTCAGTGCAGGAGAGACAG | | |

TABLE
Plasmid S: pBP2103--pSFG-FKBP.ΔC9.T2A-αCD19.Q.CD8stm.ζ.2A-MC

| | SEQ | | SEQ | |
|---|---|---|---|---|
| Fragment | ID # | Nucleotide | ID # | Peptide |
| Leader | 57 | ATGCTCGAGATGCTGGAG | 58 | MLEMLE |
| FKBP″ | 59 | GGAGTGCAGGTGGAGACTATTAGCCCCGGAGAT | 60 | GVQVETISPGDGRTFPKRGQ |
| | | GGCAGAACATTCCCCAAAAGAGGACAGACTTGC | | TCVVHYTGMLEDGKKFDSSR |
| | | GTCGTGCATTATACTGGAATGCTGGAAGACGGCA | | DRNKPFKFMLGKQEVIRGWE |
| | | AGAAGGTGGACAGCAGCCGGGACCGAAACAAGC | | EGVAQMSVGQRAKLTISPDY |
| | | CCTTCAAGTTCATGCTGGGGAAGCAGGAAGTGAT | | AYGATGHPGIIPPHATLVFDV |
| | | CCGGGGCTGGGAGGAAGGAGTCGCACAGATGT | | ELLKLE |
| | | CAGTGGGACAGAGGGCCAAACTGACTATTAGCC | | |
| | | CAGACTACGCTTATGGAGCAACCGGCCACCCCG | | |
| | | GGATCATTCCCCCTCATGCTACACTGGTCTTCGA | | |
| | | TGTGGAGCTGCTGAAGCTGGAA | | |
| Linker | 61 | AGCGGAGGAGGATCCGGAGTGGAC | 62 | SGGGSGVD |
| Δcaspase9 | 63 | GGGTTTGGAGATGTGGGAGCCCTGGAATCCCTG | 64 | GFGDVGALESLRGNADLAYIL |
| | | CGGGGCAATGCCGATCTGGCTTACATCCTGTCTA | | SMEPCGHCLIINNVNFCRES |
| | | TGGAGCCTTGCGGCCACTGTCTGATCATTAACAA | | GLRTRTGSNIDCEKLRRRFS |
| | | TGTGAACTTCTGCAGAGAGAGCGGGCTGCGGAC | | SLHFMVEVKGDLTAKKMVLA |
| | | CAGAACAGGATCCAATATTGACTGTGAAAAGCTG | | LLELARQDHGALDCCVVVIL |
| | | CGGAGAAGGTTCTCTAGTCTGCACTTTATGGTCG | | SHGCQASHLQFPGAVYGTDG |
| | | AGGTGAAAGGCGATCTGACCGCTAAGAAAATGG | | CPVSVEKIVNIFNGTSCPSL |
| | | TGCTGGCCCTGCTGGAACTGGCTCGGCAGGACC | | GGKPKLFFIQACGGEQKDHG |
| | | ATGGGGCACTGGATTGCTGCGTGGTCGTGATCC | | FEVASTSPEDESPGSNPEPD |
| | | TGAGTCACGGCTGCCAGGCTTCACATCTGCAGTT | | ATPFQEGLRTFDQLDAISSL |
| | | CCCTGGGGCAGTCTATGGAACTGACGGCTGTCC | | PTPSDIFVSYSTFPGFVSWR |
| | | AGTCAGCGTGGAGAAGATCGTGAACATCTTCAAC | | DPKSGSWYVETLDDIFEQWA |
| | | GGCACCTCTTGCCCAAGTCTGGGCGGGAAGCCC | | HSEDLQSLLLRVANAVSVKG |
| | | AAACTGTTCTTTATTCAGGCCTGTGGAGGCGAGC | | IYKQMPGCFNFLRKKLFFKT |
| | | AGAAAGATCACGGCTTCGAAGTGGCTAGCACCT | | SASRA |
| | | CCCCCGAGGACGAATCACCTGGAAGCAACCCTG | | |
| | | AGCCAGATGCAACCCCCTTCCAGGAAGGCCTGA | | |
| | | GGACATTTGACCAGCTGGATGCCATCTCAAGCCT | | |
| | | GCCCACACCTTCTGACATTTTCGTCTCTTACAGTA | | |
| | | CTTTCCCTGGATTTGTGAGCTGGCGCGATCCAAA | | |
| | | GTCAGGCAGCTGGTACGTGGAGACACTGGACGA | | |
| | | TATCTTTGAGCAGTGGGCCCATTCTGAAGACCTG | | |
| | | CAGAGTCTGCTGCTGCGAGTGGCCAATGCTGTC | | |
| | | TCTGTGAAGGGGATCTACAAACAGATGCCAGGAT | | |
| | | GCTTCAACTTTCTGAGAAAGAAACTGTTCTTTAAG | | |
| | | ACCTCCGCATCTAGGGCC | | |
| Linker | 17 | CCGCGG | 18 | PR |
| T2A | 65 | GAAGGCCGAGGGAGCCTGCTGACATGTGGCGAT | 20 | EGRGSLLTCGDVEENPGP |
| | | GTGGAGGAAAACCCAGGACCA | | |
| Linker | 66 | CCATGG | 22 | PW |
| Signal | 67 | ATGGAGTTTGGACTTTCTTGGTTGTTTTTGGTGG | 24 | MEFGLSWLFLVAILKGVQCS |
| Peptide | | CAATTCTGAAGGGTGTCCAGTGTAGCAGG | | R |
| FMC63 VL | 68 | GACATCCAGATGACACAGACTACATCCTCCCTGT | 69 | DIQMTQTTSSLSASLGDRVTI |
| | | CTGCCTCTCTGGGAGACAGAGTCACCATCAGTTG | | SCRASQDISKYLNWYQQKPD |
| | | CAGGGCAAGTCAGGACATTAGTAAATATTTAAAT | | GTVKLLIYHTSRLHSGVPSRF |
| | | TGGTATCAGCAGAAACCAGATGGAACTGTTAAAC | | SGSGSGTDYSLTISNLEQEDI |
| | | TCCTGATCTACCATACATCAAGATTACACTCAGG | | ATYFCQQGNTLPYTFGGGTK |
| | | AGTCCCATCAAGGTTCAGTGGCAGTGGGTCTGGA | | LEIT |
| | | ACAGATTATTCTCTCACCATTAGCAACCTGGAGC | | |
| | | AAGAAGATATTGCCACTTACTTTTGCCAACAGGG | | |
| | | TAATACGCTTCCGTACACGTTCGGAGGGGGGAC | | |
| | | TAAGTTGGAAATAACA | | |
| Flex | 27 | GGCGGAGGAAGCGGAGGTGGGGGC | 28 | GGGSGGGG |
| FMC63 VH | 70 | GAGGTGAAACTGCAGGAGTCAGGACCTGGCCTG | 71 | EVKLQESGPGLVAPSQSLSV |
| | | GTGGCGCCCTCACAGAGCCTGTCCGTCACATGC | | TCTVSGVSLPDYGVSWIRQP |
| | | ACTGTCTCAGGGGTCTCATTACCCGACTATGGTG | | PRKGLEWLGVIWGSETTYYN |
| | | TAAGCTGGATTCGCCAGCCTCCACGAAAGGGTC | | SALKSRLTIIKDNSKSQVFLK |
| | | TGGAGTGGCTGGGAGTAATATGGGGTAGTGAAA | | MNSLQTDDTAIYYCAKHYYY |
| | | CCACATACTATAATTCAGCTCTCAAATCCAGACT | | GGSYAMDYWGQGTSVTVSS |
| | | GACCATCATCAAGGACAACTCCAAGAGCCAAGTT | | |
| | | TTCTTAAAAATGAACAGTCTGCAAACTGATGACA | | |
| | | CAGCCATTTACTACTGTGCCAAACATTATTACTA | | |
| | | CGGTGGTAGCTATGCTATGGACTACTGGGGTCAA | | |
| | | GGAACCTCAGTCACCGTCTCCTCA | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 72 | CCCGCCCCAAGACCCCCCACACCTGCGCCGACC | 38 | PAPRPPTPAPTIASQPLSLRP |
| | | ATTGCTTCTCAACCCCTGAGTTTGAGACCCGAG | | EACRPAAGGAVHTRGLDFAC |
| | | GCCTGCCGGCCAGCTGCCGGCGGGGCCGTGCAT | | D |
| | | ACAAGAGGACTCGATTTCGCTTGCGAC | | |
| CD8 | 39 | ATCTATATCTGGGCACCTCTCGCTGGCACCTGTG | 40 | IYIWAPLAGTCGVLLLSLVITL |
| transmembrane | | GAGTCCTTCTGCTCAGCCTGGTTATTACTCTGTA | | YCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKR |
| | | GAGCTCAATCTAGGACGAAGAGAGGAGTACGAT | | RGRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTATK |
| | | GAAGGCCTGTACAATGAACTGCAGAAAGATAAG | | DTYDALHMQALPPR |
| | | ATGGCGGAGGCCTACAGTGAGATTGGGATGAAA | | |
| | | GGCGAGCGCCGGAGGGGCAAGGGGCACGATGGC | | |
| | | CTTTACCAGGGTCTCAGTACAGCCACCAAGGAC | | |
| | | ACCTACGACGCCCTTCACATGCAAGCTCTTCCA | | |
| | | CCTCGT | | |
| Leader | 57 | ATGCTCGAGATGCTGGAG | 58 | MLEMLE |
| P2A | 73 | GCTACTAACTTCAGCCTGCTGAAGCAGGCTGGA | 74 | ATNFSLLKQAGDVEENPGP |
| | | GACGTGGAGGAGAACCCCGGGCCT | | |
| Myristoylation | 51 | ATGGGGAGTAGCAAGAGCAAGCCTAAGGACCCC | 76 | MGSSKSKPKDPSQRLD |
| targeting | | AGCCAGCGC | | |
| peptide | | | | |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGGA | | RPGASVGRLLDLLTKLGRDD |
| | | CTTTGAGTACTTGGAGATCCGGCAACTGGAGAC | | VLLELGPSIEEDCQKYILKQ |
| | | ACAAGCGGACCCCACTGGCAGGCTGCTGGACGC | | QQEEAEKPLQVAAVDSSVPR |
| | | CTGGCAGGGACGCCCTGGCGCCTCTGTAGGCC | | TAELAGITTLDDPLGHMPER |
| | | GACTGCTCGATCTGCTTACCAAGCTGGGCCGCG | | FDAFICYCPSDI |
| | | ACGACGTGCTGCTGGAGCTGGGACCCAGCATTG | | |
| | | AGGAGGATTGCCAAAAGTATATCTTGAAGCAGC | | |
| | | AGCAGGAGGAGGCTGAGAAGCCTTTACAGGTGG | | |
| | | CCGCTGTAGACAGCAGTGTCCCACGGACAGCAG | | |
| | | AGCTGGCGGGCATCACCACACTTGATGACCCCC | | |
| | | TGGGGCATATGCCTGAGCGTTTCGATGCCTTCA | | |
| | | TCTGCTATTGCCCCAGCGACATC | | |
| Linker | 77 | GCGGCCGCT | 78 | AAA |
| 41BB | 79 | AAACGGGGCAGAAAGAAACTCCTGTATATATTCA | 80 | KRGRKKLLYIFKQPFMRPVQ |
| | | AACAACCATTTATGAGACCAGTACAAACTACTCA | | TTQEEDGCSCRFPEEEEGG |
| | | AGAGGAAGATGGCTGTAGCTGCCGATTTCCAGAA | | CEL |
| | | GAAGAAGAAGGAGGATGTGAACTG | | |

TABLE
Plasmid T: pBP2104--pSFG-FKBP.ΔC9.T2A-αCD19.Q.CD8stm.ζ.2A-MC

| | SEQ | | SEQ | |
|---|---|---|---|---|
| Fragment | ID # | Nucleotide | ID # | Peptide |
| Leader | 57 | ATGCTCGAGATGCTGGAG | 58 | |
| FKBP″ | 59 | GGAGTGCAGGTGGAGACTATTAGCCCCGGAGAT | 60 | |
| | | GGCAGAACATTCCCCAAAAGAGGACAGACTTGC | | |
| | | GTCGTGCATTATACTGGAATGCTGGAAGACGGC | | |
| | | AAGAAGGTGGACAGCAGCCGGGACCGAAACAAG | | |
| | | CCCTTCAAGTTCATGCTGGGGAAGCAGGAAGTG | | |
| | | ATCCGGGGCTGGGAGGAAGGAGTCGCACAGATG | | |
| | | TCAGTGGGACAGAGGGCCAAACTGACTATTAGC | | |
| | | CCAGACTACGCTTATGGAGCAACCGGCCACCCC | | |
| | | GGGATCATTCCCCCTCATGCTACACTGGTCTTC | | |
| | | GATGTGGAGCTGCTGAAGCTGGAA | | |
| Linker | 61 | AGCGGAGGAGGATCCGGAGTGGAC | 62 | SGGGSGVD |
| Δcaspase9 | 63 | GGGTTTGGAGATGTGGGAGCCCTGGAATCCCTG | 64 | GFGDVGALESLRGNADLAYIL |
| | | CGGGGCAATGCCGATCTGGCTTACATCCTGTCTA | | SMEPCGHCLIINNVNFCRES |
| | | TGGAGCCTTGCGGCCACTGTCTGATCATTAACAA | | GLRTRTGSNIDCEKLRRRFS |
| | | TGTGAACTTCTGCAGAGAGAGCGGGCTGCGGACC | | SLHFMVEVKGDLTAKKMVLA |
| | | AGAACAGGATCCAATATTGACTGTGAAAAGCTGC | | LLELARQDHGALDCCVVVILS |
| | | GGAGAAGGTTCTCTAGTCTGCACTTTATGGTCG | | HGCQASHLQFPGAVYGTDG |
| | | AGGTGAAAGGCGATCTGACCGCTAAGAAAATGG | | CPVSVEKIVNIFNGTSCPSLG |
| | | TGCTGGCCCTGCTGGAACTGGCTCGGCAGGACC | | GKPKLFFIQACGGEQKDHGF |
| | | ATGGGGCACTGGATTGCTGCGTGGTCGTGATCC | | EVASTSPEDESPGSNPEPDA |
| | | TGAGTCACGGCTGCCAGGCTTCACATCTGCAGT | | TPFQEGLRTFDQLDAISSLPT |
| | | TCCCTGGGGCAGTCTATGGAACTGACGGCTGTC | | PSDIFVSYSTFPGFVSWRDP |
| | | CAGTCAGCGTGGAGAAGATCGTGAACATCTTCA | | KSGSWYVETLDDIFEQWAHS |
| | | ACGGCACCTCTTGCCCAAGTCTGGGCGGGAAGC | | EDLQSLLLRVANAVSVKGIYK |
| | | CCAAACTGTTCTTTATTCAGGCCTGTGGAGGCG | | QMPGCFNFLRKKLFFKTSAS |
| | | AGCAGAAAGATCACGGCTTCGAAGTGGCTAGCA | | RA |
| | | CCTCCCCCGAGGACGAATCACCTGGAAGCAACC | | |
| | | CTGAGCCAGATGCAACCCCCTTCCAGGAAGGCC | | |
| | | TGAGGACATTTGACCAGCTGGATGCCATCTCAA | | |
| | | GCCTGCCCACACCTTCTGACATTTTCGTCTCTT | | |
| | | ACAGTACTTTCCCTGGATTTGTGAGCTGGCGCG | | |
| | | ATCCAAAGTCAGGCAGCTGGTACGTGGAGACAC | | |
| | | TGGACGATATCTTTGAGCAGTGGGCCCATTCTG | | |
| | | AAGACCTGCAGAGTCTGCTGCTGCGAGTGGCCA | | |
| | | ATGCTGTCTCTGTGAAGGGGATCTACAAACAGA | | |
| | | TGCCAGGATGCTTCAACTTTCTGAGAAAGAAAC | | |
| | | TGTTCTTTAAGACCTCCGCATCTAGGGCC | | |
| Linker | 17 | CCGCGG | 18 | PR |
| T2A | 65 | GAAGGCCGAGGGAGCCTGCTGACATGTGGCGAT | 20 | EGRGSLLTCGDVEENPGP |
| | | GTGGAGGAAAACCCAGGACCA | | |
| Linker | 66 | CCATGG | 22 | PW |
| Signal | 67 | ATGGAGTTTGGACTTTCTTGGTTGTTTTTGGTGG | 24 | MEFGLSWLFLVAILKGVQCS |
| Peptide | | CAATTCTGAAGGGTGTCCAGTGTAGCAGG | | R |
| FMC63 VL | 68 | GACATCCAGATGACACAGACTACATCCTCCCTGT | 69 | DIQMTQTTSSLSASLGDRVTI |
| | | CTGCCTCTCTGGGAGACAGAGTCACCATCAGTTG | | SCRASQDISKYLNWYQQKPD |
| | | CAGGGCAAGTCAGGACATTAGTAAATATTTAAATT | | GTVKLLIYHTSRLHSGVPSRF |
| | | GGTATCAGCAGAAACCAGATGGAACTGTTAAACT | | SGSGSGTDYSLTISNLEQEDI |
| | | CCTGATCTACCATACATCAAGATTACACTCAGGA | | ATYFCQQGNTLPYTFGGGTK |
| | | GTCCCATCAAGGTTCAGTGGCAGTGGGTCTGGA | | LEIT |
| | | ACAGATTATTCTCTCACCATTAGCAACCTGGAGC | | |
| | | AAGAAGATATTGCCACTTACTTTTGCCAACAGGG | | |
| | | TAATACGCTTCCGTACACGTTCGGAGGGGGGAC | | |
| | | TAAGTTGGAAATAACA | | |
| Flex | 27 | GGCGGAGGAAGCGGAGGTGGGGGC | 28 | GGGSGGGG |
| FMC63 VH | 70 | GAGGTGAAACTGCAGGAGTCAGGACCTGGCCTG | 71 | EVKLQESGPGLVAPSQSLSV |
| | | GTGGCGCCCTCACAGAGCCTGTCCGTCACATGC | | TCTVSGVSLPDYGVSWIRQP |
| | | ACTGTCTCAGGGGTCTCATTACCCGACTATGGTG | | PRKGLEWLGVIWGSETTYYN |
| | | TAAGCTGGATTCGCCAGCCTCCACGAAAGGGTC | | SALKSRLTIIKDNSKSQVFLK |
| | | TGGAGTGGCTGGGAGTAATATGGGGTAGTGAAA | | MNSLQTDDTAIYYCAKHYYY |
| | | CCACATACTATAATTCAGCTCTCAAATCCAGACTG | | GGSYAMDYWGQGTSVTVSS |
| | | ACCATCATCAAGGACAACTCCAAGAGCCAAGTTT | | |
| | | TCTTAAAAATGAACAGTCTGCAAACTGATGACAC | | |
| | | AGCCATTTACTACTGTGCCAAACATTATTACTACG | | |
| | | GTGGTAGCTATGCTATGGACTACTGGGGTCAAG | | |
| | | GAACCTCAGTCACCGTCTCCTCA | | |
| Linker | 33 | GGATCC | 34 | GS |
| CD34 | 35 | GAACTTCCTACTCAGGGGACTTTCTCAAACGTTA | 36 | ELPTQGTFSNVSTNVS |
| epitope | | GCACAAACGTAAGT | | |
| CD8 stalk | 72 | CCCGCCCCAAGACCCCCCACACCTGCGCCGACC | 38 | PAPRPPTPAPTIASQPLSLRP |
| | | ATTGCTTCTCAACCCCTGAGTTTGAGACCCGAGG | | EACRPAAGGAVHTRGLDFAC |
| | | CCTGCCGGCCAGCTGCCGGCGGGGCCGTGCAT | | D |
| | | ACAAGAGGACTCGATTTCGCTTGCGAC | | |
| CD8 | 39 | ATCTATATCTGGGCACCTCTCGCTGGCACCTGTG | 40 | IYIWAPLAGTCGVLLLSLVITL |
| transmembrane | | GAGTCCTTCTGCTCAGCCTGGTTATTACTCTGTA | | YCNHRNRRRVCKCPR |
| | | CTGTAATCACCGGAATCGCCGCCGCGTTTGTAAG | | |
| | | TGTCCCAGG | | |
| Linker | 41 | GTCGAC | 42 | VD |
| CD3ζ | 43 | AGAGTGAAGTTCAGCAGGAGCGCAGACGCCCCC | 44 | RVKFSRSADAPAYQQGQNQ |
| | | GCGTACCAGCAGGGCCAGAACCAGCTCTATAAC | | LYNELNLGRREEYDVLDKRR |
| | | GAGCTCAATCTAGGACGAAGAGAGGAGTACGAT | | GRDPEMGGKPRRKNPQEGL |
| | | GTTTTGGACAAGAGACGTGGCCGGGACCCTGAG | | YNELQKDKMAEAYSEIGMKG |
| | | ATGGGGGGAAAGCCGAGAAGGAAGAACCCTCAG | | ERRRGKGHDGLYQGLSTATK |
| | | GAAGGCCTGTACAATGAACTGCAGAAAGATAAGA | | DTYDALHMQALPPR |
| | | TGGCGGAGGCCTACAGTGAGATTGGGATGAAAG | | |
| | | GCGAGCGCCGGAGGGGCAAGGGGCACGATGGC | | |
| | | CTTTACCAGGGTCTCAGTACAGCCACCAAGGACA | | |
| | | CCTACGACGCCCTTCACATGCAAGCTCTTCCACC | | |
| | | TCGT | | |
| Leader | 57 | ATGCTCGAGATGCTGGAG | 58 | MLEMLE |
| P2A | 73 | GCTACTAACTTCAGCCTGCTGAAGCAGGCTGGA | 74 | ATNFSLLKQAGDVEENPGP |
| | | GACGTGGAGGAGAACCCCGGGCCT | | |
| MyD88 | 1 | ATGGCTGCAGGAGGTCCCGGCGCGGGGTCTGC | 2 | MAAGGPGAGSAAPVSSTSSL |
| | | GGCCCCGGTCTCCTCCACATCCTCCCTTCCCCT | | PLAALNMRVRRRLSLFLNVR |
| | | GGCTGCTCTCAACATGCGAGTGCGGCGCCGCCT | | TQVAADWTALAEEMDFEYLE |
| | | GTCTCTGTTCTTGAACGTGCGGACACAGGTGGC | | IRQLETQADPTGRLLDAWQG |
| | | GGCCGACTGGACCGCGCTGGCGGAGGAGATGG | | RPGASVGRLLDLLTKLGRDD |
| | | ACTTTGAGTACTTGGAGATCCGGCAACTGGAGAC | | VLLELGPSIEEDCQKYILKQQ |
| | | ACAAGCGGACCCCACTGGCAGGCTGCTGGACGC | | QEEAEKPLQVAAVDSSVPRT |
| | | CTGGCAGGGACGCCCTGGCGCCTCTGTAGGCC | | AELAGITTLDDPLGHMPERFD |
| | | GACTGCTCGATCTGCTTACCAAGCTGGGCCGCG | | AFICYCPSDI |
| | | ACGACGTGCTGCTGGAGCTGGGACCCAGCATTG | | |
| | | AGGAGGATTGCCAAAAGTATATCTTGAAGCAGCA | | |
| | | GCAGGAGGAGGCTGAGAAGCCTTTACAGGTGGC | | |
| | | CGCTGTAGACAGCAGTGTCCCACGGACAGCAGA | | |
| | | GCTGGCGGGCATCACCACACTTGATGACCCCCT | | |
| | | GGGGCATATGCCTGAGCGTTTCGATGCCTTCATC | | |
| | | TGCTATTGCCCCAGCGACATC | | |
| Linker | 77 | GCGGCCGCT | 78 | AAA |
| 41BB | 79 | AAACGGGGCAGAAAGAAACTCCTGTATATATTCA | 80 | KRGRKKLLYIFKQPFMRPVQ |
| | | AACAACCATTTATGAGACCAGTACAAACTACTCAA | | TTQEEDGCSCRFPEEEEGG |
| | | GAGGAAGATGGCTGTAGCTGCCGATTTCCAGAA | | CEL |
| | | GAAGAAGAAGGAGGATGTGAACTG | | |

### Example 3: Inducible Activation of MyD88/CD40 Via a Small Molecule Rimiducid Enhances NKs Expansion and Antitumor Efficacy Against Hematological Malignancies

NK cell-based therapy is a promising strategy for adoptive cancer immunotherapy but is limited by poor post-infusion persistence. Here, a novel inducible MyD88/CD40 platform was developed to activate NKs using the small molecule Rimiducid (Rim). Activated iMC modified NKs showed enhanced proliferation, prolonged persistence, and augmented antitumor efficacy in both MHC class I expression high and low tumor cells, which correlated with increased levels cytokine productions such as IFN-γ and TNF-α, and increased levels perforin and granzyme B as well as degranulation levels. Furthermore, IL-15, an inducible caspase-9 suicide gene (iRC9: a safety feature to preclude potential toxicity), and chimeric antigen receptors (CAR) targeting tumor specific antigens were also incorporated into the cells via γ-retroviral vectors. The results presented below demonstrated that NKs were able to be efficiently transduced, and that engineered NKs had dramatically enhanced antitumor activity and prolonged persistence in a xenograft THP-1 lymphoma murine model. Engineered NKs with a CAR significantly increased efficacy of NKs against leukemia/myeloma. The gene modified NKs were rapidly eliminated upon pharmacologic activation of the iRC9.

### Materials and Methods

### Cell Lines, Media, and Reagents

HEK293T, K562, HPAC, THP-1, NCIH929, U266, RPMI8226 were purchased from ATCC (Manassa, VA) and maintained in media per the suppliers' recommendation. CD56+NK cells were enriched with an NK isolation kit (Miltenyi Biotec, Inc., San Diego, CA), from peripheral blood mononuclear cells (PBMCs). PBMCs were isolated by a density-gradient technique (lymphoprep, Accurate Chemical & Scientific Corporation, Westbury, NY) from buffy coat blood obtained from the Gulf Coast Blood Bank. NKs were cultured in Stem cell growth medium (SCGM) (CellGenix GmbH, Freiburg, Germany) supplemented with 10% FBS, 1% pen/strep, 2 mM GlutaMax, and 200 U/ml recombinant human IL-2 (Miltenyi Biotec, Inc., San Diego, CA). Clinical-grade rimiducid was diluted in ethanol as a 100 μM solution for in vitro assays. For animal studies, rimiducid was further diluted into 0.9% saline. Research grade Temsirolimus (Sigma, St. Louis, MO) was dissolved in ethanol for in vitro assays and in Injection diluent (10% polyethylene glycol [PEG]-400+5% Tween-80, Sigma, St. Louis, MO) for animal studies.

### Retroviral and Plasmid Constructs

iMC contains a TIR domain-deleted version of the TLR adaptor protein MyD88, CD40 cytoplasmic region, and two tandem ligand-binding FKBP domains. iRC9 comprises tandem FRB and FKBPv domain fused with truncated caspase-9 (Duong, M. T., et al., Two-Dimensional Regulation of CAR-T Cell Therapy with Orthogonal Switches. Mol Ther Oncolytics, 2019. 12: p. 124-137). Synthetic DNA (Integrated DNA Technology, San Diego, CA) encoding human IL-15 was cloned into SFG iRC9.iMC using the enzyme site Sacll/PfIMI to generate pSFG-iRC9.IL15.iMC. These SFG retroviral vectors also consist a truncated human CD19 as a marker. G2A and T2A sequences were used to separate encoding genes. FIG. 1 shows a schematic representations of expression constructs that can be used to transduce NK cells.

A first generation CD123 CAR was generated with the 32716 scFV targeting CD123 (Foster, A. E., et al., Regulated Expansion and Survival of Chimeric Antigen Receptor-Modified T Cells Using Small Molecule-Dependent Inducible MyD88/CD40. Mol Ther, 2017. 25 (9): p. 2176-2188; Mardiros, A., et al., T cells expressing CD123-specific chimeric antigen receptors exhibit specific cytolytic effector functions and antitumor effects against human acute myeloid leukemia. Blood, 2013. 122 (18): p. 3138-48), CD8a stalk transmembrane domain, and the cytoplasmic CD3ζ domain. iMC.BCMA. ζ.IL 15 was generated by subcloning ScFV targeting BCMA (C12.A3.2L) and IL15 genes into pSFG iMC.CAR. ζ with Xhol/BamHI and SaII/MIul. All vectors contain CAR also had the QBEnd-10 minimal epitope (CD34 epitope) (Betts, M. R., et al., Sensitive and viable identification of antigen-specific CD8+ T cells by a flow cytometric assay for degranulation. J Immunol Methods, 2003. 281 (1-2): p. 65-78) as the marker. Tumor cells or NKs were transduced with pSFG-eGFP-Firefly luciferase (eGFPFfluc) or pSFG-orange Nano-lantern Renilla luciferase (ONLRluc) for labelling.

### Transduction of NK Cells or T Cells

Retroviral supernatants were produced by transient transfection of 293T cells as previously described (Foster, A. E., et al., Mol Ther, 2017. 25 (9): p. 2176-2188). NKs were stimulated with recombinant human IL-15 (15 ng/ml) for 1 day. The next day, NKs were further activated with irradiated (100 Gy) K562 at the ratio of 2:1 feeder: NK, and 200 U/ml recombinant human IL-2 (all cytokines from Miltenyi Biotec, Inc., San Diego, CA). Four days later, NKs were subsequently transduced using retronectin (Takara Bio, Mountain View, CA) and spinfection technique and were stimulated again with irradiated K562 and IL-2. For double transduction, NKs were transduced at day 4 and day 5 after IL-2 and K562 stimulations. On day 14, transduced NKs were harvested for use. Transduction of T cells was performed as described before (Foster, A. E., et al., Mol Ther, 2017. 25 (9): p. 2176-2188).

FIG. 3 shows methods to isolate, culture and transduce Natural Killer cells. Cell components from human blood sourced from healthy adult donors was separated by centrifugation in Ficoll with the ‘buffy coat’ further fractionated to provide peripheral blood mononuclear cells (T and B lymphocytes, NK cells and monocytes). NK cells were isolated by selection with a column of magnetic beads fused with antibody to CD56 expressed on NK cells.

### Plasmids:

TABLE
PBP2261-SFG-αCD123ScFV.CD34e.CD8stm.ZETA

| | | SEQ | | SEQ |
|---|---|---|---|---|
| FRAGMENT | NUCLEOTIDE | ID NO: | PEPTIDE | ID NO: |
| SIGNAL | ATGGAGTTCGGATTGAGCTGGCTGT | 23 | MEFGLSWLFLVAILKGVQCSR | 24 |
| PEPTIDE | TCCTGGTGGCAATACTCAAGGGCGT | | | |
| | TCAATGTTCACGG | | | |
| CD123 (32716) | CAGATCCAACTGGTGCAGTCAGGCC | 81 | QIQLVQSGPELKKPGETVKISCKASG | 82 |
| VH | CGGAACTGAAGAAGCCAGGGGAGA | | YIFTNYGMNWVKQAPGKSFKWMGWIN | |
| | CAGTCAAAATAAGTTGTAAAGCCAG | | TYTGESTYSADFKGRFAFSLETSAST | |
| | CGGCTACATATTTACTAATTACGGG | | AYLHINDLKNEDTATYFCARSGGYDP | |
| | ATGAATTGGGTGAAGCAAGCGCCGG | | MDYWGQGTSVTV | |
| | GCAAATCCTTTAAATGGATGGGGTG | | | |
| | GATAAACACATACACAGGAGAGTCA | | | |
| | ACGTACAGCGCGGACTTCAAAGGTC | | | |
| | GATTCGCGTTCAGTCTCGAGACCAG | | | |
| | CGCGAGTACAGCTTACCTCCACATC | | | |
| | AACGATCTTAAAAACGAAGACACGG | | | |
| | CAACCTATTTTTGCGCCCGGTCAGG | | | |
| | CGGTTACGACCCTATGGACTATTGG | | | |
| | GGCCAAGGGACCTCCGTTACGGTA | | | |
| FLEX | CTTCAGGCGGTGGCGGGAGTGGTG | 83 | SSGGGGSGGGGSGGGGS | 84 |
| | GAGGAGGCTCAGGCGGCGGGGGAT | | | |
| | CA | | | |
| CD123 (32716) | GACATCGTACTGACCCAATCTCCCG | 85 | DIVLTQSPASLAVSLGQRATISCRAS | 86 |
| VL | CTAGCCTTGCAGTATCCTTGGGTCA | | ESVDNYGNTFMHWYQQKPGQPPKLLI | |
| | ACGCGCTACAATAAGTTGCCGGGCT | | YRASNLESGIPARFSGSGSRTDFTLT | |
| | AGTGAGTCCGTAGACAACTATGGCA | | INPVEADDVATYYCQQSNEDPPTFGA | |
| | ACACCTTCATGCATTGGTACCAACAA | | GTKLELKESKYGPPCP | |
| | AAACCAGGTCAGCCACCCAAACTTC | | | |
| | TCATTTACAGAGCGTCTAATCTCGA | | | |
| | AAGCGGCATCCCTGCTCGATTCTCT | | | |
| | GGAAGCGGAAGTAGAACCGACTTTA | | | |
| | CACTGACTATAAACCCCGTCGAAGC | | | |
| | CGATGATGTTGCCACTTATTACTGT | | | |
| | CAACAGAGCAATGAGGACCCACCGA | | | |
| | CATTCGGTGCTGGTACCAAGCTGGA | | | |
| | GTTGAAGGAGTCAAAATACGGGCCT | | | |
| | CCCTGTCCC | | | |
| LINKER | GGATCC | 33 | GS | 34 |
| CD34 EPITOPE | GAACTTCCTACTCAGGGGACTTTCT | 35 | ELPTQGTFSNVSTNVS | 36 |
| | CAAACGTTAGCACAAACGTAAGT | | | |
| CD8 STALK | GAACTTCCTACTCAGGGGACTTTCT | 37 | PAPRPPTPAPTIASQPLSLRPEACRP | 38 |
| | CAAACGTTAGCACAAACGTAAGT | | AAGGAVHTRGLDFACD | |
| CD8 | ATCTATATCTGGGCACCTCTCGCTG | 39 | IYIWAPLAGTCGVLLLSLVITLYCNH | 40 |
| TRANSMEMBRANE | GCACCTGTGGAGTCCTTCTGCTCAG | | RNRRRVCKCPR | |
| | CCTGGTTATTACTCTGTACTGTAAT | | | |
| | CACCGGAATCGCCGCCGCGTTTGTA | | | |
| | AGTGTCCCAGG | | | |
| LINKER | GTCGAC | 41 | VD | 42 |
| CD3Z | AGAGTGAAGTTCAGCAGGAGCGCA | 43 | RVKFSRSADAPAYQQGQNQLYNELN | 44 |
| | GACGCCCCCGCGTACCAGCAGGGC | | LGRREEYDVLDKRRGRDPEMGGKPR | |
| | CAGAACCAGCTCTATAACGAGCTC | | RKNPQEGLYNELQKDKMAEAYSEIG | |
| | AATCTAGGACGAAGAGAGGAGTAC | | MKGERRRGKGHDGLYQGLSTATKDT | |
| | GATGTTTTGGACAAGAGACGTGGC | | YDALHMQALPPR | |
| | CGGGACCCTGAGATGGGGGGAAAG | | | |
| | CCGAGAAGGAAGAACCCTCAGGAA | | | |
| | GGCCTGTACAATGAACTGCAGAAA | | | |
| | GATAAGATGGCGGAGGCCTACAGT | | | |
| | GAGATTGGGATGAAAGGCGAGCGC | | | |
| | CGGAGGGGCAAGGGGCACGATGGC | | | |
| | CTTTACCAGGGTCTCAGTACAGCC | | | |
| | ACCAAGGACACCTACGACGCCCTT | | | |
| | ACACATGCAGCTCTTCCACCTCGT | | | |
| STOP | TGA | | STOP | |

TABLE
PBP2818-pSFG-MYD88.CD40.Fv.Fv.T2A.αBCMAScFV.CD34e.CD8stm.ZETA

| | | SEQ | | SEQ |
|---|---|---|---|---|
| FRAGMENT | NUCLEOTIDE | ID NO: | PEPTIDE | ID NO: |
| MYD88 | ATGGCTGCAGGAGGTCCCGGCGCG | 1 | MAAGGPGAGSAAPVSSTSSLPLAALN | 2 |
| | GGGTCTGCGGCCCCGGTCTCCTCC | | MRVRRRLSLFLNVRTQVAADWTALAE | |
| | ACATCCTCCCTTCCCCTGGCTGCTC | | EMDFEYLEIRQLETQADPTGRLLDAW | |
| | TCAACATGCGAGTGCGGCGCCGCC | | QGRPGASVGRLLDLLTKLGRDDVLLE | |
| | TGTCTCTGTTCTTGAACGTGCGGAC | | LGPSIEEDCQKYILKQQQEEAEKPLQ | |
| | ACAGGTGGCGGCCGACTGGACCGC | | VAAVDSSVPRTAELAGITTLDDPLGH | |
| | GCTGGCGGAGGAGATGGACTTTGA | | MPERFDAFICYCPSDI | |
| | GTACTTGGAGATCCGGCAACTGGAG | | | |
| | ACACAAGCGGACCCCACTGGCAGG | | | |
| | CTGCTGGACGCCTGGCAGGGACGC | | | |
| | CCTGGCGCCTCTGTAGGCCGACTG | | | |
| | CTCGATCTGCTTACCAAGCTGGGCC | | | |
| | GCGACGACGTGCTGCTGGAGCTGG | | | |
| | GACCCAGCATTGAGGAGGATTGCCA | | | |
| | AAAGTATATCTTGAAGCAGCAGCAG | | | |
| | GAGGAGGCTGAGAAGCCTTTACAGG | | | |
| | TGGCCGCTGTAGACAGCAGTGTCCC | | | |
| | ACGGACAGCAGAGCTGGCGGGCAT | | | |
| | CACCACACTTGATGACCCCCTGGGG | | | |
| | CATATGCCTGAGCGTTTCGATGCCT | | | |
| | TCATCTGCTATTGCCCCAGCGACAT | | | |
| | C | | | |
| LINKER | GTCGAG | 13 | VE | 14 |
| CD40 | AAAAAGGTGGCCAAGAAGCCAACCA | 75 | KKVAKKPTNKAPHPKQEPQEINFPD | 56 |
| | ATAAGGCCCCCCACCCCAAGCAGGA | | DLPGSNTAAPVQETLHGCQPVTQED | |
| | GCCCCAGGAGATCAATTTTCCCGAC | | GKESRISVQERQ | |
| | GATCTTCCTGGCTCCAACACTGCTG | | | |
| | CTCCAGTGCAGGAGACTTTACATGG | | | |
| | ATGCCAACCGGTCACCCAGGAGGAT | | | |
| | GGCAAAGAGAGTCGCATCTCAGTGC | | | |
| | AGGAGAGACAG | | | |
| LINKER | GTCGAG | 13 | VE | 14 |
| FKBPV′ | GGCGTCCAAGTCGAAACCATTAGTC | 11 | GVQVETISPGDGRTFPKRGQTCVVH | 12 |
| | CCGGCGATGGCAGAACATTTCCTAA | | YTGMLEDGKKVDSSRDRNKPFKFML | |
| | AAGGGGACAAACATGTGTCGTCCAT | | GKQEVIRGWEEGVAQMSVGQRAKLT | |
| | TATACAGGCATGTTGGAGGACGGCA | | ISPDYAYGATGHPGIIPPHATLVFD | |
| | AAAAGGTGGACAGTAGTAGAGATCG | | VELLKLE | |
| | CAATAAACCTTTCAAATTCATGTTG | | | |
| | GGAAAACAAGAAGTCATTAGGGGAT | | | |
| | GGGAGGAGGGCGTGGCTCAAATGTC | | | |
| | CGTCGGCCAACGCGCTAAGCTCACC | | | |
| | ATCAGCCCCGACTACGCATACGGCG | | | |
| | CTACCGGACATCCCGGAATTATTCC | | | |
| | CCCTCACGCTACCTTGGTGTTTGAC | | | |
| | GTCGAACTGTTGAAGCTCGAA | | | |
| LINKER | GTCGAG | 13 | VE | 14 |
| FKBPV | GGAGTGCAGGTGGAGACTATCTCCC | 15 | GVQVETISPGDGRTFPKRGQTCVVH | 16 |
| | CAGGAGACGGGCGCACCTTCCCCA | | YTGMLEDGKKVDSSRDRNKPFKFML | |
| | AGCGCGGCCAGACCTGCGTGGTGC | | GKQEVIRGWEEGVAQMSVGQRAKLT | |
| | ACTACACCGGGATGCTTGAAGATGG | | ISPDYAYGATGHPGIIPPHATLVFD | |
| | AAAGAAAGTTGATTCCTCCCGGGAC | | VELLKLE | |
| | AGAAACAAGCCCTTTAAGTTTATGCT | | | |
| | AGGCAAGCAGGAGGTGATCCGAGG | | | |
| | CTGGGAAGAAGGGGTTGCCCAGAT | | | |
| | GAGTGTGGGTCAGAGAGCCAAACTG | | | |
| | ACTATATCTCCAGATTATGCCTATGG | | | |
| | TGCCACTGGGCACCCAGGCATCATC | | | |
| | CCACCACATGCCACTCTCGTCTTCG | | | |
| | ATGTGGAGCTTCTAAAACTGGAA | | | |
| LINKER | CCGCGG | 17 | PR | 18 |
| T2A | GAGGGCAGAGGCAGCCTCCTGACA | 19 | EGRGSLLTCGDVEENPGP | 20 |
| | TGTGGGGACGTCGAGGAGAACCCT | | | |
| | GGCCCA | | | |
| LINKER | CCTTGG | 21 | PW | 22 |
| SIGNAL | ATGGAGTTCGGATTGAGCTGGCTGT | 23 | MEFGLSWLFLVAILKGVQCSR | 24 |
| PEPTIDE | TCCTGGTGGCAATACTCAAGGGCGT | | | |
| | TCAATGTTCACGG | | | |
| BCMA | GATATCGTGCTGACCCAGTCCCCCC | 87 | DIVLTQSPPSLAMSLGKRATISCRASE | 88 |
| (C123.A3.2) VL | CTAGCCTGGCCATGTCCCTGGGCAA | | SVTILGSHLIYWYQQKPGQPPTLLIQL | |
| | ACGGGCCACCATCTCCTGCAGAGCC | | ASNVQTGVPARFSGSGSRTDFTLTID | |
| | TCCGAGTCCGTGACCATCCTCGGCT | | PVEEDDVAVYYCLQSRTIPRTFGGGT | |
| | CCCACCTGATCTACTGGTACCAGCA | | KLEIK | |
| | GAAGCCCGGCCAGCCTCCCACCCT | | | |
| | CCTTATCCAGCTGGCCAGCAACGTG | | | |
| | CAGACCGGCGTGCCCGCTAGATTCT | | | |
| | CCGGCAGCGGCTCTAGAACCGACTT | | | |
| | CACCCTGACCATCGACCCCGTGGAA | | | |
| | GAGGACGATGTCGCCGTGTACTATT | | | |
| | GCCTGCAGTCCAGAACCATCCCTAG | | | |
| | GACATTCGGCGGAGGAACCAAGCT | | | |
| | GGAGATCAAA | | | |
| FLEX | GGGGGCGGTGGCAGCGGTGGCGG | 89 | GGGGSGGGGSGGGGS | 90 |
| | TGGGTCTGGGGGCGGAGGCTCT | | | |
| BCMA | CAGATCCAGCTGGTGCAGTCCGGC | 91 | QIQLVQSGPELKKPGETVKISCKASGY | 92 |
| (C123.A3.2) VH | CCCGAGCTGAAGAAACCCGGCGAG | | TFRHYSMNWVKQAPGKGLKWMGRIN | |
| | ACCGTGAAGATCTCCTGCAAGGCCA | | TESGVPIYADDFKGRFAFSVETSASTA | |
| | GCGGCTACACCTTCAGACACTACAG | | YLVINNLKDEDTASYFCSNDYLYSLDF | |
| | CATGAACTGGGTGAAGCAGGCCCCT | | WGQGT ALTVSS | |
| | GGCAAGGGCCTGAAGTGGATGGGC | | | |
| | CGGATCAACACCGAGTCCGGCGTG | | | |
| | CCCATCTACGCCGACGATTTCAAGG | | | |
| | GCAGATTCGCCTTCAGCGTGGAGAC | | | |
| | CTCCGCCTCTACCGCCTACCTGGTG | | | |
| | ATCAACAATCTGAAGGACGAGGACA | | | |
| | CCGCCTCCTACTTCTGCAGCAACGA | | | |
| | CTACCTGTACAGCCTGGACTTCTGG | | | |
| | GGCCAGGGCACCGCCCTGACCGTG | | | |
| | AGCTCCG | | | |
| LINKER | GGATCC | 33 | GS | 34 |
| CD34 EPITOPE | GAACTTCCTACTCAGGGGACTTTCT | 35 | ELPTQGTFSNVSTNVS | 36 |
| | CAAACGTTAGCACAAACGTAAGT | | | |
| CD8 STALK | GAACTTCCTACTCAGGGGACTTTCT | 37 | PAPRPPTPAPTIASQPLSLRPEACRPA | 38 |
| | CAAACGTTAGCACAAACGTAAGT | | AGGAVHTRGLDFACD | |
| CD8 | ATCTATATCTGGGCACCTCTCGCTG | 39 | IYIWAPLAGTCGVLLLSLVITLYCNHRN | 40 |
| TRANSMEMBRANE | GCACCTGTGGAGTCCTTCTGCTCAG | | RRRVCKCPR | |
| | CCTGGTTATTACTCTGTACTGTAATC | | | |
| | ACCGGAATCGCCGCCGCGTTTGTAA | | | |
| | GTGTCCCAGG | | | |
| LINKER | GTCGAC | 41 | VD | 42 |
| CD3Z | AGAGTGAAGTTCAGCAGGAGCGCA | 43 | RVKFSRSADAPAYQQGQNQLYNELN | 44 |
| | GACGCCCCCGCGTACCAGCAGGGC | | LGRREEYDVLDKRRGRDPEMGGKPR | |
| | CAGAACCAGCTCTATAACGAGCTCA | | RKNPQEGLYNELQKDKMAEAYSEIGM | |
| | ATCTAGGACGAAGAGAGGAGTACGA | | KGERRRGKGHDGLYQGLSTATKDTY | |
| | TGTTTTGGACAAGAGACGTGGCCGG | | DALHMQALPPR | |
| | GACCCTGAGATGGGGGGAAAGCCG | | | |
| | AGAAGGAAGAACCCTCAGGAAGGC | | | |
| | CTGTACAATGAACTGCAGAAAGATA | | | |
| | AGATGGCGGAGGCCTACAGTGAGAT | | | |
| | TGGGATGAAAGGCGAGCGCCGGAG | | | |
| | GGGCAAGGGGCACGATGGCCTTTA | | | |
| | CCAGGGTCTCAGTACAGCCACCAAG | | | |
| | GACACCTACGACGCCCTTCACATGC | | | |
| | AAGCTCTTCCACCTCGT | | | |
| LINKER | GGATCTGGCGGCCGC | 93 | GSGGR | 94 |
| T2A | GAGGGAAGGGGAAGTCTTCTAACAT | 95 | EGRGSLLTCGDVEENPGP | 20 |
| | GCGGGGACGTGGAGGAAAATCCCG | | | |
| | GGCCC | | | |
| IL-15 | GAGGGAAGGGGAAGTCTTCTAACAT | 96 | MRISKPHLRSISIQCYLCLLLNSHFLTE | 97 |
| | GCGGGGACGTGGAGGAAAATCCCG | | AGIHVFILGCFSAGLPKTEANWVNVIS | |
| | GGCCCATGAGAATTTCGAAACCACA | | DLKKIEDLIQSMHIDATLYTESDVHPSC | |
| | TTTGAGAAGTATTTCCATCCAGTGCT | | KVTAMKCFLLELQVISLESGDASIHDT | |
| | ACTTGTGTTTACTTCTAAACAGTCAT | | VENLIILANNSLSSNGNVTESGCKECE | |
| | TTTCTAACTGAAGCTGGCATTCATGT | | ELEEKNIKEFLQSFVHIVQMFINT | |
| | CTTCATTTTGGGCTGTTTCAGTGCAG | | | |
| | GGCTTCCTAAAACAGAAGCCAACTG | | | |
| | GGTGAATGTAATAAGTGATTTGAAAA | | | |
| | AAATTGAAGACCTTATTCAATCTATG | | | |
| | CACATTGATGCTACTTTATATACGGA | | | |
| | AAGTGATGTTCACCCCAGTTGCAAA | | | |
| | GTAACAGCAATGAAGTGCTTTCTCTT | | | |
| | GGAGTTACAAGTTATTTCACTTGAGT | | | |
| | CCGGAGATGCAAGTATTCATGATAC | | | |
| | AGTAGAAAATCTGATCATCCTAGCAA | | | |
| | ACAACAGTTTGTCTTCTAATGGGAAT | | | |
| | GTAACAGAATCTGGATGCAAAGAAT | | | |
| | GTGAGGAACTGGAGGAGAAGAACAT | | | |
| | CAAGGAATTTTTGCAGAGTTTTGTAC | | | |
| | ATATTGTCCAAATGTTCATCAACACT | | | |
| STOP | TGA | | STOP | |

TABLE
PBP1385-PSFG-FRB.FKBP.ΔC9.T2A-ΔCD19

| | | SEQ | | SEQ |
|---|---|---|---|---|
| FRAGMENT | NUCLEOTIDE | ID NO: | PEPTIDE | ID NO: |
| LEADER | ATGCTCGAGCAATTG | 98 | MLEQL | 99 |
| PEPTIDE | | | | |
| FRB | GAAATGTGGCATGAAGGGTTGGAAG | 100 | EMWHEGLEEASRLYFGERNVKGMFE | 101 |
| | AAGCTTCAAGGCTGTACTTCGGAGA | | VLEPLHAMMERGPQTLKETSFNQAY | |
| | GAGGAACGTGAAGGGCATGTTTGAG | | GRDLMEAQEWCRKYMKSGNVKDLTQ | |
| | GTTCTTGAACCTCTGCACGCCATGA | | AWDLYYHVFRRISK | |
| | TGGAACGGGGACCGCAGACACTGA | | | |
| | AAGAAACCTCTTTTAATCAGGCCTA | | | |
| | CGGCAGAGACCTGATGGAGGCCCAA | | | |
| | GAATGGTGTAGAAAGTATATGAAAT | | | |
| | CCGGTAACGTGAAAGACCTGACTCA | | | |
| | GGCCTGGGACCTTTATTACCATGTG | | | |
| | TTCAGGCGGATCAGTAAG | | | |
| LINKER | GGCGGGCAATTG | 102 | GGQL | 103 |
| FKBP WT | GGCGTCCAAGTCGAAACCATTAGTC | 104 | GVQVETISPGDGRTFPKRGQTCVVHY | 105 |
| | CCGGCGATGGCAGAACATTTCCTAA | | TGMLEDGKKFDSSRDRNKPFKFMLG | |
| | AAGGGGACAAACATGTGTCGTCCAT | | KQEVIRGWEEGVAQMSVGQRAKLTIS | |
| | TATACAGGCATGTTGGAGGACGGCA | | PDYAYGATGHPGIIPPHATLVFDVEL | |
| | AAAAGTTCGACAGTAGTAGAGATCG | | LKL | |
| | CAATAAACCTTTCAAATTCATGTTG | | | |
| | GGAAAACAAGAAGTCATTAGGGGAT | | | |
| | GGGAGGAGGGCGTGGCTCAAATGTC | | | |
| | CGTCGGCCAACGCGCTAAGCTCACC | | | |
| | ATCAGCCCCGACTACGCATACGGCG | | | |
| | CTACCGGACATCCCGGAATTATTCC | | | |
| | CCCTCACGCTACCTTGGTGTTTGAC | | | |
| | GTCGAACTGTTGAAGCTC | | | |
| LINKER | TCAGGCGGTGGCTCAGGTCCATGG | 106 | SGGGSGPW | 107 |
| ΔCASPASE9 | GGATTTGGTGATGTCGGTGCTCTTG | 108 | GFGDVGALESLRGNADLAYILSMEPC | 64 |
| | AGAGTTTGAGGGGAAATGCAGATTT | | GHCLIINNVNFCRESGLRTRTGSNID | |
| | GGCTTACATCCTGAGCATGGAGCCC | | CEKLRRRFSSLHFMVEVKGDLTAKKM | |
| | TGTGGCCACTGCCTCATTATCAACA | | VLALLELARQDHGALDCCVVVILSHG | |
| | ATGTGAACTTCTGCCGTGAGTCCGG | | CQASHLQFPGAVYGTDGCPVSVEKIV | |
| | GCTCCGCACCCGCACTGGCTCCAAC | | NIFNGTSCPSLGGKPKLFFIQACGGE | |
| | ATCGACTGTGAGAAGTTGCGGCGTC | | QKDHGFEVASTSPEDESPGSNPEPDA | |
| | GCTTCTCCTCGCTGCATTTCATGGT | | TPFQEGLRTFDQLDAISSLPTPSDIF | |
| | GGAGGTGAAGGGCGACCTGACTGC | | VSYSTFPGFVSWRDPKSGSWYVETLD | |
| | CAAGAAAATGGTGCTGGCTTTGCTG | | DIFEQWAHSEDLQSLLLRVANAVSVK | |
| | GAGCTGGCGCGGCAGGACCACGGT | | KQMPGCFNFLRKKLFFKTSASRA | |
| | GCTCTGGACTGCTGCGTGGTGGTCA | | GIY | |
| | TTCTCTCTCACGGCTGTCAGGCCAG | | | |
| | CCACCTGCAGTTCCCAGGGGCTGTC | | | |
| | TACGGCACAGATGGATGCCCTGTGT | | | |
| | CGGTCGAGAAGATTGTGAACATCTT | | | |
| | CAATGGGACCAGCTGCCCCAGCCT | | | |
| | GGGAGGGAAGCCCAAGCTCTTTTTC | | | |
| | ATCCAGGCCTGTGGTGGGGAGCAG | | | |
| | AAAGACCATGGGTTTGAGGTGGCCT | | | |
| | CCACTTCCCCTGAAGACGAGTCCCC | | | |
| | TGGCAGTAACCCCGAGCCAGATGCC | | | |
| | ACCCCGTTCCAGGAAGGTTTGAGGA | | | |
| | CCTTCGACCAGCTGGACGCCATATC | | | |
| | TAGTTTGCCCACACCCAGTGACATC | | | |
| | TTTGTGTCCTACTCTACTTTCCCAGG | | | |
| | TTTTGTTTCCTGGAGGGACCCCAAG | | | |
| | AGTGGCTCCTGGTACGTTGAGACCC | | | |
| | TGGACGACATCTTTGAGCAGTGGGC | | | |
| | TCACTCTGAAGACCTGCAGTCCCTC | | | |
| | CTGCTTAGGGTCGCTAATGCTGTTT | | | |
| | CGGTGAAAGGGATTTATAAACAGAT | | | |
| | GCCTGGTTGCTTTAATTTCCTCCGG | | | |
| | AAAAAACTTTTCTTTAAAACATCAGC | | | |
| | TAGCAGAGCC | | | |
| LINKER | GGATCTGGACCGCGG | 109 | GSGPR | 110 |
| T2A | GAAGGCCGAGGGAGCCTGCTGACA | 65 | EGRGSLLTCGDVEENPGP | 20 |
| | TGTGGCGATGTGGAGGAAAACCCAG | | | |
| | GACCA | | | |
| ΔCD19 | ATGCCACCACCTCGCCTGCTGTTCT | 111 | MPPPRLLFFLLFLTPMEVRPEEPLV | 112 |
| | TTCTGCTGTTCCTGACACCTATGGA | | VKVEEGDNAVLQCLKGTSDGPTQQL | |
| | GGTGCGACCTGAGGAACCACTGGT | | TWSRESPLKPFLKLSLGLPGLGIHM | |
| | CGTGAAGGTCGAGGAAGGCGACAA | | RPLAIWLFIFNVSQQMGGFYLCQPG | |
| | TGCCGTGCTGCAGTGCCTGAAAGGC | | PPSEKAWQPGWTVNVEGSGELFRWN | |
| | ACTTCTGATGGGCCAACTCAGCAGC | | VSDLGGLGCGLKNRSSEGPSSPSGK | |
| | TGACCTGGTCCAGGGAGTCTCCCCT | | LMSPKLYVWAKDRPEIWEGEPPCLP | |
| | GAAGCCTTTTCTGAAACTGAGCCTG | | PRDSLNQSLSQDLTMAPGSTLWLSC | |
| | GGACTGCCAGGACTGGGAATCCACA | | GVPPDSVSRGPLSWTHVHPKGPKSL | |
| | TGCGCCCTCTGGCTATCTGGCTGTT | | LSLELKDDRPARDMWVMETGLLLPR | |
| | CATCTTCAACGTGAGCCAGCAGATG | | ATAQDAGKYYCHRGNLTMSFHLEIT | |
| | GGAGGATTCTACCTGTGCCAGCCAG | | ARPVLWHWLLRTGGWKVSAVTLAYL | |
| | GACCACCATCCGAGAAGGCCTGGC | | IFCLCSLVGILHLQRALVLRRKRKR | |
| | AGCCTGGATGGACCGTCAACGTGGA | | MTDPTRRF | |
| | GGGGTCTGGAGAACTGTTTAGGTGG | | | |
| | AATGTGAGTGACCTGGGAGGACTGG | | | |
| | GATGTGGGCTGAAGAACCGCTCCTC | | | |
| | TGAAGGCCCAAGTTCACCCTCAGGG | | | |
| | AAGCTGATGAGCCCAAAACTGTACG | | | |
| | TGTGGGCCAAAGATCGGCCCGAGAT | | | |
| | CTGGGAGGGAGAACCTCCATGCCT | | | |
| | GCCACCTAGAGACAGCCTGAATCAG | | | |
| | AGTCTGTCACAGGATCTGACAATGG | | | |
| | CCCCCGGGTCCACTCTGTGGCTGTC | | | |
| | TTGTGGAGTCCCACCCGACAGCGTG | | | |
| | TCCAGAGGCCCTCTGTCCTGGACCC | | | |
| | ACGTGCATCCTAAGGGGCCAAAAAG | | | |
| | TCTGCTGTCACTGGAACTGAAGGAC | | | |
| | GATCGGCCTGCCAGAGACATGTGG | | | |
| | GTCATGGAGACTGGACTGCTGCTGC | | | |
| | CACGAGCAACCGCACAGGATGCTG | | | |
| | GAAAATACTATTGCCACCGGGGCAA | | | |
| | TCTGACAATGTCCTTCCATCTGGAG | | | |
| | ATCACTGCAAGGCCCGTGCTGTGGC | | | |
| | ACTGGCTGCTGCGAACCGGAGGAT | | | |
| | GGAAGGTCAGTGCTGTGACACTGGC | | | |
| | ATATCTGATCTTTTGCCTGTGCTCCC | | | |
| | TGGTGGGCATTCTGCATCTGCAGAG | | | |
| | AGCCCTGGTGCTGCGGAGAAAGAG | | | |
| | AAAGAGAATGACTGACCCAACAAGA | | | |
| | AGGTTT | | | |
| STOP | TGA | | STOP | |

### NK Cell Proliferation Assay

NKs from healthy donors were activated by IL-15, followed by k562 and IL-2. On the day of transduction, activated NKs were labeled with CellTrace Violet Cell proliferation dye (ThermoFisher Scientific, Grand Island, NY) per the manufacturer's protocol. NKs were subsequently transduced with iRC9 or iRC9.iMC and stimulated with K562 and IL-2 in the absence or presence of 1 nM Rimiducid. After culturing 9 days, viable NKs (CD56+CD3−) were then analyzed for dilution of dye.

### CD107a and Intracellular Staining

CD107a degranulation and intracellular cytokine IFN-γ, TNF-α, perforin, granzyme B productions were measured as reported (Betts, M. R., et al., J Immunol Methods, 2003. 281 (1-2): p. 65-78]. Briefly, NKs modified with iRC9 or iRC9.iMC vectors were co-cultured with or without THP-1 tumor targets at 1:1 E/T ratio in the presence or absence of 1 nM Rimiducid for 4-hour incubation. Anti CD107a-PE antibody (eBiosciense, San Diego, CA) was added during the first hour, followed by the secretion inhibitor monensin (2 μM, Biolegend, San Diego, CA) and Brefeldin A (5 g/ml, Biolegend, San Diego, CA) treatment for the next 3 hrs. Cells were then washed, stained with surface antibodies anti-CD19-BV421, anti-CD56-APCcy7, anti-CD3-FITC (Biolegend, San Diego, CA). For intracellular staining, gene modified NKs were co-cultured with or without THP-1 and rimiducid for overnight. Secretion inhibitors were added. Cells were harvested, washed, stained with surface antibodies anti-CD56-APCcy7 and anti-CD19-PE or anti-CD56 PE and anti-CD19 BV421 (Biolegend, San Diego, CA), fixed, permeabilized and stained intracellularly with anti-IFN-γ-APC, anti-TNF-α-APC (BD Bioscience, San Jose, CA), anti-Perforin-APC/cy7 (Biolegend, San Diego, CA), or anti-Granzyme B-AF647 (BD Bioscience, San Jose, CA).

### Immunophenotype of Transduced Cells

Transduced NKs or T cells were stained with anti-CD56-APCcy7 (NK) or anti-CD3-Percep.Cy5 (T) and anti-CD19-BV421 for iMC or iRC9 transgene expression. For CAR-transduced cells, anti-CD34 QBEnd-10-PE (Abnova) was used to determine transduction efficiency. Phenotypic analysis of NKs none-transduced (NT), iRC9, iRC9.iMC, or iRC9.IL15.iMC transduced cells were assessed by multi-color flow cytometry using the following three sets antibody panels: anti-NKP30-AF647, anti-CD16-AF488, anti-CD19-PE, anti-CD56-APC cy7, and anti-CD178 (Fas-L)-BV421; anti-DNAM-1-FITC, anti-NKG2D-APC, anti-CD56-APCcy7, anti-CD19-PE, and anti-NKP46 BV421; anti-CD95 (Fas)-PEcy7, anti-CD56-APCcy7, anti-CD19-BV421, and anti-NKP44 Percep/cy5.5. All antibodies were purchased from Biolegend except otherwise stated. Flow cytometry was performed using NovoCyte flow cytometer and the data were analyzed using ACEA NovoExpress software (ACEA Biosciences Inc, San Diego, CA).

### Cytokine Production

Production of IFN-γ by NKs transduced with iRC9, iRC9.iMC, or iRC9.IL15.iMC in the presence of various concentration rimiducid was analyzed by ELISA (eBioscience, San Diego, CA). In some experiments, cytokines production was analyzed using a 29-cytokine/chemokine mutiplex array (Milliplex MAP; Millipore) system with a multiplex reader (Bio-Plex MAGPIX, Bio-rad)

### Immunoblotting

Details of immunoblotting were described before (Foster, A. E., et al., Mol Ther, 2017. 25 (9): p. 2176-2188; Duong, M. T., et al., Mol Ther Oncolytics, 2019. 12: p. 124-137; Collinson-Pautz, M. R., et al., Leukemia, 2019). Antibodies specific for phospho-NF-κB p65, phospho-IκBα, phospho-p38, phospho-ERK1/2, phospho-SAPK/JNK, TRAF3, and TRAF6 were purchased from Cell signaling Technology Inc (Danvers, MA). Antibodies specific for MyD88 and β-actin were purchased from Santa Cruz Biotechnology (Dallas, Texas).

### Cytotoxicity and Co-Culture Assays

To access cytotoxicity, gene-modified NK cells were co-cultured with THP-1 or other targets labeled with eGFPFfluc as noted in figure legend at multiple E:T ratios in the presence or absence of 1 nM Rimiducid (for short term assay, NKs were pretreated with rimiducid for 6 days). Cytotoxicity was measured by luciferase activity assay 24 hrs later following the manufacturer's protocols. The findings are reported as specific lysis relative to THP-1 or other tumor cell targets. Additional co-culture assays were analyzed by flow cytometry for the frequency of tumor cells GFP+ populations. In some long term assays, gene modified NKs were co-cultured with tumor cell targets either labeled with eGFP or RFP for 7 days in the presence of 200 U/ml IL-2. Tumor cells growths were monitored by real-time fluorescent microscopy (IncuCyte; Essen Biosciences).

### In Vivo Studies

NOD/SCID/γc−/−(NSG) mice (Jackson laboratory, Bar Harbor, ME) were maintained at the Bellicum Pharmaceuticals AAALAC approved vivarium. These studies were approved by the Bellicum Pharmaceuticals Institutional Animal Care and Use Committee (IACUC). For NK persistence study, 107 double transduced NKs (indicated transgenes and eGFPFfluc) in 100 μLPBS were tail vein injected into NSG mice (8 weeks old female). Rimiducid or vehicle were administered via i.p. injection at the dose of 1 mg/kg weekly. In the NK persistence study with THP-1 tumor targets, two times 5×10⁶ double transduced NKs (indicated transgenes and ONLRluc) were tail vein injected 5 days before and 12 days after 10⁶ THP-1-eGFPFfluc engraftment. For CAR-NK experiments, 10⁷ gene modified NKs were tail vein injected 3 days after 10⁶ THP-1-eGFPFfluc engraftment. In vivo NK presence or tumor growth was measured by weekly bioluminescence imaging (BLI) by i.p. injection of 150 mg/kg D-luciferin (firefly luciferase) or 150 ng Coelenterazine-h (renilla luciferase; Perkin Elmer, Waltham, MA) and imaged using the IVIS imaging system (Perkin Elmer). Photon whole body emission in a region of interest was assessed. Signal quantitation was measured as average radiance (photons/second/cm²/steradian). Weight measurement was performed at least once a week. Endpoint analysis involved FACS analysis of splenocyte, bone marrow, or peripheral blood.

### Statistics

All statistical tests (noted in figure legends) were carried out and analyzed using GraphPad Prism software (version 8.0, GraphPad, software). Data are presented as means±SEM. All t tests were 2-tailed. All ANOVA were two-way. P values of less than 0.05 were considered significant.

### Results

### iMC Modified NKs Show Enhanced Proliferation Upon Rimiducid Activation

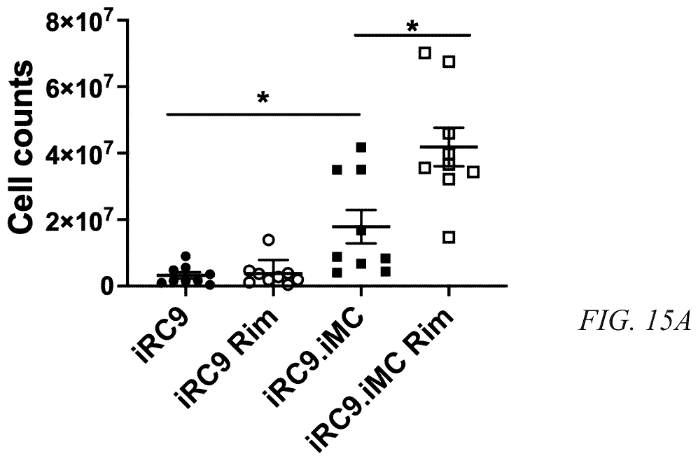

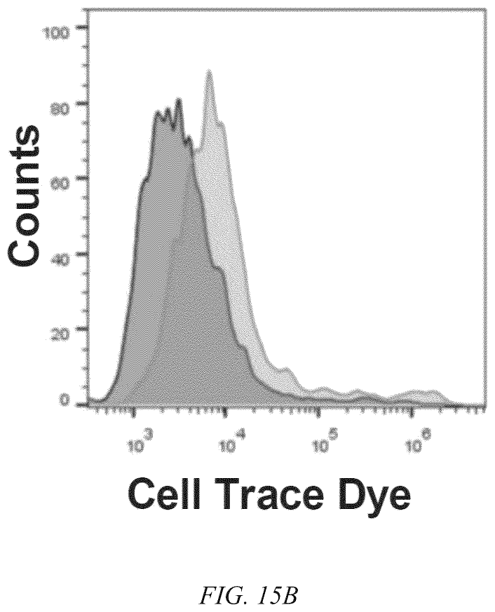

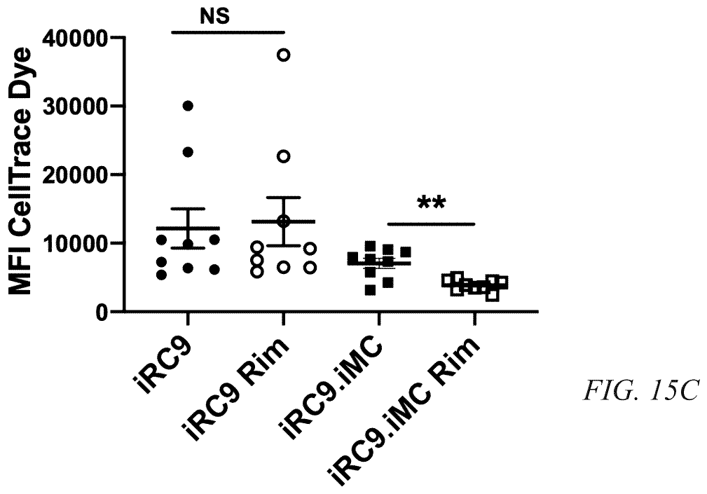

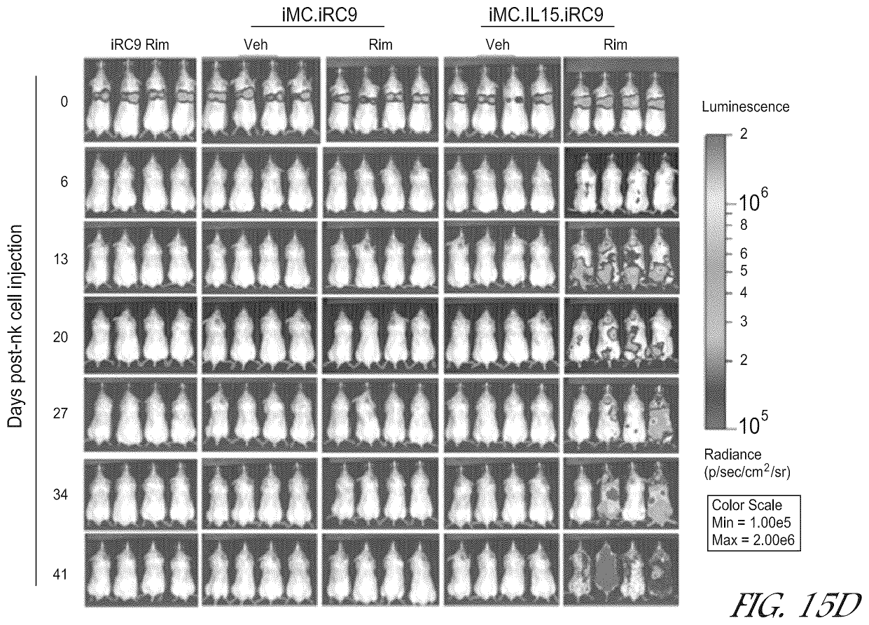

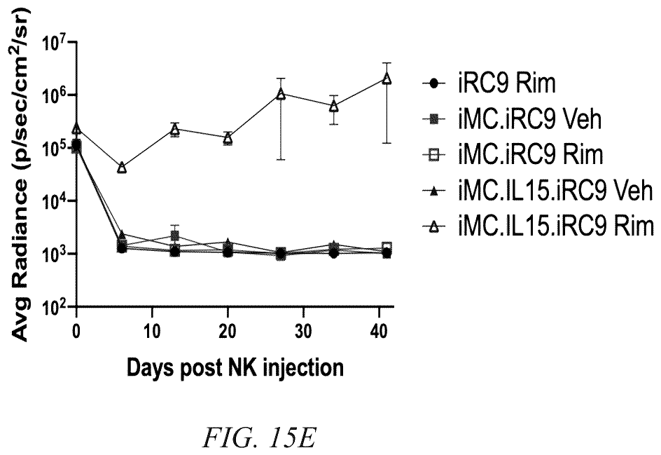

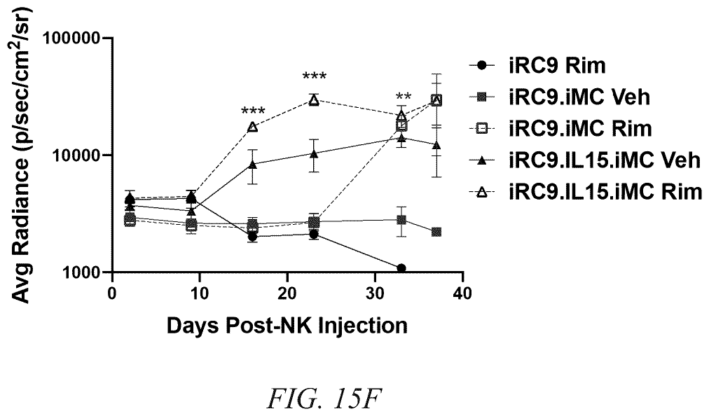

iMC effects on NKs proliferation were tested. A bicistronic retroviral vector encoding iRC9 followed by iMC were used to transduce activated NKs on day 4. iRC9 encoding vector served as the control. At day 9, those gene modified NKs were re-stimulated with irradiated k562 in the presence of 0 or 1 nM Rim. At day 14 of culture, iRC9.iMC modified group contained increased numbers of viable NK cells compared with the iRC9 group. Furthermore, rimiducid treatment resulted in significantly enhancement of NK expansion (FIG. 15A). These findings were confirmed by Cell Trace dye dilution data-showing markedly increased proliferation in the rimiducid-treated compared with non-rimiducid administrated iRC9.iMC NKs (FIGS. 15B and 15C). In contrast, there is no significant Rimiducid effect observed in iRC9 gene modified NK group (FIG. 15C). These observations indicated that basal iMC signal by itself increases NK proliferation, further enhancement would be achieved by dimerization of iMC molecule controlled by Rimiducid (Rim). Additionally, the expression level of iRC9 or iRC9.iMC were similar. The mean iRC9 NK transduction efficiency was 67.8% (range, 37.2-93.2%; n=9), statistically not different from the iRC9.iMC NKs (mean, 73.7%; range, 50.3-93.6%; n=9), at day 14 of culture as determined by hCD19+percentage.

Next it was investigated whether iMC modified NKs would have improved persistence in vivo. Here, human IL-15 gene was incorporated by a T2A sequence to generate a tricistronic vector iRC9.IL15.iMC—ectopically producing IL-15 to support NK survival and proliferation. All gene modified NKs were label with an eGFP-firefly lucifease fusion protein (eGFPFfluc) by a retroviral vector for tracking in vivo (FIG. 15D). The bioluminescent imaging (BLI) signal rapidly decreased in NOD.Cg-Prkdc^{scid}II2rg^{tm1Wjl}/SzJ (NSG) mice injected with iRC9 modified NKs, as well as iRC9.iMC modified NKs, supporting the hypothesis that IL-15 is indispensable for survival of NKs. Although mouse and human IL-15 share 65% amino acid sequence identity, there is poor interspecies cross-reactivity. Strikingly, rimiducid activation of iRC9.iMC modified NKs by rimiducid in association with IL-15 expression dramatically increased NK persistence and proliferation (FIGS. 15D and 15E) for at least one month without any tumor targets stimulation. Especially, mouse 2 had 31.9-fold BLI signal enhancement on day 41 post-injection, and mouse 4 had 18.9-fold BLI increase on day 27 post-injection. Moreover, iMC modified NKs proliferated further in response to THP-1 tumor cell targets (FIG. 15F), similar to a feeder cell effect. Rimiducid further boosted iRC9.IL15.iMC modified NK proliferation. Surprisingly, rimiducid activation markedly increased iRC9.iMC modified NKs proliferation in the absence of IL-15 beginning since day 33 post-injection (p<0.01 iRC9.iMC rimiducid group vs vehicle group). These provide strong evidence to support that iMC itself would significantly improve NK cell proliferation and persistence in vivo.

### iMC Enhances NK Cell Cytotoxicity Against Tumor Targets Upon Rimiducid Activation

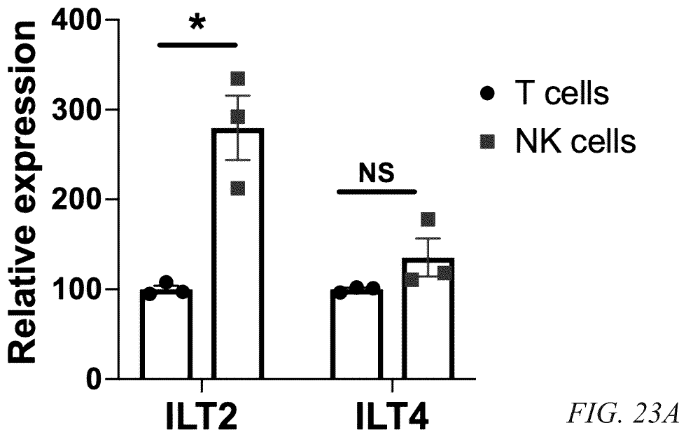

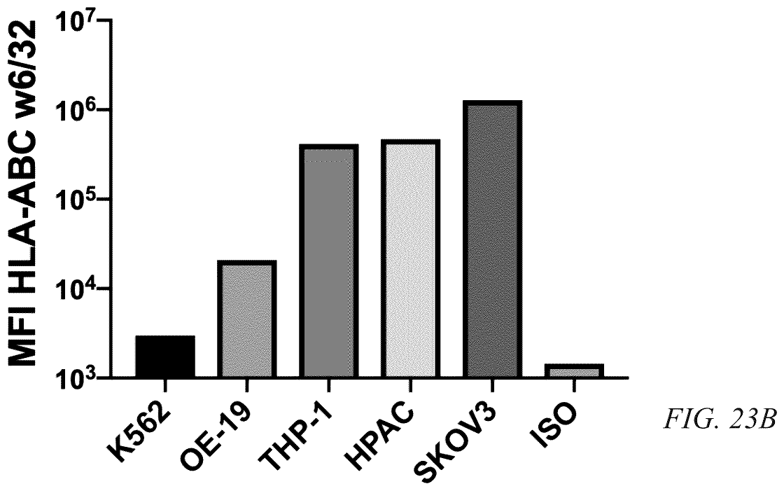

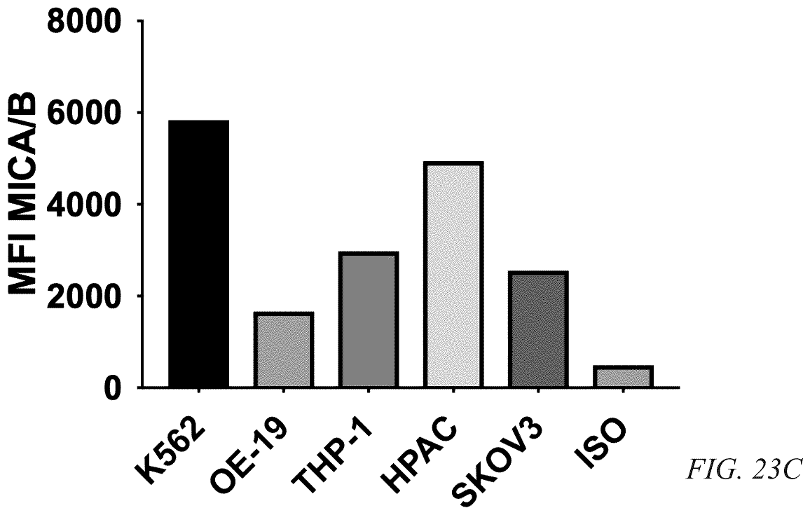

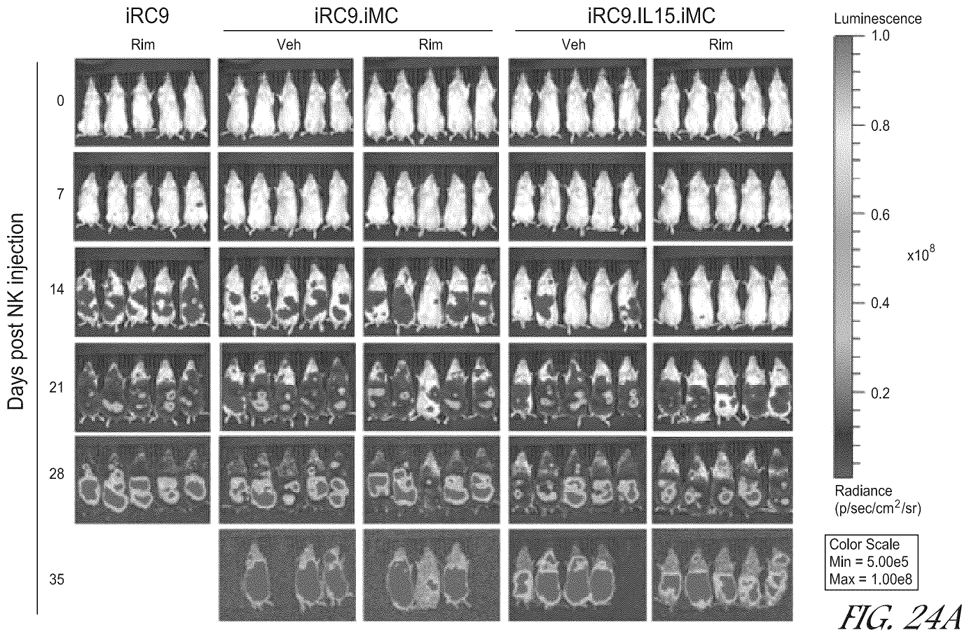

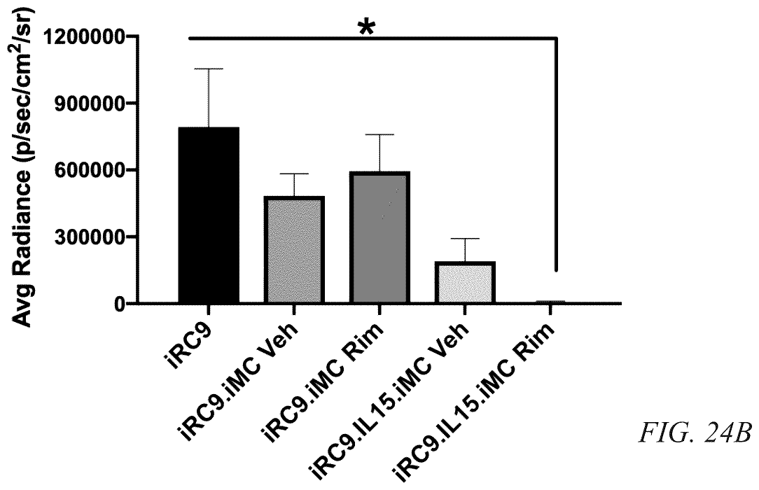

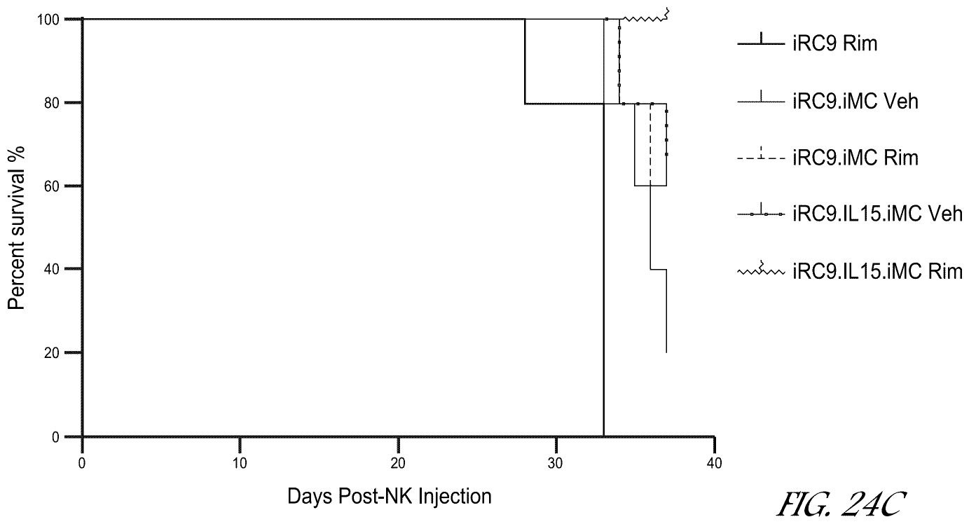

The effects of iMC on NKs function were tested. iRC9 or iRC9.iMC gene modified NK cells were exposed to eGFPFfluc modified HPAC or THP-1 tumor targets. Specific firefly luciferase activity was measured after 24 hrs. At all effector to target (E:T) ratios tested, iRC9.iMC modified NK cells had greater killing of HAPC (a NK resistant pancreatic adenocarcinoma cell line; high MHC Class I level, FIGS. 23A-23C), compared with control iRC9 modified NKs. Rimiducid treatment further augmented tumor cell killing (FIG. 24A). For a NK sensitive AML cell line THP-1 (low MICA/B expression, FIGS. 23A-23C) basal iMC was sufficient to augment cytotoxicty against tumor cells markedly. While only further dilution of iRC9.iMC modified NKs (1:4, p=0.002; 1:8, p=0.026; iRC9.iMC vs iRC9.iMC Rim) showed rimiducid-depended killing (FIG. 24B). This observation was confirmed by flow cytometry analysis of 48 hr co-culture (FIG. 24C, 24D). The percentage of THP-1 tumor cells, identified as GFP+ population, greatly decreased when comparing iRC9.iMC or iRC9.IL15.iMC with non-transduced (NT) or iRC9 modified NKs. It was noted that iRC9.IL15.iMC exhibits similar rimiducid-dependent target killing effects as iRC9.iMC except the killing capacity is slight reduced. Without being bound by any theory, one possible explanation for this result is that there is a lower level of iMC expression in iRC9.IL15.iMC modified NK cells (Data not shown). Comparable rimiducid-dependent killing of target cells by iMC modified NK cells was also observed in OE19 and K562 (data not shown). These data strongly suggested that the inducible iMC signal could be used to augment NK cytotoxicity against a variety of tumor cell targets-both NK sensitive and resistant.

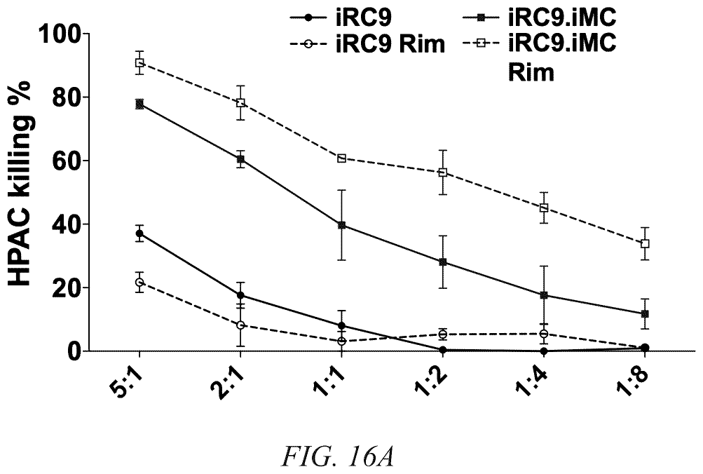

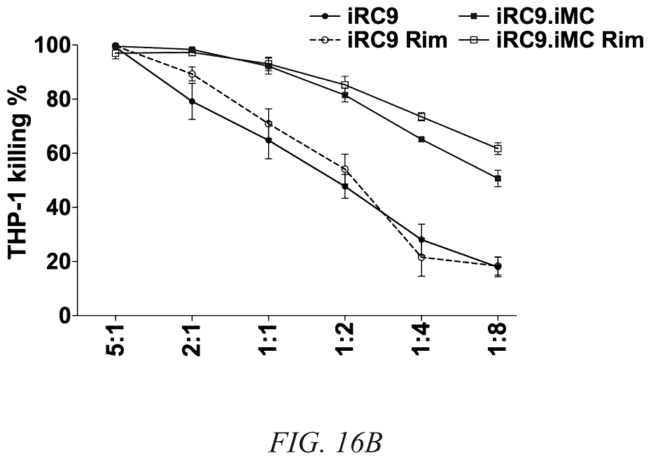

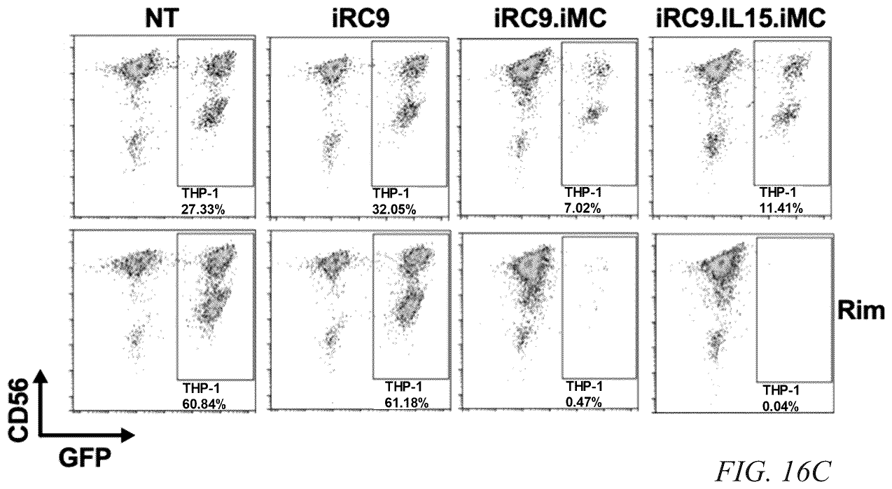

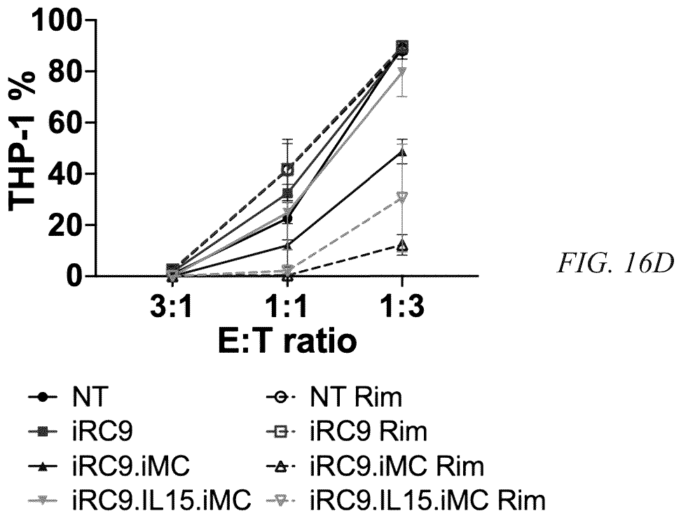

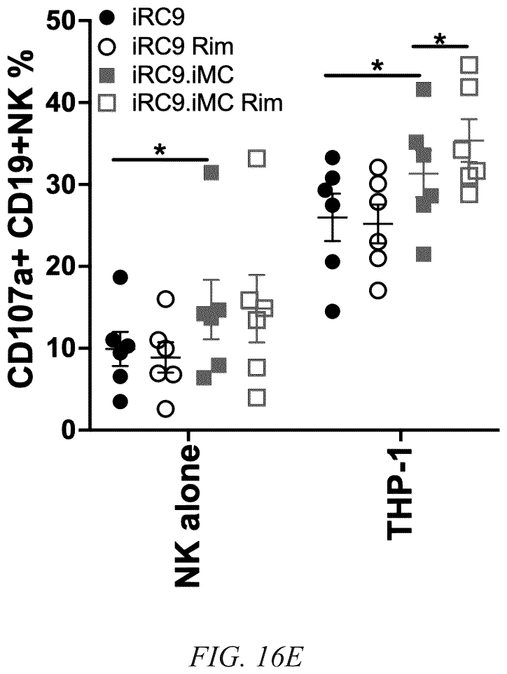

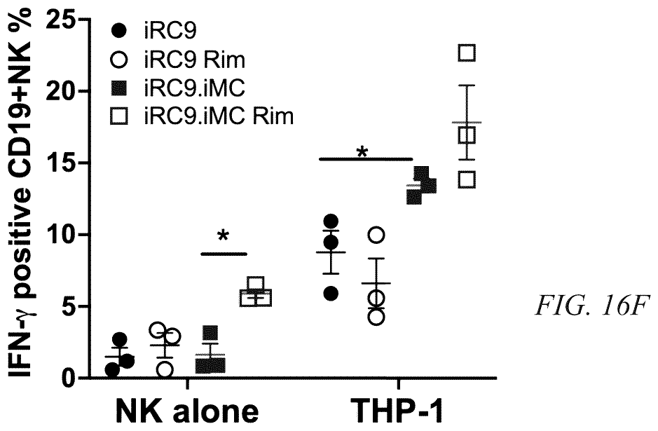

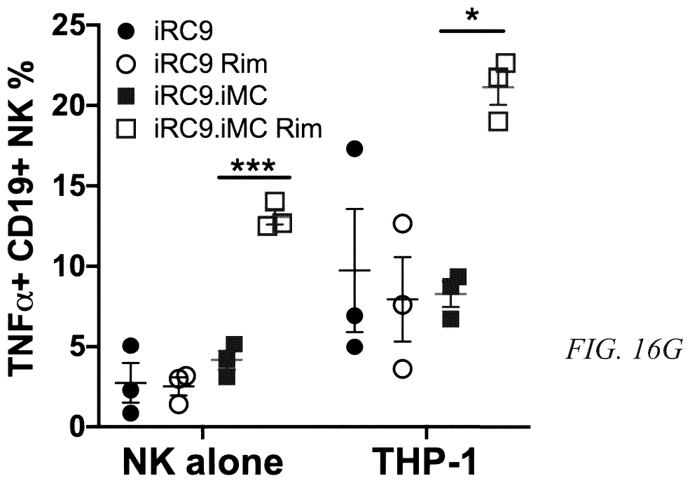

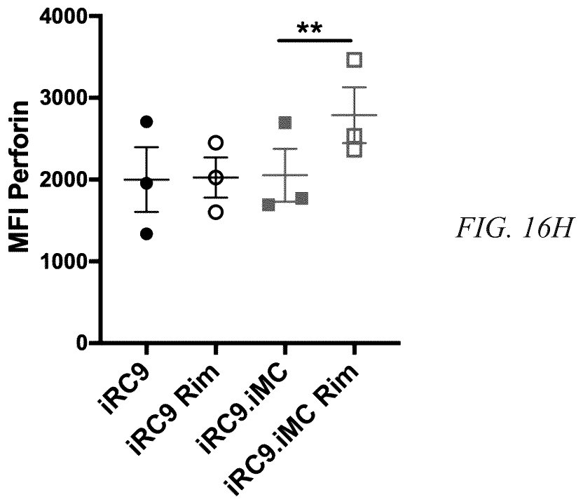

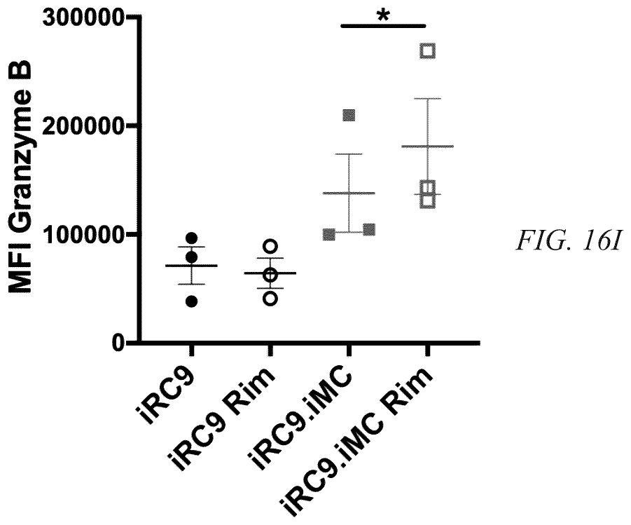

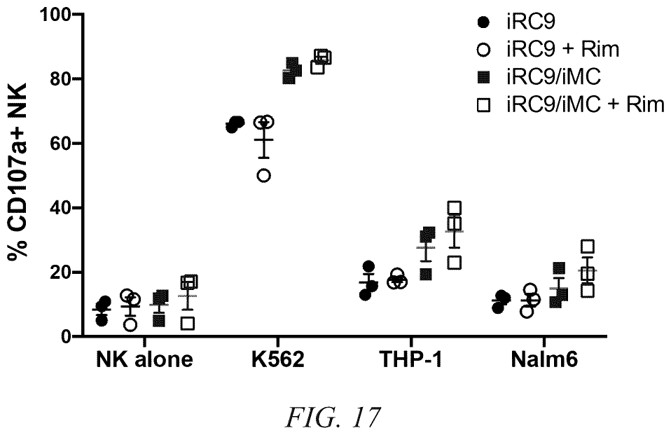

The most prominent feature of NK cells is their cytolytic activity. NK cells inject cytotoxic granules to their target. This process is termed degranulation and is marked by the expression of CD107a on the cell surface of NK cells. Therefore, the CD107a surface expression, a marker of degranulation, was examined. When cultured alone, a low proportion of transduced NK cells have surface expression of CD107a. Co-culture of transduced cells with the tumor targets K562, THP1 or Nalm6 increases the proportion of NK cells that degranulate. This proportion is elevated in iMC-expressing cells, indicating that even tonic iMC signaling is sufficient to sensitize the NK cells to respond to tumors. iRC9.iMC modified NK cells showed an increased percentage of surface CD107a+ population compared with iRC9 modified NK cells in response to THP-1 tumor targets; and these effects were further enhanced by rimiducid treatment (FIG. 16E). Basal CD107a surface expression was upregulated in iRC9.iMC group in the absence of tumor targets (FIG. 16E). The observations were likewise perceived in responses to other tumor targets k562 and Nalm6 (data not shown; FIG. 17), suggesting that iMC increases NK cells' secretion of cytolytic granules.

IFN-γ (FIG. 16F) and TNF-α (FIG. 16G) production were next evaluated. IMC with rimiducid activation markedly enhanced NK cells' producing ability of these effector cytokines.

One mechanism used by NK cells to kill target cells is the direct secretion of cytotoxic granules that include the protease granzyme B through pores formed by the protein perforin. Perforin (FIG. 16H) and granzyme B (FIG. 16I) production in responses to THP-1 targets were assesse by intracellular staining. As expected, rimiducid activated iRC9.iMC modified NK cells showed significant increased levels of granzyme B and perforin within the NK cells, which correlates with the enhancement of NK cell cytotoxicity with iMC activity. Interestingly, granzyme B but not perforin had a slightly upregulated basal production in iRC9.iMC modified NKs, compared with iRC9 modified NK cells, although it was not statistically significant (FIG. 16H, 16I). Taken together, these data suggest that iMC with rimiducid stimulation resulted in actively enhanced production of effector granules (perforin and granzyme B) and cytokines (IFN-γ and TNF-α) in response to tumor cell targets, which correlates with increased NK cell cytotoxicity with iMC rimiducid activation.

### Phenotypic and Functional Profiling of iMC Modified NKs Cells

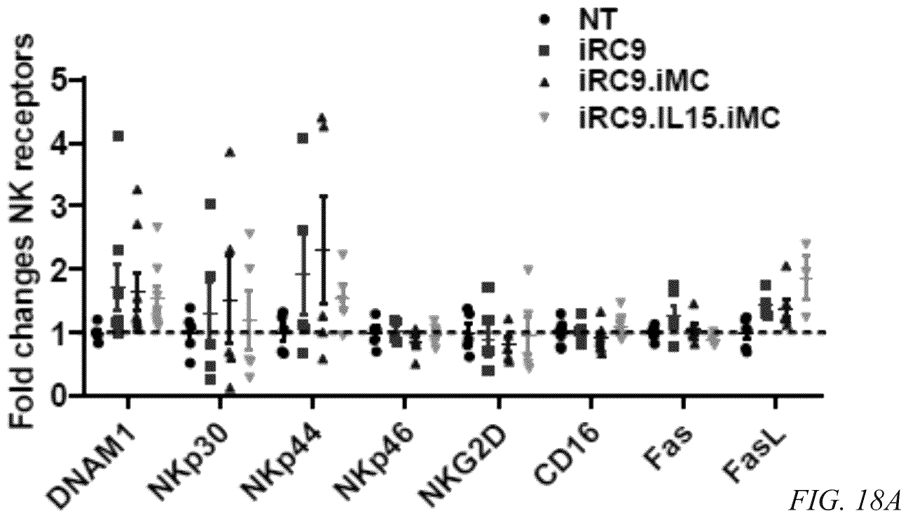

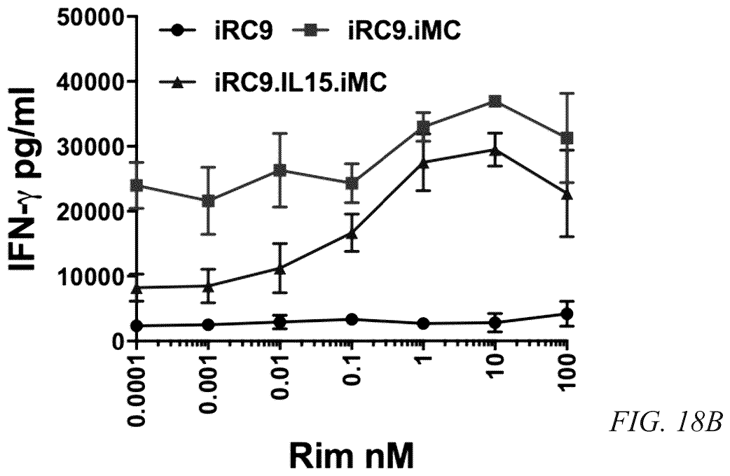

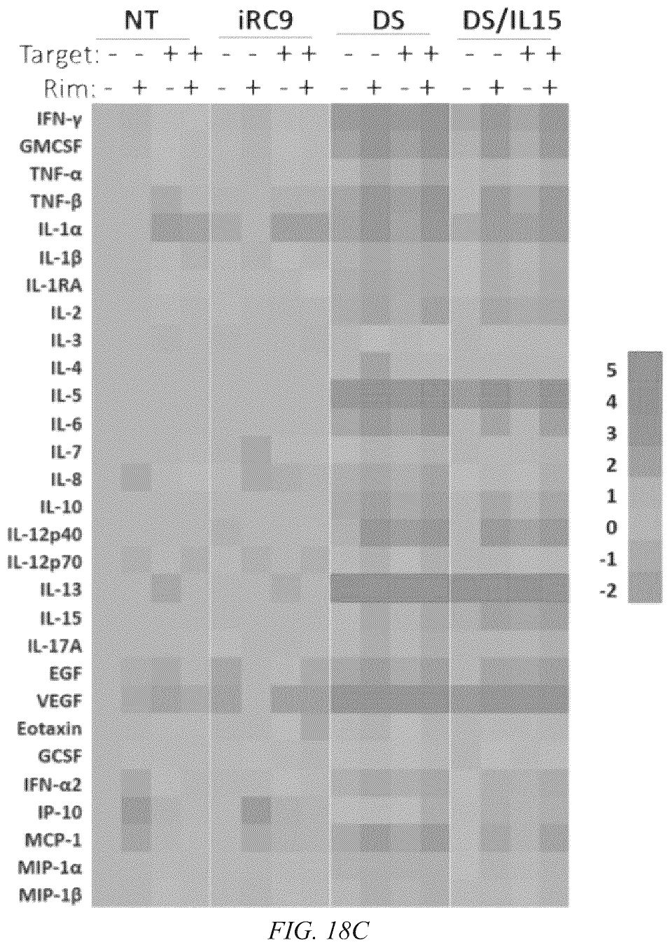

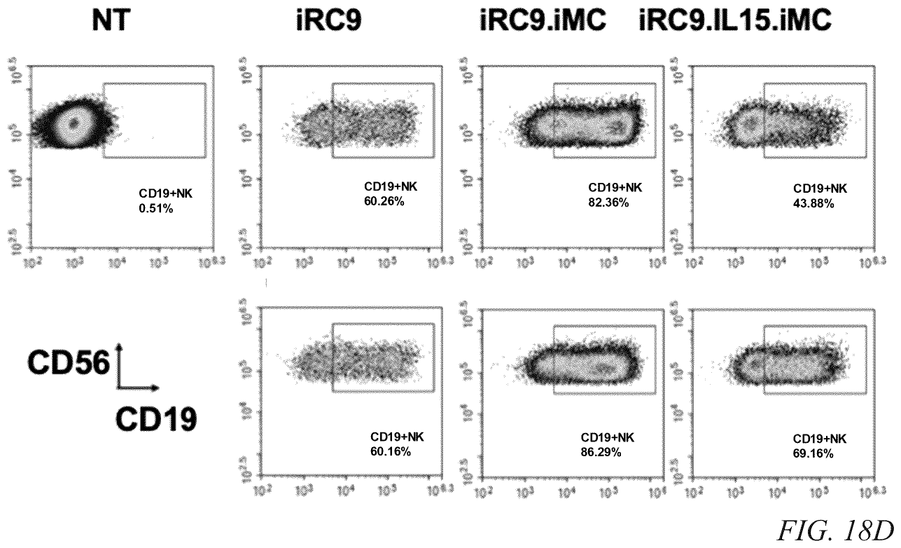

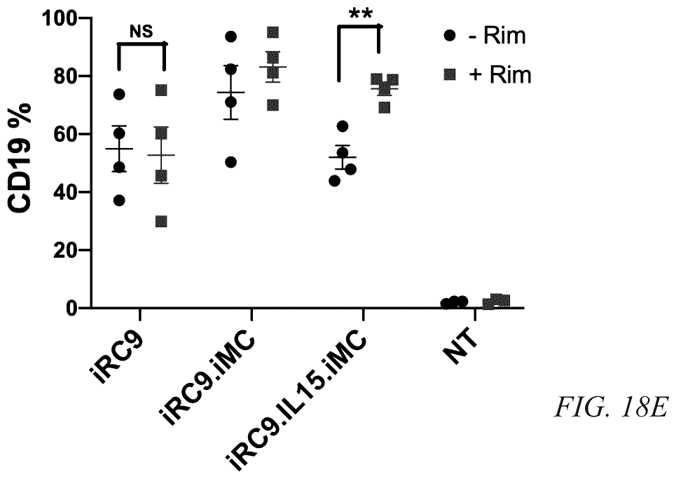

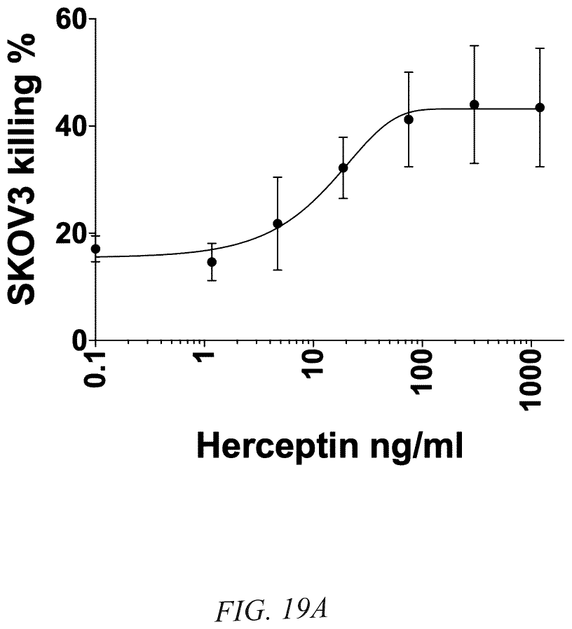

NK cell activating receptors are essential for NK cell activation and recognition of targets. Upon infection or malignant transformation, these ligands typically upregulate and activate NK cells for specific attack and elimination of target cells. To explore the iMC effects on various NK receptors, mutiparameter flow cytometry was performed. iRC9.iMC or iRC9.IL15.iMC modified NKs actually exhibited a phenotype very similar to that of iRC9 modified NKs (FIG. 18A). Rimiducid administration also did not increase surface NK cell receptor expression (data not shown), indicating that iMC enhancement of NK cell cytotoxicity is unlikely due to better identification of tumor targets via up-regulation of NK cell activating receptors. Interestingly, compared with NT group, gene modified NK cells had increased DNAM1, natural cytotoxicity receptors (NCR) NKP30, NKP44, and FasL surface levels (FIG. 18A), possibly due to retroviral infection, adding a rationale for retroviral vector approach in NK cell-based therapy. Previous reports regarding the effects of IL-15 on NK receptors are controversial (Liu, E., et al., Cord blood NK cells engineered to express IL-15 and a CD19-targeted CAR show long-term persistence and potent antitumor activity. Leukemia, 2018. 32 (2): p. 520-531; Elpek, K. G., et al., Mature natural killer cells with phenotypic and functional alterations accumulate upon sustained stimulation with IL-15/IL-15Ralpha complexes. Proc Natl Acad Sci USA, 2010. 107 (50): p. 21647-52). The present study suggests that NK cells, at least when transduced with iMC display no signs of dysfunction or anergy with transgene level IL-15 presence.

iMC modified NK cells were further characterized in terms of cytokine production. IFN-γ is the major effector cytokine produced by NK cells and plays important roles in modulating the immune responses (Hammer, Q., T. Ruckert, and C. Romagnani, Natural killer cell specificity for viral infections. Nat Immunol, 2018. 19 (8): p. 800-808). Rimiducid administration dramatically increased IFN-γ production at the co-culture supernatant in a dose-dependent manner (FIG. 18B). With 1 nM rimiducid treatment, IFN-γ production of iRC9.iMC modified NK cells was 12.3+1.42 fold that of iRC9; iRC9.IL15.iMC modified NK cells had 10.3±2.83 fold IFN-γ productions compared with iRC9 modified NK cells. An extensive cytokine profiling analysis was perfomed using a multiplex assay. Among the 29 cytokines/chemokines tested, iRC9.iMC modified NK cells generally demonstrated more cytokine production compared with iRC9 modified or non-transduced NK cells in a rimiducid-dependent fashion, especially certain cytokines/chemokines had greater fold change including IFN-γ, GM-CSF, TNF-α, TNF-β, IL-5, IL-12p40, IL-13, EGF, VEGF, and MCP-1 (FIG. 18C). iRC9.IL15.iMC modified NK cells demonstrated a similar cytokine production pattern, but produced slightly lower amounts as compared to iRC9.iMC modified NK cells except for IL-15. IL-15 was undetectable in supernatants collected from non-transduced or iRC9 modified NK cells. iRC9.IL15.iMC modified NK cells produced moderate amounts of IL-15 (12.52±2.38 pg/ml.10⁶ cells); the transgene expression significantly increased with rimiducid activation (27.88±21.66 pg/ml.10⁶ cells), in keeping with the enhanced proliferation of NK cells in culture. Surprisingly, iRC9.iMC modified NK cells produced small amounts of IL-15 (9.97±1.23 pg/ml.10⁶ cells) with rimiducid activation, providing one explanation for the in vivo finding that iRC9.iMC with rimiducid activation and tumor target stimulation showed an NK cell expansion at 30 days post injection in the absence of external IL-15 support (FIG. 15F).

### Rimiducid Facilitates Selective Enrichment of iMC Gene Modified NK Cells

Interestingly, the transduction efficiency increased in iMC gene modified NK cells with rimiducidactivation (FIGS. 18D and E). Briefly, transduced or non-transduced NK cells were treated with or without 1 nM rimiducid together with irradiated k562 and IL-2 at day 9 after activation. At day 14 of culture, the transduction efficiency was determined by hCD19 surface staining, since a truncated version of hCD19 was incorporated as a marker in the constructs. CD19+ percentage did not change much in iRC9 modified NK cells; whereas the CD19+ rate of iRC9.IL 15.iMC modified NK cells increased dramatically from 52%±8.2%, to 75.7%±4.6% (p=0.002) upon 1 nM rimiducid administration, indicating that iMC gene modified NK cells outgrow the non-modified NK cells. Upregulation of transgene expression as the main reason can be excluded since short time treatment (1 or 2 days rimiducid treatment) did not result in significant CD19+ cell population changes (data not shown). This observation further supports that iMC activation by rimiducid leads to enhanced NK cell proliferation. CD19+ percentage also increased in iRC9.iMC modified NK cells (74.3%±18.5% to 83.1%±10.5%), but not reached statistical significance (FIG. 18E).

### Improving iMC Gene Modified NK Cell Cytotoxicity Via ADCC Effects

Next, the in vivo efficacy of iMC modified NK cells was evaluated in an established leukemia xenograft model in NSG mice. Animals were intravenously injected with two doses of 5×10⁶ NKs modified with iRC9, iRC9.iMC, or iRC9.IL15.iMC, at day 5 and day 17 post luciferase-expressing THP-1 cells (THP-1eGFPFfluc) infusion. Animals also received intraperitoneal either 1 mg/kg rimiducid or vehicle weekly. Leukemia development was monitored by in vivo BLI. Only iRC9.IL15.iMC modified NK cell group with rimiducid treatment showed significant tumor control compared with iRC9 modified NK cells (p=0.017) at day 14 post NK cell infusion (FIGS. 24A and 24B). However, the effects were not durable. Despite no significant difference of BLI signals at late time points, significant prolonged survival was observed (p<0.0001) in iRC9.IL15.iMC modified NK cells with rimiducid therapy FIG. 24C).

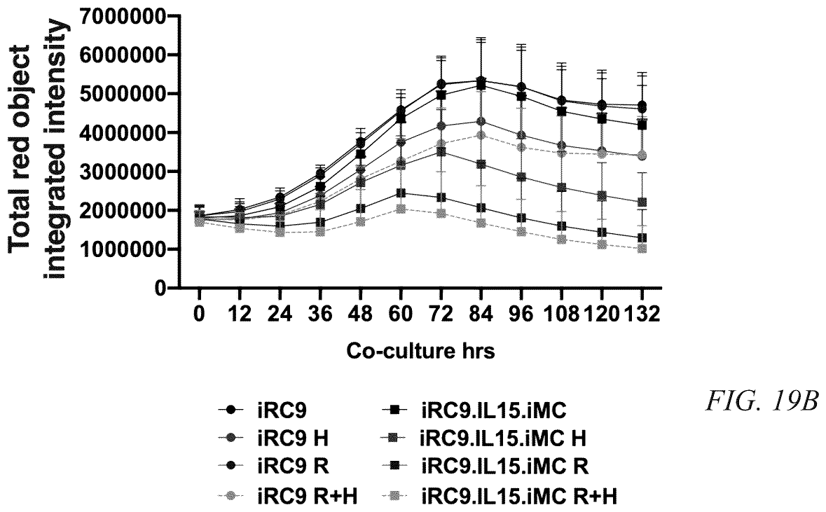

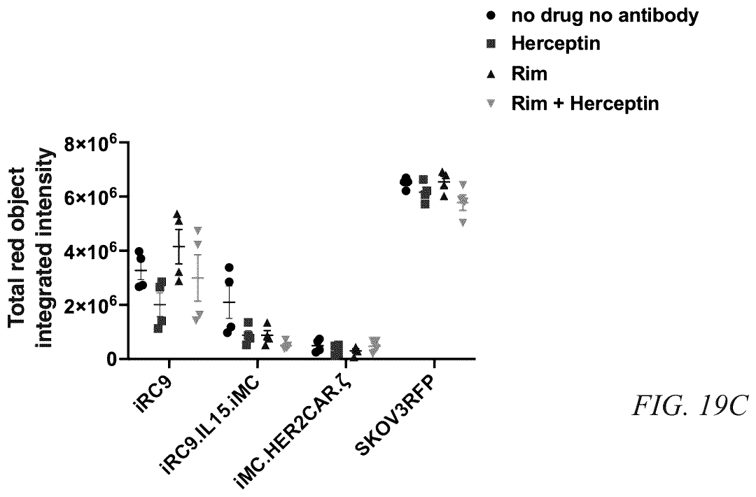

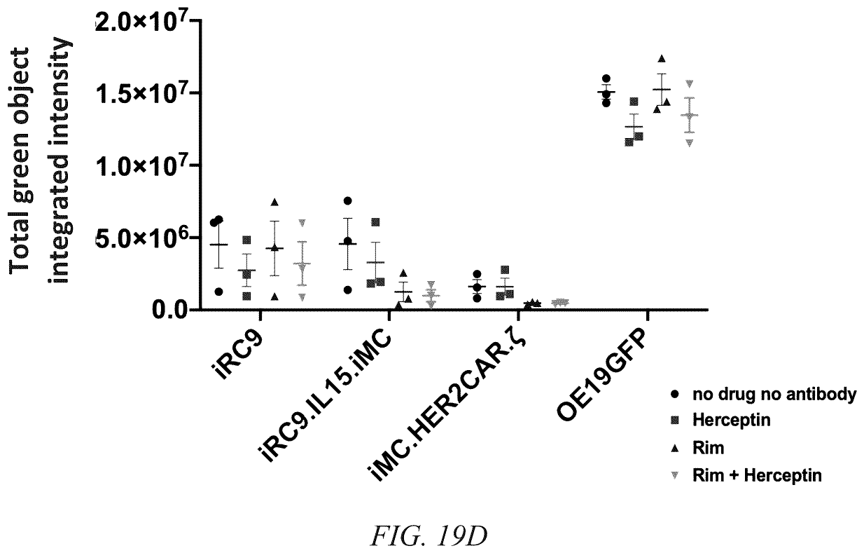

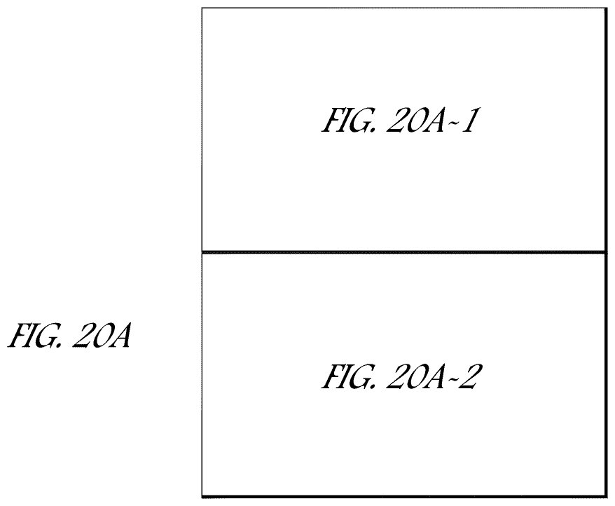

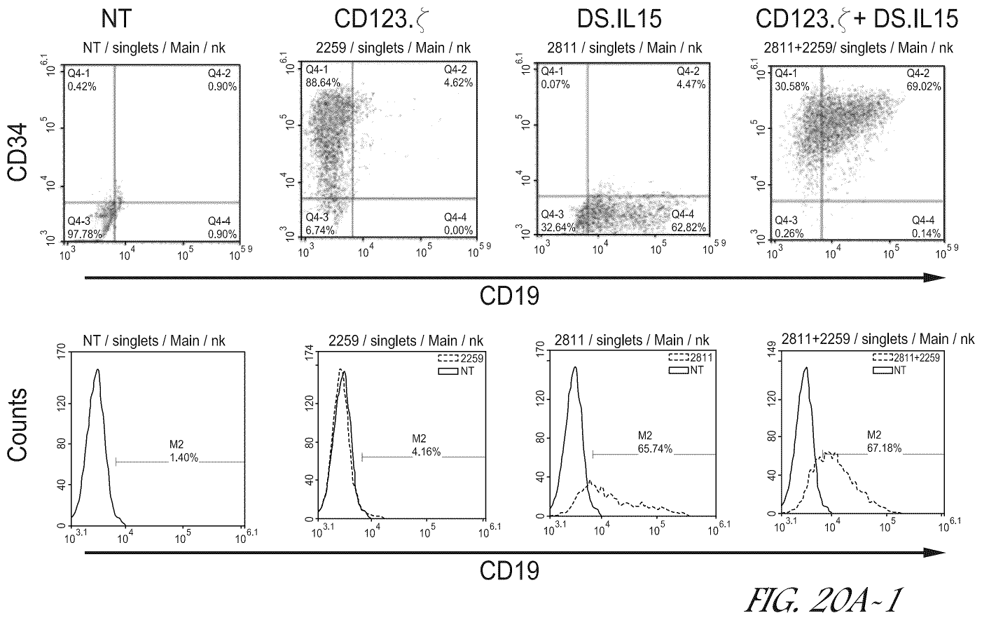

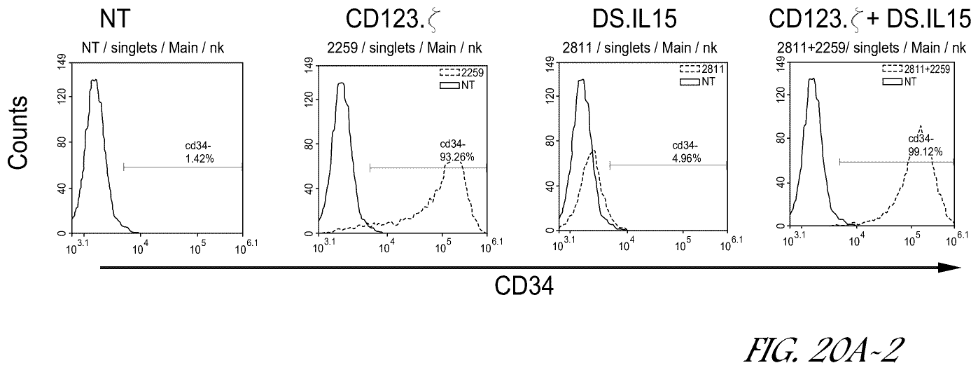

Antibody-dependent cell-mediated cytotoxicity (ADCC) is one of the mechanisms by which NK cells are involved in tumor cells killing. Therefore, the ability of Herceptin, a monoclonal antibody, to improve iMC modified NK cell cytotoxicity against human epidermal growth factor receptor 2-positive (HER2+) tumor cells was investigated. As shown in FIG. 19A, Herceptin indeed improved iRC9.IL15.iMC modified NK cell killing of SKOV3, a HER2+ ovarian adenocarcinoma cell line (High MHC class I expression, FIGS. 23B-23C), in a dose dependent manner (FIG. 19A). Even in iRC9 modified NK cells, Herceptin improved NK cell-mediated tumor target killing. However, Herceptin enhancement was moderate compared with rimiducid activation (FIGS. 19B, 19C, and 19D). There were little synergistic effects of Herceptin Rimiducid combination treatments. Interestingly, here a bicistronic vector iMC.HER2CAR.ζ was tested. Expressing a single chain variable fragment (scFv) targeting HER2 on the NK cell surface dramatically improved SKOV3 killing compared with iRC9.IL15.iMC modified NK cells, in the presence or absence of rimiducid (FIG. 19C). A similar trend was also observed in an NK sensitive gastric adenocarcinoma cell line (MHC class I expression low, MICA/B expression low, FIGS. 23A-23B) OE19 (FIG. 19D). Adding Herceptin did not affect iMC.HER2CAR.ζ cytotoxicity against SKOV3 or OE19 (FIGS. 19C and 19D). Taken together, these data indicate that expressing tumor-specific chimeric antigen receptors (CAR) in NK cells might be a good strategy for efficient and selective killing of cancer cells expressing the respective target antigen on cell surface.

Augmentation of iMC Modified NK Tumor Controlling Efficacy with Antigen Specific CAR

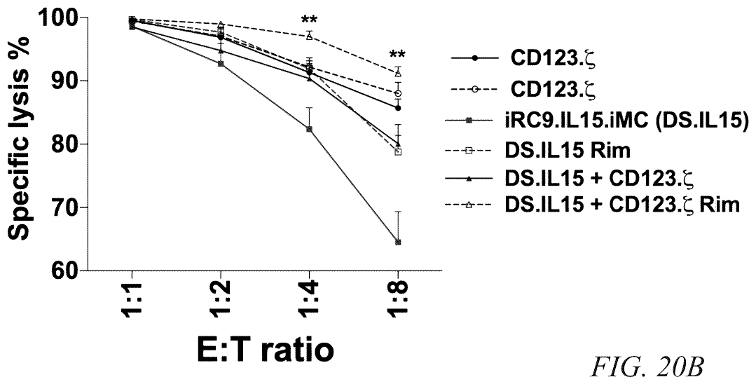

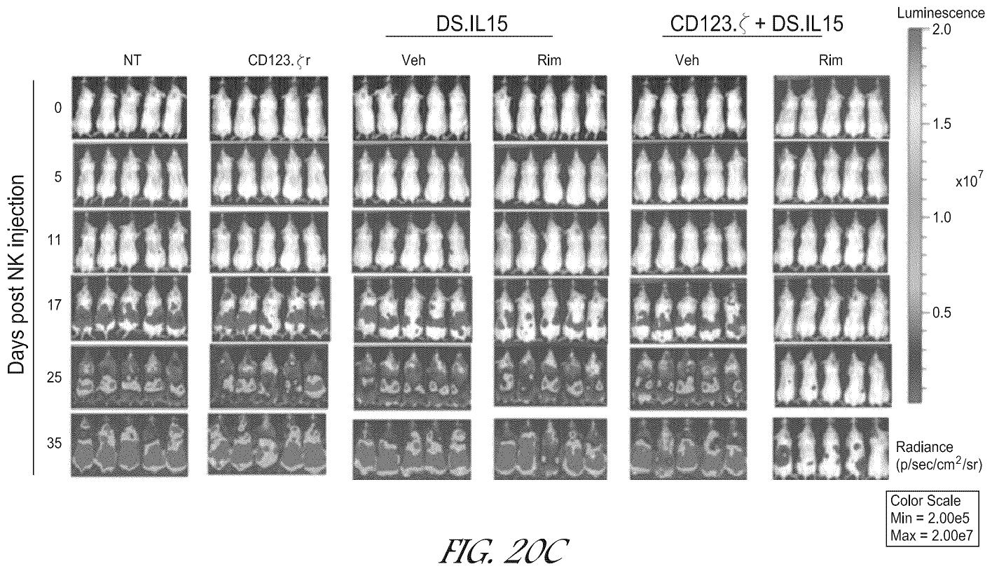

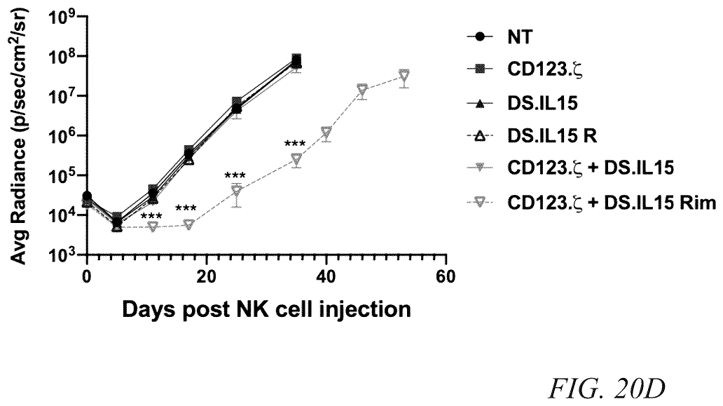

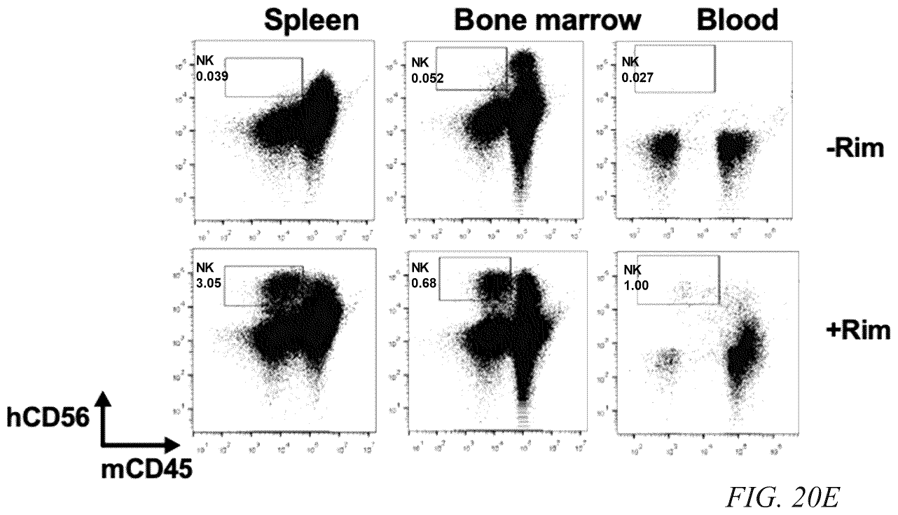

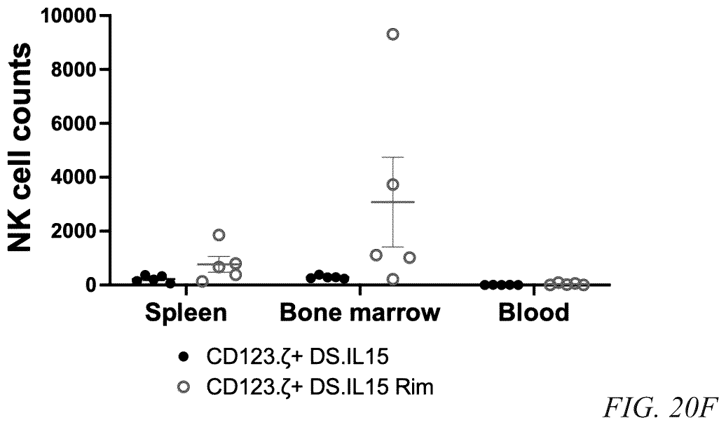

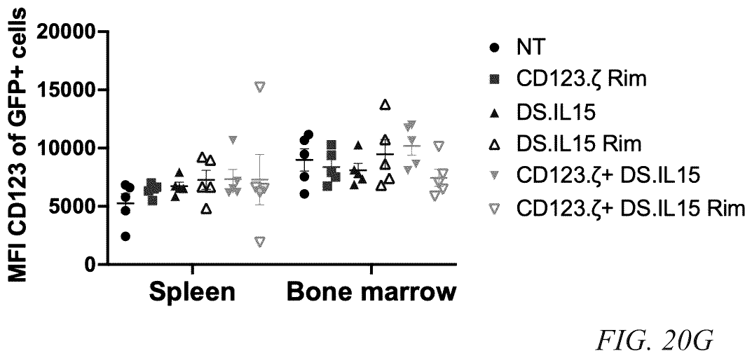

Next it was evaluated whether adding a CAR would improve the tumor controlling efficacy of iMC modified NK cells. CARs comprise a fusion of an extracellular targeting domain and an intracellular signaling domain to direct an effector cell specifically to a target cell expressing a cell surface marker expressed on the cell surface. The targeting domain is most commonly a single chain variable fragment (scFV) derived from an antibody specific for the expressed target antigen. The signaling domain is commonly derived from the T cell receptor (TCR) zeta (2) chain in a ‘first generation’ CAR. Second generation CARs contain an additional signaling moiety designed to enhance the proliferation and survival of cells expressing the CAR. NK cells were transduced with retroviral vectors encoding a first-generation CAR targeting CD123 (BP2259) alone, or co-transduced with BP2811 encoding iRC9 (FRB-FKBP12-Caspase-9), IL-15, the ΔCD19 marker, and iMC (FKBP12v36-FKBP12v36-MyD88-CD40), referred below as Dual switch. IL15 or dual-switch (DS) NK cells. NK cells were also transduced with BP2811 alone. Transgene expression of CD123 CAR was accesses by hCD34, whereas expression of iRC9.IL15.iMC (DS.IL15) was determined by hCD19 staining (FIG. 20A). Transduction with CD123.ζ retrovirus was highly efficient (>90%), comparable to that of double transduction. Dual switch. IL15 transgene expression (˜65%) was also similar between single and double transductions (FIG. 20A).

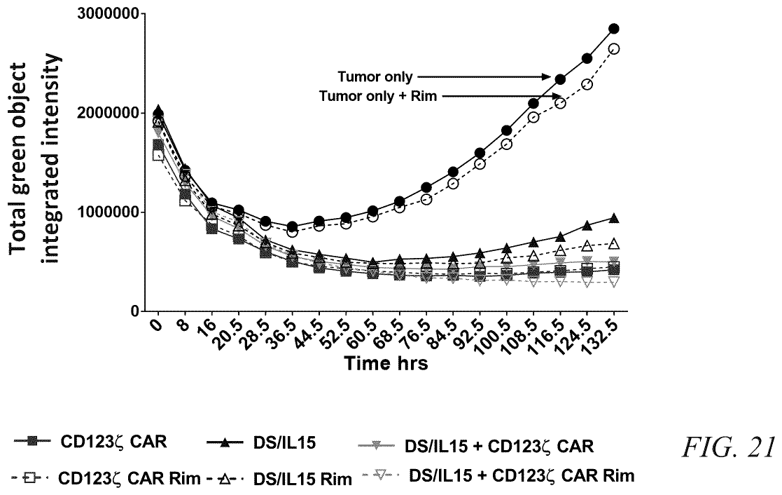

Transduced healthy donor NK cells were expanded in IL-2 and were co-cultured with CD123-expressing THP1 target cells engineered to express the marker protein GFP-ffluc with which cell growth can be measured by green fluorescence or luciferase activity. The co-cultures were performed at decreasing effector (the NK cells) to target (THP1-GFPffluc) ratios (E:T ratios). After 24 hours of drug treatment the amount of luciferase activity remaining in the GFP-ffluc target cells relative to untreated controls was an indirect assay for NK cell-directed cell killing. The results are displayed in FIG. 20B. At low E:T ratios in the absence of iMC activation by rimiducid, dual-switch (DS) NK cells displayed modest cytotoxicity that increased with rimiducid treatment. Coexpression of the CD123 CAR increased cytotoxicity of cells containing the dual-switch construct and this cytotoxicity was also elevated by incubation of rimiducid to activate iMC. Expression of the CD123CAR alone also exhibited substantial cytotoxicity, but this was not stimulated by rimiducid treatment because of the absence of iMC. Similar results were observed when target cell growth over six days of co-culture was examined in an Incucyte incubator/microscope (FIG. 21). The expansion of THP-1 target cells was controlled substantially by incubation with dual-switch NK cells without the CD123 CAR and this tumor cell control was enhanced by treatment with rimiducid or by co-expression of the CD123CAR.

Control of THP1 tumor expansion was also examined in a mouse xenograft model. Immunosuppressed NOD-Scid-IL2RY/(NSG) mice were engrafted intravenously (i.v.) with 10⁷ human NKs that were non-transduced (NT) or transduced with retrovirus encoding CD123ζ CAR, DS (iMC+iRC9)/IL15, or DS (iMC+iRC9)/IL15+CD123ζ CAR; 3 days following i.v. implantation of 10⁶ THP-1.GFPffluc tumor cells. Rimiducid (1 mg/kg/week) or vehicle was administered i.p. weekly. Tumor expansion was measured weekly by bioluminescent intensity (BLI) of a D-luciferin substrate reactive with the GFPffluciferase expressed in the THP1 cells in an IVIS imager (Perkin-Elmer). The results are displayed in FIGS. 20C and 20D. Controlled, or reduced expansion of the tumor cells was observed only in the mice that had been engrafted with NK cells expressing the dual-switch, IL-15, and CD123ζ CAR construct, where the mice were also treated with rimiducid. As observed with CD123.ζ+DS.IL15, NKs frequency and numbers were increased by rimiducid treatment, especially in spleen and bone marrow (FIGS. 20E and 20F). Given the tumor was not completely eliminated, CD123 expression on GFP+ cells were determined (FIG. 20G). No significant changes of CD123 expression ruled out selection of tumor cells antigen-loss, indicative of feasible multiple dose therapy for better efficacy.

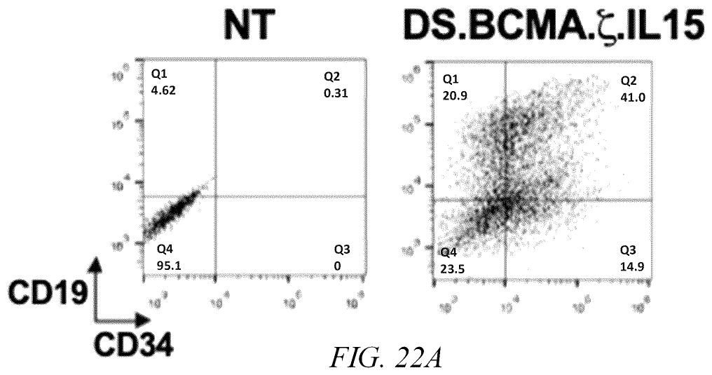

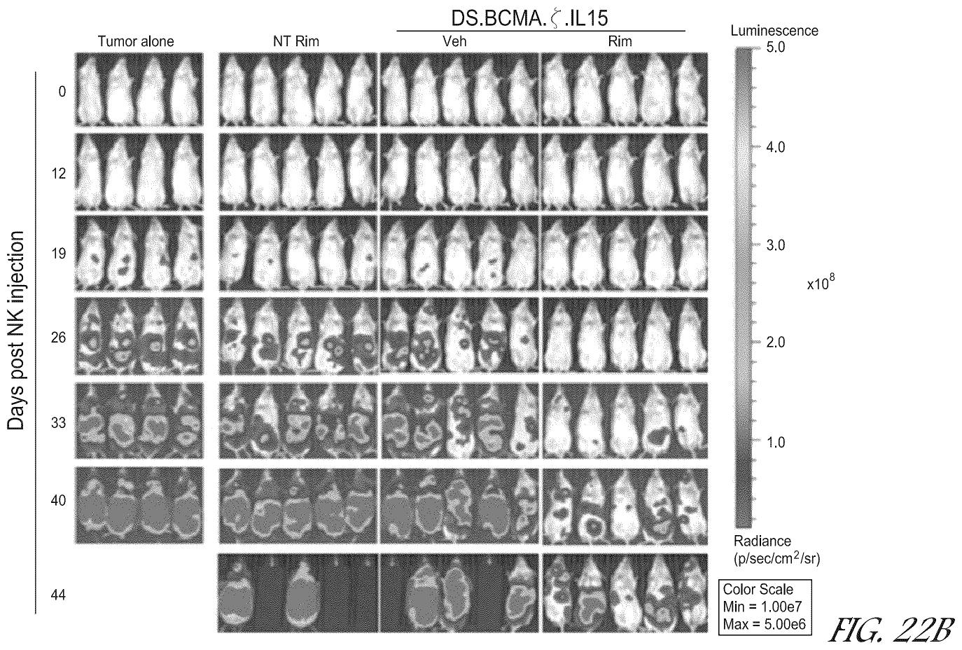

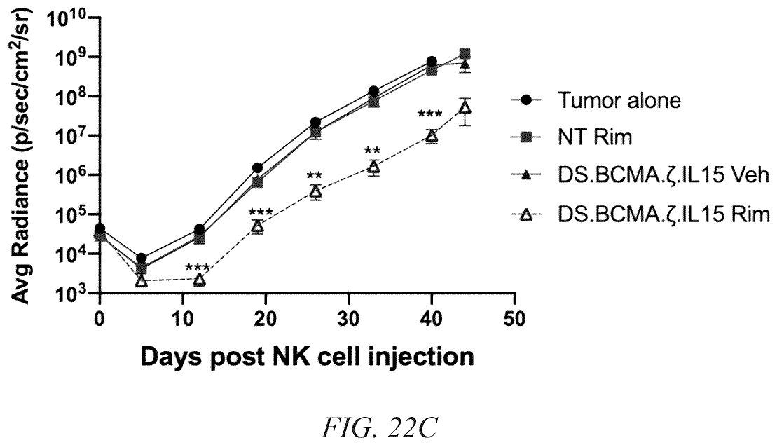

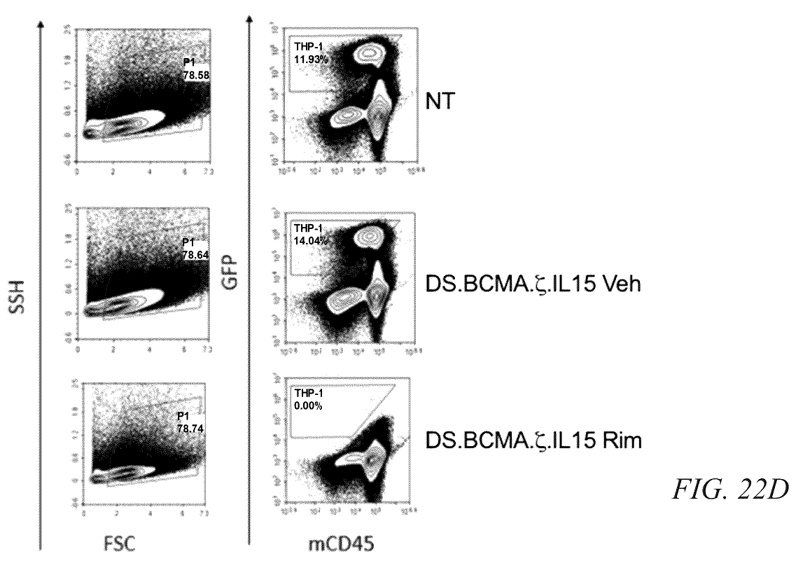

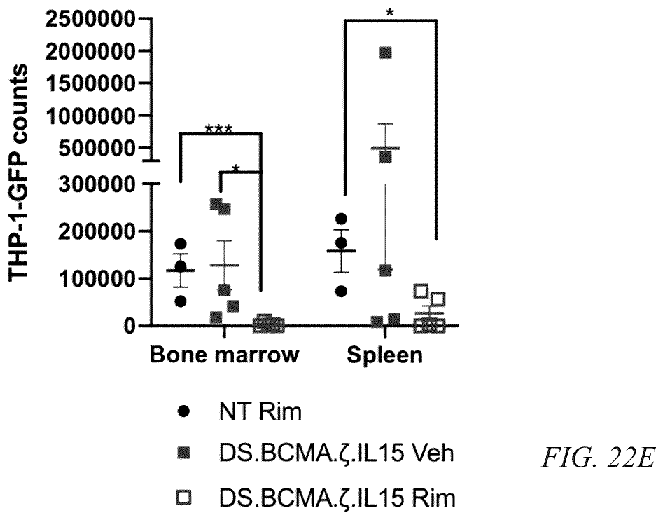

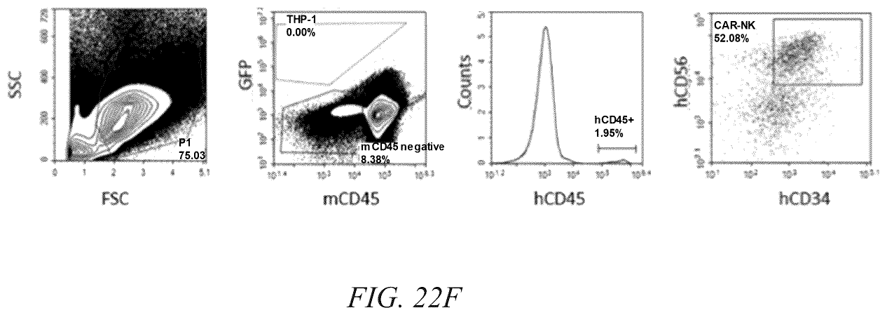

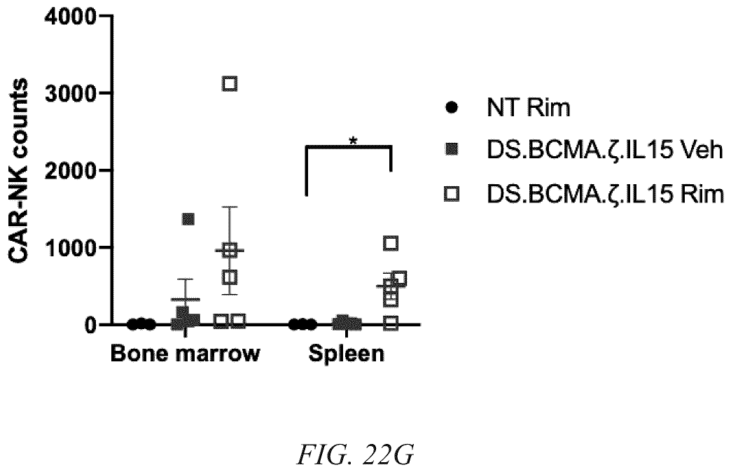

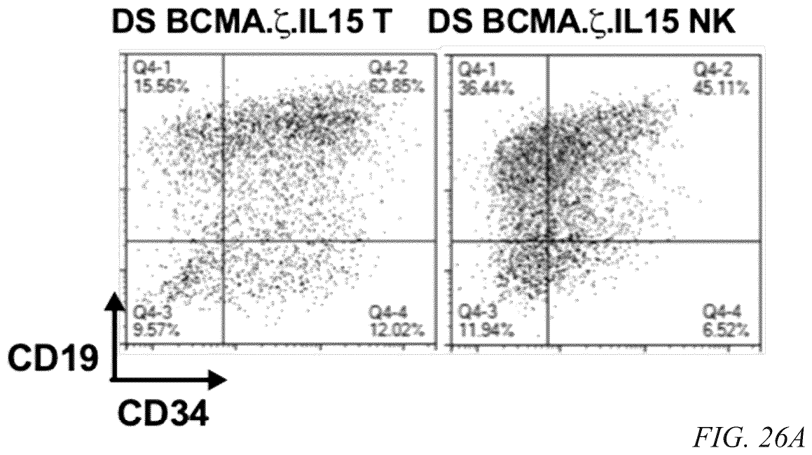

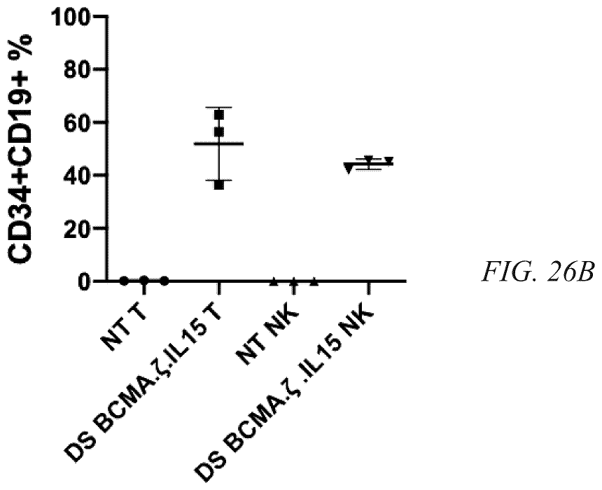

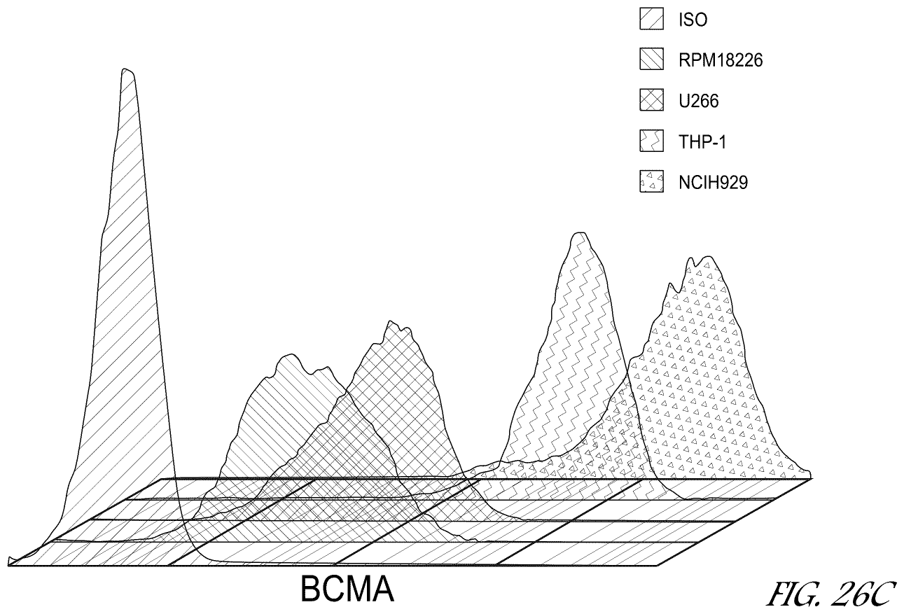

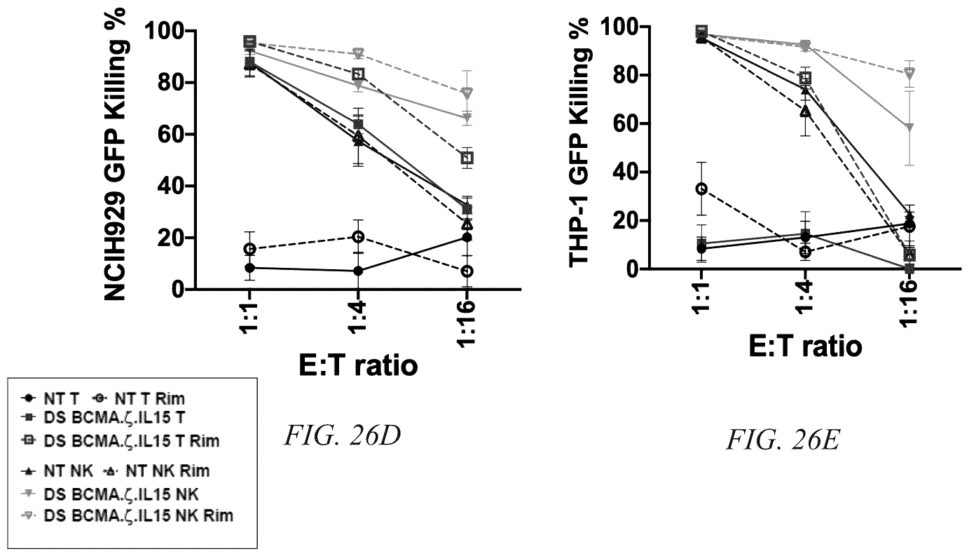

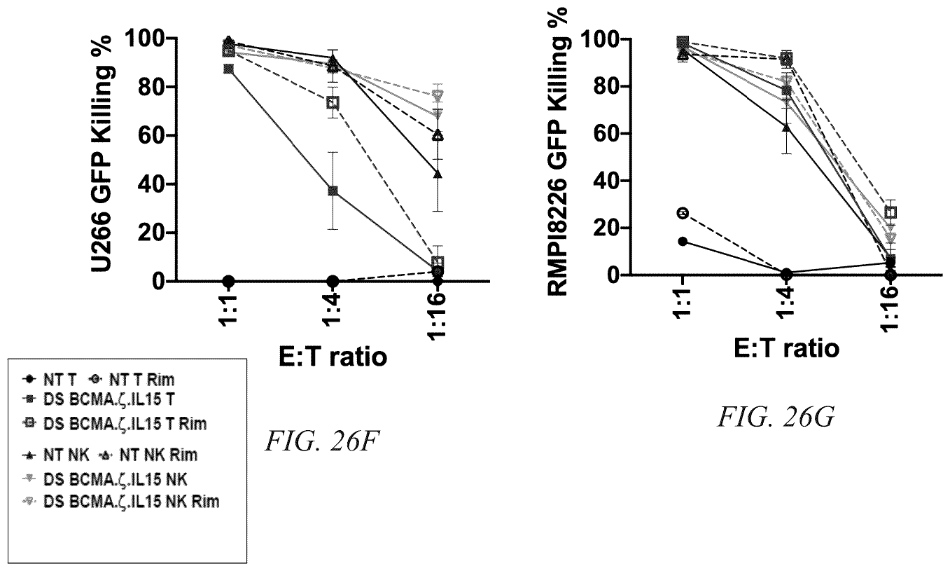

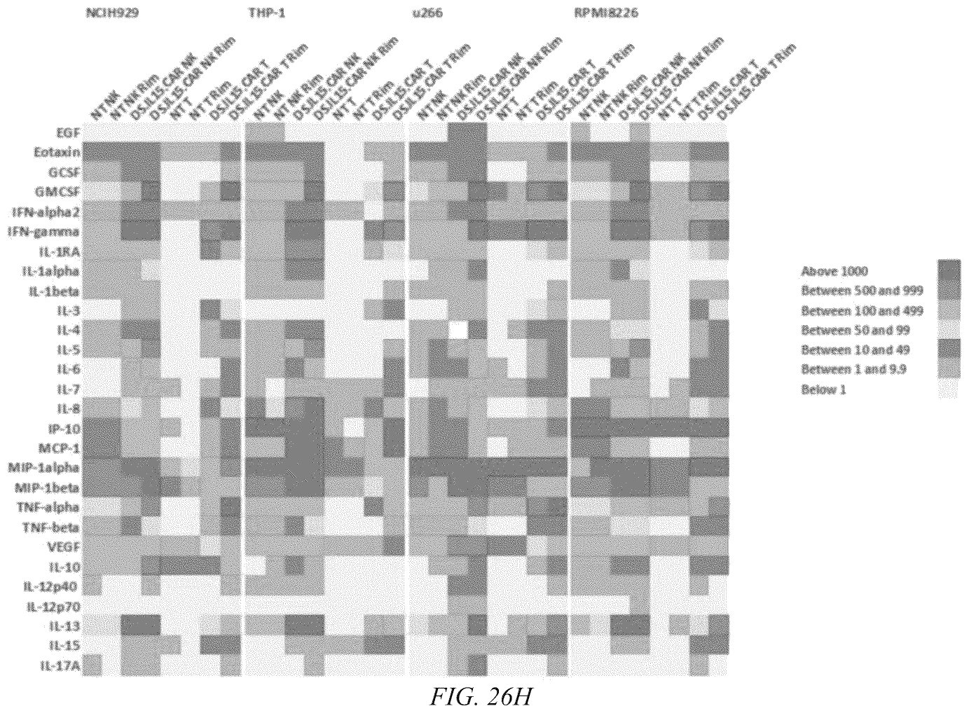

The efficacy of CAR-NK cells with iMC was also investigated in a different platform. THP-1 cells expressed high level of B cell maturation antigen (BCMA) on their surface (FIG. 26C). A tricistronic vector encoding iMC followed by a scFv targeting BCMA and IL15 (iMC.BCMA.ζ.IL15) was generated. Activated NK cells were first transduced with a retroviral vector encoding iMC.BCMA.ζ.IL15, followed by second transduction with iRC9 encoding retrovirus (Duong, M. T., et al., Two-Dimensional Regulation of CAR-T Cell Therapy with Orthogonal Switches. Mol Ther Oncolytics, 2019. 12: p. 124-137), to generate DS.BCMA.ζ.IL 15 modified NK cells with a transduction efficiency>40% for double positive cells (FIG. 22A). To determine the in vivo efficacy, THP-1eGFPFfluc bearing mice were intravenously injected with 107 NT or DS.BCMA.ζ.IL 15 modified NKs. Certain groups also received i.p. rimiducid (1 mg/kg). As shown in FIGS. 22B and 22C, rimiducid-dependent iMC activation was required for effective tumor controlling for 44 days post NKs injections. From day 40 to day 48, mice were euthanized, no or little frequency/numbers of GFP+tumor cells was detected in bone marrow and spleen of DS.BCMA.ζ.IL15 modified NK cell group with rimiducid, compared with the non-rimiducid-group or NT group (FIGS. 22D and 22E), consistent with efficient rimiducid-dependent control of tumor by DS.BCMA.ζ.IL 15 modified NK cells. Around day 48, higher frequencies/numbers of CAR-expressing NKs were identified in the bone marrow and spleens of mice treated with DS.BCMA.ζ.IL15 modified NKs with rimiducid (FIGS. 22F and 22G), suggesting proliferation and successful homing of gene modified NK cells to sites of disease in a rimiducid-dependent fashion.

### Control of iMC Enhanced NK Activity by an Inducible Safety Switch

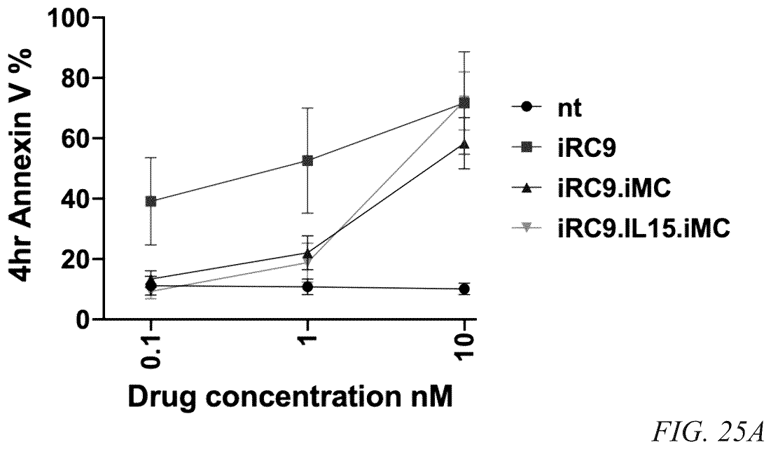

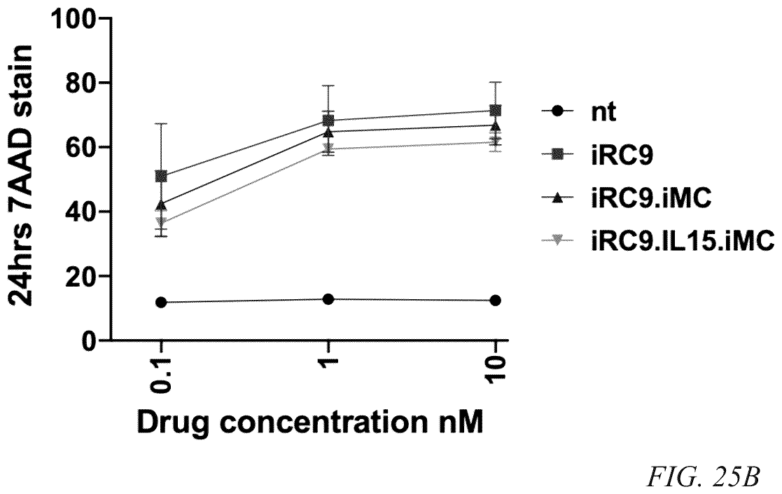

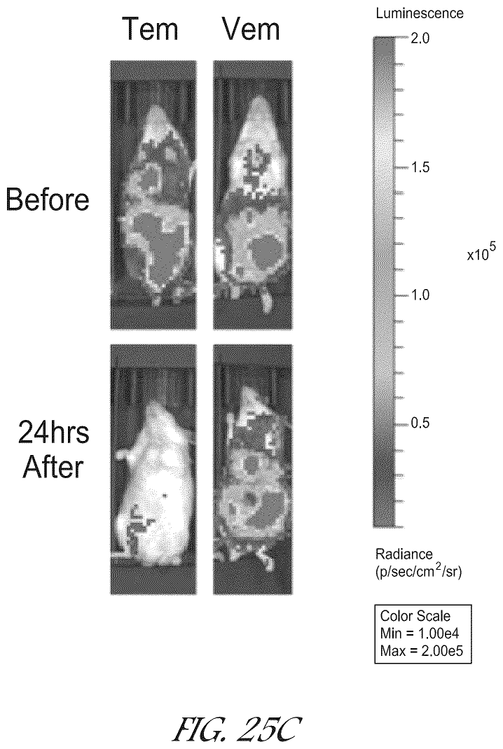

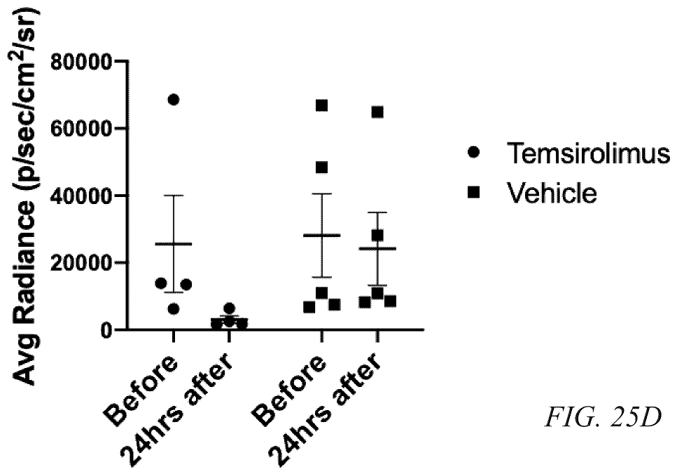

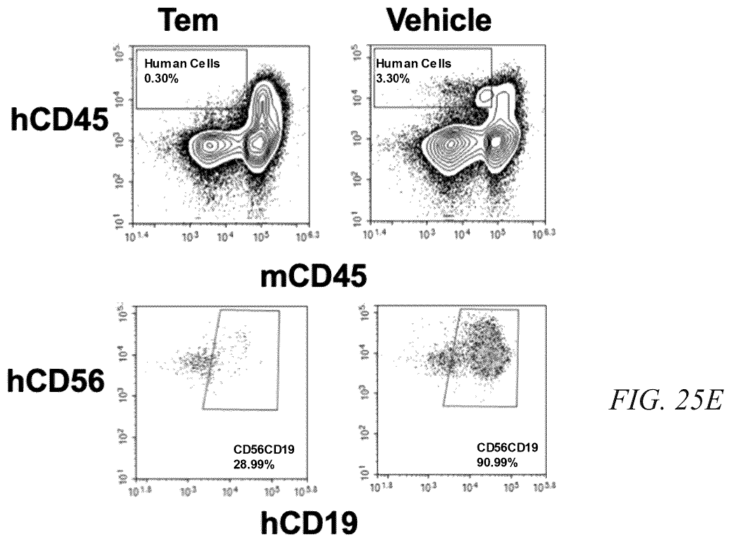

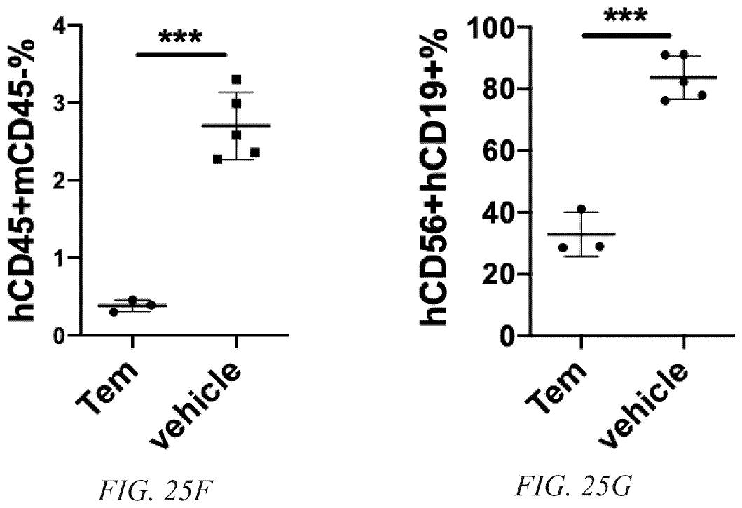

To prevent potential uncontrolled expansion of the gene modified NK cells, as well as associated cytokine release syndrome (CRS) and GvHD, a suicide gene iRC9 was incorporated into the constructs. The addition of 10 nM temsirolimus to cultures induced apoptosis as early as 4 hrs but had no effect on the viability of non-transduced NK cells (FIGS. 25A and 25B). The suicide gene was also effective in vivo. Mice engrafted with THP-1 tumor received iRC9.IL15.iMC-transduced NKs labeled with oNL renilla luciferase. Mice were then either treated with the dimerizer temsirolimus or vehicle control. Within 24 hrs, BLI signal dramatically reduced by temsirolimus but not vehicle control (FIGS. 25C and 25D). Frequency of gene modified NK cells was significantly reduced in the spleens of temsirolimus treated mice (FIGS. 25E, 25F, and 25G).

### Comparisons of DS.CAR-NK Vs DS.CAR-T Therapy in BCMA Expression Tumor Cells

CAR-T therapy against lymphoid malignancies has produced striking clinical results. Therefore, DS.BCMA.ζ.IL15 modified NK cell cytotoxicity, in comparisons with same donors DS.BCMA.ζ.IL 15 modified T cells was evaluated. T cells and NK cells showed equivalent transduction efficiencies (FIGS. 26A and 26B). Four plasmacytoma/leukemia cell lines were tested with BCMA antigen expression level ranging from high to moderate: NCIH929, THP-1, U266, and RPMI8226 (FIG. 26C). Untransduced or transduced NK or T cells were co-cultured with BCMA expressing tumor targets each at decreasing E:T ratios and tumor cell growth was monitored for 140 hours in an Incucyte microscope incubator. The fluorescence of the tumor cell co-cultures at 140 hours relative to that of the tumor cells incubated alone at 140 hours was measured and the degree of tumor control calculated as percentage of killing by T or NK cells. Non-transduced T cells (NT) displayed poor cytotoxicity in this assay, which was not improved with rimiducid treatment. Dual switch CAR-T cells had increased cytotoxicity relative to non-transduced T cells and this toxicity was enhanced by activation of iMC with rimiducid. Non-transduced NK cells exhibited innate anti-tumor cytotoxicity that was similar to that observed with dual switch-CAR-T cells. Interestingly, DS.BCMA.ζ.IL 15 modified NKs showed superior rimiducid-dependent killing compared with DS.BCMA.ζ.IL15 modified T cells in BCMA high antigen expression cell lines; whereas comparable or a little less effective in BCMA moderate antigen expression tumor cell lines U266 and RPMI8226 (FIGS. 26D, 26E, 26F, and 26G). Cytokine profiling data further demonstrated that DS.BCMA.ζ.IL15 modified NK cells generally produced more cytokines compared with T cells (FIG. 26H). These data indicate that NK cell-based therapy poses the potential for better efficacy than CAR-T therapy in vitro. Moreover, ILT2, the inhibitory receptors, expression was higher in NKs compared with T cells (but not ILT4, FIG. 23A), suggesting a safe feature of NK cells for less GVHD.

### iMC Blocks NK Cell Inefficacy Following Cryostorage

FIG. 13A shows that iMC blocks NK cell inefficacy following cryostorage. Transduced NK cells were maintained in standard culture conditions. Cells were slowly frozen in 90% foetal calf serum/10% dimethylsulfoxide and stored below −150° C. for up to four weeks. Overall NK cell viability after freeze-thaw was similar for each group. The graphs provided in FIGS. 13B and 13C relate to efficacy. Cell viability was poor in for each transductant immediately following thawing and replating but recovered over three days of standard culture. NK cells prior to and after the indicated period of recovery from freeze/thaw were cultured with THP1-luciferase targets for 24 hours at an E:T of 3:1 (FIG. 13B) or 1:1 (FIG. 13C) to assess killing efficacy. Dual switch NK cells with iMC were capable of regaining their potency over 48 to 72 hours after cryostorage while cells lacking iMC lacked efficacy.

### Example 4.—Expression of a Constitutively Active MyD88/CD40 with an Inducible Proapoptotic Safety Switch Confers Rimiducid Inducible Ablation of CAR-NK Expansion and Anti-Tumor Efficacy Against Hematological Malignancies

In certain applications, it is desirable to maintain the activity of MyD88-CD40 in a constitutive fashion to promote stable NK cell expansion and anti-tumor activity. A possible limitation of such a strategy is toxicity resulting from MC overactivity through excessive cytokine release or off tumor targeting. Retroviral constructs driving the expression of a first-generation chimeric antigen receptor (scFv-transmembrane liner-CD3ζ) and MyD88/CD40 separated by a P2A sequence engineered to have inefficient cotranslational cleavage provides expression of the first-generation CAR and a fusion protein between the CAR and MC that provides constitutive activation of MC at the cell membrane (Collinson-Pautz et al., Leukemia Feb. 28, 2019 (doi: 10.1038/s41375-019-0417-9)). Inclusion of inducibe caspase-9 in (FKBPv-ΔC9) the retroviral vector permits apoptotic cell ablation upon the addition of rimiducid. The present example demonstrates that constitutive activation through MC drives NK cell expansion, antitumor efficacy and cytokine production to a degree similar to that of inducible MC when activated by rimiducid but that activation of the iCaspase-9 safety switch blocks the activity of the NK cells.

### Materials and Methods

Methods for determination of NK cell proliferation, cytokine release into cell supernatant and control of tumor cell growth in coculture assays were as described in the previous examples. Additional plasmid material is described below.

### Plasmids:

TABLE
PBP2819-SFG-FKBPv.ΔCaspase9-T2A-αBCMA(C12.A3.2).CD8STM.zeta-P2A-
MyD88.CD40-T2A-IL15

| | | SEQ | | SEQ |
|---|---|---|---|---|
| Fragment | Nucleotide | ID NO: | Peptide | ID NO: |
| Leader peptide | ATGCTCGAGATGCTGGAG | 57 | MLEMLE | 58 |
| FKBPv | GGAGTGCAGGTGGAGACTATTAGCC | 113 | GVQVETISPGDGRTFPKRGQTCVVHY | 16 |
| | CCGGAGATGGCAGAACATTCCCCAA | | TGMLEDGKKVDSSRDRNKPFKFMLG | |
| | AAGAGGACAGACTTGCGTCGTGCAT | | KQEVIRGWEEGVAQMSVGQRAKLTIS | |
| | TATACTGGAATGCTGGAAGACGGCA | | PDYAYGATGHPGIIPPHATLVFDVELL | |
| | AGAAGGTGGACAGCAGCCGGGACC | | KLE | |
| | GAAACAAGCCCTTCAAGTTCATGCT | | | |
| | GGGGAAGCAGGAAGTGATCCGGGG | | | |
| | CTGGGAGGAAGGAGTCGCACAGAT | | | |
| | GTCAGTGGGACAGAGGGCCAAACT | | | |
| | GACTATTAGCCCAGACTACGCTTAT | | | |
| | GGAGCAACCGGCCACCCCGGGATC | | | |
| | ATTCCCCCTCATGCTACACTGGTCTT | | | |
| | CGATGTGGAGCTGCTGAAGCTGGAA | | | |
| Linker | AGCGGAGGAGGATCCGGAGTGGAC | 61 | SGGGSGVD | 62 |
| Δcaspase9 | GGGTTTGGAGATGTGGGAGCCCTG | 63 | GFGDVGALESLRGNADLAYILSMEPC | 64 |
| | GAATCCCTGCGGGGCAATGCCGATC | | GHCLIINNVNFCRESGLRTRTGSNIDC | |
| | TGGCTTACATCCTGTCTATGGAGCC | | EKLRRRFSSLHFMVEVKGDLTAKKMV | |
| | TTGCGGCCACTGTCTGATCATTAAC | | LALLELARQDHGALDCCVVVILSHGC | |
| | AATGTGAACTTCTGCAGAGAGAGCG | | QASHLQFPGAVYGTDGCPVSVEKIVNI | |
| | GGCTGCGGACCAGAACAGGATCCA | | FNGTSCPSLGGKPKLFFIQACGGEQK | |
| | ATATTGACTGTGAAAAGCTGCGGAG | | DHGFEVASTSPEDESPGSNPEPDATP | |
| | AAGGTTCTCTAGTCTGCACTTTATGG | | FQEGLRTFDQLDAISSLPTPSDIFVSY | |
| | TCGAGGTGAAAGGCGATCTGACCGC | | STFPGFVSWRDPKSGSWYVETLDDIF | |
| | TAAGAAAATGGTGCTGGCCCTGCTG | | EQWAHSEDLQSLLLRVANAVSVKGIY | |
| | GAACTGGCTCGGCAGGACCATGGG | | KQMPGCFNFLRKKLFFKTSASRA | |
| | GCACTGGATTGCTGCGTGGTCGTGA | | | |
| | TCCTGAGTCACGGCTGCCAGGCTTC | | | |
| | ACATCTGCAGTTCCCTGGGGCAGTC | | | |
| | TATGGAACTGACGGCTGTCCAGTCA | | | |
| | GCGTGGAGAAGATCGTGAACATCTT | | | |
| | CAACGGCACCTCTTGCCCAAGTCTG | | | |
| | GGCGGGAAGCCCAAACTGTTCTTTA | | | |
| | TTCAGGCCTGTGGAGGCGAGCAGA | | | |
| | AAGATCACGGCTTCGAAGTGGCTAG | | | |
| | CACCTCCCCCGAGGACGAATCACCT | | | |
| | GGAAGCAACCCTGAGCCAGATGCAA | | | |
| | CCCCCTTCCAGGAAGGCCTGAGGA | | | |
| | CATTTGACCAGCTGGATGCCATCTC | | | |
| | AAGCCTGCCCACACCTTCTGACATT | | | |
| | TTCGTCTCTTACAGTACTTTCCCTGG | | | |
| | ATTTGTGAGCTGGCGCGATCCAAAG | | | |
| | TCAGGCAGCTGGTACGTGGAGACAC | | | |
| | TGGACGATATCTTTGAGCAGTGGGC | | | |
| | CCATTCTGAAGACCTGCAGAGTCTG | | | |
| | CTGCTGCGAGTGGCCAATGCTGTCT | | | |
| | CTGTGAAGGGGATCTACAAACAGAT | | | |
| | GCCAGGATGCTTCAACTTTCTGAGA | | | |
| | AAGAAACTGTTCTTTAAGACCTCCG | | | |
| | CATCTAGGGCC | | | |
| Linker | CCGCGG | 17 | PR | 18 |
| T2A | GAGGGCAGAGGCAGCCTCCTGACA | 19 | EGRGSLLTCGDVEENPGP | 20 |
| | TGTGGGGACGTCGAGGAGAACCCT | | | |
| | GGCCCA | | | |
| Linker | CCTTGG | 21 | PW | 22 |
| Signal Peptide | ATGGAGTTCGGATTGAGCTGGCTGT | 23 | MEFGLSWLFLVAILKGVQCSR | 24 |
| | TCCTGGTGGCAATACTCAAGGGCGT | | | |
| | TCAATGTTCACGG | | | |
| BCMA | GATATCGTGCTGACCCAGTCCCCCC | 87 | DIVLTQSPPSLAMSLGKRATISCRASE | 88 |
| (C123.A3.2) V_{L} | CTAGCCTGGCCATGTCCCTGGGCAA | | SVTILGSHLIYWYQQKPGQPPTLLIQL | |
| | ACGGGCCACCATCTCCTGCAGAGCC | | ASNVQTGVPARFSGSGSRTDFTLTID | |
| | TCCGAGTCCGTGACCATCCTCGGCT | | PVEEDDVAVYYCLQSRTIPRTFGGGT | |
| | CCCACCTGATCTACTGGTACCAGCA | | KLEIK | |
| | GAAGCCCGGCCAGCCTCCCACCCT | | | |
| | CCTTATCCAGCTGGCCAGCAACGTG | | | |
| | CAGACCGGCGTGCCCGCTAGATTCT | | | |
| | CCGGCAGCGGCTCTAGAACCGACTT | | | |
| | CACCCTGACCATCGACCCCGTGGAA | | | |
| | GAGGACGATGTCGCCGTGTACTATT | | | |
| | GCCTGCAGTCCAGAACCATCCCTAG | | | |
| | GACATTCGGCGGAGGAACCAAGCT | | | |
| | GGAGATCAAA | | | |
| Flex | GGGGGCGGTGGCAGCGGTGGCGG | 89 | GGGGSGGGGSGGGGS | 90 |
| | TGGGTCTGGGGGCGGAGGCTCT | | | |
| BCMA | CAGATCCAGCTGGTGCAGTCCGGC | 91 | QIQLVQSGPELKKPGETVKISCKASG | 92 |
| (C123.A3.2) V_{H} | CCCGAGCTGAAGAAACCCGGCGAG | | YTFRHYSMNWVKQAPGKGLKWMGRIN | |
| | ACCGTGAAGATCTCCTGCAAGGCCA | | TESGVPIYADDFKGRFAFSVETSAST | |
| | GCGGCTACACCTTCAGACACTACAG | | AYLVINNLKDEDTASYFCSNDYLYSL | |
| | CATGAACTGGGTGAAGCAGGCCCCT | | DFWGQGTALTVSS | |
| | GGCAAGGGCCTGAAGTGGATGGGC | | | |
| | CGGATCAACACCGAGTCCGGCGTG | | | |
| | CCCATCTACGCCGACGATTTCAAGG | | | |
| | GCAGATTCGCCTTCAGCGTGGAGAC | | | |
| | CTCCGCCTCTACCGCCTACCTGGTG | | | |
| | ATCAACAATCTGAAGGACGAGGACA | | | |
| | CCGCCTCCTACTTCTGCAGCAACGA | | | |
| | CTACCTGTACAGCCTGGACTTCTGG | | | |
| | GGCCAGGGCACCGCCCTGACCGTG | | | |
| | AGCTCCG | | | |
| Linker | GGATCC | 33 | GS | 34 |
| CD34 epitope | GAACTTCCTACTCAGGGGACTTTCT | 35 | ELPTQGTFSNVSTNVS | 36 |
| | CAAACGTTAGCACAAACGTAAGT | | | |
| CD8 stalk | GAACTTCCTACTCAGGGGACTTTCT | 37 | PAPRPPTPAPTIASQPLSLRPEACRPA | 38 |
| | CAAACGTTAGCACAAACGTAAGT | | AGGAVHTRGLDFACD | |
| CD8 | ATCTATATCTGGGCACCTCTCGCTG | 39 | IYIWAPLAGTCGVLLLSLVITLYCNHRN | 40 |
| transmembrane | GCACCTGTGGAGTCCTTCTGCTCAG | | RRRVCKCPR | |
| | CCTGGTTATTACTCTGTACTGTAAT | | | |
| | CACCGGAATCGCCGCCGCGTTTGTA | | | |
| | AGTGTCCCAGG | | | |
| Linker | GTCGAC | 41 | VD | 42 |
| CD3ζ | AGAGTGAAGTTCAGCAGGAGCGCA | 43 | RVKFSRSADAPAYQQGQNQLYNELN | 44 |
| | GACGCCCCCGCGTACCAGCAGGGC | | LGRREEYDVLDKRRGRDPEMGGKPR | |
| | CAGAACCAGCTCTATAACGAGCTCA | | RKNPQEGLYNELQKDKMAEAYSEIG | |
| | ATCTAGGACGAAGAGAGGAGTACGA | | MKGERRRGKGHDGLYQGLSTATKDT | |
| | TGTTTTGGACAAGAGACGTGGCCGG | | YDALHMQALPPR | |
| | GACCCTGAGATGGGGGGAAAGCCG | | | |
| | AGAAGGAAGAACCCTCAGGAAGGC | | | |
| | CTGTACAATGAACTGCAGAAAGATA | | | |
| | AGATGGCGGAGGCCTACAGTGAGAT | | | |
| | TGGGATGAAAGGCGAGCGCCGGAG | | | |
| | GGGCAAGGGGCACGATGGCCTTTA | | | |
| | CCAGGGTCTCAGTACAGCCACCAAG | | | |
| | GACACCTACGACGCCCTTCACATGC | | | |
| | AAGCTCTTCCACCTCGT | | | |
| P2A | GCAACGAATTTTTCCCTGCTGAAACA | 114 | ATNFSLLKQAGDVEENPGP | 74 |
| | GGCAGGGGACGTAGAGGAAAATCC | | | |
| | TGGTCCT | | | |
| MYD88 | ATGGCTGCAGGAGGTCCCGGCGCG | 1 | MAAGGPGAGSAAPVSSTSSLPLAALN | 2 |
| | GGGTCTGCGGCCCCGGTCTCCTCC | | MRVRRRLSLFLNVRTQVAADWTALAE | |
| | ACATCCTCCCTTCCCCTGGCTGCTC | | EMDFEYLEIRQLETQADPTGRLLDAW | |
| | TCAACATGCGAGTGCGGCGCCGCC | | QGRPGASVGRLLDLLTKLGRDDVLLE | |
| | TGTCTCTGTTCTTGAACGTGCGGAC | | LGPSIEEDCQKYILKQQQEEAEKPLQ | |
| | ACAGGTGGCGGCCGACTGGACCGC | | VAAVDSSVPRTAELAGITTLDDPLGH | |
| | GCTGGCGGAGGAGATGGACTTTGA | | MPERFDAFICYCPSDI | |
| | GTACTTGGAGATCCGGCAACTGGAG | | | |
| | ACACAAGCGGACCCCACTGGCAGG | | | |
| | CTGCTGGACGCCTGGCAGGGACGC | | | |
| | CCTGGCGCCTCTGTAGGCCGACTG | | | |
| | CTCGATCTGCTTACCAAGCTGGGCC | | | |
| | GCGACGACGTGCTGCTGGAGCTGG | | | |
| | GACCCAGCATTGAGGAGGATTGCCA | | | |
| | AAAGTATATCTTGAAGCAGCAGCAG | | | |
| | GAGGAGGCTGAGAAGCCTTTACAGG | | | |
| | TGGCCGCTGTAGACAGCAGTGTCCC | | | |
| | ACGGACAGCAGAGCTGGCGGGCAT | | | |
| | CACCACACTTGATGACCCCCTGGGG | | | |
| | CATATGCCTGAGCGTTTCGATGCCT | | | |
| | TCATCTGCTATTGCCCCAGCGACAT | | | |
| | C | | | |
| Linker | GTCGAG | 13 | VE | 14 |
| CD40 | AAAAAGGTGGCCAAGAAGCCAACCA | 115 | KKVAKKPTNKAPHPKQEPQEINFPDD | 56 |
| | ATAAGGCCCCCCACCCCAAGCAGGA | | LPGSNTAAPVQETLHGCQPVTQEDG | |
| | GCCCCAGGAGATCAATTTTCCCGAC | | KESRISVQERQ | |
| | GATCTTCCTGGCTCCAACACTGCTG | | | |
| | CTCCAGTGCAGGAGACTTTACATGG | | | |
| | ATGCCAACCGGTCACCCAGGAGGAT | | | |
| | GGCAAAGAGAGTCGCATCTCAGTGC | | | |
| | AGGAGAGACA | | | |
| Linker | GGATCTGGCGGCCGC | 93 | GSGGR | 94 |
| T2A | GAGGGAAGGGGAAGTCTTCTAACAT | 95 | EGRGSLLTCGDVEENPGP | 20 |
| | GCGGGGACGTGGAGGAAAATCCCG | | | |
| | GGCCC | | | |
| STOP | TGA | | STOP | |
| IL-15 | ATGAGAATTTCGAAACCACATTTGAG | 116 | MRISKPHLRSISIQCYLCLLLNSHFLT | 97 |
| | AAGTATTTCCATCCAGTGCTACTTGT | | EAGIHVFILGCFSAGLPKTEANWVNVI | |
| | GTTTACTTCTAAACAGTCATTTTCTA | | SDLKKIEDLIQSMHIDATLYTESDVHP | |
| | ACTGAAGCTGGCATTCATGTCTTCAT | | SCKVTAMKCFLLELQVISLESGDASIH | |
| | TTTGGGCTGTTTCAGTGCAGGGCTT | | DTVENLIILANNSLSSNGNVTESGCKE | |
| | CCTAAAACAGAAGCCAACTGGGTGA | | CEELEEKNIKEFLQSFVHIVQMFINT | |
| | ATGTAATAAGTGATTTGAAAAAAATT | | | |
| | GAAGACCTTATTCAATCTATGCACAT | | | |
| | TGATGCTACTTTATATACGGAAAGTG | | | |
| | ATGTTCACCCCAGTTGCAAAGTAAC | | | |
| | AGCAATGAAGTGCTTTCTCTTGGAG | | | |
| | TTACAAGTTATTTCACTTGAGTCCGG | | | |
| | AGATGCAAGTATTCATGATACAGTAG | | | |
| | AAAATCTGATCATCCTAGCAAACAAC | | | |
| | AGTTTGTCTTCTAATGGGAATGTAAC | | | |
| | AGAATCTGGATGCAAAGAATGTGAG | | | |
| | GAACTGGAGGAGAAGAACATCAAGG | | | |
| | AATTTTTGCAGAGTTTTGTACATATT | | | |
| | GTCCAAATGTTCATCAACACT | | | |
| STOP | TGA | STOP | STOP | |

### Results

### Constitutive Activation of MC Supports Enhanced Proliferation of NK Cells

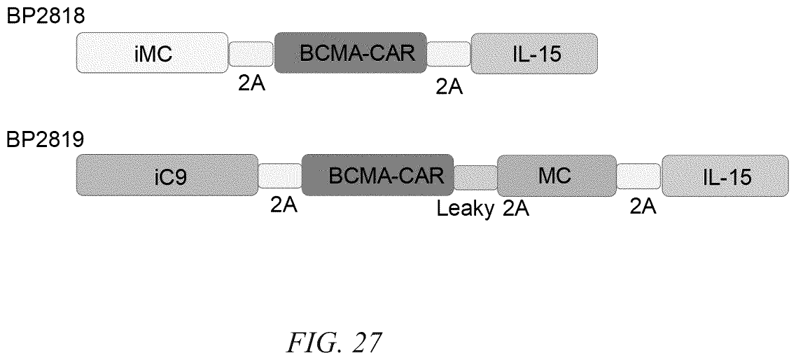

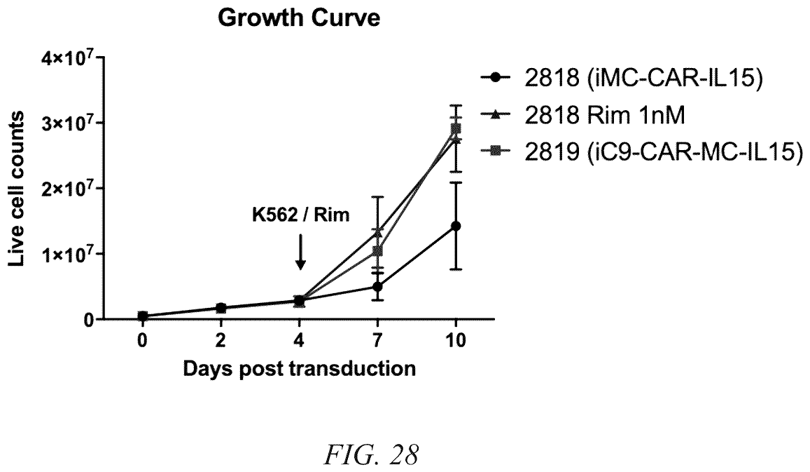

Activated NK cells were prepared and transduced with retroviral constructs encoding iMC, IL-15 and a first-generation BCMA-directed CAR or with a four cistron construct encoding iC9 (rimiducid inducible Caspase-9), IL-15, the same CAR and MC lacking FKBP sequences (FIG. 27). The 2A cotranslational cleavage site separating the CAR and MC was engineered to support cotranslational cleavage inefficiently by removing the GSG containing linker 5′ to the 2A sequence. This ‘leaky’ cotranslational cleavage produces a fusion CAR with MC expressed at the cell membrane. This MC at the membrane is constitutively active. In the absence of rimiducid, this constitutively-active MC supported NK cell growth to a degree similar to CAR-NK cells expressing iMC when stimulated with rimiducid, and greater than iMC CAR-NK cells not treated with rimiducid (FIG. 28). During the growth assay, cells were co-cultured with K562 cells to further activate the transduced NK cells.

Constitutively Activated MC Supports Enhanced Anti-Tumor Cell Killing that is Blocked by Activation of the iC9 Safety Switch

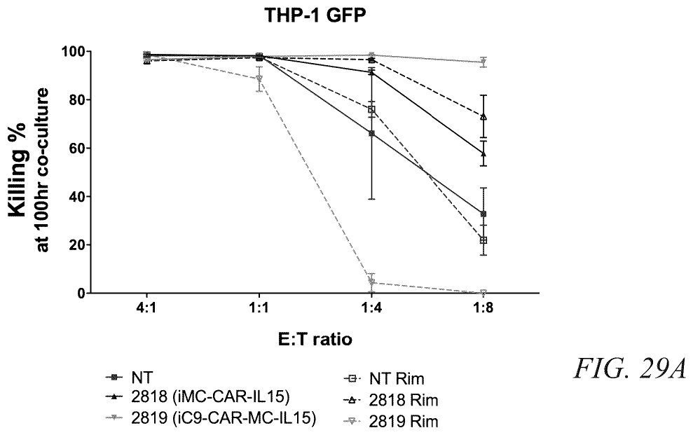

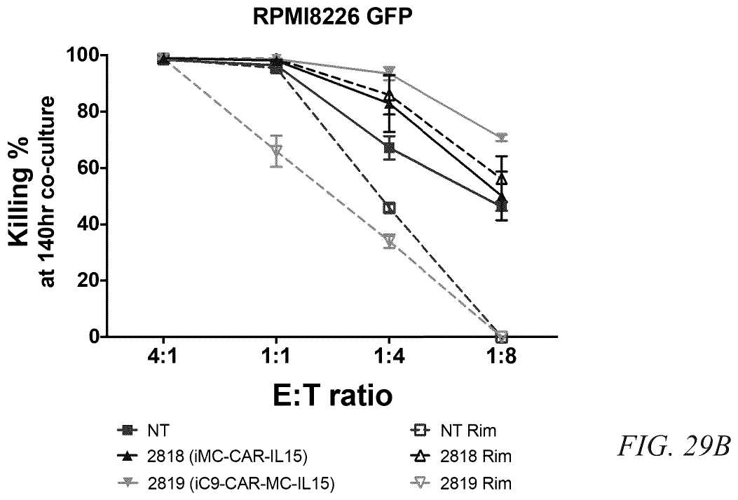

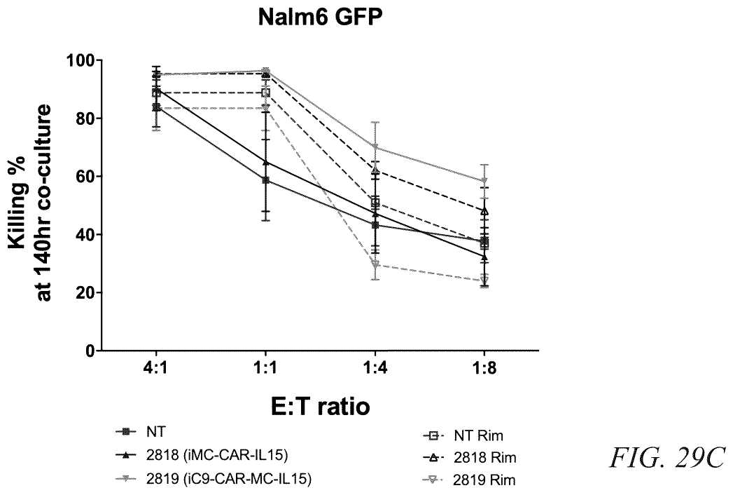

Activated NK cells were prepared from three donors and transduced with the CAR constructs encoding inducible MC (BP2818) or the constitutively active MC species that also encodes the rimiducid-inducible Caspase-9 (BP2819). Co-culture assays were prepared with three different tumor lines, THP-1 which expresses BCMA, the target protein to which the CAR is directed, RPMI8226 another BCMA expressing cell line or Nalm6 which does not express BCMA. Use of a cell line that is not specifically targeted by the CAR permitted an assessment of the intrinsic capacity of the NK cells to target tumors and whether inducible or constitutive MC activity augments this anti-tumor activity. Co-culture assays were performed at decreasing effector (NK cells) to target (the tumor cells) ratios (E:T) ratios such that increased anti-tumor efficacy will be evident as greater tumor cell killing when the numbers of NK cells presented are low. In co-culture assays with THP-1 targets in an Incucyte microscope/incubator, non-transduced (NT) NK cells showed robust killing activity up to an E:T ratio of 1:1 with activity reduced at lower E:T ratios (FIG. 29) This intrinsic NK cell killing against the target served as a standard for the capacity of the CAR-containing NK cells. At low E:T ratios CAR-NK cells with inducible MC demonstrated enhanced killing activity relative to non-transduced NK cells and this activity was further enhanced by rimiducid activation of iMC. Constitutively active MC (2819) supported the greatest antitumor efficacy with essentially complete eradication of the target at a low E:T ratio of 1:8. Activation of the iC9 safety switch with rimiducid in NK cells transduced with 2819 caused greatly reduced anti-THP1 efficacy as only the untransduced NK cells remained in the coculture. Similar results were observed when RPMI8226 were targeted. When Nalm6 cells that were not targeted by the CAR were co-cultured, a similar pattern was observed, but the degree of enhanced activity from constitutive MC relative to iMC/rimiducid was less evident. This may indicate that the constitutive activity of MC when fused to the CAR is enhanced by the ligation of the CAR with its protein target.

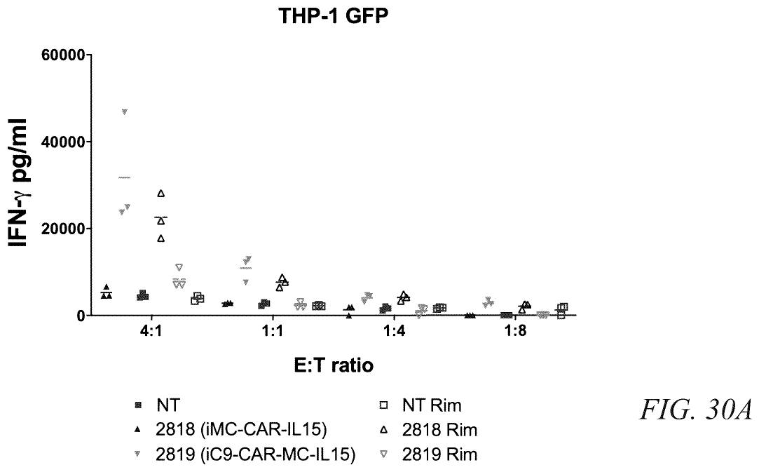

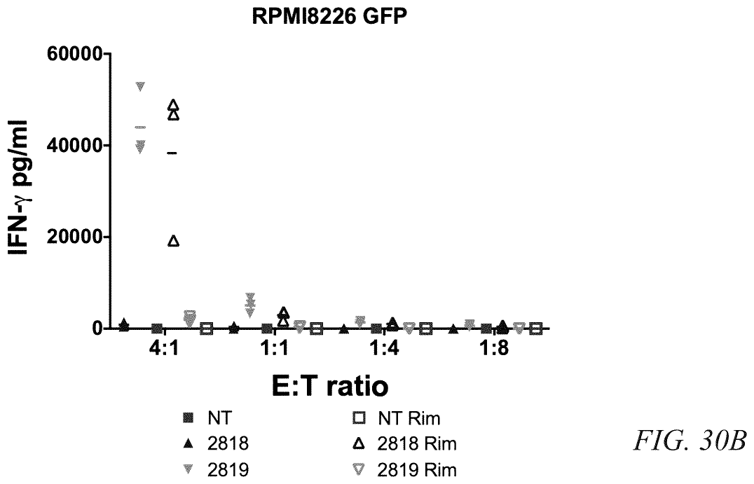

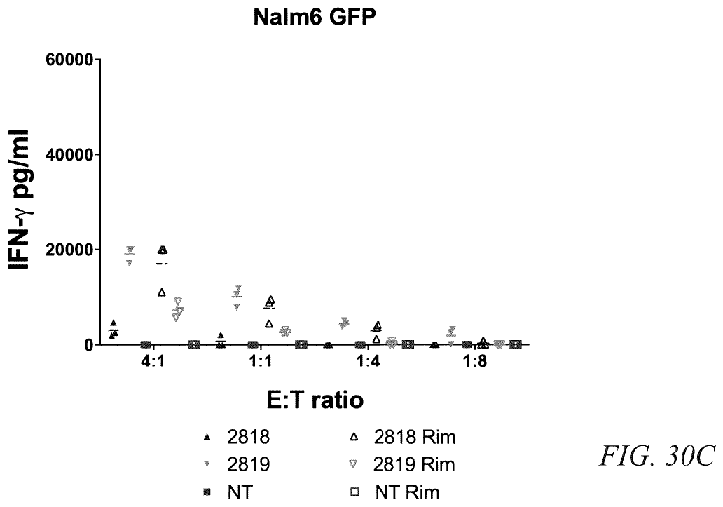

The activation of CAR-NK cells containing constitutive and inducible MC was also assessed by the release of interferon-γ over 48 hours in response to engagement with tumor targets (FIG. 30). When challenged with either THP-1 or RPMI8266 cells that are targeted by the CAR, high levels of this cytokine were produced and released into the media. Cells with constitutive MC produced slightly more IFN-γ than cells with rim-activated iMC while low levels were produced by cells containing iMC but uninduced with rimiducid. MC activity still generated elevated cytokine release in the absence of CAR engagement by coculturing with Nalm6 cells, but levels were lower than observed with BCMA expressing cells as expected.

### Example 5: Representative Embodiments

Provided hereafter are examples of certain embodiments of the technology.

A1. A modified natural killer (NK) cell, comprising a polynucleotide encoding a chimeric polypeptide, wherein the chimeric polypeptide comprises
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

A2. The method of A1 wherein the NK cells are cryostored.

A3. The method of A1 or A2 wherein the modified NK cell has been stored at a temperature of −150° C. or below.

A4-A120. Reserved.

B1. A method for cryopreserving NK cells, comprising storing modified NK cells at a temperature below 0° C., wherein the NK cells comprise a polynucleotide encoding a chimeric polypeptide, wherein the chimeric polypeptide comprises
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

B2-B120. Reserved.

C1. A method for growing NK cells ex vivo, comprising incubating modified NK cells in cell culture medium, wherein the NK cells comprise a polynucleotide encoding a chimeric polypeptide, wherein the chimeric polypeptide comprises
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

C2-C120. Reserved.

D1. A method for thawing NK cells comprising thawing frozen modified NK cells, wherein the NK cells comprise a polynucleotide encoding a chimeric polypeptide, wherein the chimeric polypeptide comprises
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40
   wherein the modified NK cells have been stored at a temperature of 0° C. or below.

D2-D120. Reserved

E1. A method for stimulating an immune response comprising transfecting or transducing a NK cell ex vivo with a nucleic acid comprising a polynucleotide encoding a chimeric polypeptide, wherein the chimeric polypeptide comprises
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

E2. The method of embodiment E1, comprising the step of transfecting or transducing NK cells with a second nucleic acid comprising a polynucleotide encoding a second chimeric polypeptide.

E3. The method of embodiment E1 or E2, comprising contacting the modified NK cells with an antibody.

E4. The method of any one of embodiments E1-E3, wherein the immune response is a cytotoxic response.

E5. The method of any one of embodiments E1-E4, wherein the immune response is a cytolytic response.

E6. The method of any one of embodiments E1-E5, wherein the immune response is an anti-tumor response.

E7. The method of any one of embodiments E1-E6, comprising contacting the modified NK cells with an antibody that binds to an antigen on a tumor.

E8-E120. Reserved.

F1. A method comprising administering modified NK cells to a subject, wherein the modified NK cells comprise a polynucleotide encoding a chimeric polypeptide, wherein the chimeric polypeptide comprises
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

F2. The method of embodiment F1, wherein the subject has a disease or condition associated with an elevated expression of a target antigen expressed by a target cell.

F3. The method of embodiment F1, wherein a tumor has been detected in the subject.

F4. The method of any one of embodiments F1 to F3, comprising administering a ligand to the subject, wherein the ligand binds to the ligand binding region of a first chimeric polypeptide and the ligand binding region of a second chimeric polypeptide, resulting in the multimerization of the first chimeric polypeptide and the second chimeric polypeptide.

F5. The method of embodiment F2 or F4, comprising administering an effective amount of a ligand that binds to the ligand binding region of the chimeric polypeptide to reduce the number or concentration of target antigen or target cells in the subject.

F6. The method of embodiment F3 or F4, comprising administering an effective amount of a ligand that binds to the first ligand binding region and the second ligand binding region of the chimeric polypeptide to reduce the size of the tumor in the subject.

F7. The method of any one of embodiments F4-F6, wherein the ligand is administered after the modified NK cells are administered to the subject.

F8. The method of embodiment F2, F4, F5, or F7, wherein the modified NK cells have been primed with an antibody that binds to the target antigen before administration of the modified NK cells to the subject.

F9. The method of embodiment F3, F4, F6, or F7, wherein the modified NK cells have been primed with an antibody that binds to a tumor antigen before administration of the modified NK cells to the subject.

F10. The method of embodiment F2, F4, F5, F7, or F8, comprising measuring the number or concentration of target cells in a first sample obtained from the subject before administering the modified NK cells, measuring the number or concentration of target cells in a second sample obtained from the subject after administering the modified NK cells, and determining an increase or decrease of the number or concentration of target cells in the second sample compared to the number or concentration of target cells in the first sample.

F11. The method of embodiment F4, F5, F7, or F8, comprising measuring the number or concentration of target cells in a first sample obtained from the subject before administering the ligand, measuring the number or concentration of target cells in a second sample obtained from the subject after administering the ligand, and determining an increase or decrease of the number or concentration of target cells in the second sample compared to the number or concentration of target cells in the first sample.

F12. The method of embodiment F3, F4, F6, F7, or F9, comprising measuring the size of a tumor in the subject before administering the modified NK cells, measuring the size of a tumor in the subject after administering the modified NK cells, and determining an increase or decrease of the size of the tumor following administration of the modified NK cells.

F13. The method of embodiment F4, F6, F7, or F9, comprising measuring the size of a tumor in the subject before administering the ligand, measuring the size of a tumor in the subject after administering the ligand, and determining an increase or decrease of the size of the tumor following administration of the ligand.

F14. The method of any one of embodiments F1-F13, wherein the subject has received a stem cell transplant before or at the same time as administration of the modified NK cells.

F15. The method of any one of embodiments F1-F14, wherein the modified NK cells wherein the ligand binding region comprises a first polynucleotide encoding a first FKBP12v36 ligand binding region and a second FKBP12v36 ligand binding region, and wherein said signaling region comprises a truncated MyD88 polypeptide lacking the TIR domain and a CD40 cytoplasmic polypeptide region lacking the CD40 extracellular domain,

F16. The method of embodiment F15, wherein the NK cell further comprises a second polynucleotide encoding an IL-15 polypeptide, and a third polynucleotide encoding a chimeric antigen receptor.

F17. The method of any one of embodiments F1-F15, wherein the modified NK cells further comprise a nucleic acid comprising a polynucleotide encoding a chimeric apoptotic polypeptide comprising an FKBP12 binding region, an FRB binding region or an FRB variant binding region, and a Caspase-9 polypeptide lacking the CARD domain.

F18. The method of any one of embodiments F1-F14, wherein the modified NK cells comprise a nucleic acid comprising a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric apoptotic polypeptide comprising an FKBP12 binding region, an FRB binding region or an FRB variant binding region, and a Caspase-9 polypeptide lacking the CARD domain.

F19. The method of embodiment F18, wherein the modified NK cells comprise a nucleic acid comprising a polynucleotide encoding a chimeric antigen receptor.

F20. The method of embodiment F16 or F19, wherein the CAR targets HER2, PSCA, CD123 or BCMA.

F21. The method of any one of embodiments F1-F20, wherein the subject has an immune response.

F22. The method of any one of embodiments F1-F21, comprising contacting the modified NK cells with an antibody.

F23. The method of embodiment F21 or F22, wherein the immune response is a cytotoxic response.

F24. The method of any one of embodiments F21-F23, wherein the immune response is a cytolytic response.

F25. The method of any one of embodiments F21-F24, wherein the immune response is an anti-tumor response.

F26. The method of any one of embodiments F21-F25, comprising contacting the modified NK cells with an antibody that binds to an antigen on a tumor.

F27. The method of any one of embodiments F21-F26, wherein IL-15 is not administered to the subject within one week of administration of the modified NK cells.

F28. The method of any one of embodiments F21-F26, wherein IL-15 is not administered to the subject within two weeks of administration of the modified NK cells.

F29. The method of any one of embodiments F21-F28, wherein one dose of the modified NK cells is administered.

F30. The method of embodiment F29, wherein an immune response is detected in the subject following administration of the modified NK cells.

F31. The method of embodiment F30, comprising administering to the subject a ligand that binds to the ligand binding region in said chimeric polypeptide, and an immune response is detected in the subject following administration of the ligand.

F32. The method of embodiment F30- or F31, wherein the immune response is directed against a tumor in the subject.

F33. The method of any one of embodiments F1-F31, wherein the subject has cancer.

F34. The method of any one of embodiments F1-F33, wherein the subject has been diagnosed as having one or more tumors, and number of tumor cells is reduced following administration of the modified NK cell.

F35. The method of any one of embodiments F1-F34, wherein the subject has been diagnosed as having one or more tumors, and the size of one or more tumors is reduced following administration of the modified NK cell.

F36. The method of any one of embodiments F1-F35, wherein the subject has been diagnosed as having a hyperproliferative disease.

F37. The method of embodiment F34 or F35, comprising administering to the subject a ligand that binds to the ligand biding region, and a reduction in the number of tumor cells or the size of one or more tumors is detected in the subject following administration of the ligand.

F38. The method of embodiment F34, F35, or F37, wherein the modified NK cells are contacted with an antibody that binds to an antigen on the tumor before administration of the modified NK cells to the subject.

G1. A kit comprising modified natural killer (NK) cells, wherein the modified NK cells comprise a polynucleotide encoding a chimeric polypeptide, comprising
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40
   wherein the modified NK cells have been stored at a temperature of −0° C. or below.

G2. The kit of embodiment of G1, wherein the modified NK cells have been stored at a temperature of −150° C. or below.

G3. The kit of any embodiment G1 or G2, comprising a ligand that binds to the ligand binding region.

G4. The kit of any one of embodiments G1-G3, comprising an antibody.

G5. The kit of embodiment G4, wherein the antibody binds to an antigen on a target cell.

I6. The kit of embodiment G5, wherein the antibody is formulated for priming the NK cells stimulate an immune response against the target cell.

G7. The kit of embodiment G5 or G6 wherein the target cell is a tumor cell.

G10. The kit of any one of embodiments G1-G9, wherein the ligand binding region comprising a first ligand binding region and a second ligand binding region wherein the first ligand binding region has a different amino acid sequence than the second ligand binding region, and the first and second ligand binding regions bind to a heterodimeric ligand.

G11. The kit of embodiment G10, wherein the first ligand binding region binds to a first portion of the heterodimeric ligand, and the second ligand binding region binds to a second portion of the heterodimeric ligand.

H1. A modified natural killer (NK) cell, comprising a first and a second polynucleotide, wherein the first polynucleotide encodes a chimeric polypeptide comprising
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40; and
      the second polynucleotide encodes a IL-15 polypeptide.

H2. The modified NK cell of embodiment HI, wherein the ligand binding region comprises a first polynucleotide encoding a first FKBP12v36 ligand binding region and a second FKBP12v36 ligand binding region, and wherein said signaling region comprises a truncated MyD88 polypeptide lacking the TIR domain and a CD40 cytoplasmic polypeptide region lacking the CD40 extracellular domain.

H3. The modified NK cell of embodiment H1 or H2 comprising a third polynucleotide encoding a chimeric apoptotic polypeptide comprising a ligand binding region, and a Caspase-9 polypeptide lacking the CARD domain.

H4. The modified NK cell of embodiment H3, wherein said ligand binding region in chimeric apoptotic polypeptide is an FKBP12 binding region, an FRB binding region or an FRB variant binding region.

H5. The modified NK cell of any one of embodiments H1-H4, wherein the modified cell comprises a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric apoptotic polypeptide comprising an FKBP12 binding region, an FRB binding region or an FRB variant binding region, and a Caspase-9 polypeptide lacking the CARD domain.

H6. The modified NK cell of any one of embodiments H1-5, comprising a nucleic acid comprising a polynucleotide encoding a chimeric antigen receptor.

H7. The modified NK cell of embodiment H6, wherein the CAR targets PSMA, PSCA, Muc1 CD19, ROR1, Mesothelin, GD2, CD123, Muc16, CD33, CD38, CD44v6, Her2/Neu, CD20, CD30, BCMA, PRAME, NY-ESO-1, or EGFRvIII.

I1. A nucleic acid, comprising a first and a second polynucleotide, wherein the first polynucleotide encodes a chimeric polypeptide comprising
a) a ligand binding region; and
b) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40 and the second polynucleotide encodes a IL-15 polypeptide.

I2. The nucleic acid of embodiment I1, wherein the nucleic acid comprises a polynucleotide that encodes a chimeric antigen receptor (CAR) or a T-cell receptor.

I3. The nucleic acid of embodiment I1 or I2, wherein the nucleic acid comprises a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric antigen receptor.

I4. The nucleic acid of embodiment I2 or I3, wherein the CAR targets HER2, PSCA, CD123 or BCMA.

I5. The nucleic acid of embodiment I1, wherein the nucleic acid comprises a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric apoptotic polypeptide comprising an FKBP12 binding region, an FRB binding region or an FRB variant binding region, and a Caspase-9 polypeptide lacking the CARD domain.

I6. The nucleic acid of any one of embodiments I1-15, wherein the nucleic acid comprises a polynucleotide encoding a marker polypeptide.

I7. The nucleic acid of embodiment I6, wherein the marker polypeptide is a ΔCD19 polypeptide.

I8. A modified NK cell comprising a nucleic acid of any one of embodiments I1-I7.

19. A pharmaceutical composition comprising a modified NK cell or a nucleic acid of any one of embodiments A1-A3, H1-H7 or 11-18.

I10. The modified NK cell, method, kit, nucleic acid, or pharmaceutical composition of any one of preceding embodiments, wherein the ligand is rimiducid, AP20187, or AP1510.

J1. A modified natural killer (NK) cell, comprising a first and a second polynucleotide, wherein the first polynucleotide encodes a chimeric polypeptide comprising
a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40; and
      the second polynucleotide encodes a IL-15 polypeptide.

J2. The modified NK cell of embodiment J1, wherein the chimeric polypeptide does not comprise a membrane-targeting region.

J3. The modified NK cell of embodiment J1, wherein the chimeric polypeptide does not comprise a ligand binding region.

J2. The modified NK cell of embodiment J1, wherein the chimeric polypeptide comprises a membrane-targeting region.

K1. The modified NK cell, method, kit, nucleic acid, or pharmaceutical composition of any one of embodiments A1-J2, wherein the cells are not grown on feeder cells.

K2. The modified NK cell, method, kit, nucleic acid, or pharmaceutical composition of any one of embodiments A1-K1, wherein the cell has been stored at a temperature of −150° C. or below for more than 24 hours.

K3. The modified NK cell, method, kit, nucleic acid, or pharmaceutical composition of any one of embodiments A1-K2, wherein the cell has been stored at a temperature of −150° C. or below for more than one week.

K4. The modified NK cell, method, kit, nucleic acid, or pharmaceutical composition of any one of embodiments A1-K3, wherein the cell has been stored at a temperature of −150° C. or below for more than three weeks.

K5. The modified NK cell, method, kit, nucleic acid, or pharmaceutical composition of any one of embodiments A1-K4, wherein the cells have not been contacted with exogenous IL-15.

K6. The modified NK cell, method, kit, nucleic acid, or pharmaceutical composition of any one of embodiments A1-K5, wherein the cell has been thawed following cryostorage.

K7. The modified NK cell, method, kit, nucleic acid, or pharmaceutical composition of any one of embodiments A1-K6, wherein the cell has been primed with an antibody following cryostorage.

K8. The modified NK cell, method, kit, nucleic acid, or pharmaceutical composition of any one of embodiments A1-K7, wherein the signaling region comprises a truncated MyD88 polypeptide lacking the TIR domain.

K9. The modified NK cell, method, kit, nucleic acid, or pharmaceutical composition of any one of embodiments A1-K7, wherein the signaling region comprises a truncated MyD88 polypeptide lacking the TIR domain and a CD40 cytoplasmic polypeptide region lacking the CD40 extracellular domain.

K10. The modified NK cell, method, kit, nucleic acid, or pharmaceutical composition of any one of embodiments A1-K7, wherein the signaling region comprises a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions.

K11. The modified NK cell, method, kit, nucleic acid, or pharmaceutical composition of embodiment K10, wherein the co-stimulatory polypeptide cytoplasmic signaling region is selected from the group consisting of CD27, CD28, ICOS, 4-1BB, RANK/TRANCE-R, and OX40.

K12. The modified NK cell, method, kit, nucleic acid, or pharmaceutical composition of embodiment K10, wherein the co-stimulatory polypeptide cytoplasmic signaling region is selected from the group consisting of CD28, 4-1BB, OX40, and ICOS.

K13. The modified NK cell, method, kit, nucleic acid, or pharmaceutical composition of embodiment K10, wherein the co-stimulatory polypeptide cytoplasmic signaling region is selected from the group consisting of CD27, CD28, ICOS, 4-1BB, and OX40.

K14. The modified NK cell, method, kit, nucleic acid, or pharmaceutical composition of any one of embodiments K8-K13, wherein the co-stimulatory polypeptide lacks an extracellular domain or lacks a functional extracellular domain.

K15. The modified NK cell, method, kit, nucleic acid, or pharmaceutical composition of any one of embodiments A1-K10, wherein the truncated MyD88 polypeptide comprises the amino acid sequence of the full length MyD88 sequence of SEQ ID NO: 118, wherein the truncated MyD88 polypeptide lacks the TIR domain, or a functional fragment thereof.

K16. The modified NK cell, method, kit, nucleic acid, or pharmaceutical composition of any one of embodiments A1-K10, wherein the truncated MyD88 polypeptide does not comprise contiguous amino acid residues 156 to the C-terminus of the full length MyD88 polypeptide.

K17. The modified NK cell, method, kit, nucleic acid, or pharmaceutical composition of any one of embodiments A1-K10, wherein the truncated MyD88 polypeptide does not comprise contiguous amino acid residues 152 to the C-terminus of the full length MyD88 polypeptide.

K18. The modified NK cell, method, kit, nucleic acid, or pharmaceutical composition of any one of embodiments A1-K10, wherein the truncated MyD88 polypeptide does not comprise contiguous amino acid residues 173 to the C-terminus of the full length MyD88 polypeptide.

K19. The modified NK cell, method, kit, nucleic acid, or pharmaceutical composition of any one of embodiments A1-K10, or K15-K18, wherein the full length MyD88 polypeptide comprises the amino acid sequence of SEQ ID NO: 118.

K20. The modified NK cell, method, kit, nucleic acid, or pharmaceutical composition of any one of embodiments A1-N3 or K15-K19, wherein the truncated MyD88 polypeptide consists of the amino acid sequence of SEQ ID NO: 119 or SEQ ID NO: 2, or a functional fragment thereof.

K21. The modified NK cell, method, kit, nucleic acid, or pharmaceutical composition of any one of embodiments A1-K10 or K15-K19, wherein the truncated MyD88 polypeptide has the amino acid sequence of SEQ ID NO: 119 or SEQ ID NO: 2, or an amino acid sequence 90% or more identical to SEQ ID NO: 119 or SEQ ID NO: 2.

K22. The modified NK cell, method, kit, nucleic acid, or pharmaceutical composition of any one of embodiments A1-K10 or K15-K19, wherein the truncated MyD88 polypeptide has the amino acid sequence of SEQ ID NO: 119 or SEQ ID NO: 2, or an amino acid sequence 95% or more identical to SEQ ID NO: 119 or SEQ ID NO: 2.

K23. The modified NK cell, method, kit, nucleic acid, or pharmaceutical composition of any one of embodiments A1-K10 or K15-K19, wherein the truncated MyD88 polypeptide has the amino acid sequence of SEQ ID NO: 119 or SEQ ID NO: 2.

K24. The modified NK cell, method, kit, nucleic acid, or pharmaceutical composition of any one of embodiments A1-K23, wherein the signaling region comprises a CD40 cytoplasmic polypeptide and lacks the CD40 extracellular domain.

K25. The modified NK cell, method, kit, nucleic acid, or pharmaceutical composition of any one of embodiments A1-K24, wherein the CD40 cytoplasmic polypeptide has the amino acid sequence of SEQ ID NO: 56, or an amino acid sequence 90% or more identical to SEQ ID NO: 56.

K26. The modified NK cell, method, kit, nucleic acid, or pharmaceutical composition of any one of embodiments A1-K24, wherein the CD40 cytoplasmic polypeptide has the amino acid sequence of SEQ ID NO: 56 or an amino acid sequence 95% or more identical to SEQ ID NO: 56.

K27. The modified NK cell, method, kit, nucleic acid, or pharmaceutical composition of any one of embodiments A1-K24, wherein the CD40 cytoplasmic polypeptide has the amino acid sequence of SEQ ID NO: 56.

K28. The modified NK cells, methods, pharmaceutical compositions, or kits of any one of embodiments A1-K27, wherein the modified NK cells comprise a polynucleotide that encodes a chimeric antigen receptor (CAR).

K29. The modified NK cells, methods, pharmaceutical compositions, or kits of embodiment K28, wherein the CAR comprises a transmembrane region, a cell activation region, and an antigen recognition region.

K30. The modified NK cells, methods, pharmaceutical compositions, or kits of embodiment K29, wherein the cell activation region is a T-cell activation region.

K31. The modified NK cells, methods, pharmaceutical compositions, or kits of embodiment K29, wherein the T-cell activation region is a CD3 zeta-chain region.

K32. The modified NK cells, methods, pharmaceutical compositions, or kits of any one of embodiments K29-K31, wherein the antigen recognition region specifically binds to a molecule chosen from PSMA, PSCA, Muc1 CD19, ROR1, Mesothelin, GD2, CD123, Muc16, CD33, CD38, CD44v6, and Her2/Neu.

K33. The modified NK cells, methods, pharmaceutical compositions, or kits of any one of embodiments K29-K32, wherein the antigen recognition moiety binds to an antigen on a cell involved in a hyperproliferative disease or to a viral or bacterial antigen.

K34. The modified NK cells, methods, pharmaceutical compositions, or kits of any one of embodiments K29-K33, wherein the antigen recognition moiety binds to an antigen selected from the group consisting of PSMA, PSCA, MUC1, CD19, ROR1, Mesothelin, GD2, CD123, MUC16, Her2/NE, CD20, CD30, BCMA, PRAME, NY-ESO-1, and EGFRvIII.

Non-limiting examples are provided herein of methods, sequences, and ligands, for expressing chimeric proteins in immune system cells, measuring levels of cytokines, assaying immune activity, activating chimeric proteins by contacting modified immune cells with multimeric ligands, assaying anti-tumor activity, and treatment of subjects using modified immune cells. In some examples, these methods are exemplified using immune cells other than natural killer cells, such as, for example, T cells. These methods may be applied essentially as described, to obtain, assay, and use, the modified cells, for example modified NK cells, of the present application.

### Example 6: Representative Embodiments

Provided hereafter are examples of certain embodiments of the technology.

A1. A cryostored and modified natural killer (NK) cell, comprising a polynucleotide encoding a chimeric polypeptide, wherein the chimeric polypeptide comprises
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40 wherein the modified NK cell has been stored at a temperature of −150° C. or below.

A2-A120. Reserved.

B1. A method for cryopreserving NK cells, comprising storing modified NK cells at a temperature below 0° C., wherein the NK cells comprise a polynucleotide encoding a chimeric polypeptide, wherein the chimeric polypeptide comprises
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

B2-B120. Reserved.

C1. A method for growing NK cells ex vivo, comprising incubating modified NK cells in cell culture medium, wherein the NK cells comprise a polynucleotide encoding a chimeric polypeptide, wherein the chimeric polypeptide comprises
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

C2-C120. Reserved.

D1. A method for thawing NK cells comprising thawing frozen modified NK cells, wherein the NK cells comprise a polynucleotide encoding a chimeric polypeptide, wherein the chimeric polypeptide comprises
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40 wherein the modified NK cells have been stored at a temperature of 0° C. or below.

D2. The method of embodiment D1, comprising the step of transfecting or transducing NK cells with a nucleic acid comprising a polynucleotide encoding the chimeric polypeptide.

D3. The method of any one of embodiments D1-D2, comprising cooling the modified NK cells to a temperature of 0° C. or below.

D4. The method of any one of embodiments D1-D3, comprising cooling the modified NK cells to a temperature of −150° C. or below.

D5. The method of any one of embodiments D1-D4, comprising thawing the modified NK cells.

D6-D120. Reserved.

E1. A method for stimulating an immune response comprising transfecting or transducing a NK cell ex vivo with a nucleic acid comprising a polynucleotide encoding a chimeric polypeptide, wherein the chimeric polypeptide comprises:
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

E2. The method of embodiment E1, comprising the step of transfecting or transducing NK cells with a nucleic acid comprising a polynucleotide encoding the chimeric polypeptide.

E3. The method of any one of embodiments E1-E2, comprising contacting the modified NK cells with an antibody.

E4. The method of any one of embodiments E1-E3, wherein the immune response is a cytotoxic response.

E5. The method of any one of embodiments E1-E4, wherein the immune response is a cytolytic response.

E6. The method of any one of embodiments E1-E5, wherein the immune response is an anti-tumor response.

E7. The method of any one of embodiments E1-E6, comprising contacting the modified NK cells with an antibody that binds to an antigen on a tumor.

E8-E120. Reserved.

F1. A method for stimulating an immune response comprising administering modified NK cells to a subject, wherein the modified NK cells comprise a polynucleotide encoding a chimeric polypeptide, wherein the chimeric polypeptide comprises
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

F2. The method of embodiment F1, wherein the subject has a disease or condition associated with an elevated expression of a target antigen expressed by a target cell.

F3. The method of embodiment F1, wherein a tumor has been detected in the subject.

F4. The method of any one of embodiments F1 to F3, comprising administering a ligand to the subject, wherein the ligand binds to the first ligand binding region of a first chimeric polypeptide and a second ligand binding region of a second chimeric polypeptide, resulting in the multimerization of the first chimeric polypeptide and the second chimeric polypeptide.

F4.1. The method of any one of embodiments F2 or F4, comprising administering an effective amount of a ligand that binds to the first ligand binding region and the second ligand binding region of the chimeric polypeptide to reduce the number or concentration of target antigen or target cells in the subject.

F4.2 The method of any one of embodiments F3 or F4, comprising administering an effective amount of a ligand that binds to the first ligand binding region and the second ligand binding region of the chimeric polypeptide to reduce the size of the tumor in the subject.

F5. The method of any one of embodiments F4-F4.2, wherein the ligand is administered after the modified NK cells are administered to the subject.

F6. The method of any one of embodiments F2, F4, F4.1, or F5, wherein the modified NK cells have been primed with an antibody that binds to the target antigen before administration of the modified NK cells to the subject.

F7. The method of any one of embodiments F3, F4, F4.2, or F5, wherein the modified NK cells have been primed with an antibody that binds to a tumor antigen before administration of the modified NK cells to the subject.

F8. The method of any one of embodiments F2, F4, F4.1, F5, or F6, comprising measuring the number or concentration of target cells in a first sample obtained from the subject before administering the modified NK cells, measuring the number or concentration of target cells in a second sample obtained from the subject after administering the modified NK cells, and determining an increase or decrease of the number or concentration of target cells in the second sample compared to the number or concentration of target cells in the first sample.

F8.1. The method of any one of embodiments F4, F4.1, F5, or F6, comprising measuring the number or concentration of target cells in a first sample obtained from the subject before administering the ligand, measuring the number or concentration of target cells in a second sample obtained from the subject after administering the ligand, and determining an increase or decrease of the number or concentration of target cells in the second sample compared to the number or concentration of target cells in the first sample.

F8.2. The method of any one of embodiments F8 or F8.1, wherein the concentration of target cells in the second sample is decreased compared to the concentration of target cells in the first sample.

F8.3. The method of any one of embodiments F8 or F8.1, wherein the concentration of target cells in the second sample is increased compared to the concentration of target cells in the first sample.

F8.4. The method of any one of embodiments F3, F4, F4.2, F5, or F7, comprising measuring the size of a tumor in the subject before administering the modified NK cells, measuring the size of a tumor in the subject after administering the modified NK cells, and determining an increase or decrease of the size of the tumor following administration of the modified NK cells.

F8.5. The method of any one of embodiments F4, F4.2, F5, or F7, comprising measuring the size of a tumor in the subject before administering the ligand, measuring the size of a tumor in the subject after administering the ligand, and determining an increase or decrease of the size of the tumor following administration of the ligand.

F8.6. The method of any one of embodiments F8.4 or F8.5, wherein the size of the tumor is decreased following administration of the modified NK cells.

F8.7. The method of any one of embodiments F8.4 or F8.5, wherein the size of the tumor is increased following administration of the modified NK cells.

F8.8. The method of any one of embodiments F8.4 or F8.5, wherein the size of the tumor is decreased following administration of the ligand.

F8.9. The method of any one of embodiments F8.4 or F8.5, wherein the size of the tumor is increased following administration of the ligand.

F8.10. The method of any one of embodiments F1-F8.9, wherein the subject has received a stem cell transplant before or at the same time as administration of the modified NK cells.

F11. The method of any one of embodiments F1-F10, wherein the modified NK cells comprise a nucleic acid comprising a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric antigen receptor.

F12. The method of embodiment F11, wherein the CAR targets CD123 or BCMA.

F13. The method of any one of embodiments F11-F11, wherein the modified NK cells comprise a nucleic acid comprising a polynucleotide encoding a chimeric apoptotic polypeptide comprising an FKBP12 binding region, an FRB binding region or an FRB variant binding region, and a Caspase-9 polypeptide lacking the CARD domain.

F14. The method of any one of embodiments F1-F10, wherein the modified NK cells comprise a nucleic acid comprising a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric apoptotic polypeptide comprising an FKBP12 binding region, an FRB binding region or an FRB variant binding region, and a Caspase-9 polypeptide lacking the CARD domain.

F15. The method of embodiment F14, wherein the modified NK cells comprise a nucleic acid comprising a polynucleotide encoding a chimeric antigen receptor.

F16. The method of embodiment F15, wherein the CAR targets CD123 or BCMA.

F17-F99. Reserved.

F100. The method of any one of embodiments F1-F99, wherein the subject has an immune response.

F101. The method of any one of embodiments F1-F100, comprising contacting the modified NK cells with an antibody.

F102. The method of any one of embodiments F100-F101, wherein the immune response is a cytotoxic response.

F103. The method of any one of embodiments F100-F102, wherein the immune response is a cytolytic response.

F104. The method of any one of embodiments F100-F103, wherein the immune response is an anti-tumor response.

F105. The method of any one of embodiments F100-F104, comprising contacting the modified NK cells with an antibody that binds to an antigen on a tumor.

F106. The method of any one of embodiments F100-F105, wherein IL-15 is not administered to the subject within one week of administration of the modified NK cells.

F107. The method of any one of embodiments F100-F105, wherein IL-15 is not administered to the subject within two weeks of administration of the modified NK cells.

F108. The method of any one of embodiments F100-F107, wherein one dose of the modified NK cells is administered.

F109. The method of embodiment F108, wherein an immune response is detected in the subject following administration of the modified NK cells.

F110. The method of embodiment F109, comprising administering a ligand that binds to the first ligand domain and the second ligand domain to the subject, and an immune response is detected in the subject following administration of the ligand.

F111. The method of any one of embodiments F109-F111, wherein the immune response is directed against a tumor in the subject.

F112. The method of any one of embodiments F1-F111, wherein the subject has cancer.

F113. The method of any one of embodiments F1-F112, wherein the subject has been diagnosed as having one or more tumors, and number of tumor cells is reduced following administration of the modified NK cell.

F114. The method of any one of embodiments F1-F113, wherein the subject has been diagnosed as having one or more tumors, and the size of one or more tumors is reduced following administration of the modified NK cell.

F115. The method of any one of embodiments F1-F114, wherein the subject has been diagnosed as having a hyperproliferative disease.

F116. The method of any one of embodiments F113 or F114, comprising administering a ligand that binds to the first ligand domain and the second ligand domain to the subject, and a reduction in the number of tumor cells or the size of one or more tumors is detected in the subject following administration of the ligand.

F117. The method of any one of embodiments F113, F114, or F116, wherein the modified NK cells are contacted with an antibody that binds to an antigen on the tumor before administration of the modified NK cells to the subject.

F118-F120. Reserved.

G1. A method for treating a subject having a disease or condition associated with an elevated expression of a target antigen expressed by a target cell, comprising transplanting an effective amount of modified NK cells into the subject; wherein the modified NK cells comprise a polynucleotide encoding a chimeric polypeptide, wherein the chimeric polypeptide comprises
a) a first ligand binding region;
b) a second ligand binding region; and
c) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

G2. The method of embodiment G1, wherein the target antigen is a tumor antigen.

G3. A method for reducing the size of a tumor in a subject, comprising transplanting an effective amount of modified NK cells into the subject; wherein the modified NK cells comprise a polynucleotide encoding a chimeric polypeptide, wherein the chimeric polypeptide comprises
a) a first ligand binding region;
b) a second ligand binding region; and
c) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.
   viii) G2. The method of embodiment G1, comprising administering an effective amount of a ligand that binds to the FKBP12 variant region of the chimeric costimulating polypeptide to reduce the number or concentration of target antigen or target cells in the subject.

G3.1. The method of any one of embodiments F1 to F3, comprising administering a ligand to the subject, wherein the ligand binds to the first ligand binding region of a first chimeric polypeptide and a second ligand binding region of a second chimeric polypeptide, resulting in the multimerization of the first chimeric polypeptide and the second chimeric polypeptide.

G4. The method of any one of embodiments G1-G3.1, comprising administering an effective amount of a ligand that binds to the first ligand binding region and the second ligand binding region of the chimeric costimulating polypeptide to reduce the number or concentration of target antigen or target cells in the subject.

G5. The method of embodiment G4, wherein the ligand is administered after the modified NK cells are administered to the subject.

G6. The method of any one of embodiments G1-G2 or G4-G5, wherein the modified NK cells have been primed with an antibody that binds to the target antigen before administration of the modified NK cells to the subject.

G7. The method of any one of embodiments G2-G6, wherein the modified NK cells have been primed with an antibody that binds to a tumor antigen before administration of the modified NK cells to the subject.

G8. The method of any one of embodiments G1-G2, or G4-G7, comprising measuring the number or concentration of target cells in a first sample obtained from the subject before administering the modified NK cells, measuring the number or concentration of target cells in a second sample obtained from the subject after administering the modified NK cells, and determining an increase or decrease of the number or concentration of target cells in the second sample compared to the number or concentration of target cells in the first sample.

G8.1. The method of any one of embodiments G4-G8, comprising measuring the number or concentration of target cells in a first sample obtained from the subject before administering the ligand, measuring the number or concentration of target cells in a second sample obtained from the subject after administering the ligand, and determining an increase or decrease of the number or concentration of target cells in the second sample compared to the number or concentration of target cells in the first sample.

G8.2. The method of any one of embodiments G8 or G8.1, wherein the concentration of target cells in the second sample is decreased compared to the concentration of target cells in the first sample.

G8.3. The method of any one of embodiments G8 or G8.1, wherein the concentration of target cells in the second sample is increased compared to the concentration of target cells in the first sample.

G8.4. The method of any one of embodiments G3-G5, or G7, comprising measuring the size of a tumor in the subject before administering the modified NK cells, measuring the size of a tumor in the subject after administering the modified NK cells, and determining an increase or decrease of the size of the tumor following administration of the modified NK cells.

G8.5. The method of any one of embodiments G4-G5, G7, or G8.4, comprising measuring the size of a tumor in the subject before administering the ligand, measuring the size of a tumor in the subject after administering the ligand, and determining an increase or decrease of the size of the tumor following administration of the ligand.

G8.6. The method of any one of embodiments G8.4 or G8.5, wherein the size of the tumor is decreased following administration of the modified NK cells.

G8.7. The method of any one of embodiments G8.4 or G8.5, wherein the size of the tumor is increased following administration of the modified NK cells.

G8.8. The method of any one of embodiments G8.4 or G8.5, wherein the size of the tumor is decreased following administration of the ligand.

G8.9. The method of any one of embodiments G8.4 or G8.5, wherein the size of the tumor is increased following administration of the ligand.

G8.10. The method of any one of embodiments G1-G8.9, wherein the subject has received a stem cell transplant before or at the same time as administration of the modified NK cells.

G8.11. The method of any one of embodiments G1-G8.10, wherein IL-15 is not administered to the subject within forty eight hours before or one week after administration of the modified NK cells.

G8.12. The method of any one of embodiments G1-G8.11, wherein IL-2 is not administered to the subject within forty eight hours before or one week after administration of the modified NK cells.

G9-G99. Reserved.

G100. The method of any one of embodiments G1-G99, wherein the subject has an immune response.

G102. The method of any one of embodiments G100-G101, wherein the immune response is a cytotoxic response.

G103. The method of any one of embodiments G100-G102, wherein the immune response is a cytolytic response.

G104. The method of any one of embodiments G100-G103, wherein the immune response is an anti-tumor response.

G105. The method of any one of embodiments G100-G104, comprising contacting the modified NK cells with an antibody that binds to an antigen on a tumor.

G106. The method of any one of embodiments G100-G105, wherein IL-15 is not administered to the subject within one week of administration of the modified NK cells.

G107. The method of any one of embodiments G100-G105, wherein IL-15 is not administered to the subject within two weeks of administration of the modified NK cells.

G108. The method of any one of embodiments G100-G107, wherein one dose of the modified NK cells is administered.

G109. The method of embodiment G108, wherein an immune response is detected in the subject following administration of the modified NK cells.

G110. The method of embodiment G109, comprising administering a ligand that binds to the first ligand domain and the second ligand domain to the subject, and an immune response is detected in the subject following administration of the ligand.

G111. The method of any one of embodiments G109-G111, wherein the immune response is directed against a tumor in the subject.

G112. The method of any one of embodiments G1-G111, wherein the subject has cancer.

G113. The method of any one of embodiments G1-G112, wherein the subject has been diagnosed as having one or more tumors, and number of tumor cells is reduced following administration of the modified NK cell.

G114. The method of any one of embodiments G1-G113, wherein the subject has been diagnosed as having one or more tumors, and the size of one or more tumors is reduced following administration of the modified NK cell.

G115. The method of any one of embodiments G1-G114, wherein the subject has been diagnosed as having a hyperproliferative disease.

G116. The method of any one of embodiments G113 or G114, comprising administering a ligand that binds to the first ligand domain and the second ligand domain to the subject, and a reduction in the number of tumor cells or the size of one or more tumors is detected in the subject following administration of the ligand.

G117. The method of any one of embodiments G113, G114, or G116, wherein the modified NK cells are contacted with an antibody that binds to an antigen on the tumor before administration of the modified NK cells to the subject.

G118-G120. Reserved.

H1. A method of administering a ligand to a human subject who has undergone cell therapy using modified NK cells, wherein the modified NK cells comprise a polynucleotide encoding a chimeric polypeptide, wherein the chimeric polypeptide comprises
a) a first ligand binding region;
b) a second ligand binding region; and
c) a signaling region, comprising
   i) a MyD88 polypeptide;
   ii) a truncated MyD88 polypeptide lacking the TIR domain;
   iii) a MyD88 polypeptide and a CD40 cytoplasmic polypeptide region lacking the CD40 extracellular domain;
   iv) a truncated MyD88 polypeptide lacking the TIR domain and a CD40 cytoplasmic polypeptide region lacking the CD40 extracellular domain;
   v) a MyD88 polypeptide and a co-stimulatory polypeptide cytoplasmic signaling region selected from the group consisting of CD27, CD28, ICOS, 4-1BB, signaling regions;
   vi) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   vii) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40,
      wherein the method comprises administering a ligand to the subject, wherein the ligand binds to the first ligand binding region of a first chimeric polypeptide and the second ligand binding region of a second chimeric polypeptide, resulting in multimerization of the first and the second chimeric polypeptides.

H2. The method of embodiment H1, wherein the subject has a disease or condition associated with an elevated expression of a target antigen expressed by a target cell.

H3. The method of embodiment H1, wherein a tumor has been detected in the subject.

H4-H5. Reserved.

H6. The method of embodiment H2, wherein the modified NK cells have been primed with an antibody that binds to the target antigen before administration of the modified NK cells to the subject.

H7. The method of embodiments H3, wherein the modified NK cells have been primed with an antibody that binds to a tumor antigen before administration of the modified NK cells to the subject.

H8. The method of any one of embodiments H2 or H6, comprising measuring the number or concentration of target cells in a first sample obtained from the subject before administering the ligand, measuring the number or concentration of target cells in a second sample obtained from the subject after administering the ligand, and determining an increase or decrease of the number or concentration of target cells in the second sample compared to the number or concentration of target cells in the first sample.

H8.1. The method of embodiment H8, wherein the concentration of target cells in the second sample is decreased compared to the concentration of target cells in the first sample.

H8.2. The method of embodiment H8, wherein the concentration of target cells in the second sample is increased compared to the concentration of target cells in the first sample.

H8.3. The method of any one of embodiments H3 or H7, comprising measuring the size of a tumor in the subject before administering the ligand, measuring the size of a tumor in the subject after administering the ligand, and determining an increase or decrease of the size of the tumor following administration of the ligand.

H8.4. The method of embodiment H8.3, wherein the size of the tumor is decreased following administration of the ligand.

H8.5. The method of embodiment H8.3, wherein the size of the tumor is increased following administration of the ligand.

H8.6. The method of any one of embodiments H1-H8.5, wherein the subject has received a stem cell transplant before or at the same time as administration of the modified NK cells.

H8.7. The method of any one of embodiments H1-H8.6, wherein IL-15 is not administered to the subject within forty eight hours before or one week after administration of the modified NK cells.

H9-H99. Reserved.

H100. The method of any one of embodiments H1-H99, wherein the subject has an immune response.

H101. The method of any one of embodiments H1-H100, comprising contacting the modified NK cells with an antibody.

H102. The method of any one of embodiments H100-H101, wherein the immune response is a cytotoxic response.

H103. The method of any one of embodiments H100-H102, wherein the immune response is a cytolytic response.

H104. The method of any one of embodiments H100-H103, wherein the immune response is an anti-tumor response.

H105. The method of any one of embodiments H100-H104, comprising contacting the modified NK cells with an antibody that binds to an antigen on a tumor.

H106. The method of any one of embodiments H100-H105, wherein IL-15 is not administered to the subject within one week of administration of the modified NK cells.

H107. The method of any one of embodiments H100-H105, wherein IL-15 is not administered to the subject within two weeks of administration of the modified NK cells.

H108. The method of any one of embodiments H100-H107, wherein one dose of the modified NK cells is administered.

H109. The method of embodiment H108, wherein an immune response is detected in the subject following administration of the modified NK cells.

H110. The method of embodiment H109, comprising administering a ligand that binds to the first ligand domain and the second ligand domain to the subject, and an immune response is detected in the subject following administration of the ligand.

H111. The method of any one of embodiments H109-H111, wherein the immune response is directed against a tumor in the subject.

H112. The method of any one of embodiments H1-H111, wherein the subject has cancer.

H113. The method of any one of embodiments H1-H112, wherein the subject has been diagnosed as having one or more tumors, and number of tumor cells is reduced following administration of the modified NK cell.

H114. The method of any one of embodiments H1-H113, wherein the subject has been diagnosed as having one or more tumors, and the size of one or more tumors is reduced following administration of the modified NK cell.

H115. The method of any one of embodiments H1-H114, wherein the subject has been diagnosed as having a hyperproliferative disease.

H116. The method of any one of embodiments H113 or H114, comprising administering a ligand that binds to the first ligand domain and the second ligand domain to the subject, and a reduction in the number of tumor cells or the size of one or more tumors is detected in the subject following administration of the ligand.

H117. The method of any one of embodiments H113, H114, or H116, wherein the modified NK cells are contacted with an antibody that binds to an antigen on the tumor before administration of the modified NK cells to the subject.

H118-H120. Reserved.

I1. A kit comprising modified natural killer (NK) cells, wherein the modified NK cells comprise a polynucleotide encoding a chimeric polypeptide, comprising
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40
   wherein the modified NK cells have been stored at a temperature of −150° C. or below.

I3. The kit of any one of embodiments I1-I2, comprising a ligand that binds to the first ligand binding region and binds to the second ligand binding region.

I4. The kit of any one of embodiments I1-I3, comprising an antibody.

I5. The kit of embodiment I4, wherein the antibody binds to an antigen on a target cell.

I6. The kit of embodiment I5, wherein the antibody is formulated for priming the NK cells stimulate an immune response against the target cell.

I7. The kit of any one of embodiments I5 or I6 wherein the target cell is a tumor cell.

I8-I9. Reserved.

I10. The kit of any one of embodiments I1-I9, wherein the first ligand binding region has a different amino acid sequence than the second ligand binding region, and the first and second ligand binding regions bind to a heterodimeric ligand.

I11. The kit of embodiment I10, wherein the first ligand binding region binds to a first portion of the heterodimeric ligand, and the second ligand binding region binds to a second portion of the heterodimeric ligand.

I12. The kit of embodiment I11, wherein the chimeric polypeptide of any one of embodiments I1-I11 is a first chimeric polypeptide, the cell comprises a second chimeric polypeptide, the first ligand binding region of the first chimeric polypeptide and the second ligand binding region of the second chimeric polypeptide binds to the second portion of the heterodimeric ligand.

I12-I120. Reserved.

I200. A modified natural killer (NK) cell, comprising a first and a second polynucleotide, wherein the first polynucleotide encodes a chimeric polypeptide comprising
c) a first ligand binding region;
d) a second ligand binding region; and
e) a signaling region, comprising
   xv) a MyD88 polypeptide;
   xvi) a truncated MyD88 polypeptide lacking the TIR domain;
   xvii) a MyD88 polypeptide and a CD40 cytoplasmic polypeptide region lacking the CD40 extracellular domain;
   xviii) a truncated MyD88 polypeptide lacking the TIR domain and a CD40 cytoplasmic polypeptide region lacking the CD40 extracellular domain;
   xix) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   xx) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xxi) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40; and the second polynucleotide encodes a IL-15 polypeptide.

I201-I220. Reserved.

I400. A nucleic acid, comprising a first and a second polynucleotide, wherein the first polynucleotide encodes a chimeric polypeptide comprising
c) a first ligand binding region;
d) a second ligand binding region; and
e) a signaling region, comprising
   viii) a MyD88 polypeptide;
   ix) a truncated MyD88 polypeptide lacking the TIR domain;
   x) a MyD88 polypeptide and a CD40 cytoplasmic polypeptide region lacking the CD40 extracellular domain;
   xi) a truncated MyD88 polypeptide lacking the TIR domain and a CD40 cytoplasmic polypeptide region lacking the CD40 extracellular domain;
   xii) a MyD88 polypeptide and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions;
   xiii) a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions; or
   xiv) a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40 and the second polynucleotide encodes a IL-15 polypeptide.

I401. The nucleic acid of any one of embodiments I401-I494, wherein the nucleic acid comprises a polynucleotide that encodes a chimeric antigen receptor (CAR) or a T-cell receptor.

I402. The nucleic acid of embodiment I401, wherein the nucleic acid comprises a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric antigen receptor.

I403. The nucleic acid of any one of embodiments I401 or I402, wherein the CAR targets CD123 or BCMA.

I404. A modified NK cell comprising a nucleic acid of any one of embodiments I400-I403.

I405. The modified NK cell of embodiment I404 comprising a nucleic acid comprising a polynucleotide encoding a chimeric apoptotic polypeptide comprising an FKBP12 binding region, an FRB binding region or an FRB variant binding region, and a Caspase-9 polypeptide lacking the CARD domain.

I406. The nucleic acid of embodiment I400, wherein the nucleic acid comprises a first polynucleotide encoding a first FKBP12v36 ligand binding region and a second FKBP12v36 ligand binding region, a truncated MyD88 polypeptide lacking the TIR domain and a CD40 cytoplasmic polypeptide region lacking the CD40 extracellular domain, a second polynucleotide encoding an IL-15 polypeptide, and a third polynucleotide encoding a chimeric apoptotic polypeptide comprising an FKBP12 binding region, an FRB binding region or an FRB variant binding region, and a Caspase-9 polypeptide lacking the CARD domain.

I407. A modified NK cell comprising a nucleic acid of embodiment I406.

I408. The modified NK cell of embodiment I407, comprising a nucleic acid comprising a polynucleotide encoding a chimeric antigen receptor.

I409. The modified NK cell of embodiment 408, wherein the CAR targets CD123 or BCMA.I410. The nucleic acid of any of embodiments I400-I403, or I406, wherein the nucleic acid comprises a polynucleotide encoding a marker polypeptide.

I411. The nucleic acid of embodiment I410, wherein the marker polypeptide is a ΔCD19 polypeptide.

I412. The modified NK cell of any one of embodiments I404-I405, or I407-I409, wherein the nucleic acid encoding the chimeric antigen receptor or the nucleic acid encoding the chimeric apoptotic polypeptide comprises a polynucleotide encoding a marker polypeptide.

I413. The nucleic acid of embodiment I412, wherein the marker polypeptide is a ΔCD19 polypeptide.

I414-420. Reserved.

J1. A pharmaceutical composition prepared by a method of any one of embodiments B1-B120, C1-C120, or D1-D120.

J2. A pharmaceutical composition comprising a modified NK cell or a nucleic acid of any one of embodiments A1-A120, 1200-1220 or 1401-420.

J3-J20. Reserved.

L1. The modified NK cell, method, kit, nucleic acid, or pharmaceutical composition of any one of embodiments A1-J20, comprising a polynucleotide encoding a IL-15 polypeptide.

L2-L9. Reserved.

L10. The modified NK cell, method, kit, nucleic acid, or pharmaceutical composition of any one of embodiments A1-L9, wherein the first ligand binding region has a different amino acid sequence than the second ligand binding region, and the first and second ligand binding regions bind to a heterodimeric ligand.

L11. The modified NK cell, method, kit, nucleic acid, or pharmaceutical composition of embodiment L10, wherein the first ligand binding region binds to a first portion of the heterodimeric ligand, and the second ligand binding region binds to a second portion of the heterodimeric ligand.

L12. The modified NK cell, method, kit, nucleic acid, or pharmaceutical composition of embodiment L11, wherein the chimeric polypeptide of any one of embodiments L1-L11 is a first chimeric polypeptide, the cell comprises a second chimeric polypeptide, the first ligand binding region of the first chimeric polypeptide binds to the first portion of the heterodimeric ligand and the second ligand binding region of the second chimeric polypeptide binds to the second portion of the heterodimeric ligand.

L13. The modified NK cell, method, kit, nucleic acid, or pharmaceutical composition of embodiment L12, wherein the first and second chimeric polypeptides dimerize upon binding of the heterodimeric ligand to the first ligand binding region of the first chimeric polypeptide and the second ligand binding region of the second chimeric polypeptide.

L14. The modified NK cell, method, kit, nucleic acid, or pharmaceutical composition of any one of embodiments L10-L13, wherein the heterodimeric ligand is rapamycin or a rapamycin analog.

L15. The modified NK cell, method, kit, nucleic acid, or pharmaceutical composition of any one of embodiments L10-L14, wherein the first ligand binding region is an FK506 binding protein 12 (FKBP12) region and the second ligand binding region is an FKBP12-Rapamycin-binding domain of mTOR (FRB) region, or an FRB variant polypeptide region.

L16. The modified NK cell, method, kit, nucleic acid, or pharmaceutical composition of any one of embodiments L10-L15, wherein the first portion of the heterodimeric ligand binds to the first ligand binding region with 100 times or more affinity than it binds to the second ligand binding region.

L17. The modified NK cell, method, kit, nucleic acid, or pharmaceutical composition of any one of embodiments L10-L16, wherein the second portion of the heterodimeric ligand binds to the second ligand binding region with 100 times or more affinity than it binds to the first ligand binding region.

L18. Reserved.

L19. The modified NK cell, method, kit, nucleic acid, or pharmaceutical composition of any one of embodiments A1-L9, wherein the first ligand binding region and the second ligand binding region are the same.

L20. The modified NK cell, method, kit, nucleic acid, or pharmaceutical composition of embodiment L19, wherein the first ligand binding region and the second ligand binding region bind to a homodimeric ligand.

L21. The modified NK cell, method, kit, nucleic acid, or pharmaceutical composition of embodiment L20, wherein the first ligand binding region of a first chimeric polypeptide binds to a first portion of the homodimeric ligand and the second ligand binding region of a second chimeric polypeptide binds to the second portion of the homodimeric ligand.

L22. The modified NK cell, method, kit, nucleic acid, or pharmaceutical composition of embodiment L21, wherein the first and second chimeric polypeptides dimerize upon binding of the homodimeric ligand to the first ligand binding region of the first chimeric polypeptide and the second ligand binding region of the second chimeric polypeptide.

L23. The modified NK cell, method, kit, nucleic acid, or pharmaceutical composition of any one of embodiments L20-L22, wherein the first portion of the homodimeric ligand binds to the first ligand binding region with an affinity from 1-10 times the affinity that it binds to the second ligand binding region.

L24. The modified NK cell, method, kit, nucleic acid, or pharmaceutical composition of any one of embodiments L20-L22, wherein the second portion of the homodimeric ligand binds to the second ligand binding region with an affinity from 1-10 times the affinity than it binds to the first ligand binding region.

L25. The modified NK cell, method, kit, nucleic acid, or pharmaceutical composition of any one of embodiments L20-L24, wherein the ligand is rimiducid, AP20187, or AP1510.

L26. The modified NK cell, method, kit, nucleic acid, or pharmaceutical composition of any one of embodiments L20-L25, wherein the first ligand binding region and the second ligand binding region are FKBP12 variant regions.

L27. The modified NK cell, method, kit, nucleic acid, or pharmaceutical composition of any one of embodiments A1-L26, wherein the chimeric polypeptide does not comprise a membrane-targeting region.

L28. The modified NK cell, method, kit, nucleic acid, or pharmaceutical composition of any one of embodiments A1-L27, wherein the chimeric polypeptide does not comprise a first ligand binding region.

L29. The modified NK cell, method, kit, nucleic acid, or pharmaceutical composition of any one of embodiments A1-L28, wherein the chimeric polypeptide does not comprise a first ligand binding region.

L30. The modified NK cell, method, kit, nucleic acid, or pharmaceutical composition of any one of embodiments A1-L19, wherein the chimeric polypeptide comprises a FKBP12 binding region, a FRB binding region, and a truncated MyD88 polypeptide lacking the TIR domain.

L31. The modified NK cell, method, kit, nucleic acid, or pharmaceutical composition of any one of embodiments A1-L19, wherein the chimeric polypeptide comprises a FKBP12 binding region, a FRB variant binding region, and a truncated MyD88 polypeptide lacking the TIR domain.

L32. The modified NK cell, method, kit, nucleic acid, or pharmaceutical composition of any one of embodiments A1-L19, wherein the chimeric polypeptide comprises a FKBP12 binding region, a FRB_{L} binding region, and a truncated MyD88 polypeptide lacking the TIR domain.

L33. The modified NK cell, method, kit, nucleic acid, or pharmaceutical composition of any one of embodiments A1-L19, wherein the chimeric polypeptide comprises a FKBP12 binding region, a FRB binding region, a truncated MyD88 polypeptide lacking the TIR domain, and a CD40 cytoplasmic polypeptide region lacking the CD40 extracellular domain.

L34. The modified NK cell, method, kit, nucleic acid, or pharmaceutical composition of any one of embodiments A1-L19, wherein the chimeric polypeptide comprises a FKBP12 binding region, a FRB variant binding region, a truncated MyD88 polypeptide lacking the TIR domain, and a CD40 cytoplasmic polypeptide region lacking the CD40 extracellular domain.

L34. The modified NK cell, method, kit, nucleic acid, or pharmaceutical composition of any one of embodiments A1-L19, wherein the chimeric polypeptide comprises a FKBP12 binding region, a FRB_{L} binding region, a truncated MyD88 polypeptide lacking the TIR domain, and a CD40 cytoplasmic polypeptide region lacking the CD40 extracellular domain.

L35-L39. Reserved.

L40. The modified NK cell, method, kit, nucleic acid, or pharmaceutical composition of any one of embodiments A1-L9 or L20-L29, wherein the chimeric polypeptide comprises a first FKBP12v36 region, a second FKBP12v36 region, and a truncated MyD88 polypeptide lacking the TIR domain.

L41. The modified NK cell, method, kit, nucleic acid, or pharmaceutical composition of any one of embodiments A1-L9 or L20-L29, wherein the chimeric polypeptide comprises a first FKBP12v36 region, a second FKBP12v36 region, a truncated MyD88 polypeptide lacking the TIR domain, and a CD40 cytoplasmic polypeptide region lacking the CD40 extracellular domain.

L41.1. The modified NK cell, method, kit, nucleic acid, or pharmaceutical composition of any one of embodiments A1-L41, wherein the modified NK cell is contacted with antibody to direct antibody-directed cellular cytotoxicity.

L41.2. The modified NK cell, method, kit, nucleic acid, or pharmaceutical composition of embodiment L41.1, wherein the antibody is Herceptin (4D5) or Rituxan.

L42. The modified NK cell, method, kit, nucleic acid, or pharmaceutical composition of any one of embodiments A1-A41.2, wherein the cell comprises a polynucleotide that encodes a chimeric antigen receptor (CAR).

M1. The modified NK cell, method, kit, nucleic acid, or pharmaceutical composition of any one of embodiments A1-L42, wherein the cells are not grown on feeder cells.

M2. The modified NK cell, method, kit, nucleic acid, or pharmaceutical composition of any one of embodiments A1-M1, wherein the cell has been stored at a temperature of −150° C. or below for more than 24 hours.

M3. The modified NK cell, method, kit, nucleic acid, or pharmaceutical composition of any one of embodiments A1-M2, wherein the cell has been stored at a temperature of −150° C. or below for more than one week.

M4. The modified NK cell, method, kit, nucleic acid, or pharmaceutical composition of any one of embodiments A1-M3, wherein the cell has been stored at a temperature of −150° C. or below for more than three weeks.

M5. The modified NK cell, method, kit, nucleic acid, or pharmaceutical composition of any one of embodiments A1-M4, wherein the cells have been contacted with a ligand that binds to the first and second ligand binding regions.

M6. The modified NK cell, method, kit, nucleic acid, or pharmaceutical composition of any one of embodiments A1-M5, wherein the cells have not been contacted with a ligand that binds to the first and second ligand binding regions.

M7. The modified NK cell, method, kit, nucleic acid, or pharmaceutical composition of any one of embodiments A1-M6, wherein the cells have not been contacted with exogenous IL-15.

M8. The modified NK cell, method, kit, nucleic acid, or pharmaceutical composition of any one of embodiments A1-M7, comprising the step of transfecting or transducing NK cells with a nucleic acid comprising a polynucleotide encoding the chimeric polypeptide.

M8-M49. Reserved.

M50. The modified NK cell, method, kit, nucleic acid, or pharmaceutical composition of any one of embodiments A1-M4, wherein the cell has been contacted with a ligand that binds to the first and second ligand binding regions before cryostorage.

M51. The modified NK cell, method, kit, nucleic acid, or pharmaceutical composition of any one of embodiments A1-M4, wherein the cell has not been contacted with a ligand that binds to the first and second ligand binding regions before cryostorage.

M52. The modified NK cell, method, kit, nucleic acid, or pharmaceutical composition of any one of embodiments A1-A51, wherein the cell has not been contacted with exogenous IL-15 before cryostorage.

M53-M59. Reserved.

M60. The modified NK cell, method, kit, nucleic acid, or pharmaceutical composition of any one of embodiments A1-M59, wherein the cell has been thawed following cryostorage.

M61. The modified NK cell, method, kit, nucleic acid, or pharmaceutical composition of any one of embodiments A1-M59, wherein the cell has been primed with an antibody following cryostorage.

M62. The modified NK cell, method, kit, nucleic acid, or pharmaceutical composition of embodiment M61, wherein the antibody binds to a tumor antigen.

M63. The modified NK cell, method, kit, nucleic acid, or pharmaceutical composition of any one of embodiments A1-M62, wherein the antibody is Herceptin or Rituxan.

M64-M69. Reserved.

M70. The modified NK cell, method, kit, nucleic acid, or pharmaceutical composition of any one of embodiments M60-M69, wherein the thawed NK cells have a 20 percent or greater efficacy than thawed NK cells that do not express the chimeric protein of any one of embodiments A1-M69.

M71. The modified NK cell, method, kit, nucleic acid, or pharmaceutical composition of any one of embodiments M60-M69, wherein the thawed NK cells have a 50 percent or greater efficacy than thawed NK cells that do not express a chimeric protein of any one of embodiments A1-M69.

M72. The modified NK cell, method, kit, nucleic acid, or pharmaceutical composition of any one of embodiments M70-M71, wherein efficacy is determined by assaying NK cell-mediated tumor cell death, NK cell proliferation, or NK cell survival.

M73. The modified NK cell, method, kit, nucleic acid, or pharmaceutical composition of embodiment M72, wherein the tumor cell is a THP1 tumor cell.

M74. The modified NK cell, method, kit, nucleic acid, or pharmaceutical composition of any one of embodiments M60-M73, wherein the thawed NK cells have the same level of viability as thawed NK cells that do not express the chimeric protein of any one of embodiments A1-M69.

M75. The modified NK cell, method, kit, nucleic acid, or pharmaceutical composition of any one of embodiments M60-M73, wherein the thawed NK cells have within 10 percent of the viability of thawed NK cells that do not express the chimeric protein of any one of embodiments A1-M69.

M76. The modified NK cell, method, kit, nucleic acid, or pharmaceutical composition of any one of embodiments M71-M75, wherein the efficacy is measured two days or more after the thawed NK cells are placed in growth medium under growing conditions.

M77. The modified NK cell, method, kit, nucleic acid, or pharmaceutical composition of any one of embodiments M71-M75, wherein the efficacy is measured three days or more after the thawed NK cells are placed in growth medium under growing conditions.

M78. The modified NK cell, method, kit, nucleic acid, or pharmaceutical composition of any one of embodiments of any one of embodiments A1-M77, comprising contacting the modified NK cells with an antibody.

M79. The method of any one of embodiments B1-M78, further comprising contacting the modified NK cell with a ligand that binds to the first ligand binding region of a first chimeric polypeptide and to the second ligand binding region of a second chimeric polypeptide, resulting in multimerization of the chimeric polypeptides.

M80. The modified NK cell, method, kit, nucleic acid, or pharmaceutical composition of any one of embodiments A1-M77, wherein the modified NK cell secretes IL-15.

M81. The modified NK cell, method, kit, nucleic acid, or pharmaceutical composition of embodiment M80, wherein the modified NK cell secretes two fold or more IL-15 than an NK cell that does not express the chimeric protein of any one of embodiments A1-M69.

M82. The modified NK cell, method, kit, nucleic acid, or pharmaceutical composition of embodiment M80, wherein the modified NK cell secretes 100 times or more IL-15 than an NK cell that does not express the chimeric protein of any one of embodiments A1-M69.

M83. The modified NK cell, method, kit, nucleic acid, or pharmaceutical composition of any one of embodiments A1-M82, wherein the modified NK cell secretes ten times or more IFN-γ than an NK cell that does not express the chimeric protein of any one of embodiments A1-M69.

M84. The modified NK cell, method, kit, nucleic acid, or pharmaceutical composition of any one of embodiments A1-M82, wherein the modified NK cell secretes 100 times or more IFN-γ than an NK cell that does not express the chimeric protein of any one of embodiments A1-M69.

M85. The modified NK cell, method, kit, nucleic acid, or pharmaceutical composition of any one of embodiments A1-M84, wherein the modified NK cell secretes ten times or more GM-CSF than an NK cell that does not express the chimeric protein of any one of embodiments A1-M69.

M86. The modified NK cell, method, kit, nucleic acid, or pharmaceutical composition of any one of embodiments A1-M84, wherein the modified NK cell secretes 100 times or more GM-CSF than an NK cell that does not express the chimeric protein of any one of embodiments A1-M69.

M87. The modified NK cell, method, kit, nucleic acid, or pharmaceutical composition of any one of embodiments A1-M86, wherein the modified NK cell secretes ten times or more TNF-α than an NK cell that does not express the chimeric protein of any one of embodiments A1-M69.

M88. The modified NK cell, method, kit, nucleic acid, or pharmaceutical composition of any one of embodiments A1-M86, wherein the modified NK cell secretes 100 times or more TNF-α than an NK cell that does not express the chimeric protein of any one of embodiments A1-M69.

M89. The modified NK cell, method, kit, nucleic acid, or pharmaceutical composition of any one of embodiments A1-M88, wherein the modified NK cell secretes ten times or more MIP-1α than an NK cell that does not express the chimeric protein of any one of embodiments A1-M69.

M90. The modified NK cell, method, kit, nucleic acid, or pharmaceutical composition of any one of embodiments A1-M88, wherein the modified NK cell secretes 100 times or more MIP-1α than an NK cell that does not express the chimeric protein of any one of embodiments A1-M69.

M91. The modified NK cell, method, kit, nucleic acid, or pharmaceutical composition of any one of embodiments A1-M90, wherein the modified NK cell secretes ten times or more MIP-1β than an NK cell that does not express the chimeric protein of any one of embodiments A1-M69.

M92. The modified NK cell, method, kit, nucleic acid, or pharmaceutical composition of any one of embodiments A1-M90, wherein the modified NK cell secretes 100 times or more MIP-1β than an NK cell that does not express the chimeric protein of any one of embodiments A1-M69.

M93. The modified NK cell, method, kit, nucleic acid, or pharmaceutical composition of any one of embodiments M80-M92, wherein the modified NK cell is contacted with a tumor cell or tumor cell line before determining the amount of a secreted cytokine, wherein the cytokine is selected from the group consisting of IL-15, IFN-γ, GM-CSF, TNF-α, MIP-1α, and MIP-1β.

M94. The modified NK cell, method, kit, nucleic acid, or pharmaceutical composition of any one of embodiments M80-M93, wherein the modified NK cell is contacted with a ligand that binds to the first and second ligand binding regions before determining the amount of a secreted cytokine, wherein the cytokine is selected from the group consisting of IL-15, IFN-γ, GM-CSF, TNF-α, MIP-1α, and MIP-1β.

M95. The modified NK cell, method, kit, nucleic acid, or pharmaceutical composition of any one of embodiments A1-M94, wherein the modified NK cells proliferate at a quicker rate than NK cells that do not express the chimeric protein of any one of embodiments E1-E69.

M96. The modified NK cell, method, kit, nucleic acid, or pharmaceutical composition of any one of embodiments A1-M95, wherein the modified NK cells have a greater survival rate than NK cells that do not express the chimeric protein of any one of embodiments E1-E69.

M97. The modified NK cell, method, kit, nucleic acid, or pharmaceutical composition method of any one of embodiments M95 or M96, wherein the NK cells are grown in the presence of exogenous IL-2.

N1. The modified NK cell, method, kit, nucleic acid, or pharmaceutical composition of any one of embodiments A1-M97, wherein the signaling region comprises a truncated MyD88 polypeptide lacking the TIR domain.

N2. The modified NK cell, method, kit, nucleic acid, or pharmaceutical composition of any one of embodiments A1-M97, wherein the signaling region comprises a truncated MyD88 polypeptide lacking the TIR domain and a CD40 cytoplasmic polypeptide region lacking the CD40 extracellular domain.

N3. The modified NK cell, method, kit, nucleic acid, or pharmaceutical composition of any one of embodiments A1-M97, wherein the signaling region comprises a truncated MyD88 polypeptide lacking the TIR domain and a co-stimulatory polypeptide cytoplasmic signaling region selected from the group consisting of CD27, CD28, ICOS, 4-1BB, RANK/TRANCE-R, OX40, CD30, TweakR, TAC1, BCMA and HVEM cytoplasmic signaling regions.

N3.1. The modified NK cell, method, kit, nucleic acid, or pharmaceutical composition of embodiment N4, wherein the co-stimulatory polypeptide cytoplasmic signaling region is selected from the group consisting of CD27, CD28, ICOS, 4-1BB, RANK/TRANCE-R, and OX40.

N3.2. The modified NK cell, method, kit, nucleic acid, or pharmaceutical composition of embodiment N4, wherein the co-stimulatory polypeptide cytoplasmic signaling region is selected from the group consisting of CD28, 4-1BB, OX40, and ICOS.

N3.3. The modified NK cell, method, kit, nucleic acid, or pharmaceutical composition of embodiment N4, wherein the co-stimulatory polypeptide cytoplasmic signaling region is selected from the group consisting of CD27, CD28, CD40, ICOS, 4-1BB, CD40, RANK/TRANCE-R, and OX40.

N3.4. The modified NK cell, method, kit, nucleic acid, or pharmaceutical composition of embodiment N4, wherein the co-stimulatory polypeptide cytoplasmic signaling region is selected from the group consisting of CD27, CD28, ICOS, 4-1BB, and OX40.

N3.5. The modified NK cell, method, kit, nucleic acid, or pharmaceutical composition of any one of embodiments N3-N3.4, wherein the co-stimulatory polypeptide lacks an extracellular domain or lacks a functional extracellular domain.

N4. The modified NK cell, method, kit, nucleic acid, or pharmaceutical composition of any one of embodiments A1-M97, wherein the signaling region comprises a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

N4.1. The modified NK cell, method, kit, nucleic acid, or pharmaceutical composition of embodiment N4, wherein the signaling region comprises a first co-stimulatory polypeptide cytoplasmic signaling region selected from the group consisting of CD27, CD28, ICOS, 4-1BB, RANK/TRANCE-R, and OX40, and a second co-stimulatory polypeptide cytoplasmic signaling region selected from the group consisting of CD27, CD28, ICOS, 4-1BB, RANK/TRANCE-R, and OX40.

N4.2. The modified NK cell, method, kit, nucleic acid, or pharmaceutical composition of embodiment N4, wherein the signaling region comprises a first co-stimulatory polypeptide cytoplasmic signaling region selected from the group consisting of CD28, 4-1BB, OX40, and ICOS, and a second co-stimulatory polypeptide cytoplasmic signaling region selected from the group consisting of CD28, 4-1BB, OX40, and ICOS.

N4.3. The modified NK cell, method, kit, nucleic acid, or pharmaceutical composition of embodiment N4, wherein the signaling region comprises a first co-stimulatory polypeptide cytoplasmic signaling region selected from the group consisting of CD27, CD28, CD40, ICOS, 4-1BB, CD40, RANK/TRANCE-R, and OX40, and a second co-stimulatory polypeptide cytoplasmic signaling region selected from the group consisting of CD27, CD28, CD40, ICOS, 4-1BB, CD40, RANK/TRANCE-R, and OX40.

N4.4. The modified NK cell, method, kit, nucleic acid, or pharmaceutical composition of embodiment N4, wherein the signaling region comprises a first co-stimulatory polypeptide cytoplasmic signaling region selected from the group consisting of CD27, CD28, ICOS, 4-1BB, and OX40, and a second co-stimulatory polypeptide cytoplasmic signaling region selected from the group consisting of CD27, CD28, ICOS, 4-1BB, and OX40.

N4.5. The modified NK cell, method, kit, nucleic acid, or pharmaceutical composition of any one of embodiments N4-N4.4, wherein the first and second co-stimulatory polypeptides lack an extracellular domain or lacks a functional extracellular domain.

N5. The modified NK cell, method, kit, nucleic acid, or pharmaceutical composition of any one of embodiments A1-N3, wherein the truncated MyD88 polypeptide comprises the amino acid sequence of the full length MyD88 sequence of SEQ ID NO: 118, wherein the truncated MyD88 polypeptide lacks the TIR domain, or a functional fragment thereof.

N6. The modified NK cell, method, kit, nucleic acid, or pharmaceutical composition of any one of embodiments A1-N3, wherein the truncated MyD88 polypeptide does not comprise contiguous amino acid residues 156 to the C-terminus of the full length MyD88 polypeptide.

N7. The modified NK cell, method, kit, nucleic acid, or pharmaceutical composition of any one of embodiments A1-N3, wherein the truncated MyD88 polypeptide does not comprise contiguous amino acid residues 152 to the C-terminus of the full length MyD88 polypeptide.

N8. The modified NK cell, method, kit, nucleic acid, or pharmaceutical composition of any one of embodiments A1-N3, wherein the truncated MyD88 polypeptide does not comprise contiguous amino acid residues 173 to the C-terminus of the full length MyD88 polypeptide.

N9. The modified NK cell, method, kit, nucleic acid, or pharmaceutical composition of any one of embodiments A1-N3, or N5-N8, wherein the full length MyD88 polypeptide comprises the amino acid sequence of SEQ ID NO: 118.

N10. The modified NK cell, method, kit, nucleic acid, or pharmaceutical composition of any one of embodiments A1-N3, or N5-N9, wherein the truncated MyD88 polypeptide consists of the amino acid sequence of SEQ ID NO: 119, or a functional fragment thereof.

N11. The modified NK cell, method, kit, nucleic acid, or pharmaceutical composition of any one of embodiments A1-N3, or N5-N9, wherein the truncated MyD88 polypeptide has the amino acid sequence of SEQ ID NO: 119 or an amino acid sequence 90% or more identical to SEQ ID NO: 119.

N12. The modified NK cell, method, kit, nucleic acid, or pharmaceutical composition of any one of embodiments A1-N3, or N5-N9, wherein the truncated MyD88 polypeptide has the amino acid sequence of SEQ ID NO: 119 or an amino acid sequence 95% or more identical to SEQ ID NO: 119.

N13. The modified NK cell, method, kit, nucleic acid, or pharmaceutical composition of any one of embodiments A1-N3, or N5-N9, wherein the truncated MyD88 polypeptide has the amino acid sequence of SEQ ID NO: 119.

N14. The modified NK cell, method, kit, nucleic acid, or pharmaceutical composition of any one of embodiments A1-N13, wherein the signaling region comprises a CD40 cytoplasmic polypeptide and lacks the CD40 extracellular domain.

N15. The modified NK cell, method, kit, nucleic acid, or pharmaceutical composition of any one of embodiments A1-N14, wherein the CD40 cytoplasmic polypeptide has the amino acid sequence of SEQ ID NO: 56 or an amino acid sequence 90% or more identical to SEQ ID NO: 56.

N16. The modified NK cell, method, kit, nucleic acid, or pharmaceutical composition of any one of embodiments A1-N14, wherein the CD40 cytoplasmic polypeptide has the amino acid sequence of SEQ ID NO: 56.

N18-N39: Reserved.

N40. The modified NK cell, method, kit, nucleic acid, or pharmaceutical composition of any one of embodiments A1-N39, wherein the first binding region comprises a FKBP12 region.

N41. The modified NK cell, method, kit, nucleic acid, or pharmaceutical composition of any one of embodiments A1-N39, wherein the first binding region consists of a FKBP12 region.

N42. The modified NK cell, method, kit, nucleic acid, or pharmaceutical composition of any one of embodiments A1-N39, wherein the second binding region comprises a FKBP12-Rapamycin Binding (FRB) region or a FRB variant polypeptide region.

N43. The modified NK cell, method, kit, nucleic acid, or pharmaceutical composition of any one of embodiments A1-N39, wherein the second binding region consists of a FKBP12-Rapamycin Binding (FRB) region or a FRB variant polypeptide region.

N44. The modified NK cell, method, kit, nucleic acid, or pharmaceutical composition of any one of embodiments A1-N39, wherein the first binding region comprises a FKBP12 region and the second binding region comprises a FRB region.

N45. The modified NK cell, method, kit, nucleic acid, or pharmaceutical composition of any one of embodiments A1-N39, wherein the first binding region comprises a FKBP12 region and the second binding region comprises a FRB variant polypeptide region.

N46. The modified NK cell, method, kit, nucleic acid, or pharmaceutical composition of any one of embodiments A1-N39, wherein the first binding region consists of a FKBP12 region and the second binding region consists of a FRB region.

N47. The modified NK cell, method, kit, nucleic acid, or pharmaceutical composition of any one of embodiments A1-N39, wherein the first binding region consists of a FKBP12 region and the second binding region consists of a FRB variant polypeptide region.

N48. The modified NK cell, method, kit, nucleic acid, or pharmaceutical composition of any one of embodiments A1-N39, wherein the first multimerizing region comprises a FKBP12 region and the second multimerizing region comprises a FRB region.

N49. The modified NK cell, method, kit, nucleic acid, or pharmaceutical composition of any one of embodiments A1-N39, wherein the first multimerizing region comprises a FKBP12 region and the second multimerizing region comprises a FRB variant polypeptide region.

N50. The modified NK cell, method, kit, nucleic acid, or pharmaceutical composition of any one of embodiments

N50.1 The modified NK cell, method, kit, nucleic acid, or pharmaceutical composition of any one of embodiments A1-N48, wherein the FKBP12 region has the amino acid sequence of SEQ ID NO: 16 or an amino acid sequence 90% or more identical to SEQ ID NO: 16.

N51. The modified NK cell, method, kit, nucleic acid, or pharmaceutical composition of any one of embodiments A1-N48, wherein the FKBP12 region has the amino acid sequence of SEQ ID NO: 60 or an amino acid sequence 95% or more identical to SEQ ID NO: 60.

N52. The modified NK cell, method, kit, nucleic acid, or pharmaceutical composition of any one of embodiments A1-N48, wherein the FKBP12 region has the amino acid sequence of SEQ ID NO: 60.

N53. The modified NK cell, method, kit, nucleic acid, or pharmaceutical composition of any one of embodiments A1-N52, wherein the FRB region has the amino acid sequence of SEQ ID NO: 101 or an amino acid sequence 90% or more identical to SEQ ID NO: 101.

N54. The modified NK cell, method, kit, nucleic acid, or pharmaceutical composition of any one of embodiments A1-N52, wherein the FRB region has the amino acid sequence of SEQ ID NO: 101 or an amino acid sequence 95% or more identical to SEQ ID NO: 101.

N55. The modified NK cell, method, kit, nucleic acid, or pharmaceutical composition of any one of embodiments A1-N52, wherein the FRB region has the amino acid sequence of SEQ ID NO: 101.

N56. The modified NK cell, method, kit, nucleic acid, or pharmaceutical composition of any one of embodiments A1-N55, wherein the FRB variant polypeptide region has an amino acid substitution at position 2098 chosen from valine, leucine and isoleuceine.

N56.1. The modified NK cell, method, kit, nucleic acid, or pharmaceutical composition of any one of embodiments A1-N55, wherein the FRB variant polypeptide region binds to a C7 rapalog.

N56.2. The modified NK cell, method, kit, nucleic acid, or pharmaceutical composition of any one of embodiments A1-N55, wherein the FRB variant polypeptide region comprises an amino acid substitution at position T2098 or W2101.

N56.3. The modified NK cell, method, kit, nucleic acid, or pharmaceutical composition of any one of embodiments A1-N55, wherein the FRB variant polypeptide region is selected from the group consisting of KLW (T2098L) (FRBL), KTF (W2101F), and KLF (T2098L, W2101F).

N57. The modified NK cell, method, kit, nucleic acid, or pharmaceutical composition of any one of embodiments A1-N52, wherein the FRB variant polypeptide region has the amino acid sequence of SEQ ID NO: 121.

N58-N59. Reserved.

N60. The modified NK cell, method, kit, nucleic acid, or pharmaceutical composition of any one of embodiments A1-N39, or N53-N57, wherein the first ligand binding region comprises a FKBP12 variant polypeptide region.

N61. The modified NK cell, method, kit, nucleic acid, or pharmaceutical composition of any one of embodiments A1-N39, or N53-N57, wherein the first ligand binding region consists of a FKBP12 variant polypeptide region.

N62. The modified NK cell, method, kit, nucleic acid, or pharmaceutical composition of any one of embodiments A1-N39, wherein the second ligand binding region comprises a FKBP12 variant polypeptide region.

N63. The modified NK cell, method, kit, nucleic acid, or pharmaceutical composition of any one of embodiments A1-N39, wherein the second ligand binding region consists of a FKBP12 variant polypeptide region.

N64. The modified NK cell, method, kit, nucleic acid, or pharmaceutical composition of any one of embodiments A1-N39, wherein the first ligand binding region comprises a FKBP12 variant polypeptide region, and the second ligand binding region comprises a FKBP12 variant polypeptide region.

N65. The modified NK cell, method, kit, nucleic acid, or pharmaceutical composition of any one of embodiments A1-N39, wherein the first ligand binding region consists of a FKBP12 variant polypeptide region and the second ligand binding region consists of a FKBP12 variant polypeptide region.

N66. The modified NK cell, method, kit, nucleic acid, or pharmaceutical composition of any one of embodiments A1-N39, or N60-N65, wherein the FKBP12 variant region has an amino acid sequence of SEQ ID NO: 60, or an amino acid sequence 90% or more identical to SEQ ID NO: 60. comprising an amino acid substitution at position 36 chosen from valine, leucine, isoleuceine and alanine.

N67. The modified NK cell, method, kit, nucleic acid, or pharmaceutical composition of any one of embodiments A1-N39, or N60-N65, wherein the FKBP12 variant region has an amino acid sequence of SEQ ID NO: 60, or an amino acid sequence 95% or more identical to SEQ ID NO: 60 comprising an amino acid substitution at position 36 chosen from valine, leucine, isoleuceine and alanine.

N68. The modified NK cell, method, kit, nucleic acid, or pharmaceutical composition of any one of embodiments A1-N39, or N60-N65, wherein the FKBP12 variant region has an amino acid sequence of SEQ ID NO: 60, comprising an amino acid substitution at position 36 chosen from valine, leucine, isoleuceine and alanine.

N69. The modified NK cell, method, kit, nucleic acid, or pharmaceutical composition of any one of embodiments A1-N39, or N60-N65, wherein the FKBP12 variant region comprises an amino acid substitution at position 36 chosen from valine, leucine, isoleuceine and alanine.

N70. The modified NK cell, method, kit, nucleic acid, or pharmaceutical composition of any one of embodiments A1-N39, or N60-N69, wherein the FKBP12 variant region comprises an amino acid substitution at position 36, wherein the amino acid substitution at position 36 is valine.

N71. The modified NK cell, method, kit, nucleic acid, or pharmaceutical composition of any one of embodiments A1-N39, or N60-N69, wherein the FKBP12 variant region has an amino acid sequence of SEQ ID NO: 16, or an amino acid sequence 90% or more identical to SEQ ID NO: 16.

N72. The modified NK cell, method, kit, nucleic acid, or pharmaceutical composition of any one of embodiments A1-N39, or N60-N69, wherein the FKBP12 variant region has an amino acid sequence of SEQ ID NO: 16, or an amino acid sequence 95% or more identical to SEQ ID NO: 16.

N73. The modified NK cell, method, kit, nucleic acid, or pharmaceutical composition of any one of embodiments A1-N39, or N60-N69, wherein the FKBP12 variant region has an amino acid sequence of SEQ ID NO: 16.

N73.1. The modified NK cell, method, kit, nucleic acid, or pharmaceutical composition of any one of embodiments A1-N39, or N60-N69, wherein the first and second ligand binding regions together are Fv′Fvls.

N73.2. The modified NK cell, method, kit, nucleic acid, or pharmaceutical composition of embodiment N73.1, wherein Fv′Fvls comprises two linked polypeptides comprising the amino acid sequence of SEQ ID NO: 12 and the amino acid sequence of SEQ ID NO: 16.

N73.3. The modified NK cell, method, kit, nucleic acid, or pharmaceutical composition of embodiment N73.1, wherein Fv′Fvls comprises two linked polypeptide encoded by the nucleic acid sequences of SEQ ID NO: 11 and SEQ ID NO: 15.

N74. The modified NK cell, method, kit, nucleic acid, or pharmaceutical composition of any one of embodiments A1-N73, wherein the ligand is a heterodimeric ligand comprising a first portion and a second portion, the first portion of the ligand is capable of binding to the first ligand binding region, and the second portion is capable of binding to the second ligand binding region.

N75. The modified NK cell, method, kit, nucleic acid, or pharmaceutical composition of embodiment N74, wherein the first portion of the heterodimeric ligand binds to the first ligand binding region with 100 times or more greater affinity than the first portion binds to the second ligand binding region.

N76. The modified NK cell, method, kit, nucleic acid, or pharmaceutical composition of any one of embodiments N74 or N75, wherein the second portion of the heterodimeric ligand binds to the second ligand binding region with 100 times or more greater affinity than the second portion binds to the first ligand binding region.

N77. The modified NK cell, method, kit, nucleic acid, or pharmaceutical composition of any one of embodiments A1-N76, wherein the heterodimeric ligand is rapamycin.

N77. The modified NK cell, method, kit, nucleic acid, or pharmaceutical composition of any one of embodiments A1-N76, wherein the heterodimeric ligand is a rapalog.

N78. The modified NK cell, method, kit, nucleic acid, or pharmaceutical composition of embodiment N77, wherein the rapalog is selected from the group consisting of S-o,p-dimethoxyphenyl (DMOP)-rapamycin, R-Isopropoxyrapamycin, S-Butanesulfonamidorap, R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl) indole rapamycin, temsirolimus, everolimus, zotarolimus, and R and S C7-(7-methyl) indole rapamycin.

N79. The modified NK cell, method, kit, nucleic acid, or pharmaceutical composition of any one of embodiments A1-N73, wherein the ligand is a homodimeric ligand comprising a first portion and a second portion, the first portion of the ligand is capable of binding to the first ligand binding region and is capable of binding to the second ligand binding region, and the second portion of the ligand is capable of binding to the first ligand binding region and is capable of binding to the second ligand binding region.

N80. The modified NK cell, method, kit, nucleic acid, or pharmaceutical composition of any one of embodiments A1-N73 or N79, wherein the ligand is rimiducid.

N81. The modified NK cell, method, kit, nucleic acid, or pharmaceutical composition of any one of embodiments A1-N73, or N79, wherein the ligand is AP20187 or AP1510.

N82-N89. Reserved.

N90. The method of any one of embodiments F1-N89, wherein the cells are autologous cells.

N91. The method of any one of embodiments F1-N89, wherein the cells are allogeneic cells.

N92. The method of any one of embodiments F1-N91, wherein the cells are obtained from bone marrow, cord blood, or peripheral blood.

N93. The method of any one of embodiments F1-N92, further comprising
identifying the presence, absence or stage of a condition or disease in a subject; and
transmitting an indication to administer the ligand, maintain a subsequent dosage of the ligand, or adjust a subsequent dosage of the ligand administered to the subject based on the presence, absence or stage of the condition or disease identified in the subject.

N94. The method of any one of embodiments F1-N93, wherein the subject has been diagnosed as having a tumor.

N95. The method of any one of embodiments F1-N94, wherein the subject has cancer.

N96. The method of any one of embodiments F1-N94, wherein the subject has a solid tumor.

N97. The method of any one of embodiments N95 or N96, wherein the cancer is present in the blood or bone marrow of the subject.

N98. The method of any one of embodiments N1-N94, wherein the subject has a blood or bone marrow disease.

N99. The method of any one of embodiments F1-N98, wherein the subject has been diagnosed with any condition or condition that can be alleviated by stem cell transplantation.

N100. The method of any one of embodiments F1-N99, wherein the subject has been diagnosed with sickle cell anemia or metachromatic leukodystrophy.

N101. The method of any one of embodiments F1-N100, wherein the subject has been diagnosed with a condition selected from the group consisting of a primary immune deficiency condition, hemophagocytosis lymphohistiocytosis (HAH) or other hemophagocytic condition, an inherited marrow failure condition, a hemoglobinopathy, a metabolic condition, and an osteoclast condition.

N102. The method of any one of embodiments F1-N100, wherein the subject has been diagnosed with a disease or condition selected from the group consisting of Severe Combined Immune Deficiency (SCID), Combined Immune Deficiency (CID), Congenital T-cell Defect/Deficiency, Common Variable Immune Deficiency (CVID), Chronic Granulomatous Disease, IPEX (Immune deficiency, polyendocrinopathy, enteropathy, X-linked) or IPEX-like, Wiskott-Aldrich Syndrome, CD40 Ligand Deficiency, Leukocyte Adhesion Deficiency, DOCA 8 Deficiency, IL-10 Deficiency/IL-10 Receptor Deficiency, GATA 2 deficiency, X-linked lymphoproliferative disease (XAP), Cartilage Hair Hypoplasia, Shwachman Diamond Syndrome, Diamond Blackfan Anemia, Dyskeratosis Congenita, Fanconi Anemia, Congenital Neutropenia, Sickle Cell Disease, Thalassemia, Mucopolysaccharidosis, Sphingolipidoses, and Osteopetrosis.

N103. The method of any one of embodiments F1-N100, wherein the subject has been diagnosed with leukemia.

N104. The method of any one of embodiments F1-N100, wherein the subject has been diagnosed with an infection of viral etiology selected from the group consisting HIV, influenza, Herpes, viral hepatitis, Epstein Bar, polio, viral encephalitis, measles, chicken pox, Cytomegalovirus (CMV), adenovirus (ADV), HHV-6 (human herpesvirus 6, I), and Papilloma virus, or has been diagnosed with an infection of bacterial etiology selected from the group consisting of pneumonia, tuberculosis, and syphilis, or has been diagnosed with an infection of parasitic etiology selected from the group consisting of malaria, trypanosomiasis, leishmaniasis, trichomoniasis, and amoebiasis.

N105. The modified NK cells, methods, pharmaceutical compositions, or kits of any one of embodiments A1-N104, wherein the modified NK cells comprise a polynucleotide that encodes a chimeric antigen receptor (CAR).

N106. The modified NK cells, methods, pharmaceutical compositions, or kits of embodiment N105, wherein the CAR comprises a transmembrane region, a cell activation region, and an antigen recognition region.

N107. The modified NK cells, methods, pharmaceutical compositions, or kits of embodiment N106, wherein the cell activation region is a T-cell activation region.

N108. The modified NK cells, methods, pharmaceutical compositions, or kits of embodiment N107, wherein the T-cell activation region is a CD3 zeta-chain region.

N109. The modified NK cells, methods, pharmaceutical compositions, or kits of any one of embodiments N106-N108, wherein the antigen recognition region specifically binds to a molecule chosen from PSMA, PSCA, Muc1 CD19, ROR1, Mesothelin, GD2, CD123, Muc16, CD33, CD38, CD44v6, and Her2/Neu.

N110. The modified NK cells, methods, pharmaceutical compositions, or kits of any one of embodiments N106-N109, wherein the antigen recognition moiety binds to an antigen on a cell involved in a hyperproliferative disease or to a viral or bacterial antigen.

N111. The modified NK cells, methods, pharmaceutical compositions, or kits of any one of embodiments N106-N110, wherein the antigen recognition moiety binds to an antigen selected from the group consisting of PSMA, PSCA, MUC1, CD19, ROR1, Mesothelin, GD2, CD123, MUC16, Her2/NE, CD20, CD30, BCMA, PRAME, NY-ESO-1, and EGFRvIII.

N112. The modified NK cell, method, kit, nucleic acid, or pharmaceutical composition of any one of embodiments A1-N113, wherein the modified NK cells have been primed with an antibody.

N113. The modified NK cell, method, kit, nucleic acid, or pharmaceutical composition of embodiment N112, wherein the antibody is capable of binding to an antigen on a tumor cell.

N114. The modified NK cell, method, kit, nucleic acid, or pharmaceutical composition of embodiment N112, wherein the antibody is selected from the group of antibodies that are capable of binding to an antigen on an infected cell.

T1. The modified NK cell, method, kit, nucleic acid, or pharmaceutical composition of any one of embodiments A1-N114, wherein the modified NK cell comprises a polynucleotide encoding a chimeric pro-apoptotic polypeptide, wherein the chimeric pro-apoptotic polypeptide comprises a third ligand binding region and a pro-apoptotic polypeptide region.

T2. The modified NK cell, method, kit, nucleic acid, or pharmaceutical composition of embodiment T1, wherein the first and second ligand binding regions of the chimeric polypeptide bind to a homodimeric ligand with 100 times greater affinity or more than the homodimeric ligand binds to the third ligand binding region of the chimeric pro-apoptotic polypeptide.

T3. The modified NK cell, method, kit, nucleic acid, or pharmaceutical composition of embodiment T1, wherein the first and second ligand binding regions of the chimeric polypeptide bind to a heterodimeric ligand with 100 times greater affinity or more than the heterodimeric ligand binds to the third ligand binding region of the chimeric pro-apoptotic polypeptide.

T3. The modified NK cell, method, kit, nucleic acid, or pharmaceutical composition of any one of embodiments T1 or T2, wherein the third ligand binding region binds to a ligand with 100 times greater affinity or more than the ligand binds to the first ligand binding region.

T4. The modified NK cell, method, kit, nucleic acid, or pharmaceutical composition of any one of embodiments T1-T3, wherein the third ligand binding region binds to a ligand with 100 times greater affinity or more than the ligand binds to the second ligand binding region.

T5. The modified NK cell, method, kit, nucleic acid, or pharmaceutical composition of any one of embodiments T1-T4, wherein the pro-apoptotic polypeptide is selected from the group consisting of Caspase 1, 2, 3, 4, 5, 6, 7, 8, 9, 10, 11, 12, 13, or 14, FADD (DED), APAF1 (CARD), CRADD/RAIDD CARD), ASC (CARD), Bax, Bak, Bcl-XL, Bcl-2, RIPK3, and RIPK1-RHIM.

T6. The modified NK cell, method, kit, nucleic acid, or pharmaceutical composition of any one of embodiments T1-T5, wherein the pro-apoptotic polypeptide is a caspase polypeptide.

T6.1. The modified NK cell, method, kit, nucleic acid, or pharmaceutical composition of embodiment T6, wherein the caspase polypeptide lacks the CARD domain.

T7. The modified NK cell, method, kit, nucleic acid, or pharmaceutical composition of any one of embodiments T1-T4, wherein the pro-apoptotic polypeptide is a Caspase-9 polypeptide.

T8. The modified NK cell, method, kit, nucleic acid, or pharmaceutical composition of embodiment T7, wherein the Caspase-9 polypeptide lacks the CARD domain.

T9. The modified NK cell, method, kit, nucleic acid, or pharmaceutical composition of any one of embodiments T7-T8, wherein the Caspase-9 polypeptide has the amino acid sequence of SEQ ID NO: 64 or an amino acid sequence 90% or more identical to SEQ ID NO: 64.

T10. The modified NK cell, method, kit, nucleic acid, or pharmaceutical composition of any one of embodiments T7-T8, wherein the Caspase-9 polypeptide has the amino acid sequence of SEQ ID NO: 27 or an amino acid sequence 95% or more identical to SEQ ID NO: 64.

T11. The modified NK cell, method, kit, nucleic acid, or pharmaceutical composition of any one of embodiments T7-T8, wherein the Caspase-9 polypeptide has the amino acid sequence of SEQ ID NO: 64.

T12. The modified NK cell, method, kit, nucleic acid, or pharmaceutical composition of any one of embodiments T7-T8 the caspase polypeptide is a modified Caspase-9 polypeptide comprising an amino acid substitution selected from a catalytically active caspase variant disclosed in Table 1 of U.S. Patent Application Nos. 62/816,799, 62/668,223, and 62/756,442, or as described in U.S. Pat. Nos. 9,434,935, 9,932,572 and 9,913,882, or U.S. Patent Application Publication Nos. 2018-0251746 A1 and 2018-0243384 A1.

T13. The modified NK cell, method, kit, nucleic acid, or pharmaceutical composition of any one of embodiments T7-T10 or T12, wherein the caspase polypeptide is a modified Caspase-9 polypeptide and has an amino acid substitution selected from the group consisting of D330A, D330E, and N405Q.

T14. The modified NK cell, method, kit, nucleic acid, or pharmaceutical composition of any one of embodiments T1-T13, wherein the third ligand binding region comprises a first multimerizing region and a second multimerizing region.

T15. The modified NK cell, method, kit, nucleic acid, or pharmaceutical composition of embodiment T14, wherein the first multimerizing region has a different amino acid sequence than the second multimerizing region, and the first and second multimerizing regions bind to a heterodimeric ligand.

T16. The modified NK cell, method, kit, nucleic acid, or pharmaceutical composition of embodiment T15, wherein the first multimerizing region binds to binds to a first portion of the heterodimeric ligand, and the second multimerizing region binds to a second portion of the heterodimeric ligand.

T17. The modified NK cell, method, kit, nucleic acid, or pharmaceutical composition of embodiment T16, wherein the chimeric pro-apoptotic polypeptide of any one of embodiments T1-T16 is a first chimeric pro-apoptotic polypeptide, the cell comprises a second chimeric pro-apoptotic polypeptide, the first ligand binding region of the first chimeric pro-apoptotic polypeptide binds to a first portion of the heterodimeric ligand and the second ligand multimerizing region of the second chimeric pro-apoptotic polypeptide binds to a second portion of the heterodimeric ligand.

T18. The modified NK cell, method, kit, nucleic acid, or pharmaceutical composition of embodiment T17, wherein the first and second chimeric polypeptides dimerize upon binding of the heterodimeric ligand to the first multimerizing region of the first chimeric pro-apoptotic polypeptide and the second multimerizing region of the second chimeric pro-apoptotic polypeptide.

T19. The modified NK cell, method, kit, nucleic acid, or pharmaceutical composition of any one of embodiments T15-T18, wherein the heterodimeric ligand is rapamycin or a rapamycin analog.

T20. The modified NK cell, method, kit, nucleic acid, or pharmaceutical composition of any one of embodiments T14-T19, wherein the first multimerizing region is an FK506 binding protein 12 (FKBP12) region and the second multimerizing region is an FKBP12-Rapamycin-binding domain of mTOR (FRB) region, or an FRB variant polypeptide region.

T21. The modified NK cell, method, kit, nucleic acid, or pharmaceutical composition of any one of embodiments T14-T20, wherein the first portion of the heterodimeric ligand binds to the first multimerizing region with 100 times or more affinity than it binds to the second multimerizing region.

T22. The modified NK cell, method, kit, nucleic acid, or pharmaceutical composition of any one of embodiments T14-T21, wherein the second portion of the heterodimeric ligand binds to the multimerizing region with 100 times or more affinity than it binds to the first multimerizing region.

T23. The modified NK cell, method, kit, nucleic acid, or pharmaceutical composition of any one of embodiments T19-T22, wherein the first and second ligand binding regions of the chimeric polypeptide comprise FKBP12 variant polypeptide regions.

T24. The modified NK cell, method, kit, nucleic acid, or pharmaceutical composition of any one of embodiments T14-T23, wherein the first and second ligand binding regions of the chimeric polypeptide comprise FKBP12v36 regions.

T25-T30 Reserved.

T31. The modified NK cell, method, kit, nucleic acid, or pharmaceutical composition of any one of embodiments T1-T13, wherein the third ligand binding region binds to a homodimeric ligand.

T32. The modified NK cell, method, kit, nucleic acid, or pharmaceutical composition of any one of embodiments T1-T13, or T31, wherein the third ligand binding region of a first chimeric pro-apoptotic polypeptide binds to a first portion of the homodimeric ligand and the third ligand binding region of a second chimeric pro-apoptotic polypeptide binds to the second portion of the homodimeric ligand.

T33. The modified NK cell, method, kit, nucleic acid, or pharmaceutical composition of embodiment T31, wherein the first and second chimeric pro-apoptotic polypeptides dimerize upon binding of the homodimeric ligand to the third ligand binding region of the first chimeric pro-apoptotic polypeptide and the third ligand binding region of the second chimeric pro-apoptotic polypeptide.

T34. The modified NK cell, method, kit, nucleic acid, or pharmaceutical composition of any one of embodiments T31-T33, wherein the ligand is rimiducid, AP20187, or AP1510.

T35. The modified NK cell, method, kit, nucleic acid, or pharmaceutical composition of any one of embodiments T31-T34, wherein the third ligand binding region comprises a FKBP12 variant region.

T36. The modified NK cell, method, kit, nucleic acid, or pharmaceutical composition of any one of embodiments T31-T35, wherein the third ligand binding region comprises a FKBP12v36 region.

T37. The modified NK cell, method, kit, nucleic acid, or pharmaceutical composition of any one of embodiments T31-T36, wherein the chimeric polypeptide comprises a FKBP12 binding region, a FRB binding region

T38. The modified NK cell, method, kit, nucleic acid, or pharmaceutical composition of any one of embodiments T31-T37, wherein the chimeric polypeptide comprises a FKBP12 binding region and a FRB binding region or FRB variant binding region.

T39. Reserved.

T40. The modified NK cell, method, kit, nucleic acid, or pharmaceutical composition of any one of embodiments T1-T40, wherein
the chimeric polypeptide comprises a FKBP12 binding region, an FRB binding region or an FRB variant binding region, and a truncated MyD88 polypeptide lacking the TIR domain; and
the chimeric pro-apoptotic polypeptide comprises a FKBP12v36 region and a Caspase-9 polypeptide lacking the CARD domain.

T41. The modified NK cell, method, kit, nucleic acid, or pharmaceutical composition of any one of embodiments T1-T40, wherein
the chimeric polypeptide comprises a FKBP12 binding region, an FRB binding region or an FRB variant binding region, a truncated MyD88 polypeptide lacking the TIR domain, and a CD40 cytoplasmic polypeptide region lacking the CD40 extracellular domain; and
the chimeric pro-apoptotic polypeptide comprises a FKBP12v36 region and a Caspase-9 polypeptide lacking the CARD domain.

T42. The modified NK cell, method, kit, nucleic acid, or pharmaceutical composition of any one of embodiments T1-T40, wherein
the chimeric polypeptide comprises two FKBP12v36 regions, and a truncated MyD88 polypeptide lacking the TIR domain; and
the chimeric pro-apoptotic polypeptide comprises a FKBP12 binding region, an FRB binding region or an FRB variant binding region, and a Caspase-9 polypeptide lacking the CARD domain.

T42.1. The modified NK cell, method, kit, nucleic acid, or pharmaceutical composition of any one of embodiments T1-T40, wherein
the chimeric polypeptide comprises two FKBP12v36 regions, a truncated MyD88 polypeptide lacking the TIR domain, and a CD40 cytoplasmic polypeptide region lacking the CD40 extracellular domain; and
the chimeric pro-apoptotic polypeptide comprises a FKBP12 binding region, an FRB binding region or an FRB variant binding region, and a Caspase-9 polypeptide lacking the CARD domain.

T43. The method of any one of embodiments T1-T42.1, comprising administering to the subject a ligand that binds to the third ligand binding region in an amount effective to kill at least 30% of the cells that express the chimeric pro-apoptotic polypeptide.

T44. The method of any one of embodiments T1-T42.1, comprising administering to the subject a ligand that binds to the third ligand binding region in an amount effective to kill at least 60% of the cells that express the chimeric pro-apoptotic polypeptide.

T45. The method of any one of embodiments T1-T42.1, comprising administering to the subject a ligand that binds to the third ligand binding region in an amount effective to kill at least 90% of the cells that express the chimeric pro-apoptotic polypeptide.

T46. The method of any one of embodiments T43-T45, wherein the third ligand binding region is capable of binding to rapamycin or to a rapalog.

T47. The method of any one of embodiments T43-T46, wherein the ligand is rapamycin or a rapalog.

T48. The method of embodiment T47, wherein the rapalog is selected from the group consisting of S-o,p-dimethoxyphenyl (DMOP)-rapamycin, R-Isopropoxyrapamycin, S-Butanesulfonamidorap, R and S C7-ethyloxyrapamycin, R and S C7-isopropyloxyrapamycin, R and S C7-isobutylrapamycin, R and S ethylcarbamaterapamycin, R and S C7-phenylcarbamaterapamycin, R and S C7-(3-methyl) indole rapamycin, temsirolimus, everolimus, zotarolimus, and R and S C7-(7-methyl) indole rapamycin.

T49. The method of anyone of embodiments T43-T45, wherein the third ligand binding region is capable of binding to rimiducid.

T50. The method of any one of embodiments T43-T45, wherein the ligand is rimiducid.

T51. The method of any one of embodiments T43-T50, wherein more than one dose of the ligand is administered to the subject.

T52. The method of any one of embodiments T43-T51, comprising
identifying a presence or absence of a condition in the subject that requires the removal of the modified NK cells from the subject; and
administering a ligand that binds to the third binding region, maintaining a subsequent dosage of the ligand, or adjusting a subsequent dosage of the ligand to the subject based on the presence or absence of the condition identified in the subject.

T53. The method of any one of embodiments T43-T51 comprising
receiving information comprising presence or absence of a condition in the subject that requires the removal of the modified NK cells from the subject; and
administering a ligand that binds to the third binding region, maintaining a subsequent dosage of the ligand, or adjusting a subsequent dosage of the ligand to the subject based on the presence or absence of the condition identified in the subject.

T54. The method of any one of embodiments T43-T51, comprising
identifying a presence or absence of a condition in the subject that requires the removal of the modified NK cells from the subject; and
transmitting the presence, absence or stage of the condition identified in the subject to a decision maker who administers a ligand that binds to the third binding region, maintains a subsequent dosage of the ligand, or adjusts a subsequent dosage of the ligand administered to the subject based on the presence, absence or stage of the condition identified in the subject.

T55. The method of any one of embodiments T43-T51, further comprising
identifying a presence or absence of a condition in the subject that requires the removal of the modified NK cells from the subject; and
transmitting an indication to administer a ligand that binds to the third binding region, maintain a subsequent dosage of the ligand, or adjust a subsequent dosage of the ligand administered to the subject based on the presence, absence or stage of the condition identified in the subject.

T56-T59. Reserved.

T60. The modified NK cell, method, kit, nucleic acid, or pharmaceutical composition of any one of embodiments T1-T59, wherein the third ligand binding region comprises a FKBP12 region having the amino acid sequence of SEQ ID NO: 60 or an amino acid sequence 90% or more identical to SEQ ID NO: 60.

T61. The modified NK cell, method, kit, nucleic acid, or pharmaceutical composition of any one of embodiments T1-T59, wherein the third ligand binding region comprises a FKBP12 region having the amino acid sequence of SEQ ID NO: 60 or an amino acid sequence 95% or more identical to SEQ ID NO: 60.

T62. The modified NK cell, method, kit, nucleic acid, or pharmaceutical composition of any one of embodiments T1-T59, wherein the third ligand binding region comprises a FKBP12 region having the amino acid sequence of SEQ ID NO: 60.

T63. The modified NK cell, method, kit, nucleic acid, or pharmaceutical composition of any one of embodiments T1-T62, wherein the third ligand binding region comprises a FRB region having the amino acid sequence of SEQ ID NO: 60 or an amino acid sequence 90% or more identical to SEQ ID NO: 60.

T64. The modified NK cell, method, kit, nucleic acid, or pharmaceutical composition of any one of embodiments T1-T62, wherein the third ligand binding region comprises a FRB region having the amino acid sequence of SEQ ID NO: 101 or an amino acid sequence 95% or more identical to SEQ ID NO: 101.

T65. The modified NK cell, method, kit, nucleic acid, or pharmaceutical composition of any one of embodiments T1-T62, wherein the third ligand binding region comprises a FRB region having the amino acid sequence of SEQ ID NO: 101.

T66. The modified NK cell, method, kit, nucleic acid, or pharmaceutical composition of any one of embodiments T1-T62, wherein the FRB variant polypeptide region has an amino acid substitution at position 2098 chosen from valine, leucine and isoleuceine.

T67. The modified NK cell, method, kit, nucleic acid, or pharmaceutical composition of any one of embodiments T1-T62, wherein the third ligand binding region comprises a FRB variant polypeptide region having the amino acid sequence of SEQ ID NO: 121.

T68. The modified NK cell, method, kit, nucleic acid, or pharmaceutical composition of any one of embodiments T1-T59 wherein the third ligand binding region comprises a FKBP12 variant region having an amino acid sequence of SEQ ID NO:60, or an amino acid sequence 90% or more identical to SEQ ID NO: 60 comprising an amino acid substitution at position 36 chosen from valine, leucine, isoleuceine and alanine.

T69. The modified NK cell, method, kit, nucleic acid, or pharmaceutical composition of any one of embodiments T1-T59, wherein the third ligand binding region comprises a FKBP12 variant region having an amino acid sequence of SEQ ID NO: 60, or an amino acid sequence 95% or more identical to SEQ ID NO: 60 comprising an amino acid substitution at position 36 chosen from valine, leucine, isoleuceine and alanine.

T70. The modified NK cell, method, kit, nucleic acid, or pharmaceutical composition of any one of embodiments T1-T59, wherein the third ligand binding region comprises a FKBP12 variant region having an amino acid sequence of SEQ ID NO: 60, comprising an amino acid substitution at position 36 chosen from valine, leucine, isoleuceine and alanine.

T71. The modified NK cell, method, kit, nucleic acid, or pharmaceutical composition of any one of embodiments T1-T59, wherein the third ligand binding region comprises a FKBP12 variant region comprising an amino acid substitution at position 36 chosen from valine, leucine, isoleuceine and alanine.

T72. The modified NK cell, method, kit, nucleic acid, or pharmaceutical composition of any one of embodiments T1-T59, wherein the third ligand binding region comprises a FKBP12 variant region comprising an amino acid substitution at position 36, wherein the amino acid substitution at position 36 is valine.

T73. The modified NK cell, method, kit, nucleic acid, or pharmaceutical composition of any one of embodiments T1-T59, wherein the third ligand binding region comprises a FKBP12 variant region having an amino acid sequence of SEQ ID NO: 16, or an amino acid sequence 90% or more identical to SEQ ID NO: 16.

T74. The modified NK cell, method, kit, nucleic acid, or pharmaceutical composition of any one of embodiments T1-T59, wherein the third ligand binding region comprises a FKBP12 variant region having an amino acid sequence of SEQ ID NO: 16, or an amino acid sequence 95% or more identical to SEQ ID NO: 16.

T75. The modified NK cell, method, kit, nucleic acid, or pharmaceutical composition of any one of embodiments T1-T59, wherein the third ligand binding region comprises a FKBP12 variant region having an amino acid sequence of SEQ ID NO: 16.

U1. The modified NK cell, method, kit, nucleic acid, or pharmaceutical composition of any one of embodiments A1-T75, wherein the first and second polynucleotides are present on the same nucleic acid.

U2. The modified NK cell, method, kit, nucleic acid, or pharmaceutical composition of any one of embodiments T1-T75, wherein the first and second polynucleotides, and the polynucleotide that encodes the chimeric pro-apoptotic polypeptide are present on the same nucleic acid.

### Example 7: Additional Nucleotide and Amino Acid Sequences

(MyD88 nucleotide sequence)
SEQ ID NO: 117
atggctgcaggaggtcccggcgcggggtctgcggccccggtct
cctccacatcctcccttcccctggctgctctcaacatgcgagt
gcggcgccgcctgtctctgttcttgaacgtgcggacacaggtg
gcggccgactggaccgcgctggcggaggagatggactttgagt
acttggagatccggcaactggagacacaagcggaccccactgg
caggctgctggacgcctggcagggacgccctggcgcctctgta
ggccgactgctcgagctgcttaccaagctgggccgcgacgacg
tgctgctggagctgggacccagcattgaggaggattgccaaaa
gtatatcttgaagcagcagcaggaggaggctgagaagccttta
caggtggccgctgtagacagcagtgtcccacggacagcagagc
tggcgggcatcaccacacttgatgaccccctggggcatatgcc
tgagcgtttcgatgccttcatctgctattgccccagcgacatc
cagtttgtgcaggagatgatccggcaactggaacagacaaact
atcgactgaagttgtgtgtgtctgaccgcgatgtcctgcctgg
cacctgtgtctggtctattgctagtgagctcatcgaaaagagg
tgccgccggatggtggtggttgtctctgatgattacctgcaga
gcaaggaatgtgacttccagaccaaatttgcactcagcctctc
tccaggtgcccatcagaagcgactgatccccatcaagtacaag
gcaatgaagaaagagttccccagcatcctgaggttcatcactg
tctgcgactacaccaacccctgcaccaaatcttggttctggac
tcgccttgccaaggccttgtccctgccc
(MyD88 amino acid sequence)
SEQ ID NO: 118
M A A G G P G A G S A A P V S S T S S L P L A
A L N M R V R R R L S L F L N V R T Q V A A D
W T A L A E E M D F E Y L E I R Q L E T Q A D
P T G R L L D A W Q G R P G A S V G R L L E L
L T K L G R D D V L L E L G P S I E E D C Q K
Y I L K Q Q Q E E A E K P L Q V A A V D S S V
P R T A E L A G I T T L D D P L G H M P E R F
D A F I C Y C P S D I Q F V Q E M I R Q L E Q
T N Y R L K L C V S D R D V L P G T C V W S I
A S E L I E K R C R R M V V V V S D D Y L Q S
K E C D F Q T K F A L S L S P G A H Q K R L I
P I K Y K A M K K E F P S I L R F I T V C D Y
T N P C T K S W F W T R L A K A L S L P
(MyD88L (TIR-deleted))
SEQ ID NO: 119
MAAGGPGAGSAAPVSSTSSLPLAALNMRVRRRLSLFLNVRTQVAAD
VVTALAEEMDFEYLEIRQLETQADPTGRLLDAWQGRPGASVGRLLD
LLTKLGRDDVLLELGPSIEEDCQKYILKQQQEEAEKPLQVAAVDSS
VPRTAELAGITTLDDPLGHMPERFDAFICYCPSDIQ
(FRBL)
SEQ ID NO: 121
QLEMWHEGLEEASRLYFGERNVKGMFEVLEPLHAMMERGPQTLKET
SFNQAYGRDLMEAQEWCRKYMKSGNVKDLLQAWDLYYHVFRRISK

The entirety of each patent, patent application, publication and document referenced herein hereby is incorporated by reference. Citation of the above patents, patent applications, publications and documents are not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1-102. (canceled)

103. A nucleic acid, comprising a first polynucleotide and a second polynucleotide, wherein the first polynucleotide encodes a chimeric polypeptide comprising:
(a) a ligand binding region; and
(b) a signaling region, comprising:
(i) a MyD88 polypeptide and a co-stimulatory polypeptide cytoplasmic signaling region selected from CD27, CD28, CD3, ICOS, 4-1 BB, CD40, RANK/TRANCE-R, and OX40; or
(ii) a truncated MyD88 polypeptide lacking the TIR domain and a co-stimulatory polypeptide cytoplasmic signaling region selected from CD27,
CD28, CD3, ICOS, 4-1 BB, CD40, RANK/TRANCE-R, and OX40; and wherein the second polynucleotide encodes an IL-15 polypeptide.

104. The nucleic acid of claim 103, wherein the cytoplasmic signaling region is selected from CD28, 4-1BB, OX40, and ICOS.

105. The nucleic acid of claim 103, wherein the signaling region comprises a first co-stimulatory polypeptide cytoplasmic signaling region selected from CD27, CD28, CD3, ICOS, 4-1 BB, CD40, RANK/TRANCE-R, and OX40, and a second co-stimulatory polypeptide cytoplasmic signaling region selected from CD27, CD28, CD3, ICOS, 4-1 BB, CD40, RANK/TRANCE-R, and OX40.

106. The nucleic acid of claim 103, wherein the ligand binding region comprises two FKBP12 variant polypeptide regions.

107. The nucleic acid of claim 106, wherein the two FKBP12 variant polypeptide regions each comprise FKBP12v36.

108. The nucleic acid of claim 103, wherein the ligand binding region comprises an FKBP12 polypeptide and an FKBP-rapamycin binding (FRB) polypeptide or an FRB variant polypeptide.

109. The nucleic acid of claim 103, wherein the ligand binding region binds to a chemical inducer of dimerization or multimerization selected from rimiducid, AP20187, AP1510, rapamycin, or a rapalog.

110. The nucleic acid of claim 103, wherein the co-stimulatory polypeptide cytoplasmic signaling region lacks an extracellular domain or lacks a functional extracellular domain.

111. The nucleic acid of claim 103, wherein the MyD88 polypeptide comprises a truncated MyD88 polypeptide lacking the TIR domain.

112. The nucleic acid of claim 103, further comprising a third polynucleotide encoding a chimeric pro-apoptotic polypeptide comprising a multimeric ligand binding region and a Caspase-9 polypeptide lacking the CARD domain.

113. The nucleic acid of claim 103, contained within a viral vector selected from an adenoviral vector, a retroviral vector, or a lentiviral vector.

114. A modified natural killer (NK) cell comprising a nucleic acid comprising a first polynucleotide and a second polynucleotide, wherein the first polynucleotide encodes a chimeric polypeptide comprising:
(a) a ligand binding region; and
(b) a signaling region, comprising:
(i) a MyD88 polypeptide and a co-stimulatory polypeptide cytoplasmic signaling region selected from CD27, CD28, CD3, ICOS, 4-1 BB, CD40, RANK/TRANCE-R, and OX40; or
(ii) a truncated MyD88 polypeptide lacking the TIR domain and a co-stimulatory polypeptide cytoplasmic signaling region selected from CD27, CD28, CD3, ICOS, 4-1 BB, CD40, RANK/TRANCE-R, and OX40; and wherein the second polynucleotide encodes an IL-15 polypeptide.

115. The modified NK cell of claim 114, further comprising a nucleic acid encoding a chimeric antigen receptor (CAR) or a T cell receptor (TCR).

116. The modified NK cell of claim 114, further comprising a nucleic acid encoding an inducible chimeric pro-apoptotic polypeptide comprising an FKBP12 or FRB binding region and a Caspase-9 polypeptide lacking the CARD domain.

117. The modified NK cell of claim 114, wherein the NK cell has been cryostored, and/or wherein the NK cell has not been grown on feeder cells.

118. A pharmaceutical composition comprising the modified NK cell of claim 114.

119. A method of treating a subject diagnosed with cancer, the method comprising administering to the subject a modified natural killer (NK) cell comprising a nucleic acid comprising a first polynucleotide and a second polynucleotide, wherein the first polynucleotide encodes a chimeric polypeptide comprising:
(a) a ligand binding region; and
(b) a signaling region, comprising:
(i) a MyD88 polypeptide and a co-stimulatory polypeptide cytoplasmic signaling region selected from CD27, CD28, CD3, ICOS, 4-1 BB, CD40, RANK/TRANCE-R, and OX40; or
(ii) a truncated MyD88 polypeptide lacking the TIR domain and a co-stimulatory polypeptide cytoplasmic signaling region selected from CD27, CD28, CD3, ICOS, 4-1 BB, CD40, RANK/TRANCE-R, and OX40; and
wherein the second polynucleotide encodes an IL-15 polypeptide.

120. The method of claim 119, further comprising administering to the subject an effective amount of a ligand that binds to the ligand binding region of the chimeric polypeptide to induce multimerization of the chimeric polypeptide, wherein the ligand is selected from rimiducid, AP20187, AP1510, rapamycin, or a rapalog.

121. The method of claim 119, wherein administering the ligand reduces the number or concentration of target cells expressing a target antigen in the subject or reduces the size of a tumor in the subject.

122. The method of claim 119, wherein the modified NK cell further comprises a nucleic acid encoding a chimeric pro-apoptotic polypeptide comprising an FKBP12 binding region, an FRB binding region or an FRB variant binding region, and a Caspase-9 polypeptide lacking the CARD domain, and further comprising, upon occurrence of an adverse event, administering a ligand that binds the chimeric pro-apoptotic polypeptide to reduce the number or concentration of the modified NK cells in the subject.
